(19) United States
(12) Patent Application Publication
(10) Pub. No.: US 2021/0319872 A1
(43) Pub. Date: Oct. 14, 2021

(54) DRUG AND DEVICE COMBINATION PRODUCTS WITH IMPROVED SAFETY AND EFFICACY PROFILES

(71) Applicant: Edmund L. Valentine, Palm Beach Gardens, FL (US)

(72) Inventor: Edmund L. Valentine, Palm Beach Gardens, FL (US)

(21) Appl. No.: 16/325,603

(22) PCT Filed: Aug. 15, 2017
(86) PCT No.: PCT/US17/46997

§ 371 Date: Feb. 14, 2019

Related U.S. Application Data
(63) Continuation-in-part of application No. PCT/US17/17665, filed on Feb. 13, 2017.
(60) Provisional application No. 62/375,192, filed on Aug. 15, 2016.
(60) Provisional application No. 62/432,394, filed on Dec. 9, 2016.
(60) Provisional application No. 62/432,358, filed on Dec. 9, 2016.
(60) Provisional application No. 62/432,248, filed on Dec. 9, 2016.
(60) Provisional application No. 62/432,292, filed on Dec. 9, 2016.
(60) Provisional application No. 62/427,919, filed on Nov. 30, 2016.
(60) Provisional application No. 62/416,972, filed on Nov. 3, 2016.
(60) Provisional application No. 62/375,256, filed on Aug. 15, 2016.

(51) Int. Cl. G16H 20/13; G16H 40/67; G16H 70/40; G06F 21/32; G06F 40/58; G06N 5/00

(52) U.S. Cl. CPC G16H 20/13; G16H 40/67; G06N 5/003; G06F 21/32; G06F 40/58; G16H 70/40

(57) ABSTRACT

The present invention describes a novel oral drug dispensing system comprised of a mobile Drug Specific App which utilizes a patient and drug specific drug dispensing algorithm which incorporates the use of digitally captured and patient self-assessment, self-test, and/or self-report, prescription, and dispenser information/values to; (i) control drug dispensing to improve medication safety, (ii) personalize oral drug therapy, (iii) improve medication effectiveness while avoiding drug mediated side effects, (iv) decreases misuse, abuse, over dosing, under dosing, dependence, addiction, divergence, accidental ingestion, overdose, and deaths, (v) improve disease management, (vi) ensure prescription compliance, and (vii) promotes prescription persistence on a cost-effective real-time basis.

### CROSS REFERENCE TO RELATED APPLICATIONS

This application is a national stage entry of International Application No. PCT/US17/046997, filed on Aug. 15, 2017, which claims benefit of U.S. Provisional Patent Application No. 62/375,192 filed on Aug. 15, 2016; U.S. Provisional Patent Application No. 62/375,256 filed on Aug. 15, 2016; U.S. Provisional Patent Application No. 62/416,972 filed on Nov. 3, 2016; U.S. Provisional Patent Application No. 62/427,919 filed on Nov. 30, 2016; U.S. Provisional Patent Application No. 62/432,292 filed on Dec. 9, 2016; U.S. Provisional Patent Application No. 62/432,248 filed on Dec. 9, 2016; U.S. Provisional Patent Application No. 62/432,358 filed on Dec. 9, 2016; and U.S. Provisional Patent Application No. 62/432,394 filed on Dec. 9, 2016, and International Patent Application No. PCT/US17/17665 filed on Feb. 13, 2017, which are incorporated herein by reference in their entirety to the full extent permitted by law.

### FIELD OF THE INVENTION

The present disclosure relates to a novel system, each independent component which brings a unique attribute, that together as a drug and device combination product improves the drug's safety profile and deters/decreases drug overdoses, misuse, abuse, accidental ingestion, and drug divergence. Each drug and device combination product is comprised of a drug, an interrelated Drug Specific and Patient Tailored App™, an overdose diagnostic (OverDoseScreen™), drug specific and patient tailored dispensing algorithm, drug dispenser, drug cassette, electronic data exchange, and certain integrated support services. The closed loop system is designed to decrease drug diversion, misuse, abuse, addiction, dependence, overdose, accidental ingestion and deaths—thereby improving the quality of care, enhancing the patient's quality of life, and decreasing the total cost of care by minimizing the number of drug related medical interventions, emergency room visits, and hospitalizations. Together with patient biometric and monitoring data, such as may be provided through an IoT device in communication with the he closed loop system may also have the benefits of optimizing the required dose, minimizing over prescribing and under prescribing.

### BACKGROUND OF THE INVENTION

Deterring drug divergence, misuse, decreasing the incidence of drug addiction and dependence, avoiding drug overdoses and deaths, decreasing the incidence of accidental ingestion of drugs by children, properly titrating and dosing patients, optimizing treatment, effectively managing drug tolerance, avoiding unnecessary drug induced side effects, and ensuring patient prescription compliance, and controlling drug withdrawal represent major unmet needs.

One specific example of such unmet needs is with respect to opioid drugs. According to the United Nations Office on Drugs and Crime (UNODC), drug overdose is the top cause of drug-related deaths in the world, and opioids are the top drug type associated with those deaths. Drugs are derived from opium, which comes from opium poppy seeds. In general, the term opioid is used to refer to synthetic prescription painkillers, but heroin is also an opioid. The following are commonly prescribed opioids with example brand names of established and new products: (i) Oxycodone (OxyContin®), (ii) Hydrocodone (Vicodin®), (iii) Morphine, (iv) Buprenorphine, (v) Hydromorphone (Dilaudid®), (vi) Oxymorphone (Opana), Tapentadol, (vii) Tramadol (Ultram®), (viii) Fentanyl (Duragesic®), methadone (Dolophine®), Morphine Sulfate and Naltrexone Hydrochloride (Embeda®), Oxycodone Hydrochloride and Naltrexone Hydrochloride (Troxyca® ER), etc.

The World Health Organization (WHO) estimated that 16.4 million people used opioids in 2012. People who take prescription painkillers can become addicted with just one prescription. Once addicted, it can be hard to stop. According to the National Institute on Drug Abuse (NIDA), combined with their highly-addictive nature, opioids are dangerous because a single large dose can cause severe respiratory depression and death. In addition, a drug user's tolerance to opioids decreases during breaks from drug use. When they take the drug at the same dose as they did before a break, it often leads to an overdose.

According to the International Narcotics Control Board (INCB), North America consumes 80 percent of the world's opioids, and has the highest rate of drug-related deaths in the world. IMS Health reported that Americans accounted for 99 percent of the world's hydrocodone (Vicodin®) consumption, 80 percent of the world's oxycodone (Percocet® and Oxycontin®) consumption and 65 percent of the world's hydromorphone (Dilaudid®) consumption in 2014.

The United States' opioid statistics are alarming:
a. According to the National Institute of Drug Abuse, opioid abuse is an epidemic in the United States, with about five (5) percent of the adult population using opiate pain medications non-medically. In 2015, approximately 12.5 million people misused prescription pain relievers in the past year, representing 4.7 percent of the population aged 12 or older.¹ ¹ Prescription Drug Use and Misuse in the United States: Results from the 2015 National Survey on Drug Use and Health, SAMHSA: Arthur Hughes, Matthew R. Williams, Rachel N. Lipari, and Jonaki Bose; RTI International: Elizabeth A. P. Copello and Larry A. Kroutil, September 2016.
b. Major causes of preventable death—like car accidents—have been on the decline, but deaths from drug abuse doubled in the last decade (Deaths by overdoses involving prescription painkillers quadrupled since 1999.). More people die from overdoses of prescription opioids than from all other drugs combined, including heroin and cocaine.² In 2014, there were 18,893 overdose deaths related to prescription pain relievers—52 people per day.³ Drug painkillers are the leading cause of accidental death. ² National Institute of Drug Abuse. (2015). Drug Facts: Prescription and Over-the-Counter Medications. Bethesda, Md.: National Institute of Drug Abuse.³ CDC
c. In 2015, 2.1 million people aged 12 or older were recent initiates for pain reliever misuse (i.e., misused for the first time in the past year), an average of 5,753 per day.⁴ ⁴ Prescription Drug Use and Misuse in the United States: Results from the 2015 National Survey on Drug Use and Health, SAMHSA: Arthur Hughes, Matthew R. Williams, Rachel N. Lipari, and Jonaki Bose; RTI International: Elizabeth A. P. Copello and Larry A. Kroutil, September 2016.
d. In 2015, 2.0 million people had a pain reliever use disorder;
e. Most adolescents who misuse prescription pain relievers are given them for free by a friend or relative.⁵ ⁵ Prescription Drug Use and Misuse in the United States: Results from the 2015 National Survey on Drug Use and Health, SAMHSA: Arthur Hughes, Matthew R. Williams, Rachel N. Lipari, and Jonaki Bose; RTI International: Elizabeth A. P. Copello and Larry A. Kroutil, September 2016.
f. In 2014, 467,000 adolescents, aged 12 to 17, were current nonmedical users of pain relievers, with 168,000 having an addiction to prescription pain relievers.⁶ ⁶ Substance Abuse and Mental Health Services Administration, Center for Behavioral Health Statistics and Quality. (2015). Behavioral health trends in the United States: Results from the 2014 National Survey on Drug Use and Health. Rockville, Md.: Substance Abuse and Mental Health Services Administration.
g. According to the CDC, each day, almost 7,000 people are treated in emergency room (ER) for using opioids in a manner other than as directed (That equates to 2.6 million people annually. Of these, more than 1.4 million ER visits were related to prescription drugs).
h. There are 420,000 prescription opioid overdose ER visits annually.⁷ Fifty five percent (55%) result in hospitalizations (on average 3.8 days) costing on average $29,500; 45% treated in the ER for an average cost of $3,600.⁸ Direct medical costs related to ER prescription opioid overdose visits equal >$7.5 billion annually->$25 billion in total medical costs annually (including ER costs). ⁷ CDC⁸ Substance Abuse and Mental Health Services Administration, Center for Behavioral Health Statistics and Quality. (2015). Behavioral health trends in the United States: Results from the 2014 National Survey on Drug Use and Health. Rockville, Md.: Substance Abuse and Mental Health Services Administration.
   91% of opioid overdose patients get another opioid prescription, most from the same doctor.
   7% overdose again within 12 months.
   14% overdose again within 24 months.
      17% of high-dose patients overdose within 24 months, equal to 10.7% of total annual overdose patients.
i. Drug abusers have healthcare costs that are nearly nine times higher than non-abusers.
j. Three studies estimated the United States economic burden of the opioid epidemic at around $55 billion annually.
   In the United States, prescription opioid abuse costs were about $55.7 billion in 2007. Of this amount, 45%, $25 billion, to healthcare costs (e.g., ER visits and treatment costs), 46% was attributable to workplace costs (e.g., lost productivity), and 9% to criminal justice costs.⁹ ⁹ Alabama Teen Challenge, Prescription Painkiller Overdose Epidemic In US https://alatc.org/prescription-painkiller-overdose-epidemic-in-us/
   Hansen's study attributed 79 percent of the costs to lost productivity ($42 billion), 15 percent to criminal justice costs ($8.2 billion) and only six percent to medical costs, comprising four percent for drug abuse treatment ($2.2 billion) and two percent for medical complications ($944 million).¹⁰ ¹⁰ Ryan N. Hansen, et al., “Economic Costs of Nonmedical Use of Prescription Drugs,” Clinical Journal of Pain 27, no. 3 (2011): 194-202, doi:10.1097/AJP.0b013e3181ff04ca.
   In Birnbaum's study, healthcare costs, defined more broadly, accounted for 45 percent ($25.0 billion) of the total, while workplace costs accounted for 46 percent ($25.6 billion) and criminal justice costs for only 9 percent ($5.1 billion).¹¹ The medical expense number correlates with the estimated costs per h above. ¹¹ Howard G. Birnbaum, et al., “Societal Costs of Prescription Drug Abuse, Dependence and Misuse in the United States,” Pain Medicine 12, no. 4 (2011): 657-67, doi:10.1111/j.1526-4637.2011.01075.x.
Regardless of the cost allocations, the burden to the United States healthcare system and federal, state, and local governments is significant and growing.

This is a worldwide problem. As examples, according to the Canadian Centre on Substance Abuse (CCSA), Canada ranked second, behind the United States, for the highest opioid consumption per capita in the world. In Europe, deaths from heroin declined, but deaths from synthetic opioids rose. Estonia saw one of the worst increases in prescription opioid deaths in the world, with a 38 percent increase from 2011-12. In New Zealand, opioid abuse is on the rise, and more than 1.2 million people abuse opioids—primarily prescription painkillers. In South America, a continent known for its production and trafficking of illicit drugs, opioid abuse is low in most countries. However, Costa Rica reported about 2.8 percent of the population abuses prescription painkillers.

Many problems regarding prescription drug abuse arise when doctors prescribe highly-addictive drugs in too large of dosages or prescribe drugs based on inaccurate diagnoses. Increased scrutiny of doctors leads to another problem. Some doctors do not prescribe medications in high enough doses because of the fear of addicting their patients or because of fear of scrutiny from other doctors and/or government agencies.

Unfortunately, the population that most often suffers from chronic pain is also one of the most vulnerable to addiction. The elderly is one of the biggest groups of prescription drug addicts in the United States. Doctors wrote 55 million, 21 percent of the 260 million opioid prescriptions, for people 65 and older, in 2013, a 20 percent increase from the prior five years. The Substance Abuse and Mental Health Services Administration (SAMHSA) found that the number of seniors misusing or dependent on prescription pain relievers rose to 336,000 in 2012, up by 132,000 since 2002. From 2007-11, visits to the emergency room caused by the misuse of pharmaceuticals by people older than 65 climbed 50 percent.

Exemplifying the misconception regarding the safety of prescriptions, a SAMHSA survey found more than half of the people aged 12 and older who abused prescriptions in 2012-13 got the drugs from friends or family members for free.

A NIDA survey found about one out of every 12 high school seniors admitted to the nonmedical use of Vicodin® and one out of every 20 admitted to abusing OxyContin® in 2010. SAMHSA found 2.2 percent of youths aged 12 to 17 used prescription drugs for nonmedical purposes, and 1.7 percent used pain killers for nonmedical purposes in 2013. That's more than hallucinogens (0.6 percent), cocaine (0.2 percent) and heroin (0.1 percent) combined.

Avoiding unnecessary medical complications or death by ensuring a drug is efficacious for the patient and that the patient is compliant and persistent with their prescription(s) represents a major unmet need and a trillion-dollar global market opportunity—this is larger than the global pharmaceutical industry. As an example, Express Scripts, the largest pharmacy benefit manager in the United States, found only 25 to 30 percent of medications are taken per the Prescriber's instructions (adherence) . . . and of those taken, only 15 to 20 percent are refilled per the Prescriber's instructions (persistence). This lack of adherence and persistence is estimated to result in more than $300 billion being wasted annually for the treatment of unnecessary medical complications in the United States.

Drug-related hospitalizations account for 2.4 to 6.5 percent of all medical admissions in the general population. A meta-analysis found a fourfold increase in the rate of hospitalization related to adverse drug events (ADE) in older adults compared with younger adults (16.6 versus 4.1 percent). Myriad factors in older individuals contribute to their increased risk for developing a drug-related problem. These include frailty, coexisting medical problems, memory issues, polypharmacy, and the use of non-prescribed medications. Estimates indicate that 88 percent of the ADE hospitalizations among older adults were preventable, compared with 24 percent among young persons.

Prescription opioid overdoses in the United States illustrates the cost savings potential of controlled medication prescribing′². According to the CDC (Centers for Disease Control and Prevention), there are 420,000 prescriptions opioid overdose ER visits and 16,000 Rx opioid overdose deaths annually. Fifty-five (55) percent result in an average 3.8 days of hospitalization at an average cost of approximately $30,000. The remaining 45 percent of patients run up an average $3,600 per ER visit. That equates to an average cost of $18,000 per ER Rx opioid overdose annually—a total direct cost of $7.5 billion. If total medical costs for additional physician visits, addiction treatment, etc. are factored in, the medical costs, including the $7.5 billion, exceed $25 billion annually. This estimate correlates well with other estimates of direct medical costs associated with prescription overdose abuse.

Optimizing drug therapy is an essential part of medical care. The process of prescribing a medication is complex and includes (i) deciding that a drug is indicated, (ii) choosing the best drug, (iii) determining a dose and schedule appropriate for the patient's physiologic status, (iv) monitoring for effectiveness, tolerance and toxicity, (v) educating the patient about expected side effects, and (vi) indications for seeking consultation.

Avoidable adverse drug events (ADEs) are the serious consequence of (i) inappropriate drug prescribing, (ii) changes in the patient's reaction to the drug over time due to increased tolerance, lifestyle, other medications, other medical conditions, worsening medical condition, or changes in the patients overall well-being, etc., or (iii) addition of new prescription or OTC medications, vitamins, dietary supplements, herbal medicines (e.g., ginseng, Ginkgo biloba extract, glucosamine, St. John's wort, echinacea, garlic, saw palmetto, kava, and valerian ¹² Sources: CDC; Presentation of Prescription and Nonprescription Drug Overdoses to US Emergency Departments, JAMA Intern Med. 2014; 174(12):2034-2037. doi:10.1001/jamainternmed.2014.5413; Societal costs of prescription opioid abuse, dependence, and misuse in the United States. Pain Medicine 2011; 12: 657-667 root), and/or recreational drugs, etc. Often, clinicians do not question patients about use of herbal medicines and patients do not routinely volunteer this information. Furthermore, most patients do not inform their clinician that they were using unconventional and/or recreational medications. A study of the use of 22 supplements in a survey of 369 patients aged 60 to 99 years found potential interactions between supplements and medications for ten of the 22 supplements surveyed. As a result, any new symptom should first be considered to be drug-related until proven otherwise.

Prescribing for older patients, who consume the most medications per capita, presents unique challenges. Premarketing drug trials often exclude geriatric patients and approved doses may not be appropriate for older adults. Many medications need to be used with special caution because of age-related changes in pharmacokinetics (i.e., absorption, distribution, metabolism, and excretion) and pharmacodynamics (the physiologic effects of the drug).

Larger drug storage reservoirs and decreased clearance prolong drug half-lives and lead to increased plasma drug concentrations in older people. Particular care must be taken in determining drug dosages. The proportional increase in body fat relative to skeletal muscle that generally accompanies aging may result in the increased volume of drug distribution. Decreased drug clearance may also result from the natural decline in renal function with age, even in the absence of renal disease.

The same dose could lead to higher plasma concentrations in an older, compared to younger, patient. For example, the volume of distribution for diazepam is increased, and the clearance rate for lithium is reduced, in older adults. From the pharmacodynamic perspective, increasing age may result in an increased sensitivity to the effects of certain drugs, e.g., opioids and benzodiazepines. Prescription Drug overdose deaths often involve benzodiazepines.

The use of greater numbers of drug therapies has been independently associated with an increased risk for an adverse drug event, irrespective of age, and increased risk of hospital admission. Polypharmacy is of particular concern in older people who, compared to younger individuals, tend to have more disease conditions for which therapies are prescribed. Approximately half of the patients taking drugs take two medications and 20 percent five or more. As an example, one study found that among ambulatory older adults with cancer, 84 percent were receiving five or more and 43 percent were receiving 10 or more medications.

The risk of an adverse event due to drug-drug interactions is substantially increased when multiple drugs are taken. For example, the risk of bleeding with warfarin therapy is increased with coadministration of selective and non-selective NSAIDs, SSRIs, omeprazole, lipid-lowering agents, amiodarone, and fluorouracil. A study found hospitalizations for hypoglycemia was six times more likely in patients who had received co-trimoxazole. Digoxin toxicity was 12 times more likely for patients who had been started on clarithromycin. Hyperkalemia was 20 times more likely for patients who were treated with a potassium sparing diuretic. And rrescription painkiller overdose deaths often involve benzodiazepines.

Periodic evaluation of a patient's drug regimen is an essential component of medical care. However, a survey of Medicare beneficiaries found that more than 30 percent of patients reported they had not talked with their doctor about their different medications in the previous 12 months. Furthermore, when these reviews are done, they often overlook OTC, supplements, herbal medicines and recreational drugs that are being taken by the patient.

Multiple factors contribute to the appropriateness and overall quality of drug prescribing. These include avoidance of inappropriate medications, appropriate use of indicated medications, monitoring for side effects and drug levels/drug tolerance, avoidance of drug-drug interactions, monitoring for increasing medication tolerance and involvement of the patient and integration of patient values. Current measures of the quality of prescribing generally focus on one or some of these factors, but rarely on all.

### BRIEF SUMMARY OF THE INVENTION

The present invention describes a novel integrated system of a tamper resistant drug dispensing device, related drug cassette, firmware, software, biometric logon, a drug specific and/or patient tailored App, a drug overdose diagnostic screen (OverDoseScreen™), drug specific dose dispensing algorithm, electronic communications, data analytics, and integrated support. The system, processes and methods are integrated together so as a whole they ensure: 1) the drug is only dispensed to or (in the case of a minor) for the patient for whom the drug is prescribed (avoid divergence); 2) the drug is efficacious for the patient; 3) the drug is properly titrated to achieve the best therapeutic dose; 4) the drug is the right dose to control the patient's condition; 5) the dispensed drug dose will not overdose the patient, 6) the prescriber has the requisite drug specific digitally captured and/or patient self-assessment and/or self-test and/or self-reported physiological, psychological, lifestyle, concurrent medications, and environmental information (values) to manage drug dosing, and/or tolerance and/or side effects; 7) that in the case of withdrawing the patient from a drug, that the withdrawal is properly managed; 8) that the prescription is only written, filled, and dispensed by authorized medical professionals; 9) that the dispensing of each dose is controlled to preclude dispensing if:
i. the patient is trying to take the medication sooner than the prescribed interval,
ii. the algorithm deduces that taking the drug may result in an adverse event, e.g., a drug-drug interaction or overdose, even if it could otherwise be dispensed within the prescription's dosing instructions,
iii. the drug is past its expiration/beyond-use date,
iv. the prescription dispensing period has expired,
v. the drug was not stored properly, e.g., within the right temperature and/or humidity guidelines,
vi. the drug batch has been recalled, and/or
vii. the patient is trying to double dose or if he/she is trying to take the same prescribed dose from more than one dispenser at the same time (misuse and/or abuse).

It is designed to improve the drug's safety and efficacy profile by assisting the prescriber in managing patient specific drug tolerance, drug induced side effects (e.g., drug induced constipation), and prescription compliance and persistence through the use of digitally captured and/or patient inputted self-assessment, self-test, and/or self-reported information at the point of dispensing when an drug dose is dispensed or dispensing is attempted by the patient. This is one time and/or trended information (values) which are made available to the patient and/or prescriber via on demand reports that show the relationship between medication dosing and certain tracked values/side effect information, e.g., medication effectiveness.

By increasing the drug's efficacy/safety profile, the integrated dispensing system improves the quality of care and the patient's quality of life while saving the healthcare system money by: (i) in precluding overdose deaths, (ii) emergency room visits, (iii) hospitalizations, and (iv) physician and allied healthcare professional interventions while decreasing the incidence of drug related side effects, addiction and dependence.

In certain embodiments, the drug specific APP which contains the dispensing algorithm, uses encrypted communications to control the drug dispensing device and to communicate with the patient, the centralized data servers, and the Integrated Support Center and/or designated Prescribers, physicians, caregivers, and/or family members. The algorithm uses the prescription information, drug information, drug dispensing device information, drug cassette information, Prescriber's unique identifier number, patient self-assessment, self-test, and self-reported and/or digitally captured physiological, psychological, lifestyle, medications currently being taken and/or environmental data/information values, in a novel drug specific diagnostic algorithm to decide if the drug dispenser should dispense the drug or keep the dispenser from dispensing the medication by keeping the tamper resistant dispensing unit.

The novel Drug Specific and Patient Tailored App, which can be operated from a standalone drug dispensing device and/or interface device (smartphone, tablet and/or computer, etc.) with Bluetooth, Wi-Fi, and/or Internet communication capabilities, reads and aggregates; (i) the prescription information, (ii) drug storage requirements, (iii) Prescriber's name, unique government issued identification number, and, if applicable, DEA number (or similar government control number), (iv) the drug label information from the drug cassette, (v) the drug cassette's unique serial number, (vi) the dispensing device's unique serial number, (vii) storage temperature and humidity readings, (viii) unauthorized attempts to open the dispenser since last dispensed dose, (ix) the date and time of the last dispensed dose, (x) digital data generated by wearable devices, consumed, implanted, or ingested diagnostic devices, monitoring devices, machines, instruments, gadgets, contraptions, apparatuses, utensils, implements, tools, mechanisms, smartphones, digital cameras, and informalgizmos, etc., (xi) patient self-assessment data from input screens on the standalone dispenser or interface device (smartphone, tablet, and/or computer, etc.), (xii) patient self-test information/data from input screens on the standalone dispenser and/or interface device (smartphone, tablet, and/or computer, etc.) and (xiii) self-reported information from query screens, digital diaries (e.g., Apple iPhone Health App), and/or from input screens on the standalone dispenser and/or interface device (smartphone, tablet, and/or computer, etc.)

The drug dispensing device is designed to automatically recognize the drug when the drug specific disposable drug cassette is docked into the device. The cassette is marked to allow the drug dispenser to ascertain the name of the drug (brand and/or generic), the drug's NDC number, the drug batch number, drug's expiration/beyond-use date, and drug storage requirements, and any data required by the FDA and any governmental agencies and like country governmental agencies, etc.

The Drug Specific and Patient Tailored App may incorporate a drug specific HIPPA and/or similar healthcare information protection legislation and guidelines compliant Drug Specific Dispensing Algorithm which uses encrypted communications to control the drug dispensing device and to communicate with the patient, the centralized data servers, and the Integrated Support Center and/or designated Prescribers, physicians, caregivers, and/or family members. The algorithm uses the prescription information, dispensing device information, drug cassette information, Prescriber's DEA and/or unique identifier number, digitally captured and/or patient inputted self-assessment, and/or self-test, and/or self-reported physiological, psychological, lifestyle, concurrent medications being taken and/or environmental data (digitalized information and/or values), in a novel drug specific diagnostic algorithm to decide if the drug dispenser should dispense the drug or keep the dispenser from dispensing the drug by keeping the tamper resistant dispensing unit locked.

The Drug Specific Dispensing Algorithm utilizes an incorporated drug specific overdose screen diagnostic, OverposeScreen™, to ascertain if the drug dose should be dispensed or if it should not be dispensed, even if it would otherwise be allowed by the prescription. The drug specific overdose screen diagnostic is specific to each drug and each drug formulation. The objective of the drug specific overdose screen diagnostic is to decrease drug overdoses.

The novel integrated system is designed to be able to use various drug dispenser configurations, designs, and sizes. They can be limited to a single drug or configured to dispense multiple drugs. The drug dispenser can be a disposable unit or a reusable unit. Regardless, dose dispensing from each drug dispenser is controlled by a Drug Specific and Patient Tailored App. The App can be resident on a standalone version of the drug dispenser or can reside on an interface device (e.g., smartphone, computer, tablet, etc.). The following describe disposable versus reusable drug dispensers.

The disposable drug dispensing device (Disposable Drug Dispenser) is designed to: (i) fit in a pants pocket and/or purse, (ii) be water, and dust resistant, (iii) withstand being dropped and/or banged, (iv) operate and withstand hot and cold temperatures within defined temperature ranges, (v) be powered by a battery with a life equal to or greater than the drug's expiration date, (vi) have a drug cassette containing a drug that docks into the dispenser housing, (vii) have a tamper resistant dispenser housing that becomes an integral unit with no access to the drug in the drug cassette except for when the single drug dose is dispensed, (viii) remain locked from dispensing unless the dispensing device receives an encrypted signal authorization from the Drug Specific App to dispense a dose, (ix) one click dose dispensing. When the drug dispensing device effectuates a secure handshake with the Drug Specific App, the drug dispenser transmits: (i) its serial number, (ii) the current and historic temperatures and humidity levels since the last dispense, (iii) any alerts regarding attempts to open the drug dispenser since the last drug dose was last dispensed, (iv) any drug dispensing error messages since the drug was last dispensed, and (v) the date and time the last drug dose was last dispensed. The drug dispensing device can be configured to dispense one or more drugs from a single drug cassette.

The reusable drug dispensing device (Reusable Drug Dispenser) is designed to: (i) fit in a pants pocket (for single drug units) and/or purse and/or belt holster (single or multiple drug dispensers), (ii) be water, and dust resistant, (iii) withstand being dropped and/or banged, (iv) operate and withstand hot and cold temperatures within defined temperature ranges, (v) be reusable, (vi) be powered by a replaceable and/or rechargeable battery, (vii) have a drug cassette containing the drug only be able to be docked or removed by an authorized healthcare professional, (viii) have a tamper resistant dispenser housing that becomes an integral unit with no access to the drug in the drug cassette except for when the single drug dose is dispensed, (ix) remain locked from dispensing unless the dispensing device receives an encrypted signal authorization from the Drug Specific App to dispense a dose, and (x) one click drug dispensing. When the drug dispenser effectuates a handshake with the Drug Specific App, the drug dispenser transmits: (i) its serial number, (ii) the drug information on the drug cassette, (iii) the current and historic temperatures and humidity levels since the last dispense, (iv) any alerts regarding attempts to open the drug dispenser since the last drug dose was last dispensed, (v) any drug dispensing error messages since the drug was last dispensed, and (vi) the date and time the last drug dose was last dispensed. The drug dispensing device can be configured to dispense one or more drugs from a single drug cassette or from more than one cassette when multidrug dispensing devices are used.

The single drug dispensing device is controlled by a single App.

The multi-drug dispensing units utilize multiple drug cassettes (one each per drug) which are controlled by a consolidation App that combines the individual drug Apps into a single user interface to eliminate duplication of inputs and to facilitate one click drug dispensing for one or more medications. The handshake between Apps is controlled by the biometric security system.

The single Drug Specific App, as well as the multi-drug App, require biometric sign on by the patient and utilize a drug specific decision tree algorithm and drug specific diagnostics and/or digitally captured and/or patient entered self-assessment, and/or self-test, and/or self-reported physiological, psychological, lifestyle, concomitant medications, and/or environmental information (data/values) to make dispensing decisions. An encrypted biometric sign on and/or alternative sign on include but are not limited to an iris scan, a Logon Name and Password, finger print scan, unique sequence of hand movements, voice control, face scan, unique phrases, a unique code sent to the patient's mobile phone, etc.

Once the biometric logon has been effectuated and the required information for the dispensing diagnostic has been captured, if everything is within normal ranges, then the patient is given a message to dispense the drug and/or related medications with one click on the drug dispensers control button. No further communication with the patient are required.

However, if the algorithm ascertains that it has to keep the dispensing device locked and not to dispense, even if within prescribing guidelines, then a number of alternative messages are shown on the dispensing device's and/or interface device's screen(s). These range from telling the patient that the requested drug dose is not authorized by the prescription for a specified period of time to a message indicating that a dose, even within the prescription dosing schedule parameters, should not be taken without first talking with the integrated support center or the prescribing healthcare professional. The App facilitates calling the support center using a single click on the alert window. The Integrated Support Center is able to triage the inquiry to a physician, payer defined disease management support center, or can serve as a disease management and/or as medical support center for patients.

The App uses the biometric sign on and encrypted communications with the data center's servers and the support center to document and communicate to the appropriate parties (Integrated Support Center, care givers, authorized family members, etc.) that, for example, (i) the patient may be heading for an undesired event, (ii) that the prescription should be changed, (iii) the drug may have to be changed based upon efficacy concerns, (iv) the patient has attempted to dispense a drug dose early too many times (which will depend on the drug type (e.g., opioids, anxiolytics, antidepressants, blood thinners, etc.)), (iv) appears to be following a misuse or an abuse pattern, etc., (v) is not following prescribing guidelines, and (vi) is failing to take the medication, etc.

The App is unique to each drug and/or each patient. Certain algorithm interface screens may be selected by the Prescriber to capture information, on a defined schedule or sequence, to allow for better patient management and to individualize drug therapy. These screens may or may not be utilized by the Drug Dispensing Algorithm to make a dose dispensing decision. As an example, the Prescriber may indicate that the App presents a pain self-assessment screen to track the effectiveness of the drug to control the patient's pain and to monitor for increasing drug tolerance; for stool consistency (self-reported) and/or for the frequency of bowel (self-reported) movements to ascertain if the patient is moving towards drug induced constipation, and/or an SpO₂ (blood oxygen saturation self-test) to ascertain if the drug is leading to a depressed respiratory rate, etc. and/or pulse rate (digitally captured) as part of the dispensing procedure.

A prescriber may request the constipation self-reporting information to enable the algorithm to: 1) make a dispensing decision and/or 2) alert the patient that he/she appears to be getting constipated and that the patient should consider taking a laxative, and/or 3) to recommend the patient consult the support center to ascertain if it would be advisable to take a laxative, and/or 4) to advise the prescriber that the patient is showing signs of drug induced constipation, etc. Another example would be the Prescriber designating that the App require a speech self-test and/or a cognition self-test and completion of a concomitant medications taken self-report, etc. as part of the dispensing request procedure. The Prescriber would use this particular information to preclude a potential drug-drug interaction, etc. The respective screens may be presented every time before dispensing or based upon a defined interval or as a result of a digital reading or other screen input, etc., as defined by the Prescriber.

The Prescriber can also designate that certain drug specific side effect information be tracked via specific side effect input screens, even if they will not be required by the dispensing algorithm, to better manage the patient. If the Prescriber chooses to track a side effect or multiple side effects, the Prescriber can designate how often each should be tracked, e.g., every time before a dose is dispensed, once per day, once per week, when a tracked value exceeds or is lower than a specified range, etc.

The prescription information and Prescriber designated side-effect tracking information can either be automatically digitally uploaded or manually entered into the Drug Specific and Patient Tailored App. The system generally calls for utilization of a novel Prescription API (Application Program Interface) to allow the Prescriber to avoid having to enter information twice, once for the prescription and once for the Drug Specific App. The API syncs the data input interface with the e-prescribing programs (e.g., ADSC MedicsRx, Allscripts, DAW Systems, DrFirst Rcopia, MDT Toolbox-Rx, Medtab RxCure, OA Systems, Practice Fusion, RxNT eRx, Spectra eRx, etc.) and/or pharmacy prescription systems (e.g., OmniCare, QS/1, PharMerica, Frameworks, etc.) and/or Electronic Prescribing Networks (e.g., SureScripts—that seamlessly connects Prescribers with Pharmacies) eliminating duplicate entries. The Prescription API enables the Prescriber to prescribe his/her additional information capture and/or side effects tracking and their respective tracking frequencies and to input patient authorized care giver and medical professional alert notification authorizations and their related contact information.

Eliminating duplicate inputs may also be facilitated via the integration of data through the utilization of a network, such as SureScripts Electronic Prescribing Network, which seamlessly connects physicians with pharmacies. The networks allow the secure, reliable transmission and delivery of electronic prescription orders and renewal authorization requests between computers at the pharmacy and computers at the physician's office. Simultaneous transmission to the Patient Database resident on the Central Servers can be accommodated.

Upon submission of the electronic prescription, which can also happen at the pharmacy from a written prescription, the information from the prescription interface is sent to the Patient Database resident on the Central Servers. Upon receipt, the Drug Specific and Patient Tailored App generation program creates a patient specific database and generates certain authorization codes (e.g., patient identifier record number, dispenser opening code for reusable drug dispensers, etc.), link the prescription, patient, and pharmacy to the App, and then automatically generate the patient and Drug Specific App, a copy which is retained on the Central Server. Then, the program automatically sends an email and/or text message to the Patient with an App download link. A click on the link, or copying the link to the navigation bar on the Internet browser, will take the patient to the patient and Drug Specific App download page. Clicking on the “download button” automatically downloads the App to the interface device. Once the patient downloads the Drug Specific and Patient Tailored App, they will receive a separate email with a unique Patient Identification Number. At that point, the Patient will be able to marry the Drug Specific App with the drug dispenser. The updates can also be accomplished via a wireless connection and via a wireless connection or a USB cable interface for multi-drug reusable drug dispensers, etc.

For reusable drug dispensers, this is a procedure that would normally be done when the pharmacy plugs in the USB cable to power the mechanical open and close locking mechanism on the drug dispenser and which is required to enter the medical professional restricted Dispenser Opening Code (the reusable drug dispenser can only be loaded by an authorized medical professional).

The initial click on the App requires the Patient to provide their Patient Identification Number which was previously emailed to them, certain personal information, establish the biometric certification baseline, and tech support password and challenge questions information. Upon completion, the Patient will be able to use the biometric log on to access the drug dispensing algorithm. If this fails, they can request a password reset or can contact the Integrated Support Center for Tech Support to provide a temporary password once the patient properly answers certain challenge questions. If required, the Integrated Support Center can enable the patient to dispense a drug dose while they work with the patient to troubleshoot their biometric log on problem. The Integrated Support Center can be contacted by clicking on the dial button on the alert screen of the Drug Specific and Patient Tailored App or by calling the number listed for the Integrated Support Center.

The App allows the patient to ask certain questions regarding when they took their last dose of the drug (or drugs, for multidrug dispensers), how much medication is left, when their next dose is due, the medication's expiration/beyond-use date, and the drug's package insert information, etc. It further provides access to personalized analytical charts, some which may be downloaded from the Integrated Support Center's servers or created by the App from the limited information stored by the App, to show how the patient's symptoms are affected when the patient takes a drug dose over time. This is designed to aid in patient prescription persistence, reinforce the importance of prescription compliance, and assist the patients in discussions with the prescriber related to pain management and any drug tolerance and/or side effect issues (depends on the types of information the prescriber designates be tracked).

The Integrated Support Center's IT system (data servers) stores authorized log on information, all App history data and enables the continual update/syncing of the App history on all the patient's devices where the Drug Specific and Patient Tailored App has been downloaded. The centralized servers are also designed to: (i) via the Internet, update individual App software as required, (ii) update the patient's electronic medical record resident on the Central Servers, (iii) to, on a real time basis, update the Integrated Support Center's patient specific counselor screens, (iv) conduct metadata analysis on both the patient's individual data as well as analysis that may include information from the patient's electronic medical record, as well as de-identified patient data from other patients that share similar clinical profiles, (v) carry out comparative patient analysis against metadata across a patient population with similar characteristics, etc. The analytical output is designed to assist the Integrated Support Center and/or payer specified disease management group(s) and/or the patient's care givers in their counseling of the individual patient as well as any reporting and contacts with the Prescriber, patient and/or payer designated disease management center and/or the patient's physician, etc.

The Integrated Support Center IT systems are designed to allow the Integrated Support Center, via the patient's Drug Specific App, (i) to change a patient's prescription based on an authorized Prescriber's instructions, (ii) lock the dispenser if tampering is suspected (and to follow the established patient, Prescriber and/or disease management protocol to ascertain the next step the Integrated Support Center should follow), (iii) to lock and unlock the dispensing ability on the individual drug dispensing unit based upon a discussion with the patient and/or his care giver, and (iv) to lock all appropriate dispensing devices that contain a recalled drug and to instruct the patient via a message on their mobile phone, tablet and/or computer, by text message, by email, voice messages, by twitter, and/or any other like communication services to go to their pharmacy to get the drug replaced or to follow the recalling manufacturer's instructions.

The Integrated Support Center's support team uses metadata analysis as well as drug registry information, as requested by the prescribing medical profession, to assist them in developing the best course of therapy based on specific queries of the Integrated Support Center's databases and any authorized related electronic medical records. The Integrated Support Center can also utilize de-identified patient data in comparison with the individual patient's information to identify potential medication issues (e.g., under dosing, overdosing, increasing tolerance, addiction and/or dependence risks, potential abuse, etc.) to assist the physician, on a proactive basis, to manage/better manage the patient's pain management needs/drug therapy.

### BRIEF DESCRIPTION OF THE DRAWINGS

Advantages of embodiments of the present invention will be apparent from the following detailed description of the exemplary embodiments thereof, which description should be considered in conjunction with the accompanying drawings in which:

FIG. 1 is an exemplary embodiment of a closed loop system to control drug diversion, misuse, abuse, addiction, dependence, overdose, and deaths.

FIG. 2 is an exemplary embodiment of a closed loop drug dispensing system controlled by an Drug Specific App which incorporates a Drug Specific Dispensing Algorithm.

FIG. 3 is an exemplary embodiment of the Prescription Interface data capture between e-prescription Prescriber and/or pharmacy programs.

FIG. 4 is an exemplary embodiment of the logic to enter the Prescription information in the Pharmacy Database and in the Patient Database and to generate a Dispenser Opening Code (not required for disposable drug dispensers) and Patient Identification Number and to authorize the dispensing App to dispense the medication from the specific Drug Dispenser. Conversely, to deny dispensing of a prescribed drug dose from more than one Drug Dispenser where multiple drug dispensers containing the drug exist. The logic precludes multiple dispensing/abuse via the utilization of multiple drug dispensers.

FIG. 5 is an exemplary embodiment showing specific App Modules of an Drug Specific App which controls the Drug Dispenser.

FIG. 6 is an exemplary embodiment of the Biometric Authentication setup and log on.

FIG. 7 is an exemplary embodiment of the loading of the drug cassette into a reusable drug dispenser. The drug cassette is preloaded and sealed into disposable drug dispensers.

FIG. 8 is an exemplary embodiment of how the drug dispenser's firmware, database, and software will be updated/synced.

FIG. 9 is an exemplary embodiment of data syncing between the Patient Database and the Drug Specific App.

FIG. 10 is an exemplary embodiment of the Interface Database organizational structure and relation with the Patient Database.

FIG. 11 is an exemplary embodiment of the starting logic sequence when the patient accesses the Drug Specific App and the App logic checks to ensure the drug is efficacious.

FIG. 12 is an exemplary embodiment of the prescription dispensing compliance sequence.

FIG. 13 is an exemplary embodiment of the access and storage of digitally captured patient data/values.

FIG. 14 is an exemplary embodiment of the input and storage of patient self-assessment, self-test, and/or self-report values.

FIG. 15 is an exemplary embodiment of the information capture and storage of concomitant medications, tracked drug side effects, and additional clinical trial patient information.

FIG. 16 is an exemplary embodiment of a flow chart/decision tree used by the drug specific Drug Dispensing Algorithm.

FIG. 17 is an exemplary embodiment of the support triage used by the Integrated Call Center to respond to drug dispenser and/or medication dispensing issues.

FIG. 18 is an exemplary embodiment of a flow chart of a standard prescription log in, Patient Self-Assessment, Patient Self-Test, Patient Self-Reporting, digitally captured values and/or information, and drug dispensing or dispensing control screens.

FIG. 19 is an exemplary embodiment of different devices and the types of data that can be captured digitally by the Drug Specific App utilizing various application programming interfaces (APIs).

FIG. 20 is an exemplary embodiment of the Drug Dispenser and related Drug Cassette docking.

FIG. 21 is an exemplary embodiment of how the Drug Dispenser can be lengthen or widened to accommodate larger pills and/or more drug doses or widened to accommodate more than one Drug Cassette.

FIG. 22 is an exemplary embodiment of other drug dispenser designs as well as different drug dispenser configurations.

FIG. 23 is an exemplary embodiment of different Drug Cassettes on various exemplary pill configurations and number of tablets of different sizes that can be accommodated by an individual Drug Cassette.

FIG. 24 is an exemplary embodiment of Patient dosing times versus Patient Self-Assessment, Patient Self-Test, Patient Self-Reported, and or digitally reported Patient Values Reporting Screens.

FIG. 25 is an exemplary embodiment of the Consolidated Therapy App and various Multi-Drug Drug Dispensers designed to accommodate more than one Drug Cassette.

FIG. 26 is an exemplary embodiment of the Centralized IT System utilized to support the various drug dispensing systems.

FIG. 27 is an exemplary embodiment of an Integrated Support Center, triage center.

FIG. 28 is an exemplary embodiment of how the Reusable Drug Dispenser's clam shell design is assembled for secure, tamper resistant closing and opening.

FIG. 29 is an exemplary embodiment of the electronics schematic for the Drug Dispenser.

FIG. 30 is an exemplary embodiment of the placement of electronics and mechanical components on the outside and within the Drug Dispenser.

FIG. 31 is an exemplary embodiment, using oxycodone as an example, of the correlation of opioid plasma levels and pupil size.

FIG. 32 is an exemplary embodiment of a how pupil size can be used as an opioid overdose screen/diagnostic indicator.

FIG. 33 is an exemplary embodiment of the anatomy of eyes and the difference in dilation from bright light to dark light.

FIG. 34 is an exemplary embodiment showing how to detect, authenticate, and capture iris and pupil data to educate a dispense or do not dispense decision.

FIG. 35 is an exemplary embodiment of an algorithm used from the captured normalized pupil data to making a dispense or do not dispense decision.

FIG. 36 is an exemplary embodiment of the data capture screens and sequence for a motor skills self-test to ascertain cognitive impairment.

FIG. 37 is an exemplary embodiment of the dispense or do not dispense Oxycodone algorithm for the motor skills self-test.

FIG. 38 is a continuation of the exemplary embodiment of the dispense or do not dispense Oxycodone algorithm for the motor skills self-test depicted in FIG. 36.

FIG. 39 is an exemplary embodiment of an Oxycodone dispensing patient interface using a pupil scan and a motor skills self-test in combination to reduce the number of false positive (e.g., erroneous dispense or do not dispense decisions).

FIG. 40 is an exemplary embodiment of the opioid dispensing algorithm (using oxycodone as an example) used with a pupil scan and a motor skills self-test in a sequential order decision logic designed to reduce the number of false positives (e.g., erroneous do not dispense decisions).

FIG. 41 is an exemplary embodiment of the self-assessment and self-reporting screens used in sequence to reduce the number of erroneous opioid dispense or do not dispense decisions.

FIG. 42 is an exemplary embodiment of an algorithm that uses self-assessment and self-reporting responses to reduce the number of erroneous opioid dispense or do not dispense decisions.

### DETAILED DESCRIPTION OF THE INVENTION

### I. Terms and Acronyms

Terms used in this document, AKA denotes terms used interchangeably:

Abuse (AKA Substance Abuse, Medication Abuse, Drug Abuse, Drug Abuse, etc.) refers to the act or practice of improperly using medications (e.g., opioids, anxiolytics, antidepressants, stimulants, etc.). It also refers to the compulsive, excessive, and self-damaging use of habit forming drugs or of using drugs in harmful amounts, leading to addiction or dependence, serious physiological injury (such as damage to kidneys, liver, heart) and/or psychological harm (such as dysfunctional behavior patterns, hallucinations, memory loss), or death.

Addiction (AKA Drug Addiction, Substance Use Disorder, Drug Addiction) is defined as a chronic, relapsing disease that is characterized by compulsive drug seeking and use, despite harmful consequences. As an example, in the case of opioids, it refers to the compulsive need for and use of an opioid or opioids, e.g., a habit-forming substance, characterized by tolerance and by well-defined physiological symptoms upon withdrawal; broadly: persistent compulsive use of a substance known by the user to be harmful.

Adverse Event (AKA: AE, Adverse Event, Adverse Experience, Adverse Drug Event, ADE, Adverse Drug Reaction, ADR, or Unexpected Adverse Drug Reaction, etc.) refers to (i) a medical occurrence temporally associated with the use of a medicinal product, but not necessarily causally related, (ii) any response to a drug which is noxious and unintended, and which occurs at doses normally used in man for the prophylaxis, diagnosis, or therapy of disease, or for the modifications of physiological function, (iii) an unexpected reaction not consistent with applicable product information or characteristics of the drug, and (iv) the unintended effect occurring at normal dose related to the pharmacological properties of a medication, etc.

Allied Healthcare Professionals as used in this patent refers to all non-physician healthcare professionals engaged in caring for a patient inclusive of nursing, physician assistants, medicine, and pharmacy which may or may not have the authority to prescribe a drug.

API (AKA Application Program Interface) is a set of routines, protocols, and tools for building software applications. The API specifies how software components should interact. The API is the interface shared by two applications or programs that allow both to communicate with one another.

Beyond-use date (see Expiration Date)

Biometric Authentication (AKA Biometric Identification, Biometric Sign On) encompasses but is not limited to biometric technologies that digitally capture fingerprint, palm and full-hand scanners, voice, facial recognition systems, iris scanning technology, pupil scans, document readers, biometric software, and related services capable of wireless, mobile or stationary use to limit access to the Patient. In this document the term also incorporates any system, while not biometric, that allows access via the use of a Login Name in combination with a Password and/or any additional security information, e.g., a computer-generated password that is sent by a server via email and/or text message, as well as programs developed to allow for the personalization of motions or movements, etc. to restrict access only to the Patient.

Breakthrough Dose (AKA BTD, Rescue Dose) is an as needed additional medication dose used to control breakthrough pain (a transitory flare of pain that occurs on a background of relatively well controlled baseline pain, for the treatment/management of sporadic worsening of pain). It does not replace or delay the next routine dose.

Caregiver is defined as any individual, such as a physician, nurse, disease management representative, allied health professional, social worker or family member, guardian, or friend who assists a patient in the identification, prevention, management, and/or treatment of an illness or disability.

CDC (AKA Centers for Disease Control and Prevention).

Central Servers (AKA Cloud Servers, Centralized Server Structure) refers to the centralized computer system that stores all patient related data and communications. It refers to the hardware where programs are stored and databases reside to support all patient specific activities required to manage and control related drugs dispensing.

Cognitive Computing in this patent refers to computer systems, most notably IBM Watson, that rely on deep learning algorithms and neural networks to process information by comparing it to a teaching set of data. The more data the system is exposed to, the more it learns, and the more accurate it becomes over time. The neural network is a complex “tree” of decisions the computer can make to arrive at an answer. The programs help collate the span of knowledge around a condition, for instance the patient's pain, including patient history, journal articles, best practices, diagnostic tools, etc., analyze that vast quantity of information, and provide a recommendation regarding the best course to treatment to control the pain. Cognitive computing can also be utilized to continually analyze metadata to compare a patient's trended, for example, pupil scans, to predict if a patient is in danger of overdosing. Alternatively, it can be used to track pupil size, early dispensing attempts, drug dispenser tamper attempts, and pain self-assessment scores along with other patient data to warn the prescriber about potential drug dependence, addiction, abuse, and/or diversion problems. It can be used by the Integrated Support Center to use face recognition, via images from the patient's smart phone, and tone analysis to help educate the Support Center counselor regarding whether a requested drug dispenser lock should be unlocked and a dose dispensed.

Compliance (AKA Adherence, Capacitance, Prescription Compliance, Medication Compliance, Drug Compliance) describes the degree to which a patient correctly follows the prescription's drug dosing, dispensing and storage instructions.

Consolidation App (AKA Multiunit Dispenser App) is an App designed to recognize other Drug Specific Apps resident on a standalone dispenser or Interface Device and then to consolidate the requisite digitally captured information, Patient Self-Assessment screens and/or Patient Self-Test screens and/or Patient Self-Reported screens into a single interface for the control and dispensing of multiple drugs.

Data Input Interface (AKA Data Interface, Data Input) refers to the program designed to interface with the applicable e-prescribing programs (e.g., ADSC MedicsRx, Allscripts, DAW Systems, DrFirst Rcopia, MDT Toolbox-Rx, Medtab RxCure, OA Systems, Practice Fusion, RxNT eRx, Spectra eRx, etc.) and/or pharmacy prescription systems (e.g., OmniCare, QS/1, PharMerica, Frameworks, etc.) and/or Electronic Prescribing Networks (e.g., SureScripts—that seamlessly connects Prescribers with Pharmacies) to streamline patient data input and minimize or eliminate duplicate data entry requirements.

Dependence (AKA Physical Dependence) refers to the physiological adaptation of the body to the presence of a substance, e.g., opioids. It is defined by the development of withdrawal symptoms when the substance is discontinued or when the dose is reduced abruptly or, specifically in the case of opioids, when an antagonist (e.g., naloxone) or an agonist-antagonist (e.g., pentazocine) is administered. Physical dependence is a normal and expected aspect of certain medications and does not necessarily imply that the patient is addicted.

Digitally Captured refers to digital Patient data captured by diagnostic or monitoring devices and stored and/or transmitted in a machine-readable format. Digitally captured information can come from multiple sources such as apps resident on a smartphone or computer, data captured by the drug dispenser, from RFID chips embedded in the drug, from selfie images, from biometric logon (e.g., pupil size), from digital interfaces, from digitalized diagnostic devices, and/or from digital monitors, etc. The data may be used by the drug dispensing algorithm to make a dispense decision, and/or by the prescriber to better manage the patient, and/or by the drug's sponsor to capture clinical trial information, and/or by the patient to ascertain how effective the drug is for treating and controlling their pain, etc.

Dispensing Site (AKA Pharmacy) is defined as a pharmacy or any other medical facility, physician's officer, clinic, or the Patient's home where the prescription is dispensed.

Dispensing System (AKA Standalone Dispensing System, Disposable Dispensing System) is comprised of the drug, Drug Cassette containing the drug, a Drug Dispenser, the Drug Specific App which contains an Drug Specific Dispensing Algorithm, the Interface Device or Standalone Drug Dispenser where the Drug Specific App resides, and the Integrated Support Center with its related Data Bases and Data Servers. The standalone Dispensing System is comprised of the drug, Drug Cassette containing the drug, a Drug Dispenser, a Drug Specific App which contains a Drug Specific Dispensing Algorithm. The Disposable Dispensing System is comprised of the drug, Drug Cassette containing the drug (which may be an integral part of the Drug Dispenser, a Drug Dispenser, the Drug Specific App which contains a Drug Specific Dispensing Algorithm, the Interface Device (it can also be a Standalone Drug Dispenser) where the Drug Specific App resides, and the Integrated Support Center with its related Data Bases and Data Servers. All the Drug Dispensers interface with and relate to the Integrated Support Center with its related Data Bases and Data Servers.

Diversion (AKA Drug Diversion, Drug Diversion) is a medical and legal concept involving the transfer of any legally prescribed controlled substance from the individual for whom it was prescribed to another person for any illicit use.

Docked refers to the Drug Cassette residing in the Drug Dispensing Unit. Docked can also refer to a Drug Dispenser which is stored into a multiple Drug Dispenser Dispensing Unit.

Drug (AKA drug, opioid, pharmaceutical, medication, medicament, OTC drug, supplement, or herbal remedy, etc.)

Drug Cassette is either a reusable or disposable unit that contains a drug to be dispensed over a defined period of time and/or days per the prescription instructions that is designed to dock into a Drug Dispenser or designed to be an integral part of a Disposable Drug Dispenser. The Drug Cassette may come either prefilled with the drug from the drug manufacturer or filled by a medical professional at the pharmacy or at any location which is authorized to dispense the drug. The Drug Cassette may be an independent device designed to dock into a Drug Dispensing Unit or may be an integrated Drug Cassette in a disposable Drug Dispenser.

Drug Dispenser (AKA Dispensing Device, Drug Dispenser, Standalone Dispenser, Dispensing Unit, Disposable Drug Dispenser, Disposable Dispenser, etc.) refers to the Drug Dispensing Unit with a Docked or Integrated Drug Cassette whose dispensing is controlled by a Drug Specific App or a Standalone Drug Dispenser with a resident Drug Specific App or a drug dispensing program that controls dose drug dispensing by the dispenser.

Drug Dispensing Unit (AKA Reusable Drug Dispenser, Drug Dispensing Device, Drug Delivery Device, Standalone Drug Delivery Device, Standalone Drug Dispenser, Disposable Drug Dispenser, Multiple Drug Dispenser) is the device where the Drug Cassette is resident in the Dispensing Unit and whose dispensing mechanism (lock, unlock, and dispensing) are activated by Firmware controlled by a Drug Specific App and/or Drug Specific Apps or the resident software logic contained in the Standalone Drug Dispenser.

Drug Dispensing Software refers to dispensing software resident on an interface device or Standalone Drug Dispenser that: 1) requires Biometric Authentication, and 2) controls dose dispensing by a Drug Dispenser/Drug Dispensing Unit.

Drug Specific App (AKA Patient Tailored Drug Specific App, Drug Specific and Patient Tailored App, Medication Specific App, App) refers to an app that requires Biometric Authentication prior to a Patient being able to respond to Patient Self-Assessment screens and/or Patient Self-Tests screens and/or Patient Self-Report screens which are used by the App's Drug Specific Dispensing Algorithm to decide whether or not to signal the Dispensing Device to dispense the drug or to indicate to the patient and/or Integrated Support Center why the drug is not being dispensed.

Drug Specific Dispensing Algorithm refers to the decision tree based algorithm specifically developed for each drug and drug formulation to ascertain if the drug dose should or should not be dispensed.

Drug naïve patient refers to an individual who has either never had a drug or who has not received repeated drug dosing for a certain period specific to a drug, such as a 2 to 3-week period for opioids.

Electronic Medical Record (AKA EMR, Electronic Health Record, EHR, Patient Medical Record, PMR, etc.) refers to the systematized collection of patient and population electronically-stored health information in a digital format. These records can be shared across different health care settings. The centralized Electronic Medical Record for each Patient maintained by the Integrated Support Center on its Centralized Servers is designed to store all captured drug/medication dispensing information and all interactions between the Integrated Support Center representative and medical personnel and the Patient and/or caregivers. The information is made available to authorized providers in keeping with HIPPA guidelines and general requirements for protecting and sharing health information. It captures and stores all the information that is captured by the App as prescribed by the physician or dictated by clinical trial requirements, as required.

Encryption (AKA Encrypted, Encrypted communications) is the most effective way to achieve data security. Access requires a secret key or password that enables decryption. Unencrypted data is called plain text; encrypted data is referred to as cipher text. Encryption as used in this patent, by definition, is encryption which is designed to meet all HIPPA security standards or general requirements for protecting health information.

Expiration Date (AKA Beyond-Use Dates, Drug Expiration Date, Expiration/Beyond-Use Date) refers to the date after which a medication should not be taken (should not be dispensed) because an expected decline in quality or effectiveness. For this patent, Expiration Date is the shorter of the manufacturer assigned expiration date and the pharmacy assigned “Beyond-use date”. The “Beyond-Use Date” is the date placed on a prescription by a pharmacy noting when that prescription should no longer be used. It will often say “discard after . . . ” or “do not use after . . . ”.

Firmware is embedded systems software contained in the Drug Dispenser's memory to provide operating systems control, sensor information capture Oalerts, and handshake with the Interface Device.

Handshake (AKA Digital Handshake) refers to an exchange of signals between devices ensuring synchronization whenever a connection, as with another device, is initially established.

Integrated Support Center (AKA Call Center, Patient Support Center, Physician Support Center, Prescriber Support Center, etc.) refers to an Integrated Support Center designed to: (i) provide patient support related to the Drug Dispenser, Drug Specific App, alerts, locked dispenser screens, the drug, or health related questions. It can triage calls from dispenser and App technical support to the appropriate drug counselor, to the prescriber, to the patient's physician, and/or any contracted disease management services; and/or (ii) assist prescribers in optimizing drug therapy for a patient, and (iii) answer any business questions (e.g., customer inquiries regarding invoices, billing, orders, pricing, shipments, etc.).

Interface Device refers to the smartphone, tablet, computer, or standalone drug dispenser, etc. with Internet communications capabilities or like communications capabilities where the Drug Specific App resides which allows communications with the Integrated Support Center Servers (Central Computers).

Locked indicates the drug cannot be dispensed by the Dispensing Device until the Drug Specific App or the standalone device's dispensing software unlocks the Dispensing unit and allows it to dispense the medication.

Long Acting Medications (AKA Sustained Release, SR, Controlled Release, CR, Extended Release, ER) are slowly effective medications after initial dosage, but maintaining its effects over a long period of time, being slowly absorbed and persisting in the tissues before being excreted. These can come in various forms, but by reference in this patent, refer to oral or transdermal formulations.

Long Acting Drugs (AKA Sustained Release, SR, Controlled Release, CR or Extended Release, ER). These can come in oral or transdermal formulations.

Medication (AKA Drug, Pharmaceutical) refers to a substance used for medical treatment, especially a medicine or legal or illegal drugs, OTC medications, vitamins, dietary supplements, herbal medicines, and/or recreational drugs, etc.

Metadata Analysis (AKA Structural Metadata Analysis, Descriptive Metadata Analysis, Big Data, Big Data Analytics) as used herein refers to the use of the organization of patient data to enable analysis of both individual and patient population data to ascertain how to best manage drug therapy on a drug-by-drug and patient-by-patient basis. By reference, it incorporates the use of cognitive computing to ascertain how to best treat an individual patient's pain based upon the patient's own information as well as that of de-identified patient records, a patient's facial scan, and/or the patient's tone.

Opioids (AKA Opiates, Short Acting Opioid, Long Acting Opioid) refers to all agonists and antagonists with morphine-like activity, and also the naturally occurring and synthetic opioid peptides. This definition also includes all drug combinations which include at least one opioid in the drug combination. Examples of opioids are: buprenorphine, codeine, fentanyl, hydrocodone, hydromorphone, methadone, morphine, oxymorphone, oxycodone, tapentadol, and tramadol, etc.

Oral Patient Controlled Analgesia (AKA OPCA) is any method of allowing a person in pain to administer their own pain relief Historically, this has been limited to infusion using an infusion pump that is programmable by the prescriber. If it is programmed and functioning as intended, the machine is unlikely to deliver an overdose of medication. The embodiment of the invention expands the application to oral medications. The prescriber indicates the limitations put on the patient regarding the dispensing of his oral opioid. Dispensing may be controlled to: 1) a specified number of pills (doses) during a defined time interval; or 2) the number of pills that may be prescribed during a defined time interval with the limitation that individual doses not to be dispensed more frequently than a given timeframe between each dose, e.g., minimum of 20 minutes between doses, etc.

Patient refers to the individual that is prescribed and is taking a drug and/or medications.

Patient Database refers to all the patient data stored in the Central Servers. The data may be stored in one of more databases and on one or more servers which are collectively referred to as the Patient Database.

Patient Reported Outcomes (AKA PRO) is a method or questionnaire used in a clinical trial where the responses are collected directly from the patient. Collection may be via automatic digital capture, or via patient inputted self-assessment, self-test, or self-report physiological, psychological, lifestyle, concomitant medications, or environmental information/data.

Patient Self-Assessment (AKA Patient-Reported Outcome, PRO) covers a whole range of potential types of measurement resulting from a (i) patient responding to a question, (ii) a self-administered test, (iii) a self-report input which is digitally captured, and/or (iv) digital diaries whose information can be quantified for utilization by the Drug Specific Dispensing Algorithm and/or for utilization by the Prescriber for better Patient management and/or by a drug sponsor for clinical trial information reporting. Each self-assessment scale or question or diary input measures a single underlying characteristic(s). Examples include, but are not limited to responding/reporting, (i) the level of pain (e.g., responding to the Mosby Pain Index, Wong-Baker Facial Grimace Scale, etc.), (ii) an activity tolerance scale, (iii) a quality of life scale, (iv) a discomfort scale, (v) a physiologic value (e.g., blood pressure, heart rate, eye dilation, balance, gait, weight, food consumption), (vi) stress, (vii) blood oxygen saturation, SpO2, etc. The definition may include, but is not limited to, certain patient self-administered tests (also refer to as Patient Self-Test) or self-reported observations (also referred to Patient Self-Report).

Patient Self-Test refers to any test which is responded to by the patient and digitally captured to allow the Drug Dispensing Algorithm to make a drug dispensing decision. Examples, with specific applicability to opioids, include but are not limited to: (i) a fine typing test to ascertain cognition (speed and accuracy of movement); (ii) a walking test to ascertain balance and/or gait; (iii) voice test to ascertain the clarity and flow of speech; (iv) memory test to gauge cognitive status, motor functioning, attention/working memory, processing speed, and executive functioning, as well as learning and memory, (v) a manual acuity test to ascertain alertness and cognition, (vi) data entry test to ascertain cognition, etc. This information can be quantified for utilization by the Drug Specific Dispensing Algorithm and/or for utilization by the Prescriber for better Patient management and/or by the drug sponsor for drug clinical trials data capture.

Patient Self-Report is defined as any response made by the patient which is based upon his/her action, observation or best recollection. Examples include, but are not limited to, (i) OTC, herbal, or other medications taken over a prespecified period of time (as an example, in the case of opioids, this may be a question that deals with medications taken since the last opioid dose which would result in a medication or drug-drug interaction with the opioid), (ii) how the patient's stool composition appears (e.g., on the Bristol Stool Index), (iii) the last time the patient had a bowel movement, (iv) digital diaries (e.g., iPhone Health App, Samsung's S Health, disease specific diaries), etc. This information can be quantified for utilization by the Drug Specific Dispensing Algorithm and/or for utilization by the Prescriber for better Patient management, and/or by the drug's sponsor for use in clinical trials data reporting, etc.

Patient Specific Drug Specific App (AKA Drug Specific App) refers to a Drug Specific App used to control drug dose dispensing from a drug dispenser. The is automatically prescribed by the physician when prescribing the related drug. The prescriber can customize the App to the individual patient by requesting certain side effect information be tracked that is not required by the Drug Specific Dispensing Algorithm to make a dispensing decision. The Drug Specific Patient Tailored App can also be programmed to capture patient management and clinical trial information/data not required to make a drug dispensing decision.

Persistence (AKA Prescription Persistence, Medication Persistence) refers to the act of continuing the treatment for the prescribed duration. It may be defined as “the duration of time from initiation to discontinuation of therapy.” No overarching term combines these two distinct constructs.

Pharmacodynamics is the study of the biochemical and physiological effects of drugs on the body or on microorganisms or parasites within or on the body and the mechanisms of drug action and the relationship between drug concentration and effect.

Pharmacokinetics is the study of the bodily absorption, distribution, metabolism, and excretion of drugs.

Physician's Office is broadly defined as a medical facility, room, or rooms in which the physician and staff provide patient care; a location in which one or more medical doctors receive and treat patients. Examples include sole practitioner office, physician group practice offices, hospital clinics, out-patient ambulatory care centers, telehealth location, etc.

Polypharmacy is the practice of administering or using multiple medications especially concurrently (as in the treatment of a single disease or of several coexisting conditions).

Prescriber is defined as any healthcare professional authorized by an individual country or state to write a prescription for a drug. Examples include physicians, physician assistants, nurse practitioners, nurses, pharmacists, etc.

Prescription (AKA ePrescription, Paper Prescription, Manually Entered Prescription, Digital Prescription, Drug Prescription, Medication Prescription) is an instruction electronically entered or manually written by a medical practitioner that authorizes a patient to be provided a medicine or treatment.

Prescription Expiration Date is defined as the earlier of the drug expiration date or the prescription expiration date.

Recall refers to a drug recall issued by the manufacturer or a regulatory agency indicating that a particular drug batch or drug should not be taken.

Rotation is switching one drug for another. In the case of opioids, it is required for patients with inadequate pain relief and/or intolerable drug related toxicities or adverse effects.

Routines (AKA Software Program, Software Routines, Subroutine, Procedure, Function, Method, Subprogram) is a portion of software code within a larger program that performs a specific task and is relatively independent of the remaining code.

Serial Number (AKA Serial No., SN, S/N, Identification Number, Tracking Number, Identifier, etc.) is a unique number used for identification, tracking, and/or inventory purposes.

Short Acting Medications (AKA Immediate Release, IR) are quickly effective drugs that require regular repeated doses for long-term treatment. They are rapidly absorbed, distributed in the body, and excreted.

Short Acting Drugs are immediate release (IR) drugs.

Steady State is when the rate of drug availability and elimination equal one another.

Tamper Resistant refers to a design: 1) that precludes the drug dispenser from being opened without destroying it, 2) that makes it difficult to change, open, or remove the drug cassette from the drug dispenser except for authorized users, or 3) that results in damaging the drug dispenser when an unauthorized person attempts to open the drug dispenser.

Tethered Drug Dispenser is a Drug Dispenser that utilizes the Interface Device's capabilities to offer functionality and ease of use that would not otherwise be possible in a Standalone Drug Dispenser with the same outside dimensions, the same size.

Titration has traditionally been referred to as adjusting the dosage until a desired outcome is reached. Examples include opioids, dopamine, and nitroglycerine. Titration requires regular assessment of the patient's symptoms/values over a defined time period. In the case of opioids in particular, it requires regular assessment of the patient's pain, when and why it occurs as well as the amount of medication used in the previous 24- to 72-hour period.

Titration Cassette (AKA Titration Dispenser, Drug Titration Unit) refers to a Drug Cassette that is configured with one or more drugs for a specified period of time to ascertain the right dose and dosing schedule for a single drug or a combination of drugs.

Tolerance is a pharmacology concept where a subject's reaction to a specific drug and drug concentration is reduced following repeated use, requiring an increase in concentration to achieve the desired effect. It is a state of adaptation in which exposure to a drug induces changes that result in a diminution of drug's effect over time. It is a known pharmacologic effect of opioids. Toolerance to the analgesic effects of opioids is relatively uncommon.

Total Daily Dose (TDD) is the 24-hour total of a drug that is taken for regular and breakthrough doses.

Withdrawal is the group of symptoms that occur upon the abrupt discontinuation or decrease in intake of a drug. To experience the symptoms of withdrawal, the patient must have first developed a physical or mental dependence. Withdrawal symptoms generally last for a few days. In the case of opioids, withdrawal symptoms last for a few days and are generally the opposite of symptoms exhibited when the drug was started.

### II. List of Drugs, Drug Mechanisms of Action, and Level of Pain the Invention in its Various Embodiments is Applicable to

The invention and its various embodiments can enable the Prescriber to better manage and personalize a patient's drug therapy. The invention can assist the Prescriber in personalizing a patient's drug therapy to include: (i) establishing the lowest effective dose, (ii) on an ongoing, real-time basis, monitoring the ability of the drug or drugs to manage the patient's pain, symptoms and/or condition, (iii) adjusting the prescribed dosage if the patient develops drug tolerance sufficient to require an increased dose, (iv) facilitating the management of the drug to better manage drug withdrawal, (v) decreasing or precluding drug overdose, (vi) precluding drug abuse or misuse by restricting dispensing to the prescribed dosing interval, (vii) improving drug safety by the utilization of Patient Self-Tests to ascertain if the patient is being over or under medicated, (viii) decreasing drug mediated side effects through the utilization of Patient Self-Assessment, (ix) avoiding unnecessary medical professional interventions/office visits, related emergency room visits, and/or deaths by guarding against drug-drug adverse events, and (x) ensuring proper prescribing and prescription compliance through real time patient data capture, control of drug access by the patient, and Prescriber feedback. By improving each drug's safety profile, the invention improves the patient's quality of care, ensures the continued efficacy of the drug for each individual patient, improves the patient's quality of life by ensuring proper prescribing, and increases prescription compliance and persistence—thereby decreasing the number of drug related medical interventions, physician visits, ER visits, and hospitalizations; decreasing the total cost of patient care; and decreasing the number of drug overdose deaths. It also decreases healthcare costs by decreasing drug diversion which leads to prescription drug addiction, overdoses, misuse and abuse and their related healthcare costs.

Table 1, below, lists oral drugs with REMS programs. The listed approved drugs are encompassed in the embodiment of the invention by reference and can benefit from an improved drug safety profile. The Invention mitigates prescription risk for the drug manufacturer and Prescriber as it shifts the responsibility of prescription compliance to the patient. The listing for each drug includes, by definition, each opioid's respective indication(s), strength, dosage form, route of administration, side-effect profile, drug interactions, mechanisms of action, manufacturer, etc.).

In addition to Table 1, the embodiment incorporates by reference all marketed and in development oral drugs listed in: (i) Goodman & Gilman's, The Pharmacological Basis of Therapeutics (12th Ed) (Goodman et al. eds) (McGraw-Hill) (2011); (ii) 2015 Physician's Desk Reference; (iii) Cortellis™ Competitive Intelligence by Thomson Reuters; (iv) Adis R&D; and/or (v) Pharmaprojects by Citeline. The listing for each drug includes, by definition, each drug's respective indication(s), strength, dosage form, route of administration, side effect profile, drug interactions, mechanisms of action, manufacturer, etc.

TABLE 1
Oral Drugs with Required REMS Programs

Antipsychotics
Seroquel (Quetiapine)
Pain Relievers
Opioids
Codeine
Fentanyl and Analogs
Hydrocodone
Hydromorphone
Methadone
Oxycodone
Oxymorphone
Sedatives (Barbiturates)
Amytal (amobarbital)
Nembutal (pentobarbital)
Seconal (secobarbital)
Stimulants (ADHD)
Adderall (Amphetamine)
Methylphenidate
Daytrana
Concerta
Ritalin
Tranquilizers
A. Benzodiazepines, Like
Klonopin (clonazepam)
Valium (diazepam)
Xanax (alprazolam)
B. Non-Benzodiazepines, Like
Ambien (zolpidem)
Lunesta (eszopiclone)
Sonata (zaleplon)
Others
Chantix
Revlimid
Tracler
Xeljans (Jak Compounds)

Table 2 lists the Paragraph IV Challenged Drugs that can benefit from the increased patent protection afforded by the drug/device (Invention) combination. The following approved drugs and the FDA's Paragraph IV Drug Product Applications: Generic Drug Patent Challenge Notifications list are encompassed in the embodiment of the invention by reference. The listing for each drug includes by definition each drug's respective indication(s), strength, dosage form, route of administration, side effect profile, drug interactions, mechanisms of action, manufacturer, etc.

TABLE 2
Paragraph IV Challenged Drugs

| BRAND | GENERIC NAME |
|---|---|
| Ampyra | Dalfampridine |
| Daliresp | Roflumilast |
| Angeliq | Drospirenone and Estradiol |
| Nexavar | Sorafenib Tosylate |
| Kuvan | Sapropterin Dihydrochloride |
| Pradaxa | Dabigatran Etexilate Mesylate |
| Tradjenta | Linagliptin |
| Thalomid | Thalidomide |
| Gabitril | Tiagabine Hydrochloride |
| Zohydro ER | Hydrocodone Bitartrate |
| Viibryd | Vilazodone Hydrochloride |
| Abstral | Fentanyl Citrate |
| Letairis | Ambrisentan |
| Lamictal XR | Lamotrigine |
| Zorvolex | Diclofenac |
| Zytiga | Abiraterone Acetate |
| Ella | Ulipristal Acetate |
| Xartemis XR | Oxycodone Hydrochloride and |
| | Acetaminophen |
| Doryx | Doxycycline Hyclate |
| Noxafil | Posaconazole |
| Tekturna HCT | Aliskiren Hemifumarate and |
| | Hydrochlorothiazide |
| Promacta | Eltrombopag Olamine |
| Gilenya | Fingolimod |
| Afinitor | Everolimus |
| Gleevec | Imatinib Mesylate |
| Brisdelle | Paroxetine |
| Tikosyn | Dofetilide |
| Hysingla ER | Hydrocodone Bitartrate |
| Suboxone | Buprenorphine Hydrochloride and |
| | Naloxone Hydrochloride |
| Latuda | Lurasidone Hydrochloride |
| Trokendi XR | Topiramate |
| Contrave | Naltrexone Hydrochloride and |
| | Bupropion Hydrochloride |
| Equetro | Carbamazepine |
| Minastrin 24 Fe | Norethindrone Acetate and Ethinyl |
| | Estradiol and Ferrous Fumarate |

Table 3: Marketed Drugs lists approved drugs which are encompassed in the embodiment of the invention by reference. Drug compounds of interest are also listed in and encompassed in the embodiment of the invention by reference: Goodman & Gilman's, The Pharmacological Basis of Therapeutics (12th Ed) (Goodman et al. eds) (McGraw-Hill) (2011); and 2016 Physician's Desk Reference; Cortellis™ Competitive Intelligence databases by Thomson Reuters; Adis R&D databases; and/or Pharmaprojects databases by Citeline. The listing for each drug includes by definition each drug's respective indication(s), strength, dosage form, route of administration, side effect profile, drug interactions, mechanisms of action, manufacturer, etc.

TABLE 3
Marketed Drugs

Abilify (aripiprazole)
Abraxane (paclitaxel)
ABREVA (docosanol)
Abstral (fentanyl sublingual tablets)
Abthrax (raxibacumab)
Acarbose
Accolate
Accretropin (somatropin rDNA Original)
Aciphex (rabeprazole sodium)
Actemra (ocilizumab)
Actemra (tocilizumab)
Actiq
Activella (Estradiol/Norethindrone Acetate) Tablets
Actonel
ACTOplus met (pioglitazone hydrochloride and metformin hydrochloride)
ACTOS (pioglitazone)
Acuvail (ketorolac tromethamine)
Acyclovir Capsules
Adcetris (brentuximab vedotin)
Adcirca (tadalafil)
Adderall
Adderall XR
Addyi (flibanserin)
Adempas (riociguat)
Advicor (extended-release niacin/lovastatin)
Afinitor (everolimus)
Agenerase (amprenavir)
Aggrenox
Agrylin (anagrelide HCL)
AK-Con-A (naphazoline ophthalmic)
Akten (lidocaine hydrochloride)
Akynzeo (netupitant and palonosetron)
Alamast
Albenza (albendazole)
Aldara (imiquimod)
Aldurazyme (laronidase)
Alesse (100 mcg levonorgestrel/20 mcg ethinyl estradiol tablets)
Alimta (pemetrexed)
Alinia (nitazoxanide)
Allegra (fexofenadine hydrochloride)
Allegra-D
Alora
Aloxi (palonosetron)
Alphagan (brimonidine)
Alrex
Altabax (retapamulin)
Altocor (lovastatin)
Alvesco (ciclesonide)
Amaryl (glimepiride)
Amerge
Amevive (alefacept)
Amitiza (lubiprostone)
Amoxil (amoxicillin)
Ampyra (dalfampridine)
Amrix (cyclobenzaprine hydrochloride extended release)
Amturnide (aliskiren + amlodipine + hydrochlorothiazide)
AneuVysion Assay
Anexsia
Angiomax (bivalirudin)
Anzemet
Aphthasol
Aplenzin (bupropion hydrobromide)
Apokyn (apomorphine hydrochloride)
Apthasol (Amlexanox)
Aptiom (eslicarbazepine acetate)
Aptivus (tipranavir)
Arava
Arestin (minocycline hydrochloride)
ARICEPT (donepezil hydrochloride)
Arimidex (anastrozole)
Arixtra
Aromasin Tablets
Arranon (nelarabine)
Arthrotec
Arzerra (ofatumumab)
Asacol (mesalamine)
Atacand (candesartan cilexetil)
Atridox
Atrovent (ipratropium bromide)
Aubagio (teriflunomide)
Augmentin (amoxicillin/clavulanate)
Auryxia (Ferric citrate)
Avandamet (rosiglitazone maleate and metformin HCl)
Avandia (rosiglitazone maleate)
Avastin (bevacizumab)
Avinza (morphine sulfate)
Avycaz (ceftazidime-avibactam)
Axert (almotriptan malate) tablets
Axid AR (nizatidine)
Axona (caprylidene)
AzaSite (azithromycin)
Azor (amlodipine besylate; olmesartan medoxomil)
Azulfidine EN-tabs Tablets (sulfasalazine delayed release tablets, USP)
Banzel (rufinamide)
Baraclude (entecavir)
Baycol (cerivastatin sodium)
Bayer Extra Strength Asprin
Beleodaq (belinostat)
Belsomra (suvorexant)
Belviq (lorcaserin hydrochloride)
Benicar
Benlysta (belimumab)
Berinert (C1 Esterase Inhibitor (Human))
Betapace AF Tablet
Betaxon
Bextra
Bexxar
Biaxin XL (clarithromycin extended-release tablets)
BiDil (isosorbide dinitrate/hydralazine hydrochloride)
Boniva (ibandronate)
Bosulif (bosutinib)
Botox (onabotulinumtoxinA)
Brilinta (ticagrelor)
Brintellix (vortioxetine)
Brisdelle (low-dose paroxetine mesylate)
Bromfenac
Brovana (arformoterol tartrate)
Bunavail (buprenorphine and naloxone)
Busulflex
Byetta (exenatide)
Caduet (amlodipine/atorvastatin)
Campath
Campostar
Campral (acamprosate calcium)
Camptosar
Canasa (mesalamine)
Cancidas
Captopril and hydrochlorotiazide
Carbaglu (carglumic acid)
Carbatrol
Cedax (ceftibuten)
Ceftin (cefuroxime axetil)
Celexa
CellCept
Cenestin
Cerdelga (eliglustat)
Cernevit
Cetrotide
Chantix (varenicline)
Chloraprep (chlorhexidine gluconate)
Cholbam (cholic acid)
Cialis (tadalafil)
Cimzia (certolizumab pegol)
Cinryze (C1 Inhibitor (Human))
Cipro (ciprofloxacin HCl)
Clarinex
Clarithromycin (Biaxin)
Claritin RediTabs (10 mg loratadine rapidly-disintegrating tablet)
Claritin-D 24 Hour Extended Release Tablets (10 mg loratadine, 240 mg
pseudoephedrine sulfate)
Cleocin (clindamycin phosphate)
Cleviprex (clevidipine)
Climara
Clolar (clofarabine)
Clomipramine hydrochloride
Clonazepam
Coartem (artemether/lumefantrine)
Colazal (balsalazide disodium)
Colcrys (colchicine)
Colesevelam
Combivir
Cometriq (cabozantinib)
Complera (emtricitabine/rilpivirine/tenofovir disoproxil fumarate)
Comtan
Concerta
Confide
Contrave (naltrexone HCl and bupropion HCl)
Copaxone
Corlanor (ivabradine)
Corlopam
Cosentyx (secukinumab)
Cosopt
Covera-HS (verapamil)
Cresemba (isavuconazonium sulfate)
Crestor (rosuvastatin calcium)
Crixivan (Indinavir sulfate)
Curosurf
Cuvposa (glycopyrrolate)
Cycloset, bromocriptine mesylate
Cylert
Cymbalta (duloxetine)
Cyramza (ramucirumab)
Cystaran (cysteamine hydrochloride)
Dacogen (decitabine)
Daklinza (daclatasvir)
Daliresp (roflumilast)
Dalvance (dalbavancin)
Daptacel
Depakote (divalproex sodium)
Depakote ER (divalproex sodium)
Dermagraft-TC
Desmopressin Acetate (DDAVP)
Desonate (desonide)
Detrol (tolterodine tartrate)
Detrol LA (tolterodine tartrate)
Diabeta (glyburide)
Diclegis (doxylamine succinate + pyridoxine hydrochloride DR tablets)
Dificid (fidaxomicin)
Diltiazem HCL, Extended-Release Capsules
Diovan (valsartan)
Diovan HCT (valsartan)
Ditropan XL (oxybutynin chloride)
Doribax (doripenem)
Dostinex Tablets (cabergoline tablets)
Droxia
Duavee (conjugated estrogens/bazedoxifene)
Duetact (pioglitazone + glimepiride)
Duexis (ibuprofen and famotidine)
Dulera (mometasone furoate + formoterol fumarate dihydrate)
DuoNeb (albuterol sulfate and ipratropium bromide)
Durezol (difluprednate)
Dutasteride
Dymista (azelastine hydrochloride and fluticasone propionate)
Dynabac
DynaCirc CR
Edarbi (azilsartan medoxomil)
Edarbyclor (azilsartan medoxomil and chlorthalidone)
EDEX
Edluar (zolpidem tartrate)
Edurant (rilpivirine)
Effexor (venlafaxin HCL)
Effexor XR (venlafaxin HCI)
Efient (prasugrel)
Elaprase (idursulfase)
Elelyso (taliglucerase alfa)
Elidel
Eliquis (apixaban)
Elitek (rasburicase)
Ellence
Elmiron (pentosan polysulfate sodium)
Eloxatin (oxaliplatin/5-fluorouracil/leucovorin)
Embeda (morphine sulfate and naltrexone hydrochloride)
Emend (aprepitant)
Enbrel (etanercept)
Entereg (alvimopan)
Entocort EC (budesonide)
Entresto (sacubitril and valsartan)
Entyvio (vedolizumab)
Envarsus XR (tacrolimus extended-release)
Epanova (omega-3-carboxylic acids)
Epivir (lamivudine)
Eraxis (anidulafungin)
Erbitux (cetuximab)
Erivedge (vismodegib)
Erwinaze (asparaginase Erwinia chrysanthemi)
Esbriet (pirfenidone)
Esclim
Estradiol tablets
Estratab (.3 mg)
Estrostep (norethindrone acetate and ethinyl estradiol)
Ethyol (amifostine)
Etodolac
Eulexin (flutamide)
Evamist (estradiol)
Evista (raloxifene hydrochloride)
Evotaz (atazanavir and cobicistat)
Evoxac
Exalgo (hydromorphone hydrochloride) extended release
Excedrin Migraine
Exelon (rivastigmine tartrate)
Extina (ketoconazole)
Eylea (aflibercept)
Fabrazyme (agalsidase beta)
Famvir (famciclovir)
Fanapt (iloperidone)
Farxiga (dapagliflozin)
Farydak (panobinostat)
Faslodex (fulvestrant)
Femara (letrozole)
Femhrt Tablets
FemPatch
Femstat 3 (butoconazole nitrate 2%)
Fenofibrate
Feraheme (ferumoxytol)
Ferriprox (deferiprone)
Ferrlecit
Fetzima (levomilnacipran)
Finevin
Firazyr (icatibant)
Flagyl ER
FLOMAX
Flovent Rotadisk
Floxin Tablets (ofloxacin tablets)
Focalin (dexmethylphenidate HCl)
Fortamet
Forteo (teriparatide)
Fortovase
Fosamax (alendronate sodium)
Fosrenol (lanthanum carbonate)
Fragmin
Frova (frovatriptan succinate)
Fulyzaq (crofelemer)
Fusilev (levoleucovorin)
Fuzeon (enfuvirtide)
Fycompa (perampanel)
Galzin (zinc acetate)
GastroMARK
Gattex (teduglutide)
Gazyva (obinutuzumab)
Gelnique (oxybutynin chloride)
Gemzar (gemcitabine HCL)
Geodon (ziprasidone mesylate)
Gilenya (fingolimod)
Gilotrif (afatinib)
Gleevec (imatinib mesylate)
Glipizide Tablets
Glipizide/metformin HCl
Glucagon
Glucophage
Glucotrol (glipizide)
Glumetza
Glyburide Tablets
Glynase (micronized glyburide)
Glyset (miglitol)
Gralise (gabapentin)
Halaven (eribulin mesylate)
Harvoni (ledipasvir and sofosbuvir)
Havrix
Hepsera (adefovir dipivoxil)
Herceptin
Herceptin (trastuzumab)
Hetlioz (tasimelteon)
Horizant (gabapentin enacarbil)
Humira (adalimumab)
Hycamtin (topotecan hydrochloride)
Iamin
Ibrance (palbociclib)
Iclusig (ponatinib)
Ilaris (canakinumab)
Imagent (perflexane lipid microspheres)
Imbruvica (ibrutinib)
Imitrex (sumatriptan)
Impavido (miltefosine)
Incivek (telaprevir)
Increlex (mecasermin)
Infasurf
INFERGEN (interferon alfacon-1)
Inlyta (axitinib)
Inspra (eplerenone tablets)
Integrilin
Intelence (etravirine)
Intermezzo (zolpidem tartrate sublingual tablet)
Intuniv (guanfacine extended-release)
Invanz
Invega (paliperidone)
Invirase (saquinavir)
Invokana (canagliflozin)
Iontocaine
Iressa (gefitinib)
Isentress (raltegravir)
Istodax (romidepsin)
IvyBlock
Ixempra (ixabepilone)
Jakafi (ruxolitinib)
Jalyn (dutasteride + tamsulosin)
Janumet (sitagliptin + metformin)
Januvia (sitagliptin phosphate)
Jardiance (empagliflozin)
Jentadueto (linagliptin plus metformin hydrochloride)
Jetrea (ocriplasmin)
Jevtana (cabazitaxel)
Juvisync (sitagliptin and simvastatin)
Juxtapid (lomitapide)
Kadcyla (ado-trastuzumab emtansine)
Kadian
Kalbitor (ecallantide)
Kaletra
Kalydeco (ivacaftor)
Kapvay (clonidine hydrochloride)
Kcentra (Prothrombin Complex Concentrate)
Kengreal (cangrelor)
Keppra
Kerydin (tavaborole)
Ketek (telithromycin)
Ketoprofen
Keytruda (pembrolizumab)
Kineret
Kineret, Anakinra
Kombiglyze (saxagliptin + metformin)
Korlym (mifepristone)
Krystexxa (pegloticase)
Kuvan (sapropterin dihydrochloride)
Kybella (deoxycholic acid)
Kynamro (mipomersen sodium)
Kyprolis (carfilzomib)
Kytril (granisetron) tablets
Lamisil (terbinafine hydrochloride) Tablets
Latuda (lurasidone)
Lemtrada (alemtuzumab)
Lenvima (lenvatinib)
Lescol (fluvastatin sodium)
Lescol (fluvastatin sodium) capsules, Rx
Lescol XL (fluvastatin sodium) tablet, extended release
Letairis (ambrisentan)
Leukine (sargramostim)
Levaquin
Levitra (vardenafil)
Levo-T (levothyroxine sodium)
Levoxyl
Lexapro (escitalopram oxalate)
Lexiva (fosamprenavir calcium)
Lexxel (enalapril maleate-felodipine ER)
Linagliptin
Linzess (linaclotide)
Lipitor (atorvastatin calcium)
Liptruzet (ezetimibe and atorvastatin)
Lithobid (Lithium Carbonate)
Livalo (pitavastatin)
Lo Minastrin, (norethindrone acetate, ethinyl estradiol, ferrous fumarate)
Lodine (etodolac)
Lodine XL (etodolac)
Lotemax
Lotronex (alosetron HCL) Tablets
Lucentis (ranibizumab)
Lunesta (eszopiclone)
Lusedra (fospropofol disodium)
Lustra
LUVOX (fluvoxamine maleate)
Lynparza (olaparib)
Lyrica (pregabalin)
Lysteda (tranexamic acid)
Macugen (pegaptanib)
Malarone (atovaquone; proguanil hydrochloride) Tablet
Marplan Tablets
Mavik (trandolapril)
Maxalt
Mekinist (trametinib)
MERIDIA
Merrem I.V. (meropenem)
Mesnex
Metadate CD
Metaglip (glipizide/metformin HCl)
Metformin
Metozolv ODT (metoclopramide hydrochloride)
MetroLotion
Mevacor (lovastatin) tablets
Micardis (telmisartan)
Micardis HCT (telmisartan and hydrochlorothiazide)
Micronase (glyburide)
Microzide (hydrochlorothiazide)
Miglitol
Migranal
Miraluma test
Mirapex
Mircera (methoxy polyethylene glycol-epoetin beta)
Mircette
Mirena (levonorgestrel-releasing intrauterine system)
Mirvaso (brimonidine)
Mobic (meloxicam) Tablets
Monistat 3 (miconazole nitrate)
Monurol
Movantik (naloxegol)
Moxatag (amoxicillin)
Multaq (dronedarone)
Muse
Mylotarg (gemtuzumab ozogamicin)
Myobloc
Myozyme (alglucosidase alfa)
Myrbetriq (mirabegron)
Naglazyme (galsulfase)
Naltrexone Hydrochloride Tablets
Namenda (memantine HCl)
Namzaric (memantine hydrochloride extended-release + donepezil hydrochloride)
Naprelan (naproxen sodium)
Natazia (estradiol valerate + dienogest)
Natazia (estradiol valerate and estradiol valerate/dienogest)
Nateglinide
Natpara (parathyroid hormone)
Natrecor (nesiritide)
Nesina (alogliptin)
Neulasta
Neumega
Neupogen
Neupro (rotigotine)
Neurontin (gabapentin)
Neutroval (tbo-filgrastim)
Nexavar (sorafenib)
Nexium (esomeprazole magnesium)
Niaspan
NicoDerm CQ
Nicorette (nicotine polacrilex)
Nitrostat (nitroglycerin) Tablets
Nolvadex
NORCO tablets (Hydrocodone Bitartrate/Acetaminophen 10 mg/325 mg)
Noritate
Normiflo
Northera (droxidopa)
Norvir (ritonavir)
Novantrone (mitoxantrone hydrochloride)
Novothyrox (levothyroxine sodium)
Noxafil (posaconazole)
Nplate (romiplostim)
Nucynta (tapentadol)
Nuedexta (dextromethorphan hydrobromide and quinidine sulfate)
Nulojix (belatacept)
Nutropin (somatropin-rDNA origin)
NuvaRing
Nuvigil (armodafinil)
Nymalize (nimodipine)
OcuHist
Odomzo (sonidegib)
Ofev (nintedanib)
Oleptro (trazodone hydrochloride)
Olysio (simeprevir)
Omnicef
Omontys (peginesatide)
Onfi (clobazam)
Onglyza (saxagliptin)
Onsolis (fentanyl buccal)
Opdivo (nivolumab)
Opsumit (macitentan)
Oral Cytovene
Oralair (Sweet Vernal, Orchard, Perennial Rye, Timothy and Kentucky Blue Grass
Mixed Pollens Allergen Extract)
Oravig (miconazole)
Orbactiv (oritavancin)
Orencia (abatacept)
Orfadin (nitisinone)
Orkambi (lumacaftor and ivacaftor)
Ortho Evra
Ortho Tri-Cyclen Tablets (norgestimate/ethinyl estradiol)
Ortho-Prefest
OsmoCyte Pillow Wound Dressing
Osphena (ospemifene)
Otezla (apremilast)
Oxecta (oxycodone HCl)
Oxtellar XR (oxcarbazepine extended release)
Oxycodone and Aspirin
Oxycodone with Acetaminophen 5 mg/325 mg
OxyContin (oxycodone HCl controlled-release)
Ozurdex (dexamethasone)
Pancreaze (pancrelipase)
Patanase (olopatadine hydrochloride)
Paxil (paroxetine hydrochloride)
Paxil CR (paroxetine hydrochloride)
Pegasys (peginterferon alfa-2a)
Peg-Intron (peginterferon alfa-2b)
Pentoxifylline
Pepcid Complete
Periostat (doxycycline hyclate)
Perjeta (pertuzumab)
PhosLo
Photofrin
Pindolol
Pioglitazone
Pioglitazone + glimepiride
Pioglitazone hydrochloride + metformin hydrochloride
Plavix (clopidogrel bisulfate)
Plegridy (peginterferon beta-1a)
Pomalyst (pomalidomide)
Posicor
Potiga (ezogabine)
Pradaxa (dabigatran etexilate mesylate)
Praluent (alirocumab)
Pramipexole
Prandimet (
Prandin (repaglinide)
Pravachol (pravastatin sodium)
Precose (acarbose)
Premarin (conjugated estrogens)
Prempro
Prempro & Premphase (conjugated estrogens/medroxyprogesterone acetate tablets)
Prestalia (perindopril arginine and amlodipine besylate)
PREVACID(R) (lansopraxole)
Prevpac
Prezcobix (darunavir and cobicistat)
Prezista (darunavir)
Priftin
Prilosec (omeprazole)
Prilosec (omeprazole)/Biaxin (clarithromycin) Combination Therapy
Prinivil or Zestril (Lisinopril)
ProAmatine (midodrine)
Procanbid (procainamide hydrochloride extended-release tablets)
Prochloroperazine
Prochlorperazine
Procysbi (cysteamine bitartrate)
Prograf
Proleukin
Prolia (denosumab)
Promacta (eltrombopag)
Prometrium
Propecia
Proscar
Protonix (pantoprazole sodium) Delayed Release Tablets
Protonix (pantoprazole sodium) Delayed-Release Tablets
Protopic (tacrolimus) ointment
Provenge (sipuleucel-T)
Prozac Weekly (fluoxetine HCl)
Pulmozyme (dornase alfa)
Qsymia (phentermine + topiramate extended-release)
Quartette (levonorgestrel/ethinyl estradiol and ethinyl estradiol)
Qudexy XR (topiramate)
Quillivant XR (methylphenidate hydrochloride)
Quixin (levofloxacin)
Qutenza (capsaicin)
Qvar (beclomethasone dipropionate)
Ranexa (ranolazine)
Ranitidine Capsules
Ranitidine Tablets
Rapamune (sirolimus) Tablets
Raplon
Ravicti (glycerol phenylbutyrate)
Raxar (grepafloxacin)
Rayos (prednisone) delayed-release tablets
Rebetol (ribavirin)
REBETRON (TM) Combination Therapy
Rebif (interferon beta-1a)
Reclast (zoledronic acid)
Rectiv (nitroglycerin) ointment 0.4%
Redux (dexfenfluramine hydrochloride)
Refludan
Relenza
Relpax (eletriptan hydrobromide)
Remeron (Mirtazapine)
Remeron SolTab (mirtazapine)
Remicade (infliximab)
Reminyl (galantamine hydrobromide)
Remodulin (treprostinil)
Renagel (sevelamer hydrochloride)
RenaGelRenagel (sevelamer hydrochloride)
Renvela (sevelamer carbonate)
ReoPro
Repaglinide
Repaglinide + Metformin
Repatha (evolocumab)
Requip (ropinirole hydrochloride)
Rescriptor Tablets (delavirdine mesylate tablets)
Retavase (reteplase)
transRevlimid (lenalidomide)
Rexulti (brexpiprazole)
Reyataz (atazanavir sulfate)
Rilutek (riluzole)
Risperdal
Ritalin LA (methylphenidate HCl)
Rituxan
Rocephin
Rozerem (ramelteon)
Rytary (carbidopa and levodopa) extended-release capsules
Rythmol
Sabril (vigabatrin)
Saizen
Salagen Tablets
Samsca (tolvaptan)
Sanctura (trospium chloride)
Sancuso (granisetron)
Saphris (asenapine)
Savaysa (edoxaban)
Savella (milnacipran hydrochloride)
Saxagliptin
Saxagliptin + metformin
Seasonale, Lo Seasonale, Seasonique (ethinylestradiol + levonorgestrel)
SecreFlo (secretin)
Selegiline tablets
Selzentry (maraviroc)
Sensipar (cinacalcet)
Seprafilm
Serevent
Seroquel (R) (quetiapine fumarate) Tablets
Signifor (pasireotide diaspartate)
Signifor LAR (pasireotide)
Silenor (doxepin)
Simponi (golimumab)
Simulect
Singulair
Sirturo (bedaquiline)
Sitagliptin
Sitagliptin + metformin
Sitavig (acyclovir) buccal tablets
Sivextro (tedizolid phosphate)
Skelid (tiludronate disodium)
Soliris (eculizumab)
Somatuline Depot (lanreotide acetate)
Somavert (pegvisomant)
Sonata
Sovaldi (sofosbuvir)
Spectracef
SPORANOX (itraconazole)
Sprix (ketorolac tromethamine)
Sprycel (dasatinib)
Starlix (nateglinide)
Stavzor (valproic acid delayed release)
Stelara (ustekinumab)
Stendra (avanafil)
Stiolto Respimat (tiotropium bromide and olodaterol)
Stivarga (regorafenib)
Strattera (atomoxetine HCl)
Stribild (elvitegravir, cobicistat, emtricitabine, tenofovir disoproxil fumarate)
Striverdi Respimat (olodaterol)
Stromectol (ivermectin)
Subutex/Suboxone (buprenorphine/naloxone)
Sulfamylon
Supartz
Supprelin LA (histrelin acetate)
Surfaxin (lucinactant)
Sustiva
Sutent (sunitinib malate)
Sutent (sunitinib)
Sylatron (peginterferon alfa-2b)
Sylvant (siltuximab)
Symlin (pramlintide)
Synagis
Synercid I.V.
Synjardy (empagliflozin and metformin hydrochloride)
Synribo (omacetaxine mepesuccinate)
Synthroid (levothyroxine sodium)
Synvisc, Synvisc-One (Hylan GF 20)
Tafinlar (dabrafenib)
Tamiflu capsule
Tanzeum (albiglutide)
Tarceva (erlotinib, OSI 774)
Targiniq ER (oxycodone hydrochloride + naloxone hydrochloride) extended-release
tablets
Tasigna (nilotinib hydrochloride monohydrate)
Tasmar
Tavist (clemastine fumarate)
Taxol
Taxotere (Docetaxel)
Tecfidera (dimethyl fumarate)
Technivie, (ombitasvir, paritaprevir and ritonavir)
Teczem (enalapril maleate/diltiazem malate)
Teflaro (ceftaroline fosamil)
Tegretol (carbamazepine)
Tegretol XR (carbamazepine)
Tekamlo (aliskiren + amlodipine)
Tekturna (aliskiren)
Temodar
Tequin
Testim
Testoderm TTS CIII
Teveten (eprosartan mesylate plus hydrochlorothiazide)
Teveten (eprosartan mesylate)
Thalomid
Tiazac (diltiazem hydrochloride)
Tikosyn Capsules
Tilade (nedocromil sodium)
Timentin
Tindamax, tinidazole
Tivicay (dolutegravir)
Tivorbex (indomethacin)
Tobi
Tolmetin Sodium
Topamax (topiramate)
Toprol-XL (metoprolol succinate)
Torisel (temsirolimus)
Toviaz (fesoterodine fumarate)
Tracleer (bosentan)
Tradjenta (linagliptin)
Trazadone 150 mg
Treanda (bendamustine hydrochloride)
Trelstar Depot (triptorelin pamoate)
Trelstar LA (triptorelin pamoate)
Tribenzor (olmesartan medoxomil + amlodipine + hydrochlorothiazide)
Tricor (fenofibrate)
Trileptal (oxcarbazepine) Tablets
Trilipix (fenofibric acid)
Trisenox (arsenic trioxide)
Triumeq (abacavir, dolutegravir, and lamivudine)
Trivora-21 and Trivora-28
Trizivir (abacavir sulfate; lamivudine; zidovudine AZT) Tablet
Trokendi XR (topiramate)
Trovan
Trulicity (dulaglutide)
Twinrix
Tygacil (tigecycline)
Tykerb (lapatinib)
Tysabri (natalizumab)
Tyvaso (treprostinil)
Tyzeka (telbivudine)
Uceris (budesonide)
Uloric (febuxostat)
Ultracet (acetaminophen and tramadol HCl)
UltraJect
Ultresa (pancrelipase) delayed-release capsules
Unituxin (dinutuximab)
UroXatral (alfuzosin HCl extended-release tablets)
Urso
Valcyte (valganciclovir HCl)
Valstar
Valtrex (valacyclovir HCl)
Vancenase AQ 84 mcg Double Strength
Vandetanib (vandetanib)
Vaprisol (conivaptan)
VariZIG, Varicella Zoster Immune Globulin (Human)
Varubi (rolapitant)
Vascepa (icosapent ethyl)
Vectibix (panitumumab)
Velcade (bortezomib)
Veltin (clindamycin phosphate and tretinoin)
Veramyst (fluticasone furoate)
Verapamil
Verdeso (desonide)
Veregen (kunecatechins)
VERSED (midazolam HCI)
Vesicare (solifenacin succinate)
Vfend (voriconazole)
Viagra
Vibativ (telavancin)
Viberzi (eluxadoline)
Victoza (liraglutide)
Victrelis (boceprevir)
Vidaza (azacitidine)
Videx (didanosine)
Viekira Pak (ombitasvir, paritaprevir, ritonavir and dasabuvir) tablets
Viibryd (vilazodone hydrochloride)
Vimizim (elosulfase alfa)
Vimovo (naproxen + esomeprazole)
Vimpat (lacosamide)
Viokace (pancrelipase) tablets
Vioxx (rofecoxib)
VIRACEPT (nelfinavir mesylate)
Viramune (nevirapine)
Viread (tenofovir disoproxil fumarate)
Viroptic
Visicol Tablet
Visipaque (iodixanol)
Vistide (cidofovir)
Voraxaze (glucarpidase)
Votrient (pazopanib)
Vyvanse (Lisdexamfetamine Dimesylate)
Warfarin Sodium tablets
Welchol (colesevelam hydrochloride)
Xalkori (crizotinib)
Xarelto (rivaroxaban)
Xartemis XR (oxycodone hydrochloride and acetaminophen) extended release
Xeljanz (tofacitinib)
Xeloda
Xenazine (tetrabenazine)
Xenical/Orlistat Capsules
Xeomin (incobotulinumtoxinA)
Xgeva (denosumab)
Xiaflex (collagenase clostridium histolyticum)
Xifaxan (rifaximin)
Xigduo XR (dapagliflozin + metformin hydrochloride)
Xigris (drotrecogin alfa [activated])
Xofigo (radium Ra 223 dichloride)
Xolair (omalizumab)
Xopenex
Xtandi (enzalutamide)
Xyzal (levocetirizine dihydrochloride)
Yasmin (drospirenone/ethinyl estradiol)
Yervoy (ipilimumab)
ZADITOR
Zagam (sparfloxacin) tablets
Zaltrap (ziv-aflibercept)
Zanaflex (tizanidine hydrochloride)
Zantac 75 Efferdose
Zelboraf (vemurafenib)
Zelnorm (tegaserod maleate) Tablets
Zemaira (alpha1-proteinase inhibitor)
Zemplar
Zenapax
Zenpep (pancrelipase)
Zerbaxa (ceftolozane + tazobactam)
Zerit (stavudine)
Zevalin (ibritumomab tiuxetan)
Zingo (lidocaine hydrochloride monohydrate)
Ziprasidone (ziprasidone hydrochloride)
Zipsor (diclofenac potassium)
Zithromax (azithromycin)
Zocor
Zofran
Zohydro ER (hydrocodone bitartrate) Extended-Release Capsules
Zoloft (sertraline HCl)
Zometa (zoledronic acid)
Zomig (zolmitriptan)
Zonegran (zonisamide) Capsules
Zontivity (vorapaxar)
Zortress (everolimus)
Zosyn (sterile piperacillin sodium/tazobactam sodium)
Zubsolv (buprenorphine and naloxone)
Zyban Sustained-Release Tablets
Zyclara (imiquimod)
Zydelig (idelalisib)
Zyflo (Zileuton)
Zykadia (ceritinib)
Zyprexa
Zyrtec (cetirizine HCl)
Zytiga (abiraterone acetate)

Each drug's side effects are listed in the drug's package insert. Each side effect can be tracked using the respective Patient Self-Assessment, Patient Self-Test, and/or Patient Self-Report screens or digitally captured diagnostic, monitoring, or patient maintained information. Table 4 is an exemplary listing of drugs and their side effects that can be tracked by the prescriber using a selection on the prescription or other input media that enables a data capture screen to be incorporated into the Drug Specific App. The information may or may not be incorporated into the Patient Specific Dispensing Algorithm. The Drug Specific App can be customized to request the information each time or at specified intervals. This allows the Patient Drug Specific App to be tailored to each patient to assist in patient management. The drug specific side effects for each drug are listed in each drug's package insert and are hereby incorporated by reference.

TABLE 4
Sample Drugs and Their Side Effects

| | |
|---|---|
| Afinitor | Common: |
| | Cardiovascular: Hypertension (tumors, 4% to 13%; kidney |
| | transplant, 30%; liver transplant, 17%), Peripheral edema (tumors, |
| | 13% to 39%; kidney transplant, 45%; liver transplant, 18%) |
| | Dermatologic: Acne (tumors, 10% to 22%; transplant, 1% to less |
| | than 10%), Eczema (renal angiomyolipoma, 10%), Rash (tumors, |
| | 5% to 59%) |
| | Endocrine metabolic: Dyslipidemia (kidney transplant, 15%), |
| | Hypercholesterolemia (tumors, 66% to 85%; kidney transplant, |
| | 17%), Hyperlipidemia (kidney transplant, 21%; liver transplant, |
| | 24%), Hypertriglyceridemia (tumors, 27% to 73%), |
| | Hypoalbuminemia (breast cancer, 33%), Hypophosphatemia |
| | (tumors, 9% to 49%; kidney transplant, 13%), Increased glucose |
| | level, All grades (tumors, 14% to 75%; kidney transplant, 12%) |
| | Gastrointestinal: Constipation (tumors, 10% to 14%; kidney |
| | transplant, 38%), Decrease in appetite (tumors, 6% to 30%; |
| | transplant, 1% to less than 10%), Diarrhea (tumors, 14% to 50%; |
| | kidney transplant, 19%; liver transplant, 19%), Nausea (tumors, |
| | 16% to 29%; kidney transplant, 29%; liver transplant, 14%), |
| | Stomatitis (tumors, 44% to 78%; kidney transplant, 8%), Vomiting |
| | (tumors, 15% to 29%; kidney transplant, 15%) |
| | Hematologic: Anemia, All Grades (tumors, 41% to 86%; kidney |
| | transplant, 26%), Decreased lymphocyte count, all grades (tumors, |
| | 20% to 54%), Partial thromboplastin time increased (subependymal |
| | giant cell astrocytoma, 72%), Thrombocytopenia, All grades |
| | (tumors, 19% to 54%; transplant, up to 10%) |
| | Hepatic: Alkaline phosphatase raised (tumors, 32% to 74%; |
| | transplant, 1% to less than 10%), ALT/SGPT level raised (tumors, |
| | 18% to 51%), AST/SGOT level raised (tumors, 23% to 69%) |
| | Immunologic: Impaired wound healing (kidney transplant, 35%; |
| | liver transplant, 11%) |
| | Neurologic: Asthenia (tumors, 13% to 33%) |
| | Otic: Otitis media (renal angiomyolipoma, 6%) |
| | Psychiatric: Mental disorder (subependymal giant cell |
| | astrocytoma, 21%) |
| | Renal: Serum creatinine raised (tumors, 19% to 50%; kidney |
| | transplant, 18%), Urinary tract infectious disease (tumors, 5% to |
| | 16%; kidney transplant, 22%) |
| | Reproductive: Amenorrhea (renal angiomyolipoma, 15%), |
| | Irregular periods (tumors, 10% to 11%), Menorrhagia (renal |
| | angiomyolipoma, 10%) |
| | Respiratory: Cough (tumors, 20% to 30%; kidney transplant, 7%), |
| | Dyspnea (tumors, 20% to 24%), Sinusitis (tumors, 3% to 6%), |
| | Upper respiratory infection (tumors, 11% to 31%; kidney |
| | transplant, 16%) |
| | Other: Fatigue (tumors, 14% to 45%; kidney transplant, 9%), |
| | Fever (tumors, 15% to 31%; kidney transplant, 19%; liver |
| | transplant, 13%) |
| | Serious: |
| | Cardiovascular: Pericardial effusion (Transplant, less than 1%) |
| | Hematologic: Anemia, Grade 3 or 4 (tumors, 6.6% to 15%), |
| | Decreased lymphocyte count, Grade 3 or 4 (tumors, 1% to 16%), |
| | Hemorrhage (renal cell carcinoma, 3%), Leukopenia (tumors, 37% |
| | to 58%; kidney transplant, 3%; liver transplant, 12%), |
| | Pancytopenia, All grades (transplant, 1% to less than 10%), |
| | Thrombosis, Thrombotic microangiopathy (Transplant, less than |
| | 1%), Thrombotic thrombocytopenic purpura (Transplant, less than |
| | 1%), Venous thromboembolism (transplant, 1% to less than 10%) |
| | Immunologic: Infectious disease (tumors, 37% to 50%; kidney |
| | transplant, 62%; liver transplant, 50%) |
| | Neurologic: Seizure (renal angiomyolipoma, 5%) |
| | Renal: Hemolytic uremic syndrome (Transplant, less than 1%), |
| | Renal failure (renal cell carcinoma, 3%), Thrombosis of renal |
| | artery (transplant, 1% to less than 10%) |
| | Respiratory: Interstitial lung disease (Less than 1%), Non- |
| | infectious pneumonia (Up to 19%), Pleural effusion (tumors, 7%), |
| | Pneumocystis pneumonia, Pneumonia (renal cell carcinoma, 6%), |
| | Pulmonary embolism (PNET, 2.5%; transplant, 1% to less than |
| | 10%) |
| | Other: Angioedema (Transplant, up to 6.8%), Sepsis (tumors less |
| | than 1%; transplant, 1% to less than 10%) |
| Ampyra | Common |
| | Gastrointestinal: Abdominal pain (7%), Nausea (7% to 13%), |
| | Vomiting (13%) |
| | Musculoskeletal: Abnormal gait (5%), Backache (5%) |
| | Neurologic: Asthenia (7%), Dizziness (7%), Headache (7%), |
| | Insomnia (9%) |
| | Psychiatric: Anxiety (5%) |
| | Renal: Urinary tract infectious disease (12%) |
| | Serious |
| | Immunologic: Anaphylaxis, Hypersensitivity reaction |
| | Neurologic: Seizure |
| Angeliq | Common |
| | Gastrointestinal: Abdominal pain (3.6% to 6.5%) |
| | Neurologic: Headache (6%) |
| | Reproductive: Abnormal vaginal bleeding (3.6% to 14%), Pain of |
| | breast (3.3% to 17.9%) |
| | Serious |
| | Cardiovascular: Myocardial infarction |
| | Gastrointestinal: Disorder of gallbladder |
| | Hematologic: Deep venous thrombosis |
| | Neurologic: Cerebrovascular accident |
| | Ophthalmic: Thrombosis of retinal vein |
| | Reproductive: Breast cancer, Endometrial carcinoma, Ovarian |
| | cancer |
| | Respiratory: Pulmonary embolism |
| Brisdelle | Common |
| | Dermatologic: Diaphoresis (1% to 14%) |
| | Gastrointestinal: Constipation (4.9% to 16%), Diarrhea (7.9% to |
| | 19.2%), Loss of appetite (2% to 9%), Nausea (up to 36.3%), |
| | Xerostomia (10.8% to 20.6%) |
| | Neurologic: Asthenia (2.9% to 22%), Dizziness (6% to 14%), |
| | Headache (psychiatric conditions, 17% to 18%; menopausal |
| | vasomotor symptoms, 6.3%), Insomnia (11% to 24%), Somnolence |
| | (12.7% to 24%), Tremor (up to 14.7%) |
| | Ophthalmic: Blurred vision (2% to 7.8%) |
| | Reproductive: Abnormal ejaculation (5.8% to 28%), Disorder of |
| | female genital organs (2% to 9%), Erectile dysfunction (1.9% to |
| | 9%), Reduced libido (males, 6% to 15%; females, 0% to 9%) |
| | Serious |
| | Psychiatric: Depression, Exacerbation, Suicidal thoughts, Suicide |
| | Other: Serotonin syndrome |
| Contrave | Common: |
| | Gastrointestinal: Constipation (19.2%), Diarrhea (7.1%), Nausea |
| | (32.5%), Vomiting (10.7%), Xerostomia (8.1%) |
| | Neurologic: Dizziness (9.9%), Headache (17.6%), Insomnia (9.2%) |
| | Psychiatric: Anxiety (4.2%) |
| | Serious: |
| | Cardiovascular: Hypertension (3.2%), Increased heart rate, |
| | Myocardial infarction (Less than 2%) |
| | Dermatologic: Erythema multiforme (Rare), Stevens-Johnson |
| | syndrome (Rare) |
| | Endocrine metabolic: Hypoglycemia |
| | Gastrointestinal: Cholecystitis (Less than 2%), Hematochezia (Less |
| | than 2%) |
| | Hepatic: Hepatotoxicity |
| | Immunologic: Anaphylaxis, Delayed hypersensitivity disorder |
| | Musculoskeletal: Intervertebral disc prolapse (Less than 2%) |
| | Neurologic: Amnesia (Less than 2%), Seizure (0.1%) |
| | Ophthalmic: Angle-closure glaucoma |
| | Psychiatric: Depression (6.3% to 7.1%), Mania, Psychiatric |
| | symptom, Suicidal thoughts (0.03%) |
| | Renal: Infectious disorder of kidney (Less than 2%), Serum |
| | creatinine raised (Less than 2%) |
| | Respiratory: Pneumonia (Less than 2%) |
| Daliresp | Common: |
| | Endocrine metabolic: Weight decreased (7% to 20%) |
| | Gastrointestinal: Decrease in appetite (2.1%), Diarrhea (9.5%), |
| | Nausea (4.7%) |
| | Immunologic: Influenza (2.8%) |
| | Musculoskeletal: Backache (3.2%) |
| | Neurologic: Dizziness (2.1%), Headache (4.4%), Insomnia (2.4%) |
| | Serious: |
| | Psychiatric: Suicidal thoughts |
| Doryx | Common: |
| | Dermatologic: Rash (4%) |
| | Gastrointestinal: Diarrhea (3.3%), Loss of appetite, Nausea (8% to |
| | 13.4%), Sensitive dentin, Sore gums, Vomiting (8.1%) |
| | Musculoskeletal: Myalgia (6.4%) |
| | Reproductive: Bacterial vaginosis (3.3%) |
| | Serious: |
| | Dermatologic: Drug hypersensitivity syndrome, Erythema |
| | multiforme, Photosensitivity, Stevens-Johnson syndrome, Toxic |
| | epidermal necrolysis |
| | Gastrointestinal: Clostridium difficile diarrhea |
| | Hepatic: Hepatotoxicity (Rare) |
| | Immunologic: Anaphylaxis, Superinfection |
| | Musculoskeletal: Arrest of bone development AND/OR growth |
| | Neurologic: Pseudotumor cerebri |
| Ella | Common: |
| | Gastrointestinal: Abdominal pain (8% to 15%), Nausea (12% to |
| | 13%) |
| | Neurologic: Headache (18% to 19%) |
| Equetro | Common |
| | Cardiovascular: Hypotension |
| | Dermatologic: Pruritus (8%), Rash (7%) |
| | Gastrointestinal: Constipation (10%), Nausea (29%), Vomiting |
| | (18%), Xerostomia (8%) |
| | Neurologic: Asthenia (8%), Ataxia (15%), Dizziness (44%), |
| | Somnolence |
| | Ophthalmic: Blurred vision (6%), Nystagmus |
| | Serious |
| | Cardiovascular: Atrioventricular block, Cardiac dysrhythmia, |
| | Congestive heart failure, Eosinophilic myocarditis, |
| | Hypersensitivity, Syncope |
| | Dermatologic: Stevens-Johnson syndrome, Toxic epidermal |
| | necrolysis |
| | Endocrine metabolic: Hypocalcemia, Hyponatremia (4% to 21.7%), |
| | Water intoxication syndrome |
| | Gastrointestinal: Pancreatitis |
| | Hematologic: Agranulocytosis, Aplastic anemia, Bone marrow |
| | depression, Eosinophilia, Leukopenia, Pancytopenia, |
| | Thrombocytopenia |
| | Hepatic: Hepatitis, Hepatotoxicity, Liver failure, Vanishing bile |
| | duct syndrome |
| | Immunologic: Drug hypersensitivity syndrome |
| | Neurologic: Acute intermittent porphyria |
| | Renal: Azotemia, Renal failure |
| | Respiratory: Pulmonary hypersensitivity |
| | Other: Angioedema |
| Gabitril | Common: |
| | Dermatologic: Pruritus (2%) |
| | Gastrointestinal: Abdominal pain (5% to 7%), Nausea (11%) |
| | Neurologic: Asthenia (18% to 23%), Ataxia (5% to 9%), Confusion |
| | (5%), Disturbance in speech (4%), Dizziness (27 to 31%), Feeling |
| | nervous (10% to 14%), Insomnia (5% to 6%), Somnolence (18% to |
| | 21%), Tremor (9% to 21%), Unable to concentrate (6% to 14%) |
| | Respiratory: Pharyngitis (7% to 8%) |
| | Serious: |
| | Dermatologic: Stevens-Johnson syndrome |
| | Neurologic: Seizure, in patients without epilepsy, Status |
| | epilepticus, Status epilepticus, in patients without a history of |
| | seizure |
| | Psychiatric: Suicidal thoughts |
| Gilenya | Common: |
| | Gastrointestinal: Abdominal pain (11%), Diarrhea (13%) |
| | Hepatic: Increased liver enzymes (All elevations |
| | (ALT/GGT/AST), 15%; up to 3 times ULN (ALT, AST), 14%; 5 |
| | times ULN or greater (ALT, AST), 4.5%) |
| | Immunologic: Influenza (11%) |
| | Musculoskeletal: Backache (10%), Pain, In Extremity (10%) |
| | Neurologic: Headache (25%) |
| | Respiratory: Cough (12%), Sinusitis (11%) |
| | Serious: |
| | Cardiovascular: Atrioventricular block (up to 4.7%), |
| | Bradyarrhythmia (3%.) |
| | Dermatologic: Malignant melanoma |
| | Hematologic: Lymphocytopenia (Severe) (7%) |
| | Immunologic: Cryptococcosis, Herpesvirus infection (9%), |
| | Infectious disease (All infections, 72%; serious infections, 2.3%) |
| | Neurologic: Cryptococcal meningitis, Posterior reversible |
| | encephalopathy syndrome, Progressive multifocal |
| | leukoencephalopathy |
| | Ophthalmic: Macular retinal edema (0.5% to 1.5%) |
| Gleevec | Common: |
| | Cardiovascular: Edema |
| | Dermatologic: Night sweats (13% to 17%), Rash (Adult, 8.9% to |
| | 38.1%; pediatric, acute lymphocytic leukemia, grade 3 or 4, 4%) |
| | Endocrine metabolic: Weight increased (5% to 32%) |
| | Gastrointestinal: Diarrhea (Adult, 25% to 59.3%; pediatric, acute |
| | lymphoblastic leukemia, grade 3 or 4, 9%), Nausea (Adults, 41.7% |
| | to 73%; pediatric, acute lymphoblastic leukemia, grade 3 or 4, |
| | 16%), Vomiting (10.8% to 58%) |
| | Musculoskeletal: Arthralgia (8.8% to 40%), Cramp (28% to 62%), |
| | Musculoskeletal pain (Chronic myeloid leukemia, 20.5% to 49%), |
| | Myalgia (Adult, 9% to 33.2%; pediatric, acute lymphoblastic |
| | leukemia grade 3 or 4, 5%), Spasm (16.3% to 49%) |
| | Neurologic: Asthenia (12% to 21%), Dizziness (4.6% to 16%), |
| | Headache (8.2% to 36%), Insomnia (9.8% to 14%) |
| | Respiratory: Cough (13% to 27%), Nasopharyngitis (1% to 30.5%), |
| | Pain, Pharyngolaryngeal (Chronic myeloid leukemia, 18.1%), |
| | Pharyngitis (Chronic myeloid leukemia, 10% to 15%) |
| | Other: Fatigue (20% to 57%), Fever (6.2% to 41%), Influenza |
| | (Chronic myeloid leukemia, 0.8% to 13.8%), Rigor (10% to 12%) |
| | Serious: |
| | Cardiovascular: Cardiac tamponade, Cardiogenic shock, |
| | Congestive heart failure (0.1% to 1%) |
| | Dermatologic: Bullous eruption (0.1% to 1%), Erythema |
| | multiforme (0.01% to 0.1%), Stevens-Johnson syndrome (0.01% to |
| | 0.1%), Toxic epidermal necrolysis |
| | Gastrointestinal: Gastrointestinal perforation, Pancreatitis (0.1% to |
| | 1%) |
| | Hematologic: Anemia (Up to 42%), Febrile neutropenia (1% to |
| | 10%), Hemorrhage (All grades, 1% to 53%; grade 3 or 4, 0% to |
| | 19%), Neutropenia (Grade 3 or 4, 3.1% to 64%), Pancytopenia (1% |
| | to 10%), Thrombocytopenia (Chronic myeloid leukemia (CML), |
| | grade 3, 8.5% to 30%; CML, grade 4, up to 33%; |
| | dermatofibrosarcoma protuberans (oral route): 17%) |
| | Hepatic: ALT/SGPT level raised (grade 3 and above, up to 7%), |
| | Ascites (0.1% to 1%), AST/SGOT level raised, Hepatic necrosis |
| | (0.01% to 0.1%), Hepatotoxicity (chronic myeloid leukemia: all |
| | grades, 6% to 12%), Liver failure (0.01% to 0.1%) |
| | Neurologic: Cerebral edema, Raised intracranial pressure (0.01% to |
| | 0.1%) |
| | Ophthalmic: Optic disc edema (0.01% to 0.1%) |
| | Otic: Sensorineural hearing loss |
| | Renal: Acute renal failure (0.1% to 1%) |
| | Respiratory: Acute respiratory failure, Hypoxia (Pediatrics, acute |
| | lymphoblastic leukemia, grade 3 or 4, 9%), Pleural effusion |
| | (Pediatrics, acute lymphoblastic leukemia, grade 3 or 4, 7%), |
| | Pneumonia (Chronic myeloid leukemia, 4% to 13%), Pneumonitis |
| | (Pediatric, acute lymphoblastic leukemia, grade 3 or 4, 8%) |
| | Other: Secondary malignant neoplastic disease, Tumor lysis |
| | syndrome |
| Hysingla ER | Common: |
| | Cardiovascular: Peripheral edema (1% to less than 5%%) |
| | Dermatologic: Pruritus (0% to less than 5%) |
| | Gastrointestinal: Abdominal pain (1% to less than 5%), |
| | Constipation (3% to 11%), Nausea (7% to 10%), Vomiting (3% to |
| | 6%), Xerostomia (1% to less than 5%) |
| | Musculoskeletal: Spasm (1% to less than 5%) |
| | Neurologic: Dizziness (2% to 3%), Headache (2% to 4%), |
| | Somnolence (1% to 5%), Tremor (3%) |
| | Renal: Urinary tract infectious disease (1% to 5%) |
| | Respiratory: Upper respiratory infection (1% to 3%) |
| | Other: Fatigue (1% to 4%) |
| | Serious: |
| | Cardiovascular: Hypotension (less than 1%), Orthostatic |
| | hypotension (less than 1%), Prolonged QT interval, Syncope |
| | Gastrointestinal: Difficulty swallowing (less than 1%) |
| | Neurologic: Raised intracranial pressure, Seizure |
| | Respiratory: Respiratory depression |
| | Other: Drug withdrawal syndrome in neonate of dependent mother, |
| | Opioid withdrawal (less than 1%) |
| Kuvan | Common: |
| | Gastrointestinal: Diarrhea (4% or more), Vomiting (4% or more) |
| | Neurologic: Headache (4% or greater) |
| | Respiratory: Cough (4% or more), Nasal congestion (4% or more), |
| | Nasal discharge (4% or more), Pain in throat (4% or more), Upper |
| | respiratory infection (17%) |
| | Serious: |
| | Cardiovascular: Myocardial infarction |
| | Gastrointestinal: Gastrointestinal hemorrhage |
| | Hematologic: Hemorrhage, Post-procedural |
| | Neurologic: Seizure |
| | Respiratory: Respiratory failure |
| Lamictal XR | Common: |
| | Dermatologic: Rash (7% to 14%) |
| | Gastrointestinal: Abdominal pain (immediate-release, 5% to 10%), |
| | Diarrhea (immediate-release, 6% to 11%; extended-release, 5%), |
| | Indigestion (immediate-release, 2% to 7%), Nausea (immediate- |
| | release, 7% to 25%; extended-release, 7%), Vomiting (immediate- |
| | release, 5% to 20%; extended-release, 6%)) |
| | Neurologic: Asthenia (immediate-release, 2% to 8%; extended- |
| | release, 6%), Ataxia (immediate-release, 2% to 11%), Coordination |
| | problem (immediate-release, 6% to 7%; extended-release, 3%), |
| | Dizziness (immediate-release, 7% to 54%; extended release, 14%), |
| | Headache (immediate-release, 29%), Insomnia (immediate-release, |
| | 5% to 10%), Somnolence (immediate-release, 9% to 17%; |
| | extended-release, 5%), Tremor (immediate-release, 4% to 10%; |
| | extended-release, 6%), Vertigo (immediate-release, 2%; extended- |
| | release, 3%) |
| | Ophthalmic: Blurred vision (immediate-release, 11% to 25% |
| | (adults) and 4% (children); extended-release, 3%), Diplopia |
| | (immediate-release, 24% to 49% (adults) and 5% (children); |
| | extended-release, 5%) |
| | Psychiatric: Anxiety (immediate-release, 4%; extended-release, |
| | 3%), Depression (immediate-release, 4%; extended-release, 3%) |
| | Reproductive: Dysmenorrhea (immediate-release, 5% to 7%) |
| | Respiratory: Rhinitis (immediate-release, 7% to 14%) |
| | Other: Pain (immediate-release, 5%) |
| | Serious: |
| | Dermatologic: Erythema multiforme (less than 0.1%), Rash, |
| | Serious (0.08% to 0.8%), Stevens-Johnson syndrome (0.08% to |
| | 0.8%.), Toxic epidermal necrolysis (0.08% to 0.8%) |
| | Hematologic: Anemia (immediate release, less than 0.1%), |
| | Disseminated intravascular coagulation, Eosinophilia (immediate |
| | release, less than 0.1%), Leukopenia (immediate release, 0.1% to |
| | 1%), Thrombocytopenia (immediate release, less than 0.1%) |
| | Hepatic: Liver failure |
| | Immunologic: Drug hypersensitivity syndrome |
| | Neurologic: Aseptic meningitis |
| | Other: Angioedema (less than 0.1%), Neuroleptic malignant |
| | syndrome |
| Latuda | Common: |
| | Gastrointestinal: Diarrhea (3% to 5%), Nausea (10% to 17%), |
| | Vomiting (2% to 8%) |
| | Neurologic: Akathisia (5.6% to 22%), Extrapyramidal disease |
| | (10% to 39%), Parkinsonism (5% to 17%), Somnolence (7.3% to |
| | 26.5%) |
| | Psychiatric: Anxiety (4% to 5%) |
| | Serious: |
| | Cardiovascular: Orthostatic hypotension (0.3% to 2.1%), Syncope |
| | (0.1%) |
| | Hematologic: Agranulocytosis |
| | Neurologic: Cerebrovascular accident (0.1% to 1%), Seizure (less |
| | than 1%), Tardive dyskinesia, Transient ischemic attack |
| | Psychiatric: Suicidal thoughts (0.4% to 1.1%) |
| | Renal: Serum creatinine raised (2% to 4%) |
| | Other: Neuroleptic malignant syndrome |
| Minastrin 24 FE | Common: |
| | Dermatologic: Acne (2.7%) |
| | Endocrine metabolic: Abnormal weight gain (2%) |
| | Gastrointestinal: Nausea (4.6%), Vomiting (2% to 6%) |
| | Neurologic: Headache (6.3%) |
| | Psychiatric: Mood swings (2.2%) |
| | Reproductive: Abnormal cervical smear (3.1%), Amenorrhea (22% |
| | to 36%), Bacterial vaginosis (3.1%), Breast tenderness (3.4%), |
| | Candida vaginitis (6.1%), Intermenstrual bleeding - irregular (24 to |
| | 35%), Menstrual cramp (4.4%) |
| | Serious: |
| | Cardiovascular: Myocardial infarction |
| | Hematologic: Arterial thrombosis, Venous thromboembolism |
| | Hepatic: Adenoma of liver, Liver carcinoma |
| | Neurologic: Cerebrovascular accident |
| | Ophthalmic: Thrombosis of retinal vein |
| Nexavar | Common: |
| | Cardiovascular: Hypertension, All grades (19.1%) |
| | Dermatologic: Acral erythema (hepatocellular carcinoma, 21%; |
| | renal cell carcinoma, 30%; thyroid carcinoma, 69%), Alopecia |
| | (hepatocellular carcinoma, 14%; renal cell carcinoma, 27%; thyroid |
| | carcinoma, 67%), Peeling of skin, Rash (up to 35%) |
| | Endocrine metabolic: Hypoalbuminemia (hepatocellular carcinoma, |
| | 59%), Hypocalcemia (hepatocellular carcinoma, 27%; renal cell |
| | carcinoma, 12%; thyroid carcinoma, 36%), Hypophosphatemia |
| | (35% to 45%), Raised TSH level (thyroid carcinoma, 41%), Weight |
| | decreased (hepatocellular carcinoma, 30%; renal cell carcinoma, |
| | 10%; thyroid carcinoma, 49%) |
| | Gastrointestinal: Abdominal pain (renal cell carcinoma, 11%; |
| | hepatocellular carcinoma, 31%; thyroid carcinoma, 20%), Decrease |
| | in appetite (thyroid carcinoma, 30%), Diarrhea (43% to 68%), |
| | Increased serum lipase level (40% to 41%), Loss of appetite (16% |
| | to 29%), Nausea (21% to 24%), Serum amylase raised (30% to |
| | 34%) |
| | Hematologic: Lymphocytopenia (renal cell carcinoma, 23%; |
| | hepatocellular carcinoma, 47%), Thrombocytopenia (renal cell |
| | carcinoma; 12%; hepatocellular carcinoma, 46%) |
| | Hepatic: ALT/SGPT level raised, all grades (thyroid carcinoma, |
| | 59%), AST/SGOT level raised, All grades (thyroid carcinoma, |
| | 54%) |
| | Immunologic: Infectious disease (10% or greater) |
| | Other: Fatigue (37% to 46%), Pain (10% or greater) |
| | Serious: |
| | Cardiovascular: Congestive heart failure (1.9%), Hypertension, |
| | Grade 3 or 4 (4.3%), Hypertensive crisis (0.1% to less than 1%), |
| | Myocardial infarction, Myocardial ischemia, Prolonged QT interval |
| | (Less than 0.1%) |
| | Dermatologic: Squamous cell carcinoma of skin (thyroid |
| | carcinoma, 3%), Stevens-Johnson syndrome, Toxic epidermal |
| | necrolysis |
| | Gastrointestinal: Gastrointestinal hemorrhage, Gastrointestinal |
| | perforation (0.1% to less than 1%), Pancreatitis (0.1% to less than |
| | 1%) |
| | Hematologic: Hemorrhage (renal cell carcinoma, 15.3%; thyroid |
| | carcinoma, 17.4%) |
| | Hepatic: ALT/SGPT level raised, Grade 3 or higher (thyroid |
| | carcinoma, 4%), AST/SGOT level raised, Grade 3 or 4 (thyroid |
| | carcinoma, 2%), Hepatitis (less than 0.1%) |
| | Neurologic: Cerebral hemorrhage (0.1% to less than 1%), Posterior |
| | reversible encephalopathy syndrome (0.1% to less than 1%) |
| | Respiratory: Interstitial lung disease (0.1% to less than 1%), |
| | Respiratory tract hemorrhage |
| Noxafil | Common: |
| | Endocrine metabolic: Hypokalemia (prophylaxis, 22% to 30%) |
| | Gastrointestinal: Diarrhea (prophylaxis, 29% to 42%; |
| | oropharyngeal candidiasis, 10%; refractory oropharyngeal |
| | candidiasis, 13%), Nausea (prophylaxis, 19% to 38%; |
| | oropharyngeal candidiasis, 9%; refractory oropharyngeal |
| | candidiasis, 29%), Vomiting (prophylaxis, 12% to 29%; |
| | oropharyngeal candidiasis, 7%; refractory oropharyngeal |
| | candidiasis, 28%) |
| | Neurologic: Headache (prophylaxis, 14% to 28%; oropharyngeal |
| | candidiasis, 8%; refractory oropharyngeal candidiasis, 20%) |
| | Other: Fever (prophylaxis, 21% to 45%; oropharyngeal candidiasis, |
| | 6%; refractory oropharyngeal candidiasis, 34%) |
| | Serious: |
| | Cardiovascular: Prolonged QT interval (1% to 2%), Torsades de |
| | pointes (less than 5%) |
| | Hepatic: Cholestasis (rare), Liver failure (rare) |
| Pradaxa | Common: |
| | Gastrointestinal: Esophagitis, Gastritis, Gastroesophageal reflux |
| | disease (Atrial fibrillation, 5.5%), Gastrointestinal hemorrhage |
| | (DVT and pulmonary embolism, 0.7% to 3.1%; nonvalvular atrial |
| | fibrillation, 6.1%), Gastrointestinal ulcer, Indigestion (DVT and |
| | pulmonary embolism, 7.5%) |
| | Hematologic: Bleeding (DVT and pulmonary embolism |
| | prophylaxis, 10.5%; nonvalvular atrial fibrillation, 16.6%) |
| | Serious: |
| | Cardiovascular: Myocardial infarction (DVT and pulmonary |
| | embolism, 0.32% to 0.66%; nonvalvular atrial fibrillation, 0.7%) |
| | Gastrointestinal: Gastrointestinal hemorrhage, Major (DVT and |
| | pulmonary embolism, 0.3% to 0.6%; nonvalvular atrial fibrillation, |
| | 1.6%) |
| | Hematologic: Bleeding, Major (DVT and pulmonary embolism, |
| | 0.3% to 1.4%; nonvalvular atrial fibrillation, 3.3%), Thrombosis |
| | Immunologic: Anaphylaxis |
| | Neurologic: Epidural hematoma, Intracranial hemorrhage |
| | (nonvalvular atrial fibrillation, 0.3%; DVT and pulmonary |
| | embolism, 0.1%), Traumatic spinal subdural hematoma |
| | Respiratory: Bleeding, Alveolar |
| Promacta | Common: |
| | Gastrointestinal: Diarrhea (Chronic hepatitis C-associated |
| | thrombocytopenia, 19%; chronic idiopathic thrombocytopenic |
| | purpura, adults, 9%, pediatric, 5%; aplastic anemia, 21%), Nausea |
| | (Chronic hepatitis C-associated thrombocytopenia, 19%; chronic |
| | idiopathic thrombocytopenic purpura, 4% to 9%; aplastic anemia, |
| | 33%), Pain in throat (Chronic idiopathic thrombocytopenic |
| | purpura, 4%; aplastic anemia, 14% 4%), Pharyngitis (4%), |
| | Vomiting (6%) |
| | Hematologic: Anemia (chronic hepatitis C-associated |
| | thrombocytopenia, 40%) |
| | Hepatic: ALT/SGPT level raised (Chronic idiopathic |
| | thrombocytopenic purpura, 5% to 6%; chronic ITP, pediatric, 6%), |
| | AST/SGOT level raised (Adult, 4%; pediatric, 5%), |
| | Hyperbilirubinemia (6% to 8%) |
| | Musculoskeletal: Myalgia (2% to 12%) |
| | Neurologic: Headache (Chronic hepatitis C-associated |
| | thrombocytopenia and aplastic anemia, 21%; chronic idiopathic |
| | thrombocytopenic purpura, 10%) |
| | Ophthalmic: Cataract (4% to 7%) |
| | Renal: Urinary tract infectious disease (5%) |
| | Respiratory: Cough (Aplastic anemia, 23%; chronic ITP, pediatric, |
| | 9%), Epistaxis (13%) |
| | Other: Fatigue (Chronic hepatitis C-associated thrombocytopenia |
| | and aplastic anemia, 28%; chronic idiopathic thrombocytopenic |
| | purpura, 4%), Fever (Chronic hepatitis C-associated |
| | thrombocytopenia, 30%; aplastic anemia, 14%) |
| | Serious: |
| | Hematologic: Bleeding, Portal vein thrombosis (chronic hepatitis |
| | C-associated thrombocytopenia, 1%), Thrombosis (chronic |
| | hepatitis C-associated thrombocytopenia, 3%) |
| | Hepatic: Hepatotoxicity, Liver failure (chronic hepatitis C- |
| | associated thrombocytopenia, 7%), Liver function tests abnormal |
| | (11%) |
| | Renal: Acute renal failure |
| Suboxone | Common: |
| | Dermatologic: Hyperhidrosis (SL tablet, 14%; buccal film, 1% to |
| | less than 5%) |
| | Gastrointestinal: Abdominal pain (SL tablet, 11.2%), Constipation |
| | (SL tablet, 12.1%; buccal film, 1% to less than 5%), Nausea (SL |
| | tablet, induction phase, 5%; long-term use, 15%), Vomiting (SL |
| | tablet, 5% to 7.5%) |
| | Neurologic: Headache (SL tablet, induction phase, 7%; long-term |
| | use, 36.4%; buccal film, 5% or greater), Insomnia (SL tablet, 14%; |
| | buccal film, greater than 1% and less than 5%) |
| | Other: Drug withdrawal (SL tablet, 25.2%; buccal film, at least |
| | 5%), Pain (SL tablet, 22.4%) |
| | Serious: |
| | Hepatic: Hepatitis |
| | Immunologic: Anaphylaxis |
| | Neurologic: Central nervous system depression |
| | Respiratory: Respiratory depression |
| | Other: Drug dependence (Buccal film, 1% to less than 5%) |
| Tekturna HCT | Common: |
| | Endocrine metabolic: Hyperkalemia (0.8% to 36.9%), |
| | Hypokalemia (2.2%) |
| | Gastrointestinal: Diarrhea (1.6%) |
| | Neurologic: Dizziness (2.3%) |
| | Renal: Serum blood urea nitrogen raised (11.8%) |
| | Respiratory: Cough (1.3%) |
| | Serious: |
| | Cardiovascular: Hypotension |
| | Ophthalmic: Angle-closure glaucoma, acute, Myopia (Acute), |
| | Transient |
| Thalomid | Common: |
| | Cardiovascular: Edema (multiple myeloma, 13% to 56%), |
| | Peripheral edema (erythema nodosum leprosum, 3.1% to 8.3%; |
| | multiple myeloma, 34%) |
| | Dermatologic: Dry skin (multiple myeloma, 21%), Rash (erythema |
| | nodosum leprosum, 20.8%) |
| | Endocrine metabolic: Hypocalcemia (multiple myeloma, 72%), |
| | Weight gain (multiple myeloma, 3% to 22%), Weight loss |
| | (multiple myeloma, 23%) |
| | Gastrointestinal: Constipation (erythema nodosum leprosum, 2.8% |
| | to 9.4%; multiple myeloma, 50% to 55%), Diarrhea (erythema |
| | nodosum leprosum, 4.2% to 18.7%), Indigestion (multiple |
| | myeloma, 11%), Nausea (erythema nodosum leprosum, 4.2%; |
| | multiple myeloma, 13% to 28%) |
| | Hematologic: Leukopenia (erythema nodosum, 16.7% to 25%; |
| | multiple myeloma, 35%) |
| | Musculoskeletal: Muscle weakness (multiple myeloma, 40%) |
| | Neurologic: Asthenia (erythema nodosum leprosum, 5.6% to |
| | 21.9%; multiple myeloma, 24%), Confusional state (multiple |
| | myeloma, 28%), Dizziness (erythema nodosum leprosum, 4.2% to |
| | 19.2%; multiple myeloma, 23%), Somnolence (erythema nodosum |
| | leprosum, 36.1% to 37.5%; multiple myeloma, 3% or more), |
| | Tremor (erythema nodosum leprosum, 4.2%; multiple myeloma, |
| | 26%) |
| | Respiratory: Dyspnea (multiple myeloma, 42%), Pneumonia |
| | (multiple myeloma, 15%) |
| | Other: Fatigue (multiple myeloma, 21% to 79%), Fever (erythema |
| | nodosum leprosum, 19.4% to 21.9%; multiple myeloma, 24%) |
| | Serious: |
| | Cardiovascular: Atrial fibrillation, Grade 3/4 (multiple myeloma, |
| | 5%), Cardiac dysrhythmia, Ischemic heart disease (11.1%), |
| | Myocardial infarction (1.3%) |
| Tikosyn | Common: |
| | Cardiovascular: Chest pain (10%) |
| | Neurologic: Dizziness (8%), Headache (11%) |
| | Serious: |
| | Cardiovascular: Heart block (up to 1.2%), Prolonged QT interval, |
| | Torsades de pointes (0.8%), Ventricular arrhythmia (up to 14.5%), |
| | Ventricular fibrillation (up to 4.8%), Ventricular tachycardia (up to |
| | 12.4%) |
| Tradjenta | Common: |
| | Endocrine metabolic: Hypoglycemia (monotherapy, 6.6%; |
| | combination therapy, 22.9%) |
| | Respiratory: Nasopharyngitis (7%) |
| | Serious: |
| | Gastrointestinal: Pancreatic cancer, Pancreatitis |
| | Immunologic: Anaphylaxis, Hypersensitivity reaction |
| | Other: Angioedema, Pancreatic cancer |
| Trokendi XR | Common: |
| | Dermatologic: Flushing (pediatrics, 5%) |
| | Endocrine metabolic: Serum bicarbonate level abnormal (25% to |
| | 67%) |
| | Gastrointestinal: Loss of appetite (10% to 24%), Weight decreased |
| | (4% to 21%) |
| | Immunologic: Infectious disease (2% to 8%) |
| | Neurologic: Confusion (3% to 11%), Dizziness (4% to 25%), |
| | Impaired cognition (2% to 7%), Impaired psychomotor |
| | performance (2% to 13%), Memory impairment (3% to 12%), |
| | Paresthesia (1% to 51%), Reduced concentration span (2% to |
| | 10%), Somnolence (6% to 29%) |
| | Psychiatric: Feeling nervous (4% to 16%), Mood disorder (4% to |
| | 11%) |
| | Other: Fatigue (6% to 16%), Fever (1% to 12%) |
| | Serious: |
| | Dermatologic: Erythema multiforme, Stevens-Johnson syndrome, |
| | Toxic epidermal necrolysis |
| | Endocrine metabolic: Hyperammonemia (Adolescents, 26%), |
| | Hypohidrosis, Increased body temperature, Metabolic acidosis |
| | Hepatic: Liver failure |
| | Neurologic: Drug-induced encephalopathy |
| | Ophthalmic: Glaucoma, Myopia, Visual field defect (epilepsy, |
| | 0.1% to 1%) |
| | Psychiatric: Suicidal thoughts |
| | Renal: Nephrolithiasis (adults, 1% to 3%) |
| Viibryd | Common: |
| | Gastrointestinal: Diarrhea (26% to 29%), Nausea (22% to 24%), |
| | Vomiting (4% to 5%) |
| | Neurologic: Insomnia (6% to 7%) |
| | Serious: |
| | Cardiovascular: Ventricular premature beats (0.1% to 1%) |
| | Psychiatric: Suicidal behavior, Suicidal thoughts |
| | Other: Drug withdrawal, Serotonin syndrome (0.1%) |
| Xartemis XR | Common: |
| | Gastrointestinal: Constipation (extended-release, 4%), Nausea |
| | (extended-release, 31%), Vomiting (extended-release, 9%) |
| | Neurologic: Dizziness (extended-release, 13%), Headache |
| | (extended-release, 10%), Lightheadedness, Sedated, Somnolence |
| | (extended-release, 4%) |
| | Serious: |
| | Cardiovascular: Disorder of pulmonary circulation, Hypotension, |
| | Shock |
| | Dermatologic: Acute generalized exanthematous pustulosis, |
| | Stevens-Johnson syndrome, Toxic epidermal necrolysis |
| | Hematologic: Agranulocytosis, Neutropenia |
| | Hepatic: Hepatic necrosis, Hepatotoxicity, Liver failure |
| | Immunologic: Anaphylaxis, Hypersensitivity reaction |
| | Respiratory: Apnea, Respiratory arrest, Respiratory depression |
| | Other: Neonatal Abstinence Syndrome |
| Zohydro ER | Common: |
| | Cardiovascular: Peripheral edema (1% to less than 5%%) |
| | Dermatologic: Pruritus (0% to less than 5%) |
| | Gastrointestinal: Abdominal pain (1% to less than 5%), |
| | Constipation (3% to 11%), Nausea (7% to 10%), Vomiting (3% to |
| | 6%), Xerostomia (1% to less than 5%) |
| | Musculoskeletal: Spasm (1% to less than 5%) |
| | Neurologic: Dizziness (2% to 3%), Headache (2% to 4%), |
| | Somnolence (1% to 5%), Tremor (3%) |
| | Renal: Urinary tract infectious disease (1% to 5%) |
| | Respiratory: Upper respiratory infection (1% to 3%) |
| | Other: Fatigue (1% to 4%) |
| | Serious: |
| | Cardiovascular: Hypotension (less than 1%), Orthostatic |
| | hypotension (less than 1%), Prolonged QT interval, Syncope |
| | Gastrointestinal: Difficulty swallowing (less than 1%) |
| | Neurologic: Raised intracranial pressure, Seizure |
| | Respiratory: Respiratory depression |
| | Other: Drug withdrawal syndrome in neonate of dependent mother, |
| | Opioid withdrawal (less than 1%) |
| Zorvolex | Common |
| | Gastrointestinal: Constipation (5% to 8%), Diarrhea (6%), Nausea |
| | (6% to 7%) |
| | Hepatic: Increased liver function test (15%) |
| | Neurologic: Headache (4% to 8%) |
| | Renal: Urinary tract infectious disease (7%) |
| | Respiratory: Nasopharyngitis (6%), Sinusitis (3% to 5%), Upper |
| | respiratory infection (8%) |
| | Serious |
| | Cardiovascular: Myocardial infarction, Thrombosis |
| | Dermatologic: Erythema multiforme, Erythroderma, Stevens- |
| | Johnson syndrome, Toxic epidermal necrolysis |
| | Gastrointestinal: Gastrointestinal hemorrhage, Gastrointestinal |
| | perforation |
| | Hematologic: Aplastic anemia, Blood coagulation disorder, |
| | Hemolytic anemia, Thrombocytopenia |
| | Hepatic: Increased liver enzymes, Jaundice, Liver failure |
| | Immunologic: Anaphylactoid reaction |
| | Neurologic: Cerebrovascular accident |
| | Renal: Acute renal failure |
| | Respiratory: Bronchospasm |
| Zytiga | Common |
| | Dermatologic: Contusion (13.3%), Flushing (19% to 22.3%) |
| | Endocrine metabolic: Hypercholesterolemia (Greater than 20%), |
| | Hyperglycemia (56.6%), Hypertriglyceridemia (62.5%), |
| | Hypophosphatemia (23.8%) |
| | Gastrointestinal: Diarrhea (17.6% to 21.6%), Vomiting (10% or |
| | higher) |
| | Hematologic: Anemia (Greater than 20%), Lymphocytopenia, All |
| | grades (38.2%) |
| | Hepatic: Alkaline phosphatase raised (Greater than 20%) |
| | Musculoskeletal: Joint swelling (29.5% to 30.3%) |
| | Renal: Urinary tract infectious disease (11.5%) |
| | Respiratory: Cough (10.6% to 17.3%), Dyspnea (11.8%) |
| | Other: Fatigue (39.1%) |
| | Serious |
| | Cardiovascular: Cardiac dysrhythmia (7.2%), Cardiorespiratory |
| | arrest (0.5%), Chest discomfort, Chest pain, Edema (25.1% to |
| | 26.7%), Heart failure (2.1% to 2.3%), Hypertension (8.5% to |
| | 21.6%), Myocardial infarction, Sudden cardiac death |
| | Endocrine metabolic: Adrenal insufficiency (0.5%), Hypokalemia |
| | (17.2% to 28.3%) |
| | Hematologic: Lymphocytopenia, Grade 3 or 4 (8.7%) |
| | Hepatic: ALT/SGPT level raised (11.1% to 41.9%), AST/SGOT |
| | level raised (30.6% to 37.3%), Serum bilirubin raised (6.6%) |
| Seroquel (Quetiapine) | Common: |
| | Cardiovascular: Increased diastolic arterial pressure (Pediatric, |
| | 40.6%), Increased systolic arterial pressure (Pediatric, 15.2%), |
| | Orthostatic hypotension (Up to 7%), Tachycardia (Up to 6%) |
| | Endocrine metabolic: Serum cholesterol raised (7% to 18%), Serum |
| | triglycerides raised (8% to 28%), Weight gain (3% to 28%) |
| | Gastrointestinal: Abdominal pain (3% to 7%), Constipation (2% to |
| | 11%), Increased appetite (2% to 12%), Indigestion (2% to 7%), |
| | Nausea (Pediatric, 6% to 10%), Vomiting (Pediatric, 7% to 8%), |
| | Xerostomia (Adult, 9% to 44%; pediatric, 4% to 10%) |
| | Hepatic: Increased liver enzymes (1% to 6%) |
| | Musculoskeletal: Backache (3% to 5%) |
| | Neurologic: Asthenia (Up to 10%), Dizziness (8% to 19%), |
| | Extrapyramidal disease (1.1% to 12.9%), Headache (17% to 21%), |
| | Insomnia (8% to 12%), Lethargy (1% to 5%), Somnolence (18% to |
| | 57%), Tremor (2% to 8%) |
| | Psychiatric: Agitation (6% to 20%) |
| | Respiratory: Nasal congestion (3% to 5%), Pharyngitis (4% to 6%) |
| | Other: Fatigue (3% to 14%), Pain (7%) |
| | Serious: |
| | Cardiovascular: Prolonged QT interval (0.1% to less than 1%), |
| | Sudden cardiac death, Syncope (0.3% to 1%) |
| | Endocrine metabolic: Diabetic ketoacidosis |
| | Gastrointestinal: Pancreatitis |
| | Hematologic: Agranulocytosis, Leukopenia, Neutropenia (0.3% to |
| | 1.5%) |
| | Immunologic: Anaphylaxis |
| | Neurologic: Seizure (0.05% to 0.5%), Tardive dyskinesia |
| | Psychiatric: Suicidal thoughts |
| | Reproductive: Priapism |
| | Other: Neuroleptic malignant syndrome |

Pain Relievers - Opioids

| | |
|---|---|
| Codeine | Common |
| | Gastrointestinal: Constipation, Nausea, Vomiting |
| | Neurologic: Dizziness, Lightheadedness, Sedated, Somnolence |
| | Respiratory: Dyspnea |
| | Serious |
| | Cardiovascular: Hypotension |
| | Gastrointestinal: Bowel obstruction, Pancreatitis |
| | Neurologic: CSF pressure: raised, Seizure |
| | Respiratory: Respiratory depression |
| Fentanyl and Analogs | Common |
| | Cardiovascular: Hypotension (up to 5% (buccal film)), Peripheral |
| | edema (1% or greater (nasal spray); 5% to 32% (buccal tablet)) |
| | Dermatologic: Application site reaction (10% (buccal tablet)), Rash |
| | (3% to 8% (lozenge)) |
| | Endocrine metabolic: Abnormal weight loss (up to 13% (buccal |
| | tablet/film)), Hypokalemia (up to 15% (buccal tablet)) |
| | Gastrointestinal: Abdominal pain (1% or greater (nasal spray); up |
| | to 15%), Constipation (4% to 26%), Diarrhea (up to 16% (buccal |
| | tablet/film)), Loss of appetite (2% to 11% (buccal tablet/film)), |
| | Nausea (5.6% to 42%), Vomiting (4% to 37%) |
| | Hematologic: Anemia (1% or greater (nasal spray); 9% to 32% |
| | (buccal tablet)), Neutropenia (1% or greater (nasal spray); up to 8% |
| | (buccal tablet)) |
| | Musculoskeletal: Arthralgia (up to 8% (buccal tablet)), Backache |
| | (up to 11% (buccal tablet)) |
| | Neurologic: Asthenia (up to 30% (lozenge, buccal tablet/film)), |
| | Confusion (up to 16%), Dizziness (6% (nasal spray); up to 26% |
| | (lozenge, sublingual, buccal tablet/film)), Headache (1% or greater |
| | (nasal spray); up to 17% (lozenge, sublingual, buccal tablet/film)), |
| | Insomnia (up to 11% (lozenge, buccal tablet/film)), Somnolence |
| | (up to 15% (sublingual, buccal tablet)) |
| | Psychiatric: Anxiety (3% to 9% (buccal film, lozenge)), Depression |
| | (up to 11% (buccal tablet)) |
| | Respiratory: Cough (up to 9%), Pneumonia (1% to 16% (buccal |
| | tablet, nasal spray)) |
| | Other: Dehydration (up to 21% (buccal tablet, film)), Fatigue (1% |
| | to 20% (buccal tablet, film)) |
| | Serious |
| | Cardiovascular: Bradyarrhythmia (1% or greater (sublingual |
| | tablet)), Cardiorespiratory arrest (1% or greater (nasal spray)), |
| | Tachyarrhythmia (1% or greater (sublingual, buccal tablet/film)) |
| | Gastrointestinal: Bowel obstruction (1% or greater (buccal film); up |
| | to 4% (lozenge)) |
| | Hematologic: Deep venous thrombosis (1% or greater (nasal |
| | spray)) |
| | Musculoskeletal: Muscle rigidity |
| | Respiratory: Dyspnea (up to 19% (lozenge, buccal tablet/film)), |
| | Respiratory depression |

(Causing Overdoses)

| | |
|---|---|
| Hydrocodone | Common |
| | Cardiovascular: Peripheral edema (1% to less than 5%%) |
| | Dermatologic: Pruritus (0% to less than 5%) |
| | Gastrointestinal: Abdominal pain (1% to less than 5%), |
| | Constipation (3% to 11%), Nausea (7% to 10%), Vomiting (3% to |
| | 6%), Xerostomia (1% to less than 5%) |
| | Musculoskeletal: Spasm (1% to less than 5%) |
| | Neurologic: Dizziness (2% to 3%), Headache (2% to 4%), |
| | Somnolence (1% to 5%), Tremor (3%) |
| | Renal: Urinary tract infectious disease (1% to 5%) |
| | Respiratory: Upper respiratory infection (1% to 3%) |
| | Other: Fatigue (1% to 4%) |
| | Serious |
| | Cardiovascular: Hypotension (less than 1%), Orthostatic |
| | hypotension (less than 1%), Prolonged QT interval, Syncope |
| | Gastrointestinal: Difficulty swallowing (less than 1%) |
| | Neurologic: Raised intracranial pressure, Seizure |
| | Respiratory: Respiratory depression |
| | Other: Drug withdrawal syndrome in neonate of dependent mother, |
| | Opioid withdrawal (less than 1%) |
| Hydromorphone | Common |
| | Dermatologic: Flushing (extended-release, less than 2%), Pruritus |
| | (extended-release, 1% to 8%), Sweating |
| | Gastrointestinal: Constipation (extended-release, 7% to 31%), |
| | Nausea (extended-release, 9% to 28%.), Vomiting (extended- |
| | release, 6% to 14%) |
| | Neurologic: Asthenia (1% to 11%), Dizziness (1% to 11%), |
| | Headache (1% to 12%), Somnolence (less than 2%) |
| | Serious |
| | Cardiovascular: Hypotension (less than 2%), Syncope (less than |
| | 2%) |
| | Neurologic: Coma, Myoclonus (less than 2%), Raised intracranial |
| | pressure, Seizure (less than 2%) |
| | Psychiatric: Suicidal thoughts (extended-release, less than 2%) |
| | Respiratory: Apnea (less than 1%), Respiratory arrest, Respiratory |
| | depression (less than 2%) |
| | Other: Drug dependence (less than 1%), Drug withdrawal (less than |
| | 1%), Neonatal Abstinence Syndrome |
| Methadone | Common |
| | Cardiovascular: Hypotension |
| | Endocrine metabolic: Diaphoresis |
| | Gastrointestinal: Constipation, Nausea, Vomiting |
| | Neurologic: Asthenia, Dizziness, Lightheadedness, Sedated |
| | Serious |
| | Cardiovascular: Decreased vascular flow, left ventricle, Prolonged |
| | QT interval, Torsades de pointes |
| | Endocrine metabolic: Hypoglycemia |
| | Respiratory: Respiratory acidosis, Respiratory arrest, Respiratory |
| | depression |
| | Other: Drug dependence |
| Oxycodone | Common |
| | Dermatologic: Pruritus (Adults, controlled-release, 13%; |
| | immediate-release, 3% or greater; pediatrics, 6%), Sweating |
| | (controlled-release, 5%; immediate-release, less than 3%) |
| | Gastrointestinal: Abdominal pain (up to 5%), Constipation (Adults, |
| | controlled-release, 23%; immediate-release, 3% or greater; |
| | pediatrics, 9%), Nausea (Adults, controlled-release, 23%; |
| | immediate-release, 3% or greater; pediatrics, 15%), Vomiting |
| | (Adults, controlled-release, 12%; immediate-release, 3% or greater; |
| | pediatrics, 21%), Xerostomia (controlled-release, 6%; immediate- |
| | release, less than 3%) |
| | Neurologic: Asthenia (controlled-release, 6%; immediate-release, |
| | 3% or greater), Dizziness (Adults, controlled-release, 13%; |
| | immediate-release, 3% or greater; pediatrics, 9%), Headache |
| | (Adults, 3% or greater; pediatrics, 14%), Somnolence (controlled- |
| | release, 23%; immediate-release, 3% or greater) |
| | Other: Fever (Adults, up to 5%; pediatrics, 11%) |
| | Serious |
| | Cardiovascular: Cardiac arrest, Chest pain (less than 1%), Heart |
| | failure (less than 3%), Hypotension (less than 3%), Shock, ST |
| | segment depression (less than 1%), Syncope (less than 1%) |
| | Gastrointestinal: Bowel obstruction, Diverticulitis, Exacerbation |
| | Immunologic: Hypersensitivity reaction (less than 3%) |
| | Respiratory: Respiratory depression |
| | Other: Drug withdrawal syndrome in neonate of dependent mother, |
| | Opioid withdrawal (1% to 5%) |
| Oxymorphone | Common |
| | Cardiovascular: Hypotension (less than 10%) |
| | Dermatologic: Pruritus (less than or equal to 15.2%), Sweating |
| | symptom (1% to less than 10%) |
| | Gastrointestinal: Abdominal pain (1% to less than 10%), |
| | Constipation (4.1% to 27.6%), Nausea (2.9% to 33.1%), Vomiting |
| | (less than or equal to 15.6%), Xerostomia (1% to less than 10%) |
| | Neurologic: Confusion (1% to less than 10%), Dizziness (5% to |
| | 17%), Headache (4% to 12%), Somnolence (2% to 19%) |
| | Respiratory: Dyspnea (1% to less than 10%), Hypoxia (less than |
| | 10%) |
| | Other: Fatigue (1% to less than 10%), Fever (1% to 14.2%) |
| | Serious |
| | Gastrointestinal: Bowel obstruction (less than 1%) |
| | Neurologic: Coma |
| | Respiratory: Respiratory depression |
| | Other: Drug dependence, Drug withdrawal syndrome in neonate of |
| | dependent mother |
| Amytal (amobarbital) | Common |
| | Neurologic: Confusion, Dizziness, Headache, Somnolence |
| | Serious |
| | Dermatologic: Stevens-Johnson syndrome (rare) |
| | Hematologic: Agranulocytosis (rare), Megaloblastic anemia, With |
| | prolonged use (rare) |
| | Hepatic: Injury of liver, With prolonged use (rare) |
| | Respiratory: Apnea, Hypoventilation |
| Nembutal (pentobarbital) | Common |
| | Neurologic: Confusion (less than 1%), Dizziness (less than 1%), |
| | Somnolence (1% to 3%) |
| | Psychiatric: Agitation (less than 1%) |
| | Serious |
| | Dermatologic: Stevens-Johnson syndrome (less than 1%) |
| | Hematologic: Megaloblastic anemia (less than 1%) |
| | Hepatic: Injury of liver (less than 1%) |
| | Respiratory: Apnea (less than 1%), Hypoventilation (less than 1%) |
| Seconal (secobarbital) | Common |
| | Neurologic: Somnolence (1% to 3%) |
| | Serious |
| | Hematologic: Megaloblastic anemia, With prolonged use (less than |
| | 1%) |
| | Hepatic: Liver damage (less than 1%) |
| | Psychiatric: Complex mannerisms - behavior (Less than 1%.) |
| | Other: Drug dependence, Withdrawal sign or symptom |
| Adderall (Amphetamine) | Common |
| | Cardiovascular: Increased systolic arterial pressure (extended- |
| | release: pediatrics, 7% to 35%) |
| | Endocrine metabolic: Weight loss (extended-release: adults, 10%; |
| | pediatrics, 4% to 9%) |
| | Gastrointestinal: Abdominal pain (extended-release: pediatrics, |
| | 11% to 14%), Loss of appetite (extended-release capsules: adults, |
| | 33%; pediatrics, 22% to 36%), Xerostomia (extended-release: |
| | adults, 35%; pediatrics, 2% to 4%) |
| | Neurologic: Dizziness, Headache (extended-release: adults, 26%), |
| | Insomnia (extended-release: adults, 27%; pediatrics, 12% to 17%) |
| | Psychiatric: Feeling nervous (extended release: adults, 13%; |
| | pediatrics, 6%) |
| | Serious |
| | Cardiovascular: Cardiomyopathy, Myocardial infarction, Peripheral |
| | vascular disease, Raynaud's disease, Sudden cardiac death |
| | Dermatologic: Stevens-Johnson syndrome, Toxic epidermal |
| | necrolysis |
| | Immunologic: Hypersensitivity reaction |
| | Neurologic: Cerebrovascular accident, Seizure |
| | Psychiatric: Psychotic disorder |
| Methylphenidate | Common |
| | Cardiovascular: Tachycardia (Adult, 4.8%) |
| | Dermatologic: Diaphoresis (Adult, 5.1%) |
| | Endocrine metabolic: Weight decreased (Adult, 6.5%) |
| | Gastrointestinal: Abdominal pain (2% or greater), Decrease in |
| | appetite (Adult, 25.3%; pediatric, 2% to 9% or greater), Loss of |
| | appetite (Adult, 1.7%; pediatric, 3.1% to 9% or greater), Nausea |
| | (Adult, 12.8%), Vomiting (2% or greater), Xerostomia (Adult, |
| | 14%) |
| | Neurologic: Dizziness (Adult, 6.7%; pediatric, 1.9%.), Headache |
| | (Adult, 22.2%; pediatric, up to 12%), Insomnia (Adult, 12.3%; |
| | pediatric, 2.8% to 5%) |
| | Psychiatric: Anxiety (Adult, 8.2%), Depression (Adult, 1.7% to |
| | 3.9%), Irritability (Adult, 5.8%) |
| | Serious |
| | Cardiovascular: Myocardial infarction, Raynaud's phenomenon, |
| | Sudden cardiac death |
| | Endocrine metabolic: Decreased body growth |
| | Gastrointestinal: Gastrointestinal obstruction, with preexisting |
| | severe gastrointestinal narrowing and use of controlled-release |
| | formulations |
| | Hepatic: Abnormal liver function |
| | Neurologic: Cerebral artery occlusion, Cerebral hemorrhage, |
| | Cerebrovascular accident, Seizure |
| | Ophthalmic: Blurred vision (1.7% to 2% or greater) |
| | Psychiatric: Aggressive behavior (Adult, 1.7%), Mania, Psychotic |
| | disorder |
| | Reproductive: Priapism |
| Daytrana | See Methylphenidate |
| Concerta | See Methylphenidate |
| Ritalin | See Methylphenidate |
| Klonopin (clonazepam) | Common |
| | Neurologic: Ataxia (5% to 30%), Coordination problem (6%), |
| | Dizziness (8%), Somnolence (37% to 50%) |
| | Psychiatric: Problem behavior (25%) |
| | Respiratory: Upper respiratory infection (8%) |
| | Other: Fatigue (7%) |
| | Serious |
| | Psychiatric: Depression (7%), Suicidal thoughts |
| | Respiratory: Respiratory depression |
| Valium (diazepam) | Common |
| | Cardiovascular: Hypotension |
| | Dermatologic: Rash (3%, rectal gel) |
| | Gastrointestinal: Diarrhea (4%, rectal gel) |
| | Musculoskeletal: Muscle weakness |
| | Neurologic: Ataxia, Incoordination (3%, rectal gel), Somnolence |
| | Psychiatric: Euphoria (3%, rectal gel) |
| | Respiratory: Respiratory depression |
| | Other: Fatigue |
| | Serious |
| | Hematologic: Neutropenia |
| Xanax (alprazolam) | Common |
| | Endocrine metabolic: Decrease in appetite (7.3% to 27.8%), |
| | Increased appetite (7% to 32.7%), Weight increase (2.7% to 27.2%) |
| | Gastrointestinal: Constipation (8.1% to 26.2%), Reduced salivation |
| | (32.8%), Xerostomia (10.2% to 14.7%) |
| | Neurologic: Cognitive disorder (28.8%), Confusion (1.5% to |
| | 10.4%), Dysarthria (10.9% to 23.3%), Incoordination (9.4% to |
| | 40.1%), Lightheadedness (20.8%), Memory impairment (15.4% to |
| | 33.1%), Sedated (45.2%), Somnolence (23% to 76.8%) |
| | Psychiatric: Irritability (immediate-release, 33.1%; extended- |
| | release 1% or more) |
| | Reproductive: Reduced libido (6% to 14.4%) |
| | Other: Fatigue (13.9% to 48.6%) |
| | Serious |
| | Dermatologic: Stevens-Johnson syndrome |
| Ambien (zolpidem) | Common |
| | Gastrointestinal: Diarrhea (1% to 3%), Nausea (1% to 7%) |
| | Immunologic: Allergic reaction (4%) |
| | Neurologic: Dizziness (1% to 23.5%), Drugged state (3%), |
| | Headache (1% to 19%), Somnolence (2% to 15%) |
| | Ophthalmic: Visual disturbance (3%) |
| | Other: Fatigue (0.1% to 3%) |
| | Serious |
| | Cardiovascular: Chest pain (1%), Tachycardia (0.1% to 1%) |
| | Immunologic: Anaphylaxis (rare) |
| | Neurologic: Hepatic encephalopathy |
| | Psychiatric: Complex mannerisms - behavior, Depression, |
| | worsening, Suicidal thoughts |
| | Other: Angioedema (rare) |
| Lunesta (eszopiclone) | Common |
| | Gastrointestinal: Disorder of taste (8% to 34%), Vomiting (3%) |
| | Neurologic: Dizziness (1% to 7%), Headache (13% to 21%), |
| | Migraine (1% or greater) |
| | Respiratory: Respiratory tract infection (5% to 10%) |
| | Serious |
| | Other: Angioedema (rare) |
| Sonata (zaleplon) | Common |
| | Neurologic: Dizziness (7% to 9%), Headache (30% to 42%) |
| | Serious |
| | Immunologic: Anaphylaxis (rare) |
| | Neurologic: Drug withdrawal seizure (rare) |
| | Psychiatric: Abnormal behavior, Complex mannerisms - behavior, |
| | Depression (at least 1%), Suicidal behavior, Suicidal thoughts |
| | Other: Angioedema (rare) |
| Chantix | Common |
| | Gastrointestinal: Constipation (5% to 8%), Flatulence (6% to 9%), |
| | Nausea (30%), Vomiting (5% to 11%) |
| | Neurologic: Dream disorder (9% to 13%), Headache (11% to 19%), |
| | Insomnia (10% to 19%) |
| | Serious |
| | Cardiovascular: Angina (2.3%), Myocardial infarction (2%) |
| | Neurologic: Cerebrovascular accident |
| | Ophthalmic: Acquired night blindness (rare), Blurred vision |
| | (infrequent), Retinal vascular disorder (rare), Subcapsular cataract |
| | (rare), Transient blindness (rare), Visual disturbance (infrequent) |
| | Psychiatric: Abnormal behavior, Depression (3.5% to 11%), |
| | Hostile behavior (2%), Mood disorder (2.3%), Suicidal behavior, |
| | and/or ideation (6% to 11%) |
| Revlimid | Common |
| | Cardiovascular: Peripheral edema (multiple myeloma, 26.3%; |
| | myelodysplastic syndrome, 20.3%; mantle cell lymphoma, 16%) |
| | Dermatologic: Pruritus (Multiple myeloma, 7.6%; myelodysplastic |
| | syndrome, 41.9%; mantle cell lymphoma, 17%), Rash (Multiple |
| | myeloma, up to 26.1%; myelodysplastic syndrome, 35.8%; mantle |
| | cell lymphoma, 22%) |
| | Endocrine metabolic: Hypokalemia (Multiple myeloma, 13.6% to |
| | 17.1%; myelodysplastic syndrome, 10.8%; mantle cell lymphoma, |
| | 13%), Weight decreased (13% to 19.5%) |
| | Gastrointestinal: Constipation (Multiple myeloma, 40.5%; |
| | myelodysplastic syndrome, 23.6%; mantle cell lymphoma, 16%), |
| | Diarrhea (Multiple myeloma, 38.5% to 45.5%; myelodysplastic |
| | syndrome, 48.6%; mantle cell lymphoma, 31%), Nausea (23.6% to |
| | 30%\grave{ }) |
| | Hematologic: Anemia, All grades (Multiple myeloma, 31.4% to |
| | 43.8%; myelodysplastic syndrome, 11.5%; mantle cell lymphoma, |
| | 31%), Leukopenia, All grades (7.9% to 15%), Neutropenia, All |
| | grades (Multiple myeloma, 35% to 42.2%; myelodysplastic |
| | syndrome, 58.8%; mantle cell lymphoma, 49%), |
| | Thrombocytopenia, All grades (Multiple myeloma, 19.5% to |
| | 21.5%; myelodysplastic syndrome, 61.5%; mantle cell lymphoma, |
| | 36%) |
| | Musculoskeletal: Arthralgia (Multiple myeloma, 19%; |
| | myelodysplastic syndrome, 21.6%; mantle cell lymphoma, 8%), |
| | Backache (Multiple myeloma, 25.8% to 32%; myelodysplastic |
| | syndrome, 20.9%; mantle cell lymphoma, 13%), Cramp (Multiple |
| | myeloma, 33.4%; myelodysplastic syndrome, 18.2%) |
| | Neurologic: Asthenia (Multiple myeloma, 28.2%; myelodysplastic |
| | syndrome, 14.9%; mantle cell lymphoma, 14%), Dizziness (19.6% |
| | to 23.2%), Headache (Myelodysplastic syndrome, 19.6%), |
| | Insomnia (Multiple myeloma, 27.6%; myelodysplastic syndrome, |
| | 10.1%), Tremor (Multiple myeloma, 21.2%) |
| | Ophthalmic: Blurred vision (Multiple myeloma, 17.3%) |
| | Respiratory: Cough (Multiple myeloma, 22.7%; myelodysplastic |
| | syndrome, 19.6%; mantle cell lymphoma, 28%), Dyspnea (Multiple |
| | myeloma, 22% to 23.5%; myelodysplastic syndrome, 6.8% to |
| | 16.9%; mantle cell lymphoma, 18%), Epistaxis (Myelodysplastic |
| | syndrome, 14.9%), Nasopharyngitis (Multiple myeloma, 15% to |
| | 17.6%; myelodysplastic syndrome, 23%), Pharyngitis (13.6% to |
| | 15.5%), Upper respiratory infection (Multiple myeloma, 24.6%; |
| | myelodysplastic syndrome, 14.9%; mantle cell lymphoma, 13%) |
| | Other: Fatigue (Multiple myeloma, 32.5% to 43.9%; |
| | myelodysplastic syndrome, 31.1%; mantle cell lymphoma, 34%), |
| | Fever (Multiple myeloma, 21.4% to 27.5%; myelodysplastic |
| | syndrome, 20.9%; mantle cell lymphoma, 23%), Infectious disease |
| | Serious |
| | Cardiovascular: Atrial fibrillation, Grade 3 or 4 (multiple myeloma, |
| | 3.7%), Cerebrovascular accident (1.4% to 2.3%), Congestive heart |
| | failure, Grade 3 or 4 (multiple myeloma, 1.4%), Myocardial |
| | infarction (Less than 5%), Syncope, Grade 3 or 4 (1.4% to 2.8%) |
| | Dermatologic: Stevens-Johnson syndrome, Toxic epidermal |
| | necrolysis |
| | Hematologic: Anemia, Grade 3 or 4 (Multiple myeloma, 9.9% to |
| | 18.2%; myelodysplastic syndrome, 6.1%; mantle cell lymphoma, |
| | 11%), Deep venous thrombosis, All grades (9.3% to 10.3%), Deep |
| | venous thrombosis, Grade 3 or 4 (4% to 8.2%), Febrile neutropenia, |
| | Grade 3 or 4 (2.3% to 6%), Leukopenia, Grade 3 or 4 (4% to 7%), |
| | Neutropenia, Grade 3 or 4 (Multiple myeloma, 16% to 33.4% |
| | myelodysplastic syndrome, 53.4%; mantle cell lymphoma, 43%.), |
| | Thrombocytopenia, Grade 3 or 4 (Multiple myeloma, 8.3% to |
| | 12.2%; myelodysplastic syndrome, 50%; mantle cell lymphoma, |
| | 28%), Thrombosis |
| | Hepatic: Hepatotoxicity (15%), Liver failure |
| | Ophthalmic: Cataract, Grade 3 or 4 (Multiple myeloma, 9.6%; |
| | 1.4% (grade 3.4)) |
| | Renal: Interstitial nephritis, acute |
| | Respiratory: Hypoxia (Mantle cell lymphoma, 2%), Pleural |
| | effusion (Mantle cell lymphoma, 7%), Pneumonia (Multiple |
| | myeloma, 13.6% to 17.5%; myelodysplastic syndrome, 11.5%; |
| | mantle cell lymphoma, 14%), Pneumonitis (Myelodysplastic |
| | syndrome, grade 3 or 4, 1.4%), Pulmonary embolism, Grade 3 or 4 |
| | (2% to 4%), Pulmonary hypertension (Myelodysplastic syndrome, |
| | grade 3 or 4, 1.4%), Respiratory distress (Grade 3 or 4, 1% to 2%) |
| | Other: Angioedema, Multiple organ failure (Myelodysplastic |
| | syndrome, grade 3 or 4, 1.4%), Secondary malignant neoplastic |
| | disease, Tumor flare (Mantle cell lymphoma, 10%), Tumor lysis |
| | syndrome |
| Tracleer | Common |
| | Cardiovascular: Edema of lower extremity (5% to 8%), |
| | Hypotension (7%), Palpitations (5%) |
| | Dermatologic: Flushing (7% to 14%) |
| | Hematologic: Decreased hemoglobin (6%) |
| | Neurologic: Headache (up to 24%) |
| | Serious |
| | Hematologic: Decreased hemoglobin (Severe) (3%) |
| | Hepatic: Cirrhosis of liver, Increased liver aminotransferase level |
| | (Up to 11%), Liver failure |
| | Other: Angioedema |
| Xeljanz (Jak Compounds) | Common |
| | Endocrine metabolic: Increased HDL level (10% to 12%), Raised |
| | low density lipoprotein cholesterol (15% to 19%) |
| | Neurologic: Headache (3.4% to 4.3%) |
| | Renal: Urinary tract infectious disease (2%) |
| | Respiratory: Nasopharyngitis (2.8% to 3.8%), Upper respiratory |
| | infection (3.8% to 4.5%) |
| | Serious |
| | Dermatologic: Skin cancer, Non-melanoma |
| | Gastrointestinal: Gastrointestinal perforation |
| | Hematologic: Anemia, Decreased lymphocyte count (0.04%), |
| | Neutropenia (0.07%) |
| | Hepatic: Injury of liver |
| | Immunologic: Infectious disease (20% to 22%), Opportunistic |
| | infection, Post-transplant lymphoproliferative disorder, Epstein |
| | Barr virus associated (2.3%), Tuberculosis |
| | Other: Cancer |
| Atomoxetine (Strattera) | Common |
| | Cardiovascular: Increased diastolic arterial pressure (adult, 4.8% to |
| | 12.6%; pediatric, 9.3% to 21.5%), Increased systolic arterial |
| | pressure (adult, 4.2% to 12.4%; pediatric, 4.9% to 12.5%), |
| | Tachycardia (adult, 1.5% to 22.4%; pediatric, 0.3% to 23.4%) |
| | Endocrine metabolic: Weight decreased (adults, 2%; pediatric, 3% |
| | to 29.1%) |
| | Gastrointestinal: Abdominal pain (adult, 7%; pediatric, 17% to |
| | 18%), Constipation (adult, 8%; pediatric, 1% to 2%), Decrease in |
| | appetite (adult, 16%; pediatric, 16%), Nausea (adult, 26%; |
| | pediatric, 7% to 13%), Vomiting (adult, 4%; pediatric, 11%), |
| | Xerostomia (adult, 20%) |
| | Neurologic: Headache (pediatric, 19%), Insomnia (adult, 15%; |
| | pediatric, at least 2%), Somnolence (adult, 8%; pediatric, 11%) |
| | Renal: Delay when starting to pass urine (adult, 6%) |
| | Reproductive: Dysmenorrhea (adult, 3%), Erectile dysfunction |
| | (adult, 8%) |
| | Other: Menopausal flushing (adult, 3%) |
| | Serious |
| | Cardiovascular: Myocardial infarction, Sudden cardiac death |
| | Hepatic: Injury of liver (Severe), Liver failure |
| | Neurologic: Cerebrovascular accident, Dyskinesia, Seizure (adult, |
| | 0.1%; pediatric, 0.2%) |
| | Psychiatric: Mania, Psychotic disorder, Suicidal thoughts |
| | (pediatric, 0.4%) |
| | Reproductive: Priapism (rare) |
| Quetiapine (Seroquel) | See Seroquel above |
| Eszopiclone (Lunesta) | See Lunesta above |
| Gabapentin (Neurontin) | Common |
| | Cardiovascular: Peripheral edema (1.7% to 8.3%) |
| | Gastrointestinal: Nausea (greater than 1%), Vomiting (3.3%) |
| | Immunologic: Viral disease (10.9%) |
| | Neurologic: Ataxia (Adult, 3%; adult and adolescent, 13%), |
| | Nystagmus (Adult and adolescent, 8%) |
| | Other: Fatigue (3% to 11%), Fever (Pediatric, 10%) |
| | Serious |
| | Dermatologic: Stevens-Johnson syndrome |
| | Immunologic: Drug hypersensitivity syndrome |
| | Neurologic: Dizziness (Adults, 28%; adults and adolescents, 17%; |
| | pediatrics, 3%), Somnolence (Adults, 21%; adults and adolescents, |
| | 19%; pediatrics, 8%) |
| | Psychiatric: Disorder of form of thought (Pediatric, 1.7%), |
| | Disturbance in thinking (2% to 3%), Hostile behavior (Pediatric, |
| | 5.2%), Hyperactive behavior (Pediatric, 4.7%), Mood swings |
| | (Pediatric, 6%), Suicidal thoughts |
| Topiramate (Topamax) | Common |
| | Dermatologic: Flushing (pediatrics, 5%) |
| | Endocrine metabolic: Serum bicarbonate level abnormal (25% to |
| | 67%) |
| | Gastrointestinal: Loss of appetite (10% to 24%), Weight decreased |
| | (4% to 21%) |
| | Immunologic: Infectious disease (2% to 8%) |
| | Neurologic: Confusion (3% to 11%), Dizziness (4% to 25%), |
| | Impaired cognition (2% to 7%), Impaired psychomotor |
| | performance (2% to 13%), Memory impairment (3% to 12%), |
| | Paresthesia (1% to 51%), Reduced concentration span (2% to |
| | 10%), Somnolence (6% to 29%) |
| | Psychiatric: Feeling nervous (4% to 16%), Mood disorder (4% to |
| | 11%) |
| | Other: Fatigue (6% to 16%), Fever (1% to 12%) |
| | Serious |
| | Dermatologic: Erythema multiforme, Stevens-Johnson syndrome, |
| | Toxic epidermal necrolysis |
| | Endocrine metabolic: Hyperammonemia (Adolescents, 26%), |
| | Hypohidrosis, Increased body temperature, Metabolic acidosis |
| | Hepatic: Liver failure |
| | Neurologic: Drug-induced encephalopathy |
| | Ophthalmic: Glaucoma, Myopia, Visual field defect (epilepsy, |
| | 0.1% to 1%) |
| | Psychiatric: Suicidal thoughts |
| | Renal: Nephrolithiasis (adults, 1% to 3%) |
| Lamotrigine (Lamictal) | Common |
| | Dermatologic: Rash (7% to 14%) |
| | Gastrointestinal: Abdominal pain (immediate-release, 5% to 10%), |
| | Diarrhea (immediate-release, 6% to 11%; extended-release, 5%), |
| | Indigestion (immediate-release, 2% to 7%), Nausea (immediate- |
| | release, 7% to 25%; extended-release, 7%), Vomiting (immediate- |
| | release, 5% to 20%; extended-release, 6%)) |
| | Neurologic: Asthenia (immediate-release, 2% to 8%; extended- |
| | release, 6%), Ataxia (immediate-release, 2% to 11%), Coordination |
| | problem (immediate-release, 6% to 7%; extended-release, 3%), |
| | Dizziness (immediate-release, 7% to 54%; extended release, 14%), |
| | Headache (immediate-release, 29%), Insomnia (immediate-release, |
| | 5% to 10%), Somnolence (immediate-release, 9% to 17%; |
| | extended-release, 5%), Tremor (immediate-release, 4% to 10%; |
| | extended-release, 6%), Vertigo (immediate-release, 2%; extended- |
| | release, 3%) |
| | Ophthalmic: Blurred vision (immediate-release, 11% to 25% |
| | (adults) and 4% (children); extended-release, 3%), Diplopia |
| | (immediate-release, 24% to 49% (adults) and 5% (children); |
| | extended-release, 5%) |
| | Psychiatric: Anxiety (immediate-release, 4%; extended-release, |
| | 3%), Depression (immediate-release, 4%; extended-release, 3%) |
| | Reproductive: Dysmenorrhea (immediate-release, 5% to 7%) |
| | Respiratory: Rhinitis (immediate-release, 7% to 14%) |
| | Other: Pain (immediate-release, 5%) |
| | Serious |
| | Dermatologic: Erythema multiforme (less than 0.1%), Rash, |
| | Serious (0.08% to 0.8%), Stevens-Johnson syndrome (0.08% to |
| | 0.8%.), Toxic epidermal necrolysis (0.08% to 0.8%) |
| | Hematologic: Anemia (immediate release, less than 0.1%), |
| | Disseminated intravascular coagulation, Eosinophilia (immediate |
| | release, less than 0.1%), Leukopenia (immediate release, 0.1% to |
| | 1%), Thrombocytopenia (immediate release, less than 0.1%) |
| | Hepatic: Liver failure |
| | Immunologic: Drug hypersensitivity syndrome |
| | Neurologic: Aseptic meningitis |
| | Other: Angioedema (less than 0.1%), Neuroleptic malignant |
| | syndrome |
| Levetiracetam (Keppra) | Common |
| | Gastrointestinal: Loss of appetite (3% to 8%), Vomiting (15%) |
| | Immunologic: Infectious disease (13%) |
| | Musculoskeletal: Decreased bone mineral density (70%), Neck pain |
| | (2% to 8%) |
| | Neurologic: Asthenia (15%), Dizziness (5% to 9%), Headache |
| | (14% to 19%) |
| | Psychiatric:Abnormal behavior (7% to 37.6%), Irritability (6% to |
| | 12%) |
| | Respiratory: Cough (2% to 9%), Nasopharyngitis (7% to 15%) |
| | Other: Fatigue (10% to 11%) |
| | Serious |
| | Dermatologic: Stevens-Johnson syndrome, Toxic epidermal |
| | necrolysis due to drug |
| | Hematologic: Decreased erythrocyte production, Decreased white |
| | blood cell count (2.4% to 3.2%), Eosinophilia (8.6%), Neutropenia |
| | (partial onset seizures, adults, 2.4%), Pancytopenia, |
| | Thrombocytopenia |
| | Hepatic: Liver failure |
| | Neurologic: Somnolence (8% to 45%) |
| | Psychiatric: Suicidal intent (0.5%), Suicide |
| Olanzapine (Zyprexa) | Common |
| | Cardiovascular: Orthostatic hypotension (More than 5%), |
| | Peripheral edema (3% to 6%) |
| | Endocrine metabolic: Hypercholesterolemia (Adult, up to 26%; |
| | adolescent, up to 53%), Hyperglycemia (Adult, up to 20%; |
| | adolescent, up to 14%), Hyperprolactinemia (30% to 61.1%), |
| | Increased appetite (Adult, 3% to 24%; adolescent, 17% to 29%), |
| | Serum triglycerides raised (20.8% to 40%), Weight increased, 7% |
| | or greater (Adult, 22.2% to 64%; adolescent, 40.6% to 89%) |
| | Gastrointestinal: Constipation (4% to 11%), Xerostomia (Adult, up |
| | to 32%; adolescent, 4% to 7%) |
| | Neurologic: Akathisia (5% to 27%), Asthenia (2% to 20%), |
| | Dizziness (Adult, 1.6% to 18%; adolescent, 7% to 8%), |
| | Somnolence (IM, 6%; oral, 20% to 52%), Tremor (1% to 23%) |
| | Psychiatric: Personality disorder (8%) |
| | Serious |
| | Cardiovascular: Sudden cardiac death |
| | Endocrine metabolic: Diabetes mellitus, Diabetic coma with |
| | ketoacidosis, Diabetic ketoacidosis, Hyperglycemic hyperosmolar |
| | state |
| | Gastrointestinal: Acute hemorrhagic pancreatitis |
| | Hematologic: Leukopenia, Venous thromboembolism |
| | Immunologic: Hypersensitivity reaction |
| Risperidone (Risperdal) | Common |
| | Dermatologic: Rash (oral, adults, 1% to 4%; pediatrics, up to 11%; |
| | IM, less than 4%) |
| | Endocrine metabolic: Hyperprolactinemia (oral, adults, less than |
| | 1%; pediatrics, 49% to 87%; IM, less than 4%), Weight increased |
| | (oral, adult, 8.7% to 20.9%; pediatric, 14% to 32.6%; IM, adult, 8% |
| | to 10%) |
| | Gastrointestinal: Constipation (oral, 8% to 21%; IM, 5% to 7%), |
| | Diarrhea (oral, 1% to 8%; IM, less than 4%), Excessive salivation |
| | (oral, 1% to 10%; IM, 1% to 4%), Increased appetite (oral, adult, |
| | more than 5%; pediatric, 4% to 47%; IM, 4%), Indigestion (oral, |
| | 2% to 10%; IM, 6%), Nausea (oral, 4% to 16%; IM, 3% to 4%), |
| | Upper abdominal pain (oral, adult, more than 5%; pediatric, 13% to |
| | 16%), Vomiting (oral, 10% to 25%; IM, less than 4%), Xerostomia |
| | (oral, 4% to 15%; IM, up to 7%) |
| | Neurologic: Akathisia (oral, up to 10%; IM, 4% to 11%), Dizziness |
| | (oral, 4% to 16%; IM, 3% to 11%), Dystonia (oral, adult, 3% to |
| | 5%; pediatric, 2% to 6%; IM, adult, less than 4%), Parkinsonism |
| | (oral, 6% to 28%; IM, 8% to 15%), Sedated (oral, adult, 3% to 6%; |
| | pediatric, 8% to 29%), Tremor (oral, 2% to 12%; IM, 3% to 24%) |
| | Ophthalmic: Blurred vision (oral, 1% to 7%; IM, 2% to 3%) |
| | Psychiatric: Anxiety (oral, up to 16% IM, less than 4%) |
| | Respiratory: Cough (oral, adults, 2%; pediatrics, 24%; IM, 2% to |
| | 4%), Nasal congestion (oral, adult, 4% to 6%; pediatric, 13%), |
| | Nasopharyngitis (oral, adult, 3% to 4%; pediatric, 21%), Pain in |
| | throat (oral, adult, more than 5%; pediatric, 3% to 10%), Upper |
| | respiratory infection (oral, 2% to 8%; IM, 2% and 6%) |
| | Other: Fatigue (oral, adult, 1% to 3%; pediatric, 18% to 42%; IM, |
| | 3% to 9%), Pain, General (IM, 1% to 4%) |
| | Serious |
| | Cardiovascular: Prolonged QT interval, Sudden cardiac death, |
| | Syncope (oral, up to 1%; IM, up to 2%) |
| | Endocrine metabolic: Diabetic ketoacidosis, Hypothermia |
| | Gastrointestinal: Pancreatitis |
| | Hematologic: Agranulocytosis, Leukopenia, Neutropenia, |
| | Thrombocytopenia, Thrombotic thrombocytopenic purpura |
| | Neurologic: Cerebrovascular accident (oral, less than 5%; IM, less |
| | than 4%), Seizure (oral, 0.3%; IM, 0.3%), Tardive dyskinesia (oral, |
| | less than 5%; IM, less than 4%) |
| | Reproductive: Priapism |
| | Respiratory: Pulmonary embolism |
| | Other: Neuroleptic malignant syndrome (oral, adults, less than 1%; |
| | pediatrics, less than 5%) |
| Hydrocodone/ | Common |
| APAP (Generics) | Gastrointestinal: Nausea and vomiting |
| | Neurologic: Dizziness, Lightheadedness, Sedated |
| | Serious |
| | Dermatologic: Acute generalized exanthematous pustulosis, |
| | Stevens-Johnson syndrome, Toxic epidermal necrolysis |
| | Hematologic: Agranulocytosis, Thrombocytopenia |
| | Hepatic: Hepatotoxicity, Liver failure |
| | Respiratory: Respiratory depression |
| Tramadol (Ultram) | Common |
| | Dermatologic: Flushing (7.7% to 15.8%), Pruritus (3% to 11.9%) |
| | Gastrointestinal: Constipation (10% to 46%), Nausea (13% to |
| | 40%), Vomiting (3% to 17%), Xerostomia (1% to 10%) |
| | Neurologic: Dizziness (7% to 33%), Headache (3% to 32%), |
| | Insomnia (1% to 10.9%), Somnolence (4% to 25%) |
| | Serious |
| | Cardiovascular: Myocardial infarction (0.5% to less than 1%) |
| | Endocrine metabolic: Hypoglycemia (Very rare) |
| | Gastrointestinal: Pancreatitis (0.5% to less than 1%) |
| | Immunologic: Anaphylactoid reaction (less than 1%) |
| | Neurologic: Seizure |
| | Respiratory: Dyspnea (less than 5%), Respiratory depression |
| | Other: Serotonin syndrome (less than 1%) |
| Oxycodone/APAP (Percocet) | Common |
| | Gastrointestinal: Constipation (extended-release, 4%), Nausea |
| | (extended-release, 31%), Vomiting (extended-release, 9%) |
| | Neurologic: Dizziness (extended-release, 13%), Headache |
| | (extended-release, 10%), Lightheadedness, Sedated, Somnolence |
| | (extended-release, 4%) |
| | Serious |
| | Cardiovascular: Disorder of pulmonary circulation, Hypotension, |
| | Shock |
| | Dermatologic: Acute generalized exanthematous pustulosis, |
| | Stevens-Johnson syndrome, Toxic epidermal necrolysis |
| | Hematologic: Agranulocytosis, Neutropenia |
| | Hepatic: Hepatic necrosis, Hepatotoxicity, Liver failure |
| | Immunologic: Anaphylaxis, Hypersensitivity reaction |
| | Respiratory: Apnea, Respiratory arrest, Respiratory depression |
| | Other: Neonatal Abstinence Syndrome |
| Oxycodone (OxyContin) | See Oxycodone above |
| Codeine/APAP (Tylenol #2) | Common |
| | Gastrointestinal: Nausea, Vomiting |
| | Neurologic: Dizziness, Lightheadedness, Sedated, Somnolence |
| | Serious |
| | Dermatologic: Acute generalized exanthematous pustulosis, |
| | Stevens-Johnson syndrome, Toxic epidermal necrolysis |
| | Hematologic: Agranulocytosis, Thrombocytopenia |
| | Hepatic: Liver failure |
| | Immunologic: Hypersensitivity reaction |
| | Respiratory: Respiratory depression |
| Alprazolam (Xanax) | See Alprazolam above |
| Clonazepam (Klonopin) | See Clonazepam above |
| Diazepam (Valium) | See Diazepam above |
| Lorazepam (Ativan) | Common |
| | Neurologic: Asthenia (4.2%), Dizziness (6.9%), Sedated (15.9%), |
| | Unsteadiness present (3.4%) |
| | Psychiatric: Depression |
| | Serious |
| | Endocrine metabolic: Acidosis (less than 1%) |
| | Psychiatric: Delirium |
| Buspirone (Buspar) | Common |
| | Gastrointestinal: Nausea (8%) |
| | Neurologic: Dizziness (12%), Headache (6%), Somnolence (10%) |
| | Psychiatric: Feeling nervous (5%) |
| | Serious |
| | Cardiovascular: Congestive heart failure (less than 0.1%), |
| | Myocardial infarction (less than 0.1%) |
| | Neurologic: Cerebrovascular accident (less than 0.1%) |
| Hydroxyzine (Vistaril) | Common |
| | Gastrointestinal: Xerostomia |
| | Neurologic: Headache, Somnolence |
| Escitalopram (Lexapro) | Common |
| | Dermatologic: Diaphoresis (3% to 8%) |
| | Gastrointestinal: Abdominal pain (2%), Constipation (3% to 6%), |
| | Diarrhea (6% to 14%), Indigestion (2% to 6%), Nausea (15% to |
| | 18%), Vomiting (up to 3%), Xerostomia (4% to 9%) |
| | Neurologic: Dizziness (4% to 7%), Headache (24%), Insomnia (7% |
| | to 14%), Somnolence (4% to 13%) |
| | Reproductive: Disorder of ejaculation (9% to 14%), Erectile |
| | dysfunction (3%), Orgasm incapacity (females, 2% to 6%), |
| | Reduced libido (3% to 7%) |
| | Other: Fatigue (5% to 8%) |
| | Serious |
| | Psychiatric: Depression, worsening, Suicidal thoughts, Suicide |
| | Other: Serotonin syndrome |
| Sertraline (Zoloft) | Common |
| | Gastrointestinal: Constipation (3% to 8%), Diarrhea (13% to 24%), |
| | Indigestion (6% to 13%), Nausea (13% to 30%), Nausea and |
| | vomiting (2% to 30%) |
| | Neurologic: Dizziness (6% to 17%), Headache (25%), Insomnia |
| | (12% to 28%), Somnolence (2% to 15%), Tremor (5% to 11%) |
| | Reproductive: Abnormal ejaculation (7% to 19%), Reduced libido |
| | (up to 11%) |
| | Other: Fatigue (10% to 16%) |
| | Serious |
| | Dermatologic: Stevens-Johnson syndrome |
| | Endocrine metabolic: Hyponatremia |
| | Gastrointestinal: Gastrointestinal hemorrhage |
| | Immunologic: Anaphylaxis |
| | Musculoskeletal: Rhabdomyolysis |
| | Neurologic: Seizure (rare) |
| | Psychiatric: Depression, Exacerbation, Mania (rare), Suicidal |
| | thoughts (rare), Suicide (rare) |
| | Other: Serotonin syndrome |
| Trazodone (Desyrel) | Common |
| | Gastrointestinal: Constipation (7% to 8%), Diarrhea (up to 9%), |
| | Nausea (21%), Vomiting (at least 1%), Xerostomia (14% to 33.8%) |
| | Musculoskeletal: Backache (5%) |
| | Neurologic: Confusion (up to 5.7%), Dizziness (25%), Headache |
| | (9.9% to 33%), Insomnia (6.4% to 9.9%), Somnolence (23.9% to |
| | 46%) |
| | Ophthalmic: Blurred vision (5% to 14.7%) |
| | Psychiatric: Dream disorder (up to 5.1%), Feeling nervous (6.4% to |
| | 14.8%) |
| | Other: Fatigue (5.7% to 15%) |
| | Serious |
| | Cardiovascular: Cardiac dysrhythmia, Hypotension (3.8% to 7%), |
| | Prolonged QT interval, Torsades de pointes |
| | Immunologic: Hypersensitivity reaction (less than 1%) |
| | Neurologic: Seizure (rare), Serotonin syndrome |
| | Psychiatric: Suicidal thoughts (rare), Suicide |
| | Reproductive: Priapism |
| Duloxetine (Cymbalta) | Common |
| | Cardiovascular: Hypertension (2%) |
| | Dermatologic: Diaphoresis (Adult, 6%; pediatric, less than 2%) |
| | Gastrointestinal: Constipation (9% to 10%), Decrease in appetite |
| | (6% to 10%), Diarrhea (Adult, 9%; pediatric, 6%), Nausea (18% to |
| | 23%), Xerostomia (Adult, 11% to 14%; pediatric, 2%) |
| | Neurologic: Asthenia, Dizziness (Adult, 9%; pediatric, 8%), |
| | Headache (Adult, 13% to 14%; pediatric, 18%), Hypersomnia, |
| | Insomnia (7% to 10%), Sedated, Somnolence |
| | Other: Fatigue |
| | Serious |
| | Cardiovascular: Hypertensive crisis, Myocardial infarction (0.01% |
| | to 0.001%), Orthostatic hypotension |
| | Dermatologic: Stevens-Johnson syndrome |
| | Gastrointestinal: Gastrointestinal hemorrhage |
| | Hematologic: Bleeding, Abnormal |
| | Hepatic: Liver failure |
| | Psychiatric: Suicidal thoughts |
| | Other: Serotonin syndrome, Withdrawal sign or symptom (1% or |
| | greater) |
| Citalopram (Celexa) | Common |
| | Dermatologic: Diaphoresis (5% to 18%) |
| | Gastrointestinal: Constipation (13%), Diarrhea (8%), Nausea (20% |
| | to 21%), Vomiting (4% to 20%), Xerostomia (17% to 20%) |
| | Neurologic: Dizziness (14%), Headache (18%), Insomnia (15%), |
| | Sedated (15%), Somnolence (18%), Tremor (8% to 16%) |
| | Psychiatric: Agitation (3% to 10%) |
| | Reproductive: Disorder of ejaculation (6.1%) |
| | Other: Fatigue (5%) |
| | Serious |
| | Cardiovascular: Myocardial infarction (0.1% to 1%), Prolonged QT |
| | interval (0.5% to 1.9%), Torsades de pointes |
| | Neurologic: Cerebrovascular accident (0.1% to less than 1%) |
| | Psychiatric: Suicidal thoughts, Suicide |
| | Other: Serotonin syndrome |
| Aripiprazole (Abilify) | Common |
| | Endocrine metabolic: Weight increased, 7% or greater (2.5% to |
| | 21.5%) |
| | Gastrointestinal: Constipation (5% to 11%), Nausea (8% to 15%), |
| | Vomiting (3% to 11%) |
| | Neurologic: Akathisia (2% to 25%), Dizziness (4% to 10%), |
| | Extrapyramidal sign (2% to 27.3%), Headache (10% to 27%), |
| | Insomnia (8% to 18%), Sedated (3% to 21%), Somnolence (6% to |
| | 26.3%), Tremor (2% to 11.8%) |
| | Ophthalmic: Blurred vision (3% to 8%) |
| | Psychiatric: Anxiety (4% to 17%), Restlessness (2% to 12%) |
| | Other: Fatigue (2% to 17%) |
| | Serious |
| | Cardiovascular: Cardiorespiratory arrest (0.1% to 1%), |
| | Cardiorespiratory failure (0.1% to 1%), Myocardial infarction |
| | (0.1% to 1%), Prolonged QT interval (0.1% to 1%) |
| | Endocrine metabolic: Diabetic ketoacidosis (Less than 0.1%) |
| | Gastrointestinal: Pancreatitis (Less than 0.1%) |
| | Hematologic: Agranulocytosis, Leukopenia (Less than 1%), |
| | Neutropenia (Less than 1%) |
| | Musculoskeletal: Rhabdomyolysis (Less than 0.1%) |
| | Neurologic: Cerebrovascular accident, Seizure (Up to 0.3%), |
| | Tardive dyskinesia, Transient ischemic attack |
| | Psychiatric: At risk for suicide, Suicidal behavior |
| | Other: Angioedema (0.1% to less than 1%), Increased body |
| | temperature, Neuroleptic malignant syndrome |
| Paroxetine (Paxil) | Common |
| | Cardiovascular: Palpitations (up to 3%), Vasodilatation (2% to 4%) |
| | Dermatologic: Diaphoresis (5% to 14%) |
| | Gastrointestinal: Constipation (up to 16%), Diarrhea (up to 18%), |
| | Loss of appetite (up to 9%), Nausea (up to 26%), Xerostomia (9% |
| | to 18%) |
| | Neurologic: Asthenia (up to 22%), Dizziness (6% to 14%), |
| | Headache (17% to 27%), Insomnia (up to 24%), Somnolence (up to |
| | 24%), Tremor (4% to 11%) |
| | Ophthalmic: Blurred vision (up to 5%) |
| | Reproductive: Abnormal ejaculation (13% to 28%), Erectile |
| | dysfunction (2% to 9%), Orgasm disorder (females; 2% to 9%), |
| | Reduced libido (males: 6% to 15%; females: 0% to 9%) |
| | Respiratory: Yawning (4%) |
| | Serious |
| | Dermatologic: Stevens-Johnson syndrome, Toxic epidermal |
| | necrolysis |
| | Hepatic: Acute hepatitis (rare) |
| | Neurologic: Seizure (0.1%) |
| | Psychiatric: Depression, exacerbation, Suicidal thoughts (rare), |
| | Suicide (rare) |
| | Other: Serotonin syndrome |
| Fluoxetine (Prozac) | Common |
| | Gastrointestinal: Diarrhea (8% to 18%), Indigestion (6% to 10%), |
| | Loss of appetite (3.8% to 17%), Nausea (12% to 29%), Xerostomia |
| | (4% to 12%) |
| | Neurologic: Asthenia (7% to 21%), Dizziness (2% to 11%), |
| | Insomnia (9% to 33%), Somnolence (5% to 17%), Tremor (3% to |
| | 13%) |
| | Psychiatric: Anxiety (3% to 15%), Feeling nervous (3% to 14%) |
| | Respiratory: Pharyngitis (3% to 11%), Rhinitis (16% to 23%) |
| | Other: Influenza-like symptoms (3% to 12%) |
| | Serious |
| | Cardiovascular: Prolonged QT interval |
| | Dermatologic: Erythema multiforme |
| | Endocrine metabolic: Hyponatremia |
| | Hematologic: Bleeding |
| | Immunologic: Anaphylactoid reaction |
| | Neurologic: Seizure (0.2%) |
| | Psychiatric: Depression, worsening, Mania, Suicidal thoughts, |
| | Suicide |
| | Other: Serotonin syndrome |
| Venlafaxine (Effexor) | Common |
| | Cardiovascular: Hypertension (3% to 13%) |
| | Dermatologic: Sweating symptom (6.7% to 25%) |
| | Endocrine metabolic: Weight loss (3% to 47%) |
| | Gastrointestinal: Constipation (8% to 15%), Loss of appetite (8% to |
| | 22%), Nausea (21% to 58%), Xerostomia (12% to 22%) |
| | Neurologic: Asthenia (8% to 19%), Dizziness (11% to 23.9%), |
| | Dream disorder (3% to 7%), Headache (25% to 38%), Insomnia |
| | (14% to 24%), Somnolence (14% to 26%), Tremor (1.1% to |
| | 10.2%) |
| | Ophthalmic: Blurred vision (4% to 6%) |
| | Psychiatric: Feeling nervous (4% to 21.3%) |
| | Reproductive: Abnormal ejaculation (2.2% to 19%), Erectile |
| | dysfunction (2.1% to 6%), Orgasm disorder (2% to 5%) |
| | Serious |
| | Endocrine metabolic: Hyponatremia |
| | Gastrointestinal: Gastrointestinal hemorrhage (rare) |
| | Hematologic: Bleeding, Abnormal |
| | Hepatic: Hepatitis |
| | Neurologic: Seizure (0.3%) |
| | Psychiatric: Depression, exacerbation (rare), Hypomania, Mania, |
| | Suicidal thoughts (rare), Suicide |
| | Other: Neuroleptic malignant syndrome, Serotonin syndrome |
| Amitriptyline (Elavil) | Common |
| | Endocrine metabolic: Weight gain |
| | Gastrointestinal: Constipation, Xerostomia |
| | Neurologic: Dizziness, Headache, Somnolence |
| | Ophthalmic: Blurred vision |
| | Serious |
| | Cardiovascular: Cardiac dysrhythmia, Electrocardiogram abnormal, |
| | Myocardial infarction, Prolonged QT interval, Sudden cardiac |
| | death |
| | Hematologic: Agranulocytosis |
| | Hepatic: Hepatotoxicity, Jaundice (rare) |
| | Neurologic: Neuroleptic malignant syndrome, Seizure |
| | Psychiatric: Depression, worsening, Suicidal thoughts, Suicide |
| Bupropion (Wellbutrin) | Common |
| | Cardiovascular: Tachycardia (major depressive disorder, 11%) |
| | Endocrine metabolic: Weight gain (2% to 9%), Weight loss (major |
| | depressive disorder, 14% to 19%) |
| | Gastrointestinal: Abdominal pain (2% to 9%), Constipation (5% to |
| | 10%), Nausea (13% to 18%), Xerostomia (17% to 26%) |
| | Neurologic: Confusion (major depressive disorder, 8%), Dizziness |
| | (6% to 11%), Headache (25% to 34%), Insomnia (11% to 20%) |
| | Psychiatric: Agitation (2% to 9%) |
| | Respiratory: Nasopharyngitis (seasonal affective disorder, 13%), |
| | Pharyngitis (major depressive disorder, 3% to 11%), Upper |
| | respiratory infection (seasonal affective disorder, 9%) |
| | Serious |
| | Cardiovascular: Complete atrioventricular block, Myocardial |
| | infarction |
| | Gastrointestinal: Colitis, Pancreatitis |
| | Hematologic: Pancytopenia |
| | Hepatic: Abnormal liver function, Hepatitis, Jaundice, Liver |
| | damage |
| | Immunologic: Anaphylactoid reaction, Anaphylaxis, Delayed |
| | hypersensitivity disorder |
| | Musculoskeletal: Rhabdomyolysis |
| | Neurologic: Seizure (major depressive disorder, 0.1% to 0.4%) |
| | Psychiatric: Delusional disorder, Depression, Worsening, |
| | Hallucinations, Hostile behavior (major depressive disorder, 6%), |
| | Hypomania, Mania, Precipitation of episode, Paranoid ideation, |
| | Psychotic disorder, Activation, Suicidal behavior, Suicidal thoughts |
| | Respiratory: Pulmonary embolism |
| | Other: Angioedema |
| Nortriptyline (Pamelor) | Common |
| | Gastrointestinal: Constipation |
| | Serious |
| | Cardiovascular: Cardiac dysrhythmia, Heart block, Myocardial |
| | infarction, Prolonged QT interval, Sudden cardiac death |
| | Endocrine metabolic: Syndrome of inappropriate antidiuretic |
| | hormone secretion |
| | Gastrointestinal: Paralytic ileus |
| | Hematologic: Bone marrow depression |
| | Hepatic: Fulminant hepatic failure, Jaundice (rare) |
| | Neurologic: Cerebrovascular accident, Myoclonus, Seizure |
| | Psychiatric: Depression, worsening, Mania, Psychotic disorder, |
| | exacerbation, Suicidal thoughts, Suicide |
| | Other: Angioedema |
| Mirtazepine (Remeron) | Common |
| | Endocrine metabolic: Increased appetite (17%), Serum triglycerides |
| | raised (increases to 500 mg/dL or greater: 6%), Weight gain (body |
| | weight increase of 7% or greater: adults 7.5%; pediatrics 49%) |
| | Gastrointestinal: Constipation (13%), Xerostomia (25%) |
| | Hepatic: ALT/SGPT level raised (2%) |
| | Neurologic: Asthenia (8%), Dizziness (7%), Somnolence (54%) |
| | Psychiatric: Disturbance in thinking (3%) |
| | Serious |
| | Hematologic: Agranulocytosis, Neutropenia |
| | Hepatic: Cirrhosis of liver (less than 0.1%) |
| | Neurologic: Grand mal seizure (less than 0.1%), Status epilepticus |
| | Psychiatric: Depression, exacerbation, Suicidal thoughts, Suicide |
| | Other: Neuroleptic malignant syndrome, Serotonin syndrome (less |
| | than 0.1%) |
| Olanzapine (Zyprexa) | See Olanzapine above |
| Risperidone (Risperdal) | See Resperidone above |
| Antiepileptics | |
| Divalproex (Depakote) | Common |
| | Gastrointestinal: Abdominal pain (9% to 23%), Diarrhea (13% to |
| | 23%), Indigestion (8% to 11%), Loss of appetite (4% to 12%), |
| | Nausea (26% to 48%), Vomiting (15% to 27%) |
| | Musculoskeletal: Backache (Complex partial seizures, greater than |
| | 1% to less than 5%; migraine, 8%) |
| | Neurologic: Asthenia (6% to 27%), Dizziness (up to 25%), Feeling |
| | nervous (up to 11%), Headache (31%), Insomnia (up to 15%), |
| | Somnolence (Adult, 7% to 30%; pediatric, greater than 5%), |
| | Tremor (1% to 57%) |
| | Ophthalmic: Amblyopia, Blurred vision, Diplopia (16%) |
| | Other: Infectious disease (12% to 20%), Influenza (12%) |
| | Serious |
| | Cardiovascular: Palpitations (greater than 1% to less than 5%), |
| | Tachycardia (greater than 1% to less than 5%) |
| | Endocrine metabolic: Hyperammonemia |
| | Gastrointestinal: Pancreatitis (greater than 1% to less than 5%) |
| | Hematologic: Myelodysplastic syndrome, Thrombocytopenia, |
| | Dose-related (1% to 27%) |
| | Hepatic: Liver failure |
| | Immunologic: Drug hypersensitivity syndrome (rare) |
| | Neurologic: Hyperammonemic encephalopathy |
| | Otic: Ototoxicity - deafness (greater than 1% to less than 5%) |
| Ropinirole (Requip) | Common |
| | Cardiovascular: Hypotension (2% to 25%), Orthostatic hypotension |
| | (Up to 23%) |
| | Gastrointestinal: Abdominal pain (6% to 7%), Constipation (4% to |
| | 5%), Nausea (11% to 60%), Vomiting (7% to 12%) |
| | Neurologic: Dizziness (Parkinson disease, 6% to 40%; restless legs |
| | syndrome, 11%), Dyskinesia (13% to 34%), Headache (6%), |
| | Somnolence (Parkinson disease, 7% to 40%; restless leg syndrome, |
| | 12%) |
| | Other: Fatigue (8% to 11%) |
| | Serious |
| | Cardiovascular: Sinus node dysfunction, Syncope (Parkinson |
| | disease, 1% to 12%; restless leg syndrome, 1%) |
| | Neurologic: Sleep attack |
| | Psychiatric: Hallucinations (5% to 10%) |
| Pramipexole (Mirapex) | Common |
| | Cardiovascular: Orthostatic hypotension (Immediate release, 53%; |
| | extended-release, 3%) |
| | Gastrointestinal: Constipation (immediate-release, 4% to 14%; |
| | extended-release, 7% to 14%), Nausea (immediate-release, 11% to |
| | 28%; extended-release, 11% to 22%) |
| | Neurologic: Amnesia (4% to 6%), Asthenia (Immediate-release, |
| | 10% to 14%; extended-release, 3%), Confusion (4% to 10%), |
| | Dizziness (Immediate-release, 3% to 26%; extended-release, 2% to |
| | 12%), Dream disorder (Up to 11%), Dyskinesia (Immediate- |
| | release, 18% to 47%; extended-release, 17%), Extrapyramidal |
| | movements (28%), Headache (Immediate-release, 4% to 16%; |
| | extended-release, 7%), Insomnia (Immediate-release, 4% to 27%; |
| | extended-release, 4%) |
| | Psychiatric: Hallucinations (5% to 17%) |
| | Serious |
| | Cardiovascular: Heart failure |
| | Dermatologic: Malignant melanoma |
| | Neurologic: Sleep attack (2% to 6%), Somnolence (Immediate- |
| | release, 6% to 33%; extended-release, 15% to 36%) |
| | Psychiatric: Disturbance in thinking, Psychotic disorder |
| | Other: Malignant melanoma, Neuroleptic malignant syndrome |
| Methylphenidate (Concerta) | See Methylphenidate above |
| Lisdexamfetamine (Vyvanse) | Common |
| | Dermatologic: Rash (pediatrics, 3%) |
| | Endocrine metabolic: Decreased body growth, Weight decreased |
| | (pediatrics, 9%) |
| | Gastrointestinal: Diarrhea (adults, 7%), Loss of appetite (adults, 8% |
| | to 27%; pediatrics, 34% to 39%), Nausea (adults, 7%; pediatrics, |
| | 6%), Upper abdominal pain (pediatrics, 12%), Vomiting |
| | (pediatrics, 9%), Xerostomia (Adults, 26% to 36%; pediatrics, 4% |
| | to 5%) |
| | Neurologic: Dizziness (pediatrics, 5%), Insomnia (Adults, 20% to |
| | 27%; pediatrics, 13% to 23%) |
| | Psychiatric: Anxiety (Adults, 5% to 6%), Irritability (pediatrics, |
| | 10%) |
| | Serious |
| | Cardiovascular: Chest pain, Myocardial infarction, Peripheral |
| | vascular disease, Raynaud's disease, Sudden cardiac death, |
| | Tachycardia, Ventricular hypertrophy |
| | Immunologic: Anaphylaxis |
| | Neurologic: Cerebrovascular accident, Seizure |
| Amphetamine/Dextro- | See Adderall above |
| amphetamine (Adderall) | |
| Dalteparin (Fragmin), | Common |
| Danaparoid (Orgaran) | Dermatologic: Hematoma, Injection site (7% to 35%), Injection site |
| | pain (4.5% to 12%) |
| | Other: Irritation symptom, Local |
| | Serious |
| | Hematologic: Epidural hematoma, Hematoma, Spinal, |
| | Hemorrhage, Major (up to 13.6%), Hemorrhagic cerebral infarction |
| | (8%), Intracranial hemorrhage, Subdural hemorrhage, Intrauterine, |
| | Thrombocytopenia (Non-cancer indications, less than 1%; patients |
| | with cancer, 10.9% to 13.6%) |
| | Hepatic: Increased liver function test (up to 4.3%) |
| | Immunologic: Anaphylactoid reaction (rare) |
| | Neurologic: Paralysis |
| Enoxaparin (Lovenox) | Common |
| | Gastrointestinal: Diarrhea (2.2%), Nausea (2.5% to 3%) |
| | Hematologic: Anemia (up to 16%), Bleeding, Major (up to 4%), |
| | Thrombocytopenia (less than 3%) |
| | Hepatic: Increased liver function test (5.9% to 6.1%) |
| | Other: Fever (up to 8%) |
| | Serious |
| | Cardiovascular: Atrial fibrillation (0.7%), Heart failure (0.95%) |
| | Dermatologic: Eczematous drug eruption, Skin necrosis |
| | Hematologic: Hematoma, Hemorrhage (4% to 13%) |
| | Neurologic: Intracranial hemorrhage (0.8%), Paraplegia |
| | Respiratory: Pneumonia (0.82%) |
| Heparin (various) | Common |
| | Hematologic: Thrombocytopenia (up to 30%) |
| | Hepatic: Increased liver aminotransferase level |
| | Serious |
| | Hematologic: Hemorrhage (5% to 10%), Heparin-induced |
| | thrombocytopenia (1% to 10%), Heparin-induced |
| | thrombocytopenia with thrombosis (less than 1%) |
| | Immunologic: Hypersensitivity reaction |
| | Neurologic: Non-traumatic spinal subdural hematoma |
| Tinzaparin (Innohep) | Common |
| | Dermatologic: Erythema (16%) |
| | Hepatic: Increased liver function test, Asymptomatic (9% to 13%) |
| | Neurologic: Pain, Local (16%) |
| | Other: Irritation symptom, Local (16%) |
| | Serious |
| | Hematologic: Bleeding, Major (0.8%), Granulocytopenic disorder |
| | (rare), Hematoma, spinal/epidural, Pancytopenia (rare), |
| | Thrombocytopenia (1%), Thrombocytopenia (Severe) (0.13%) |
| | Immunologic: Anaphylaxis (rare) |
| | Neurologic: Paralysis |
| | Reproductive: Priapism (rare) |
| Warfarin (Coumadin) | Common |
| | Dermatologic: Alopecia |
| | Serious |
| | Cardiovascular: Cholesterol embolus syndrome, Gangrenous |
| | disorder (less than 0.1%) |
| | Dermatologic: Tissue necrosis (less than 0.1%) |
| | Hematologic: Bleeding, Hemorrhage |
| | Immunologic: Hypersensitivity reaction |
| | Musculoskeletal: Compartment syndrome |
| | Neurologic: Intracranial hemorrhage |
| | Ophthalmic: Intraocular hemorrhage |
| Dabigatran (Pradaxa) | Common |
| | Gastrointestinal: Esophagitis, Gastritis, Gastroesophageal reflux |
| | disease (Atrial fibrillation, 5.5%), Gastrointestinal hemorrhage |
| | (DVT and pulmonary embolism, 0.7% to 3.1%; nonvalvular atrial |
| | fibrillation, 6.1%), Gastrointestinal ulcer, Indigestion (DVT and |
| | pulmonary embolism, 7.5%) |
| | Hematologic: Bleeding (DVT and pulmonary embolism |
| | prophylaxis, 10.5%; nonvalvular atrial fibrillation, 16.6%) |
| | Serious |
| | Cardiovascular: Myocardial infarction (DVT and pulmonary |
| | embolism, 0.32% to 0.66%; nonvalvular atrial fibrillation, 0.7%) |
| | Gastrointestinal: Gastrointestinal hemorrhage, Major (DVT and |
| | pulmonary embolism, 0.3% to 0.6%; nonvalvular atrial fibrillation, |
| | 1.6%) |
| | Hematologic: Bleeding, Major (DVT and pulmonary embolism, |
| | 0.3% to 1.4%; nonvalvular atrial fibrillation, 3.3%), Thrombosis |
| | Immunologic: Anaphylaxis |
| | Neurologic: Epidural hematoma, Intracranial hemorrhage |
| | (nonvalvular atrial fibrillation, 0.3%; DVT and pulmonary |
| | embolism, 0.1%), Traumatic spinal subdural hematoma |
| | Respiratory: Bleeding, Alveolar |
| Rivaroxaban (Xarelto) | Common |
| | Hematologic: Bleeding (Hip/knee replacement, 5.8%; |
| | DVT/pulmonary embolism, 17.4% to 28.3%) |
| | Serious |
| | Cardiovascular: Syncope (1.2%) |
| | Gastrointestinal: Gastrointestinal hemorrhage (nonvalvular atrial |
| | fibrillation, 3.1%) |
| | Hematologic: Bleeding, Major (Nonvalvular atrial fibrillation, |
| | 5.6%; hip/knee replacement, 0.3%; DVT/pulmonary embolism, |
| | 1%), Epidural hematoma, Hematoma, Spinal |
| | Immunologic: Anaphylaxis, Hypersensitivity reaction |
| | Other: Drug withdrawal, Stroke and non-CNS embolism |
| Apixaban (Eliquis) | Common |
| | Dermatologic: Contusion (1.4% to 2.2%) |
| | Gastrointestinal: Bleeding gums (Less than 0.1% to 1.4%) |
| | Hematologic: Hematoma (DVT, 1.3% to 1.5%) |
| | Reproductive: Menorrhagia (1.4%) |
| | Respiratory: Epistaxis (DVT and pulmonary embolism, 1.5% to |
| | 3.6%; DVT prophylaxis, 0.1% to less than 1%), Hemoptysis (Less |
| | than 0.1% to 1.2%) |
| | Serious |
| | Gastrointestinal: Gastrointestinal hemorrhage (atrial fibrillation, |
| | 0.83%/year; DVT prophylaxis, 0.1% to less than 1%; DVT and |
| | pulmonary embolism, 0.1% to less than 1%), Hematochezia (0.1% |
| | to less than 1%), Rectal hemorrhage (Less than 0.1% to 1%) |
| | Hematologic: Bleeding (atrial fibrillation, 2.08%/year; DVT |
| | prophylaxis, 2.88% to 4.83%), Bleeding, Major (0.1% to 2.13%), |
| | Hemorrhage (0.1% to 1.4%), Hemorrhage, Operative (DVT |
| | prophylaxis, 0.1% to less than 1%) |
| | Hepatic: Alkaline phosphatase raised (DVT prophylaxis, 0.1% to |
| | less than 1%), Liver function tests abnormal (DVT prophylaxis, |
| | 0.1% to less than 1%), Serum bilirubin raised (DVT prophylaxis, |
| | 0.1% to less than 1%) |
| | Immunologic: Hypersensitivity reaction (atrial fibrillation, less than |
| | 1%) |
| | Musculoskeletal: Hemorrhage of muscle (Less than 0.1% to less |
| | than 1%) |
| | Neurologic: Epidural hematoma, Intracranial hemorrhage (atrial |
| | fibrillation, 0.33% to 0.34%/year), Non-traumatic spinal subdural |
| | hematoma, Traumatic spinal subdural hematoma |
| | Ophthalmic: Conjunctival hemorrhage (0.1% to less than 1%), |
| | Intraocular hemorrhage (Less than 0.1% to less than 1%), Retinal |
| | hemorrhage (0.1% to less than 1%) |
| | Renal: Hematuria (DVT, 1.4% to 2.1%; DVT prophylaxis, 0.1% to |
| | less than 1%) |
| Edoxaban (Savaysa) | Common |
| | Dermatologic: Rash (3.6% to 4.2%) |
| | Hematologic: Anemia (Nonvalvular atrial fibrillation, 9.6%; DVT |
| | or pulmonary embolism, 1.7%), Bleeding, Clinically Relevant, |
| | Nonmajor (Nonvalvular atrial fibrillation, 9.4%; DVT or |
| | pulmonary embolism, 7.2%) |
| | Hepatic: Liver function tests abnormal (Nonvalvular atrial |
| | fibrillation, 4.8%; DVT or pulmonary embolism, 7.8%) |
| | Serious |
| | Hematologic: Bleeding, Major (Nonvalvular atrial fibrillation, |
| | 3.1%; DVT or pulmonary embolism, 1.4%) |
| | Neurologic: Hemorrhagic cerebral infarction (Nonvalvular atrial |
| | fibrillation, 0.3%), Intracranial hemorrhage (Nonvalvular atrial |
| | fibrillation, 0.5%; DVT or pulmonary embolism, 0.1%) |
| | Respiratory: Interstitial lung disease (Nonvalvular atrial fibrillation, |
| | 0.2%) |
| Aspirin | Serious |
| | Gastrointestinal: Gastrointestinal ulcer |
| | Hematologic: Bleeding |
| | Ophthalmic: Exudative age-related macular degeneration |
| | Otic: Tinnitus |
| | Respiratory: Bronchospasm |
| | Other: Angioedema, Reye's syndrome |
| Ticlopidine | Common |
| | Dermatologic: Rash (1% to 11.8%) |
| | Gastrointestinal: Abdominal pain, Diarrhea, Indigestion, Loss of |
| | appetite, Nausea |
| | Hematologic: Hemorrhage, Leukopenia |
| | Hepatic: Liver function tests abnormal |
| | Neurologic: Dizziness |
| | Serious |
| | Hematologic: Agranulocytosis, Aplastic anemia (rare), |
| | Granulocytopenic disorder, Neutropenia (2.4%), Pancytopenia, |
| | Thrombocytopenia, Thrombotic thrombocytopenic purpura (rare) |
| Clopidogrel (Plavix ®) | Common |
| | Hematologic: Bleeding, Non-major (3.6% to 5.1%) |
| | Serious |
| | Cardiovascular: Coronary artery stent thrombosis |
| | Dermatologic: Fixed drug eruption |
| | Gastrointestinal: Colitis, Gastrointestinal hemorrhage (2%; 2.7% |
| | with aspirin) |
| | Hematologic: Agranulocytosis (Less than 1%), Aplastic anemia |
| | (less then 1%), Bleeding, Major (0.8% to 3.7%), Pancytopenia |
| | (Severe), Thrombotic thrombocytopenic purpura |
| | Hepatic: Hepatitis, Hepatotoxicity, Liver failure |
| | Immunologic: Hypersensitivity reaction |
| | Neurologic: Epidural hematoma, Intracranial hemorrhage |
| | Ophthalmic: Intraocular hemorrhage (0.05%) |
| | Other: Drug withdrawal, Rebound effect |
| Dipyridamole | Common |
| | Cardiovascular: Chest pain (IV, up to 30%), Electrocardiogram |
| | abnormal (IV, 0.8% to 7.5%) |
| | Dermatologic: Flushing (IV, 3.4%), Rash (oral, 2.3%) |
| | Gastrointestinal: Abdominal discomfort (oral, 6.1%) |
| | Neurologic: Dizziness (oral, 13.6%; IV, 11.8%), Headache (oral, |
| | 2.3%; IV, 12.2% to 20%) |
| | Respiratory: Dyspnea (IV, 2.6% to 25%) |
| | Serious |
| | Cardiovascular: Angina, Cardiac arrest, Myocardial infarction (IV, |
| | 0.1%), Myocardial ischemia, Ventricular fibrillation, Ventricular |
| | tachycardia (IV, 0.2%) |
| | Hepatic: Liver failure |
| | Immunologic: Hypersensitivity reaction |
| | Neurologic: Cerebrovascular accident, Seizure |
| | Respiratory: Bronchospasm (IV, 0.2%) |
| Benazepril (Lotensin) | Common |
| | Neurologic: Dizziness (3.6%), Headache (6.2%) |
| | Respiratory: Cough (1.2%) |
| | Other: Fatigue (2.4%) |
| | Serious |
| | Dermatologic: Stevens-Johnson syndrome (less than 1%) |
| | Gastrointestinal: Intestinal angioedema |
| | Hematologic: Agranulocytosis, Neutropenia |
| | Hepatic: Hepatic necrosis (rare), Increased liver enzymes (rare), |
| | Jaundice (rare) |
| | Immunologic: Anaphylactoid reaction |
| | Renal: Renal impairment |
| | Other: Angioedema, head and neck (0.5%) |
| Captopril (Capoten) | Common |
| | Cardiovascular: Hypotension |
| | Dermatologic: Rash |
| | Endocrine metabolic: Hyperkalemia (11%) |
| | Gastrointestinal: Disorder of taste |
| | Respiratory: Cough (0.5% to 2%) |
| | Serious |
| | Dermatologic: Stevens-Johnson syndrome |
| | Gastrointestinal: Intestinal angioedema |
| | Hematologic: Agranulocytosis (0.1% to 0.2%), Neutropenia (0.1% |
| | to 0.2%) |
| | Immunologic: Anaphylactoid reaction |
| | Other: Angioedema (0.1%) |
| Enalapril (Vasotec) | Common |
| | Endocrine metabolic: Hyperkalemia (1% to 3.8%) |
| | Neurologic: Dizziness (4.3% to 7.9%) |
| | Renal: Serum blood urea nitrogen raised (0.2% (hypertension) to |
| | 20% (hypertension with renal artery stenosis)), Serum creatinine |
| | raised (0.2% (hypertension) to 20% (hypertension with renal artery |
| | stenosis)) |
| | Other: Fatigue (3%) |
| | Serious |
| | Cardiovascular: Hypotension (0.9% to 6.7%) |
| | Gastrointestinal: Intestinal angioedema |
| | Hematologic: Agranulocytosis |
| | Hepatic: Hepatotoxicity, Liver failure |
| | Immunologic: Anaphylactoid reaction, during desensitization |
| | Renal: Acute renal failure, Renal impairment |
| | Other: Angioedema (0.1% to 1%) |
| Fosinopril (Monopril) | Common |
| | Cardiovascular: Hypotension (2.4% to 4.4%) |
| | Endocrine metabolic: Hyperkalemia (2.6%) |
| | Gastrointestinal: Nausea and vomiting (1.2% to 2.2%) |
| | Neurologic: Dizziness (1.6% to 11.9%) |
| | Respiratory: Cough (2.2% to 9.7%) |
| | Serious |
| | Gastrointestinal: Intestinal angioedema |
| | Hematologic: Agranulocytosis |
| | Immunologic: Anaphylactoid reaction |
| | Renal: Acute renal failure, Azotemia, Oliguria |
| | Other: Angioedema, Head and Neck |
| Lisinopril (Prinivil, Zestril) | Common |
| | Cardiovascular: Chest pain, Hypotension (up to 11%), Syncope |
| | (5% to 7%) |
| | Neurologic: Dizziness (12% to 19%), Headache |
| | Respiratory: Cough |
| | Serious |
| | Cardiovascular: Hypotension (Severe) (9%) |
| | Dermatologic: Stevens-Johnson syndrome (1% or more), Toxic |
| | epidermal necrolysis (1% or more) |
| | Endocrine metabolic: Hyperkalemia (2.2% to 6%) |
| | Gastrointestinal: Intestinal angioedema |
| | Immunologic: Anaphylaxis due to hymenoptera venom, Dialysis |
| | membrane-induced anaphylactoid reaction |
| | Renal: Acute renal failure, Renal impairment (2.4%) |
| | Other: Angioedema, Head and Neck |
| Moexipril (Univasc) | Common |
| | Gastrointestinal: Diarrhea (3.1%) |
| | Neurologic: Dizziness (4.3%) |
| | Respiratory: Cough (6.1%) |
| | Other: Influenza-like symptoms |
| | Serious |
| | Gastrointestinal: Intestinal angioedema |
| | Hematologic: Agranulocytosis |
| | Immunologic: Anaphylaxis due to hymenoptera venom, Dialysis |
| | membrane-induced anaphylactoid reaction |
| | Renal: Abnormal renal function |
| | Other: Angioedema, Head and Neck |
| Perindopril (Aceon) | Common |
| | Endocrine metabolic: Hyperkalemia |
| | Musculoskeletal: Backache (5.8%) |
| | Neurologic: Asthenia, Dizziness (8.2%), Headache |
| | Respiratory: Cough (12%) |
| | Serious |
| | Cardiovascular: Cardiac arrest, Orthostatic hypotension (0.8%) |
| | Gastrointestinal: Intestinal angioedema, Pancreatitis |
| | Hematologic: Agranulocytosis, Bone marrow depression, |
| | Neutropenia |
| | Hepatic: Liver failure |
| | Renal: Acute renal failure |
| | Other: Angioedema (0.1% to 0.5%) |
| Quinapril (Accupril) | Common |
| | Cardiovascular: Chest pain (2.4%), Hypotension (2.9%) |
| | Gastrointestinal: Nausea and vomiting (1.4% to 2.4%) |
| | Neurologic: Dizziness (3.9% to 7.7%), Headache (1.7% to 6.9%) |
| | Respiratory: Cough (2% to 4.3%) |
| | Other: Fatigue (2.6%) |
| | Serious |
| | Gastrointestinal: Intestinal angioedema |
| | Hematologic: Agranulocytosis |
| | Immunologic: Anaphylactoid reaction (rare), Anaphylaxis due to |
| | hymenoptera venom, Dialysis membrane-induced anaphylactoid |
| | reaction |
| | Renal: Serum blood urea nitrogen raised (2% to 8%), Serum |
| | creatinine raised (2% to 11%) |
| | Other: Angioedema |
| Ramipril (Altace) | Common |
| | Cardiovascular: Hypotension (11%) |
| | Neurologic: Asthenia, Dizziness (2.2% to 4%), Headache (5.4%) |
| | Respiratory: Cough (8% to 12%) |
| | Other: Fatigue |
| | Serious |
| | Dermatologic: Stevens-Johnson syndrome |
| | Gastrointestinal: Intestinal angioedema, Pancreatitis |
| | Hepatic: Hepatic necrosis, Hepatotoxicity |
| | Immunologic: Anaphylactoid reaction |
| | Other: Angioedema, Head and Neck |
| Trandolapril (Mavik) | Common |
| | Cardiovascular: Hypotension (0.6% to 11%), Syncope (5.9%) |
| | Endocrine metabolic: Hyperkalemia (0.3% to 5.3%) |
| | Gastrointestinal: Indigestion (0.3% to 6.4%) |
| | Neurologic: Dizziness (1.3% to 23%) |
| | Renal: Serum blood urea nitrogen raised (9%) |
| | Respiratory: Cough (1.9% to 35%) |
| | Serious |
| | Cardiovascular: Cardiogenic shock (3.8%), Intermittent |
| | claudication (3.8%) |
| | Gastrointestinal: Intestinal angioedema |
| | Hematologic: Agranulocytosis |
| | Immunologic: Anaphylaxis due to hymenoptera venom, Dialysis |
| | membrane-induced anaphylactoid reaction |
| | Other: Angioedema |
| Candesartan (Atacand) | Common |
| | Cardiovascular: Hypotension (18.8%) |
| | Musculoskeletal: Backache (3%.) |
| | Neurologic: Dizziness (less than 5%) |
| | Respiratory: Pharyngitis (2%), Rhinitis (2%), Upper respiratory |
| | infection (6%) |
| Eprosartan (Teveten) | Common |
| | Gastrointestinal: Abdominal pain |
| | Musculoskeletal: Myalgia (1.9%) |
| | Neurologic: Dizziness (3.8%) |
| | Respiratory: Upper respiratory infection |
| | Other: Fatigue (1.4%) |
| | Serious |
| | Dermatologic: Edema of face (rare) |
| | Hematologic: Neutropenia (1.3%) |
| Irbesartan (Avapro) | Common |
| | Gastrointestinal: Diarrhea, Heartburn |
| | Neurologic: Headache |
| | Respiratory: Upper respiratory infection |
| | Other: Fatigue |
| | Serious |
| | Hematologic: Thrombocytopenia |
| | Hepatic: Cholestasis, Hepatitis |
| | Musculoskeletal: Rhabdomyolysis |
| | Renal: Renal failure |
| | Other: Angioedema, face, lips, throat |
| Losartan (Cozaar) | Common |
| | Cardiovascular: Chest pain (12%), Hypotension (7%) |
| | Endocrine metabolic: Hyperkalemia (7%), Hypoglycemia (14%) |
| | Gastrointestinal: Diarrhea (15%) |
| | Hematologic: Anemia (14%) |
| | Neurologic: Asthenia, Dizziness (3%) |
| | Respiratory: Cough (10%) |
| | Other: Fatigue |
| | Serious |
| | Hepatic: Hepatotoxicity |
| | Musculoskeletal: Rhabdomyolysis |
| | Renal: Acute renal failure |
| | Other: Angioedema |
| Telmisartan (Micardis)) | Common |
| | Respiratory: Cough (1.6% to 15.6%), Upper respiratory infection |
| | (7%) |
| | Serious |
| | Musculoskeletal: Rhabdomyolysis (rare) |
| Valsartan (Diovan) | Common |
| | Cardiovascular: Hypotension (5.5% to 6.9%) |
| | Neurologic: Dizziness (2% to 17%), Headache (greater than 1%) |
| | Renal: Serum blood urea nitrogen raised (heart failure, 16.6%), |
| | Serum creatinine raised (hypertension, 0.8%; heart failure, 3.9%; |
| | post-myocardial infarction, 4.2%) |
| | Respiratory: Cough |
| | Serious |
| | Renal: Acute renal failure |
| | Other: Angioedema, Face, lips, throat |
| Sacubitril/valsartan (Entresto) | Common |
| | Cardiovascular: Hypotension (18%) |
| | Endocrine metabolic: Hyperkalemia (12%) |
| | Neurologic: Dizziness (6%) |
| | Serious |
| | Renal: Renal failure (5%) |
| | Other: Angioedema (0.5%) |
| Acebutolol (Sectral) | Common |
| | Neurologic: Dizziness (6%), Headache (6%) |
| | Other: Fatigue (11%) |
| | Serious |
| | Cardiovascular: Angina (2%), Bradyarrhythmia (2%), Heart failure |
| | (2%) |
| | Hepatic: Hepatotoxicity (rare) |
| | Immunologic: Anaphylaxis, Systemic lupus erythematosus (rare.) |
| Atenolol (Tenormin) | Common |
| | Cardiovascular: Bradyarrhythmia (3% to 18%), Cold extremities |
| | (12%), Hypotension (4% to 25%) |
| | Neurologic: Dizziness (13%) |
| | Psychiatric: Depression (up to 12%) |
| | Other: Fatigue (up to 26%) |
| | Serious |
| | Cardiovascular: Heart failure, Myocardial infarction, Ventricular |
| | arrhythmia |
| | Endocrine metabolic: Thyrotoxicosis |
| | Immunologic: Anaphylaxis, Systemic lupus erythematosus |
| | Respiratory: Pulmonary embolism (1.2%) |
| | Other: Withdrawal sign or symptoms |
| Betaxolol (Kerlone) | Common |
| | Cardiovascular: Bradyarrhythmia (5.8% to 8.1%) |
| | Gastrointestinal: Indigestion (3.9% to 4.7%), Nausea (1.6% to |
| | 5.8%) |
| | Musculoskeletal: Arthralgia (3.1% to 5.2%), Chest pain (2.4% to |
| | 7.1%) |
| | Ophthalmic: Burning sensation in eye (30%, ophthalmic) |
| | Other: Fatigue (2.9% to 9.7%) |
| | Serious |
| | Cardiovascular: Atrioventricular block, Myocardial infarction |
| Bisoprolol/hydrochlorothiazide | Common |
| (Ziac) | Gastrointestinal: Diarrhea (4.3%) |
| | Neurologic: Dizziness (5.1%), Headache (4.5%) |
| | Other: Fatigue (4.6%) |
| | Serious |
| | Cardiovascular: Heart failure |
| | Ophthalmic: Angle-closure glaucoma, acute, Myopia, Acute |
| | transient |
| | Respiratory: Bronchospasm |
| Bisoprolol (Zebeta) | Common |
| | Gastrointestinal: Diarrhea (2.6% to 3.5%) |
| | Neurologic: Headache (8.8% to 10.9%) |
| | Respiratory: Rhinitis (2.9% to 4%), Upper respiratory infection |
| | (4.8% to 5%.) |
| | Other: Fatigue (6.6% to 8.2%) |
| Carteolol (Cartrol) | Common |
| | Cardiovascular: Angina |
| | Neurologic: Asthenia (7%.), Dizziness (4% to 15%.), Headache |
| | (4% to 17%.), Insomnia (2% to 12%.) |
| | Ophthalmic: Blurred vision, Burning sensation in eye (25%), |
| | Conjunctival edema (25%), Conjunctival hyperemia (25%), |
| | Epiphora (25%), Eye irritation (25%) |
| | Serious |
| | Cardiovascular: Cardiac dysrhythmia, Heart failure |
| | Respiratory: Bronchospasm |
| Metoprolol (Lopressor, Toprol | Common |
| XL) | Cardiovascular: Bradyarrhythmia (3%), Cold extremities (1%), |
| | Heart failure (1%), Hypotension (1%) |
| | Dermatologic: Pruritus (5%), Rash (5%) |
| | Gastrointestinal: Constipation (1%), Diarrhea (5%), Indigestion |
| | (1%), Nausea (1%) |
| | Neurologic: Dizziness (10%), Fatigue (10%), Headache |
| | Psychiatric: Depression (5%) |
| | Respiratory: Dyspnea (3%), Wheezing (1%) |
| | Serious |
| | Respiratory: Bronchospasm (1%) |
| Nadolol (Corgard) | Common |
| | Cardiovascular: Bradyarrhythmia (2%) |
| | Neurologic: Dizziness (2%) |
| | Other: Fatigue (2%) |
| | Serious |
| | Cardiovascular: Atrioventricular block, Cardiac dysrhythmia (1%), |
| | Heart failure (1%) |
| Propranolol (Inderal) | Common |
| | Gastrointestinal: Diarrhea, Vomiting |
| | Neurologic: Dizziness (Hypertension, 4% to 7%), Sleep disorder |
| | Other: Fatigue (5% to 7%) |
| | Serious |
| | Cardiovascular: Bradyarrhythmia, Cardiogenic shock, Congestive |
| | heart failure, Heart block, Heart failure, Hypotension, Prolonged |
| | PR interval, Shortened QT interval |
| | Dermatologic: Erythroderma, Stevens-Johnson syndrome, Toxic |
| | epidermal necrolysis |
| | Endocrine metabolic: Hypoglycemia |
| | Immunologic: Anaphylaxis |
| | Neurologic: Cerebrovascular accident |
| | Respiratory: Bronchospasm |
| | Other: Withdrawal sign or symptom |
| Sotalol (Betapace) | Common |
| | Cardiovascular: Chest pain (Adult, 16%; pediatric, 4%), |
| | Lightheadedness (12%) |
| | Dermatologic: Rash (5%) |
| | Neurologic: Disturbance of consciousness (4%), Dizziness (13.1% |
| | to 20%), Headache (3.3% to 11.5%) |
| | Respiratory: Dyspnea (9.2% to 21%) |
| | Other: Fatigue (18.9% to 20%) |
| | Serious |
| | Cardiovascular: Atrioventricular block, Bradyarrhythmia (Adult, |
| | 12.3% to 16%; pediatric, 4%), Cardiac dysrhythmia (5%), |
| | Congestive heart failure (1.2% to 3.3%), Heart failure (5%), |
| | Prolonged QT interval, Torsades de pointes (0.5% to 4%) |
| | Neurologic: Cerebrovascular accident (1%) |
| Timolol (Blocadren) | Common |
| | Cardiovascular: Angina, Bradyarrhythmia (5% to 9.1%, oral), |
| | Heart failure (8%, oral), Hypotension |
| | Dermatologic: Pruritus, Rash, Urticaria |
| | Gastrointestinal: Abdominal pain, Diarrhea, Indigestion, Nausea, |
| | Vomiting |
| | Musculoskeletal: Cramp |
| | Neurologic: Confusion (13%), Dizziness (2.3% to 6%, oral), |
| | Headache (1.7%, oral; 1% to 5%, ophthalmic) |
| | Ophthalmic: Blurred vision (Ophthalmic, 15% to 33%), Burning |
| | sensation in eye (Ophthalmic, 12.5% to 20%), Dry eyes |
| | Psychiatric: Depression (9.2%, ophthalmic), Hallucinations (11%), |
| | Psychotic disorder (3%) |
| | Respiratory: Cough, Dyspnea (1.7%, oral) |
| | Other: Fatigue (3.4% to 5%, oral), Infectious disease |
| | Serious |
| | Cardiovascular: Cardiac dysrhythmia (1%), Myocardial infarction |
| | (rare) |
| | Respiratory: Bronchospasm (0.6%, oral) |
| carvedilol, Common brand | Common |
| names - Coreg* | Cardiovascular: Bradyarrhythmia (3% to 10%), Hypotension (1.8% |
| | to 20.2%), Peripheral edema (1% to 7%) |
| | Endocrine metabolic: Abnormal weight gain (10% to 12%), |
| | Hyperglycemia (5% to 12%) |
| | Gastrointestinal: Diarrhea (2% to 12%) |
| | Neurologic: Dizziness (6% to 33%) |
| | Reproductive: Erectile dysfunction (13.5%) |
| | Other: Fatigue (24%) |
| | Serious |
| | Cardiovascular: Atrioventricular block (greater than 1% to 3%) |
| | Dermatologic: Erythema multiforme, Stevens-Johnson syndrome, |
| | Toxic epidermal necrolysis |
| | Hematologic: Aplastic anemia |
| | Ophthalmic: Intraoperative floppy iris syndrome |
| | Respiratory: Asthma with status asthmaticus (rare) |
| labetolol hydrochloride, | Common |
| Common brand names - | Cardiovascular: Orthostatic hypotension (1%, oral; 58%, IV) |
| Normodyne*, Trandate* | Dermatologic: Has tingling sensation (7%.) |
| | Gastrointestinal: Nausea (14%) |
| | Neurologic: Dizziness (9% to 20%) |
| | Respiratory: Nasal congestion (3%) |
| | Other: Fatigue (11%) |
| | Serious |
| | Cardiovascular: Heart failure |
| | Endocrine metabolic: Hyperkalemia |
| | Hepatic: Hepatotoxicity |
| | Respiratory: Bronchospasm |
| Amlodipine (Norvasc, Lotrel) | Common |
| | Cardiovascular: Flushing (0.7% to 2.6%), Palpitations (Up to |
| | 4.5%), Peripheral edema (Up to 10.8%) |
| | Gastrointestinal: Abdominal pain (1.6%), Nausea (2.9%.) |
| | Neurologic: Dizziness (Up to 3.4%), Headache (7.3%), |
| | Somnolence (1.4%) |
| | Other: Fatigue (4.5%) |
| | Serious |
| | Cardiovascular: Acute myocardial infarction, Angina |
| | Other: Angioedema |
| Bepridil (Vascor) | Common |
| | Diarrhea |
| | Dizzy |
| | Feel Like Throwing Up |
| | Infrequent side effects of Vascor: |
| | Abnormal Heart Rhythm |
| | Chronic Heart Failure |
| | Fluid in the Lungs |
| | Prolonged Q-T Interval on EKG |
| | Slow Heartbeat |
| | Very Rapid Heartbeat - Torsades de Pointes |
| | Head Pain |
| | Incomplete or Infrequent Bowel Movements |
| | Low Energy |
| | Rare side effects of Vascor: |
| | Deficiency of Granulocytes a Type of White Blood Cell |
| | Fluid Retention in the Legs, Feet, Arms or Hands |
| | Inflammation of Skin caused by an Allergy |
| | Rash |
| | Reaction due to an Allergy |
| | Low Blood Pressure |
| Diltiazem (Cardizem, Tiazac) | Common |
| | Cardiovascular: Bradyarrhythmia (1.7% to 3.6%), Peripheral |
| | edema (4.6% to 8%) |
| | Neurologic: Dizziness (3.5% to 6.4%), Headache (4.6%) |
| | Respiratory: Cough (2%) |
| | Other: Fatigue (4.8%) |
| | Serious |
| | Cardiovascular: Congestive heart failure (less than 2%), Heart |
| | block, Myocardial infarction |
| | Hepatic: Hepatotoxicity |
| Felodipine (Plendil) | Common |
| | Cardiovascular: Peripheral edema (2% to 17.4%) |
| | Dermatologic: Flushing (3.9% to 6.9%) |
| | Gastrointestinal: Indigestion (0.5% to 3.9%) |
| | Neurologic: Headache (10.6% to 14.7%) |
| | Respiratory: Upper respiratory infection (0.7% to 3.9%) |
| | Serious |
| | Cardiovascular: Angina, Hypotension (less than 0.5%), Myocardial |
| | infarction, Tachycardia |
| | Neurologic: Cerebrovascular accident |
| Nifedipine (Adalat, Procardia) | Common |
| | Cardiovascular: Hypotension (up to 5%), Palpitations (up to 7%), |
| | Peripheral edema (7% to 29%) |
| | Dermatologic: Flushing (4% to 25%) |
| | Gastrointestinal: Nausea (up to 10%) |
| | Neurologic: Dizziness (4% to 10%), Headache (19% to 23%) |
| | Psychiatric: Feeling nervous |
| | Respiratory: Cough, Dyspnea |
| | Serious |
| | Cardiovascular: Myocardial infarction (up to 4%), Ventricular |
| | arrhythmia (less than 0.5%) |
| | Gastrointestinal: Gastrointestinal obstruction, Gastrointestinal ulcer |
| | Hematologic: Aplastic anemia |
| Nimodipine (Nimotop) | Common |
| | Cardiovascular: Hypotension (up to 8.1%) |
| | Gastrointestinal: Diarrhea (up to 4.2%), Nausea (0.6% to 1.4%) |
| | Neurologic: Headache (up to 4.1%) |
| | Serious |
| | Cardiovascular: Congestive heart failure (less than 1%) |
| | Gastrointestinal: Gastrointestinal hemorrhage (less than 1%) |
| | Hematologic: Bleeding, Disseminated intravascular coagulation |
| | (less than 1%), Hematoma (less than 1%) |
| Nisoldipine (Sular) | Common |
| | Cardiovascular: Palpitations (3%), Peripheral edema (22%), |
| | Vasodilatation (4%) |
| | Dermatologic: Flushing |
| | Neurologic: Dizziness (5%), Headache (22%) |
| | Respiratory: Pharyngitis (5%), Sinusitis |
| | Serious |
| | Cardiovascular: Myocardial infarction |
| Verapamil (Calan, Isoptin, | Common |
| Verelan) | Cardiovascular: Edema (up to 3.7%), Hypotension (1.5% to 3%) |
| | Gastrointestinal: Constipation (7.3% to 13%) |
| | Neurologic: Dizziness (3% to 5.9%), Headache (2.2% to 12.1%) |
| | Respiratory: Pharyngitis (3%), Sinusitis (3%) |
| | Other: Influenza-like symptoms (3.7%) |
| | Serious |
| | Cardiovascular: Atrioventricular block, Myocardial infarction |
| | Respiratory: Pulmonary edema |
| Digoxin | Common |
| | Gastrointestinal: Nausea and vomiting |
| | Neurologic: Dizziness, Headache |
| | Psychiatric: Mental disorder |
| | Serious |
| | Cardiovascular: Cardiac dysrhythmia, Ischemia, Sinoatrial block, |
| | Sinus bradycardia, Vasoconstriction |
| | Hematologic: Thrombocytopenia |
| Digitoxin | Common |
| | Enlarged Breasts, common side effects of digitoxin: |
| | Enlarged Breasts |
| | Infrequent side effects of digitoxin: |
| | Sinus Bradycardia |
| | Rare side effects of digitoxin: |
| | Abnormal Heart Electrical Signals |
| | Atrioventricular Heart Block |
| | Decreased Blood Platelets |
| | Delirium |
| | Diarrhea |
| | Fast Heartbeat |
| | Gangrene of Intestine caused by Blood Supply Problem |
| | Severe Heart Block |
| | Inflammation of Skin caused by an Allergy |
| | Loss of Appetite |
| | Rapid Ventricular Heartbeat |
| | Rash |
| | Reaction due to an Allergy |
| | Throwing Up |
| | Ventricular Fibrillation |
| | Ventricular Premature Beats |
| | Visual Halos Around Lights |
| | Anxious |
| | Blurred Vision |
| | Confused |
| | Depression E194 |
| | Discolored Spots and Small Elevations of the Skin |
| | Dizzy |
| | Feel Like Throwing Up |
| | Feeling Weak |
| | Hallucination |
| | Head Pain |
| Lanoxin | See Digoxin above |
| Amiloride (Midamor) | Common |
| | Dermatologic: Rash (1% or less) |
| | Gastrointestinal: Diarrhea (3% to 8%), Loss of appetite (3% to 8%), |
| | Nausea (3% to 8%), Vomiting (3% to 8%) |
| | Musculoskeletal: Asthenia (greater than 1% to less than 3%), |
| | Cramp (greater than 1% to less than 3%) |
| | Neurologic: Dizziness (greater than 1% to less than 3%), Headache |
| | (3% to 8%) |
| | Respiratory: Cough (greater than 1% to less than 3%), Dyspnea |
| | (greater than 1% to less than 3%) |
| | Serious |
| | Cardiovascular: Cardiac dysrhythmia (1% or less), Palpitations (1% |
| | or less) |
| | Endocrine metabolic: Hyperkalemia (10%) |
| | Hematologic: Aplastic anemia, Neutropenia |
| | Neurologic: Encephalopathy (greater than 1% to less than 3%) |
| | Ophthalmic: Raised intraocular pressure (1% or less) |
| Bumetanide (Bumex) | Common |
| | Cardiovascular: Hypotension (0.8%) |
| | Endocrine metabolic: Hyperuricemia (18.4%), Hypochloremia |
| | (14.9%), Hypokalemia (14.7%) |
| | Gastrointestinal: Nausea (0.6%) |
| | Musculoskeletal: Cramp (1.1%) |
| | Neurologic: Dizziness (1.1%), Headache (0.6%) |
| | Renal: Azotemia (10.6%) |
| | Serious |
| | Dermatologic: Stevens-Johnson syndrome |
| | Hematologic: Thrombocytopenia (0.2%) |
| | Neurologic: Encephalopathy (0.6%) |
| Chlorothiazide (Diuril) | Common |
| | Dermatologic: Photosensitivity |
| | Endocrine metabolic: Hyperglycemia, Hyperuricemia |
| | Gastrointestinal: Diarrhea, Loss of appetite, Nausea, Vomiting |
| | Neurologic: Dizziness |
| | Serious |
| | Dermatologic: Cutaneous lupus erythematosus, Stevens-Johnson |
| | syndrome, Toxic epidermal necrolysis |
| | Endocrine metabolic: Electrolytes abnormal |
| | Gastrointestinal: Pancreatitis |
| | Hematologic: Agranulocytosis, Aplastic anemia, Hemolytic anemia |
| | Hepatic: Hepatotoxicity, Jaundice |
| | Immunologic: Anaphylaxis, Systemic lupus erythematosus |
| | Neurologic: Coma |
| | Renal: Renal failure |
| | Respiratory: Pulmonary edema, Noncardiogenic |
| Chlorthalidone (Hygroton) | Common |
| | Endocrine metabolic: Hyperuricemia |
| | Serious |
| | Cardiovascular: Cardiac dysrhythmia |
| | Dermatologic: Toxic epidermal necrolysis |
| | Gastrointestinal: Pancreatitis (rare) |
| | Hepatic: Cholestatic jaundice syndrome |
| | Respiratory: Pulmonary edema (rare) |
| Furosemide (Lasix) | Common |
| | Endocrine metabolic: Hyperuricemia (40%), Hypomagnesemia |
| | Gastrointestinal: Loss of appetite |
| | Renal: Spasm of bladder |
| | Serious |
| | Cardiovascular: Orthostatic hypotension |
| | Dermatologic: Drug hypersensitivity syndrome, Erythema |
| | multiforme, Erythroderma, Stevens-Johnson syndrome, Toxic |
| | epidermal necrolysis due to drug |
| | Gastrointestinal: Pancreatitis |
| | Hematologic: Agranulocytosis, Aplastic anemia, |
| | Thrombocytopenia |
| | Immunologic: Anaphylactoid reaction, Anaphylaxis |
| Hydro-chlorothiazide (Esidrix, | Common |
| Hydrodiuril) | Cardiovascular: Hypotension |
| | Dermatologic: Phototoxicity |
| | Neurologic: Vertigo |
| | Serious |
| | Cardiovascular: Cardiac dysrhythmia |
| | Dermatologic: Stevens-Johnson syndrome, Toxic epidermal |
| | necrolysis |
| | Endocrine metabolic: Dilutional hyponatremia, Hypercalcemia, |
| | Hyperglycemia, Hypokalemia, Hypomagnesemia, Hyponatremia, |
| | Hypophosphatemia |
| | Gastrointestinal: Cholecystitis, Pancreatitis |
| | Hepatic: Cholestatic jaundice syndrome |
| | Ophthalmic: Angle-closure glaucoma, acute, Myopia, Acute |
| | transient |
| | Renal: Renal failure, Renal impairment |
| Indapamide (Lozol) | Common |
| | Endocrine metabolic: Hypokalemia (3% to 7%) |
| | Musculoskeletal: Cramp |
| | Neurologic: Asthenia, Dizziness (Greater than or equal to 5%), |
| | Headache (Greater than or equal to 5%), Lethargy, Numbness |
| | Other: Fatigue, Malaise |
| | Serious |
| | Dermatologic: Stevens-Johnson syndrome, Toxic epidermal |
| | necrolysis |
| | Hematologic: Agranulocytosis, Aplastic anemia |
| | Hepatic: Hepatitis |
| | Immunologic: Anaphylaxis |
| Spironolactone (Aldactone) | Common |
| | Endocrine metabolic: Gynecomastia |
| | Gastrointestinal: Diarrhea, Nausea and vomiting |
| | Neurologic: Somnolence |
| | Reproductive: Disorder of menstruation, Erectile dysfunction |
| | Serious |
| | Dermatologic: Stevens-Johnson syndrome, Toxic epidermal |
| | necrolysis |
| | Endocrine metabolic: Breast cancer, Disorder of electrolytes, |
| | Hyperkalemia, Metabolic acidosis |
| | Gastrointestinal: Gastric hemorrhage, Gastritis |
| | Hematologic: Agranulocytosis |
| | Immunologic: Drug hypersensitivity syndrome, Systemic lupus |
| | erythematosus |
| | Other: Breast cancer |
| Isosorbide dinitrate (Isordil) | Common |
| | Cardiovascular: Hypotension, Lightheadedness |
| | Neurologic: Headache |
| | Serious |
| | Cardiovascular: Syncope |
| | Hematologic: Methemoglobinemia |
| Nesiritide (Natrecor) | Common |
| | Cardiovascular: Hypotension (4% to 17%) |
| | Gastrointestinal: Nausea (3%) |
| | Neurologic: Dizziness (2%), Headache (7%) |
| | Renal: Serum creatinine raised (17% to 31.4%) |
| | Serious |
| | Dermatologic: Injection site extravasation |
| | Immunologic: Hypersensitivity reaction |
| | Other: Death, Increased risk |
| Hydralazine (Apresoline) | Common |
| | Cardiovascular: Angina, Edema, Palpitations, Tachycardia |
| | Gastrointestinal: Diarrhea, Loss of appetite, Nausea, Vomiting |
| | Neurologic: Headache |
| | Serious |
| | Hematologic: Agranulocytosis, Leukopenia |
| | Hepatic: Hepatotoxicity |
| | Immunologic: Lupus pneumonia (Acute), Systemic lupus |
| | erythematosus |
| Nitrates (Nitroglycerin) | Common |
| | Cardiovascular: Hypotension (4%) |
| | Dermatologic: Flushing |
| | Neurologic: Dizziness (5%), Headache (63% to 64%), |
| | Lightheadedness (6%) |
| | Serious |
| | Dermatologic: Anaphylactoid reaction |
| | Hematologic: Methemoglobinemia |
| | Neurologic: Raised intracranial pressure |
| Minoxidil | Common |
| | Cardiovascular: Hypotension |
| | Dermatologic: Hirsutism, Hypertrichosis |
| | Endocrine metabolic: Body fluid retention (7%), Hypernatremia |
| | Serious |
| | Cardiovascular: Angina, Cardiac tamponade, Electrocardiogram |
| | abnormal (60%), Pericardial effusion (3%), Pericarditis, |
| | Tachyarrhythmia |
| | Dermatologic: Stevens-Johnson syndrome |
| | Hematologic: Leukopenia, Thrombocytopenia |

Table 5 is included by reference as the drugs that are listed as in development in the following databases: Cortellis™ Competitive Intelligence by Thomson Reuters; Adis R&D; and Pharmaprojects by Citeline. The drugs in the development pipeline can utilize the Invention to capture required clinical trial information and control drug dispensing for regulatory drug approval as well as to control drug dispensing after regulatory approval. The drugs are encompassed in the embodiment of the invention by reference. The FDA and like regulatory agency drug approval self-assessment, self-testing, self-reporting, concomitant medications, side-effects, digitally captured diagnostic testing, digital diary information, dispensing information, etc. drug approval recommended data capture and reporting guidelines are incorporated by reference. The listing for each drug includes by definition each drug's respective indication(s), strength, dosage form, route of administration, side effect profile, drug interactions, and regulatory approval guidelines, etc.

[symbol]

Table 6 is included by reference as the mechanisms of action for marketed oral drugs, oral drugs in developed, and efficacious oral drugs whose development was stopped due to a side effect(s) that can be addressed by the embodiment and thereby made approvable. The listed drugs in the following databases are encompassed in the embodiment of the invention by reference: Cortellis™ Competitive Intelligence by Thomson Reuters; Adis R&D; and Pharmaprojects by Citeline. The listing for each drug includes by definition each respective drug's respective indication(s), strength, dosage form, route of administration, side effect profile, drug interactions, etc.

[symbol]

Table 7 is included by reference as the oral drugs listed in the following databases that (i) were in development but were discontinued due to dose related side effects whose safety concerns can be addressed by the Invention or (ii) drugs that were withdrawn from the market after approval due to dose related side effects whose safety concerns can be addressed by the Invention and contained in the following databases: (i) Cortellis™ Competitive Intelligence by Thomson Reuters, and/or (ii) Adis R&D, and/or (iii) Pharmaprojects by Citeline. These drugs are encompassed in the embodiment of the invention by reference. The listing for each drug includes by definition each drug's respective indication(s), strength, dosage form, route of administration, side effect profile, drug interactions, mechanisms of action, regulatory approval guidelines, etc.

Table 8 is a sample list of diseases encompassed in the embodiment of the invention by reference. The listing for each encompasses oral drugs used to treat the disease and for each includes by definition each drug's respective indication(s), strength, dosage form, route of administration, side effect profile, drug interactions, etc.

TABLE 8
Diseases

Acinetobacter Infection
Acquired Immune Deficiency Syndrome (AIDS)
Adenovirus Infection
ADHD
Adverse Childhood Experiences (ACE)
African Trypanosomiasis
Agricultural Safety
AHF (Alkhurma hemorrhagic fever)
AIDS (Acquired Immune Deficiency Syndrome)
Alkhurma hemorrhagic fever (AHF)
ALS
Alzheimer's Disease
Amebiasis, Intestinal
American Trypanosomiasis
Amyotrophic Lateral Sclerosis
Anaplasmosis, Human
Ancylostoma duodenale Infection, Necator americanus Infection
Anemia
Angiostrongylus Infection
Anisakiasis
Anisakis Infection
Anthrax
Aortic Aneurysm
Aortic Dissection
Arenavirus Infection
Arthritis
Ascariasis
Ascaris Infection
Aseptic Meningitis
Aspergillosis
Aspergillus Infection
Asthma
Attention Deficit/Hyperactivity Disorder
Autism
autism spectrum disorders
Avian Influenza
B virus Infection
B. cepacia infection (Burkholderia cepacia Infection)
Babesia Infection
Babesiosis
Bacillus anthracis
Bacillus anthracis Infection
Back Belts
Bacterial Meningitis
Bacterial Vaginosis (BV)
Balamuthia mandrillaris Infection
Balamuthia Infection
Balantidiasis
Balantidium Infection
Bartonella bacilliformis Infection
Bartonella quintana Infection
Baylisascaris Infection
Bilharzia
Bioterrorism Agents/Diseases
Bird Flu
Black Lung
Blastocystis hominis Infection
Blastocystis Infection
Blastomycosis
Bleeding Disorders
blood clot
Blood Disorders
Borrelia burgdorferi Infection
Botulism
Bovine Spongiform Encephalopathy (BSE)
Brainerd Diarrhea
Breast Cancer
Breastfeeding
Bronchiolitis
Bronchitis
Brucella Infection
Brucellosis
BSE (Bovine Spongiform Encephalopathy)
BSE (Mad Cow Disease)
Burkholderia cepacia Infection (B. cepacia infection)
Burkholderia mallei
Burkholderia pseudomallei Infection
BV (Bacterial Vaginosis)
C. diff. Infection
C. gattii cryptococcosis
C. neoformans cryptococcosis
Campylobacter Infection
Campylobacteriosis
Cancer
Candida Infection
Candidiasis
Canine Flu
Capillaria Infection
Capillariasis
Carbapenem resistant Klebsiella pneumonia (CRKP)
Carbapenem-resistant Enterobacteriaceae (CRE)
Carrión's disease
CCHF (Crimean-Congo hemorrhagic fever)
Cercarial Dermatitis
Cerebral Palsy
Cervical Cancer
CFS (Chronic Fatigue Syndrome)
Chagas Disease
Chapare Hemorrhagic Fever (CHHF)
Chest Cold
CHHF (Chapare Hemorrhagic Fever)
Chickenpox
Chikungunya Fever (CHIKV)
CHIKV (Chikungunya Fever)
Childhood Arthritis
Chlamydia
Chlamydia trachomatis Disease
Chlamydophila (Chlamydia) pneumoniae Infection
Cholera
Chronic Fatigue Syndrome (CFS)
Chronic Kidney Disease (CKD)
Chronic Obstructive Pulmonary Disease (COPD)
Chronic Wasting Disease (CWD)
Ciguatera Fish Poisoning
Ciguatoxin
CJD, Classic (Classic Creutzfeldt-Jakob Disease)
CKD (Chronic Kidney Disease)
CKD (Kidney Disease)
Classic Creutzfeldt-Jakob Disease (CJD, Classic)
Clonorchiasis
Clonorchis Infection
Clostridium botulinim Infection
Clostridium difficile Infection
Clostridium perfringens infection
Clostridium tetani Infection
Clotting Disorders
CMV (Cytomegalovirus Infection)
Coal Workers' Pneumoconioses
Coccidioidomycosis
Cold, Common
Colorado Tick Fever (CTF)
Colorectal (Colon) Cancer
Conjunctivitis
Cooleys Anemia
COPD (Chronic Obstructive Pulmonary Disease)
Corynebacterium diphtheriae Infection
Coxiella burnetii Infection
CRE (Carbapenem-resistant Enterobacteriaceae)
Creutzfeldt-Jakob Disease, Classic
Crimean-Congo hemorrhagic fever (CCHF)
CRKP (Carbapenem resistant Klebsiella pneumonia)
Crohn's Disease
Cronobacter Infection
Cryptococcosis, C. gattii.
Cryptococcosis, C. neoformans
Cryptosporidiosis
Cryptosporidium Infection
CTF (Colorado Tick Fever)
CWD (Chronic Wasting Disease)
Cyclospora Infection
Cyclosporiasis
Cysticercosis
Cystoisospora Infection
Cystoisosporiasis
Cytomegalovirus Infection (CMV)
DBA (Diamond Blackfan Anemia)
Deep Vein Thrombosis (DVT)
Dengue Fever (DF)
Dengue Hemorrhagic Fever (DHF)
Dermatophyte Infection
Dermatophytes
Developmental Disabilities
DF (Dengue Fever)
DHF (Dengue Hemorrhagic Fever)
Diabetes
Diamond Blackfan Anemia (DBA)
Dientamoeba fragilis Infection
Diphtheria
Diphyllobothriasis
Diphyllobothrium Infection
Dipylidium Infection
Dirofilariasis
Dracunculiasis
DVT (Deep Vein Thrombosis)
Dwarf Tapeworm
E. coli Infection
Ear Infection
Eastern Equine Encephalitis (EEE)
Ebola Virus Disease (EVD)
EBV Infection (Epstein-Barr Virus Infection)
Echinococcosis
EEE (Eastern Equine Encephalitis)
Ehrlichiosis, Human
Elephantiasis
Entamoeba histolytica infection
Enteric Diseases from Animals
Enterobius vermicularis Infection
Enterovirus D68
Enterovirus Infections (Non-Polio)
Epidemic Typhus
Epilepsy
Epstein-Barr Virus Infection (EBV Infection)
Ergonomic and Musculoskeletal Disorders
Escherichia coli Infection
Esophageal Candidiasis
EVD (Ebola Virus Disease)
EV-D68
Exserohilum rostratum (Other Pathogenic Fungi)
Extensively Drug-Resistant TB (XDR TB)
Fasciitis, Necrotizing
Fasciola Infection
Fascioliasis
Fasciolopsiasis
Fasciolopsis Infection
Fetal Alcohol Syndrome
Fibromyalgia
Fifth Disease
Filariasis, Lymphatic
Flu
Folliculitis
Fragile X Syndrome (FXS)
Francisella tularensis Infection
Fungal diseases
Fungal Keratitis
Fungal Meningitis
Fungal Pneumonia
FXS (Fragile X Syndrome)
GAE (Granulomatous amebic encephalitis)
GAS (Group A Strep Infection)
Gastrointestinal Diseases from Animals
GBS (Group B Strep Infection)
Genital Candidiasis (VVC)
Genital Herpes
German Measles (Rubella Virus)
Giardia Infection
Giardiasis
Glanders
Gnathostoma Infection
Gnathostomiasis
Gonorrhea
Gout
Granulomatous amebic encephalitis (GAE)
Group A Strep Infection (GAS)
Group A Streptococcal Infection
Group B Strep Infection (GBS)
Group B Streptococcal Infection
Guillain-Barré Syndrome
Guinea Worm Disease
Gynecologic Cancers
H1N1 Flu
H3N2v influenza
H5N1
Haemophilus influenzae Serotype b
Haemophilus influenzae Infection (including Hib Infection)
Hand, Foot, and Mouth Disease (HFMD)
Hansen's Disease
Hantavirus Pulmonary Syndrome (HPS)
Heart Disease
Hemochromatosis
Hemoglobinopathies
Hemophilia
Hemophilia Treatment Centers (HTC)
Hemorrhagic Fevers, Viral
Hendra Virus Disease (HeV Infection)
Hepatitis, Viral
Hereditary Bleeding Disorders
Herpes B virus
Herpes Simplex Virus Infection
Herpes Zoster
Herpes, Genital
Herpesvirus B
Herpesvirus simiae
Heterophyes Infection
Heterophyiasis
HeV Infection (Hendra Virus Disease)
HFMD (Hand, Foot, and Mouth Disease)
Hib Infection
High Blood Pressure
Histoplasma capsulatum Infection
Histoplasmosis
Hookworm, Human
Hookworm, Zoonotic
HPIV (Human Parainfluenza Viruses)
HPS (Hantavirus Pulmonary Syndrome)
HPV Infection (Human Papillomavirus Infection)
HPV-Associated Cancers
HTC (Hemophilia Treatment Centers)
Human Ehrlichiosis
Human Hookworm
Human Immunodeficiency Virus
Human Papillomavirus Infection (HPV Infection)
Human Parainfluenza Viruses (HPIV)
Hymenolepis Infection
Hypertension
IBD (Inflammatory Bowel Disease)
Impetigo
including Hib Infection (Haemophilus influenzae Infection)
Infectious Mononucleosis
Infertility
Inflammatory Bowel Disease (IBD)
Influenza
Influenza, Avian
Influenza, Pandemic
Intestinal Amebae Infection, Nonpathogenic
Invasive Candidiasis
Iron Deficiency
Iron Overload
Iron Storage Disease
Isospora Infection
Japanese Encephalitis (JE)
Jaundice
JE (Japanese Encephalitis)
K. pneumoniae (Klebsiella pneumoniae)
Kala-Azar
Kawasaki Syndrome (KS)
Keratitis, Fungal
Kernicterus
KFD (Kyasanur Forest disease)
Kidney Disease (CKD)
Klebsiella pneumoniae (K. pneumoniae)
KS (Kawasaki Syndrome)
Kyasanur Forest disease (KFD)
La Crosse Encephalitis (LAC)
La Crosse Encephalitis virus (LACV)
Lassa Fever
Latex Allergies
LCM (Lymphocytic Choriomeningitis)
Lead Poisoning
Legionellosis
Legionnaires' Disease
Leishmania Infection
Leishmaniasis
Leprosy
Leptospira Infection
Leptospirosis
Listeria Infection
Listeriosis
Liver Disease and Hepatitis
Loa Infection
Lockjaw
Loiasis
Lou Gehrig's Disease
LUHF (Lujo Hemorrhagic Fever)
Lujo Hemorrhagic Fever (LUHF)
Lung Cancer
Lupus (SLE)
Lyme Disease
Lymphatic Filariasis
Lymphedema
Lymphocytic Choriomeningitis (LCM)
MAC (Mycobacterium avium Complex)
Mad Cow Disease (BSE)
Malaria
Marburg Hemorrhagic Fever
MD (Muscular Dystrophy)
MDR TB (Multidrug-Resistant TB)
Measles
Melioidosis
Meningitis
Meningococcal Disease
MERS-CoV (Middle East Respiratory Syndrome Coronavirus)
Methicillin Resistant Staphylococcus aureus
Micronutrient Malnutrition
Microsporidia Infection
Middle East Respiratory Syndrome Coronavirus (MERS-CoV)
Molluscum Contagiosum
Monkey B virus
Monkeypox
Mononucleosis, Infectious
MRSA
Mucormycosis
Mucus
Multidrug-Resistant TB (MDR TB)
Multiple organ dysfunction syndrome
Mumps
Muscular Dystrophy (MD)
Musculoskeletal Disorders
Mycobacterium abscessus Infection
Mycobacterium avium Complex (MAC)
Mycobacterium tuberculosis Infection
Mycoplasma pneumoniae Infection
Mycotic diseases
Myelomeningocele
Myiasis
Naegleria Infection
Nairovirus Infection
National Amyotrophic Lateral Sclerosis (ALS) Registry
Necrotizing Fasciitis
Neglected Tropical Diseases (NTD)
Neisseria gonorrhoeae Infection
Neurocysticercosis
Newborn Jaundice
Nocardia asteroides Infection
Nocardiosis
Nonpathogenic (Harmless) Intestinal Protozoa
Non-Polio Enterovirus Infections
Norovirus Infection
Norwalk-like Viruses (NLV)
NTD (Neglected Tropical Diseases)
OA (Osteoarthritis)
Obesity and Overweight
OHF (Omsk hemorrhagic fever)
Omsk hemorrhagic fever (OHF)
Onchocerciasis
Opisthorchis Infection
Oral Cancer
Orf Virus Infection
Oropharyngeal Candidiasis
Oroya fever
Osteoarthritis (OA)
Osteoporosis
Otitis Media
Ovarian Cancer
PAD (Peripheral Arterial Disease)
Pandemic Flu
Paragonimiasis
Paragonimus Infection
Parainfluenza
Parasitic Diseases
Parvovirus B19 Infection
PCP (Pneumocystis pneumonia)
PE (Pulmonary Embolism)
Pediculus humanus capitis
Pediculus humanus corporis
Pelvic Inflammatory Disease (PID)
Peripheral Arterial Disease (PAD)
Peripheral Arterial Insufficiency
Peripheral Arterial Occlusive Disease
Peripheral Vascular Disease
Pertussis (Whooping Cough)
Phthiriasis
PID (Pelvic Inflammatory Disease)
Pinworm Infection
Pneumococcal Disease
Pneumoconioses, Coal Workers'
Pneumocystis carinii Pneumonia (PCP) Infection
Pneumocystis jirovecii pneumonia (previously Pneumocystis carinii)
Pneumocystis pneumonia (PCP)
Pneumonia
Polio Infection
Poliomyelitis Infection
Pontiac Fever
Powassan (POW) virus
Poxvirus Infections
Primary Amebic Meningoencephalitis (PAM)
Prion Diseases (TSEs)
Prostate Cancer
Pseudomonas dermatitis Infection
Psittacosis
Psoriasis
Pulmonary Embolism (PE)
Pulmonary Hypertension
Q Fever
RA (Rheumatoid Arthritis)
Rabies
Raccoon Roundworm Infection
Rat-Bite Fever (RBF)
RBF (Rat-Bite Fever)
Reptiles, Infections from
Respiratory Syncytial Virus Infection (RSV)
Rheumatoid Arthritis (RA)
Rickettsia rickettsii Infection
Rickettsia, Spotted Fever Group
Rickettsial Diseases
Rift Valley Fever (RVF)
Ringworm
River Blindness
RMSF (Rocky Mountain Spotted Fever)
Rocky Mountain Spotted Fever (RMSF)
Rotavirus Infection
RSV (Respiratory Syncytial Virus Infection)
Rubeola
RVF (Rift Valley Fever)
RWI (Recreational Water Illness)
Salmonella typhi Infection
Salmonella Infection
Salmonellosis
Sappinia diploidea and Sappinia pedata
Sappinia Infection
SARS
Scabies
Scarlet Fever
Schistosoma Infection
Schistosomiasis
Seasonal Flu
Sepsis
Septic shock
Septicemia
Severe Acute Respiratory Syndrome
Sexually Transmitted Diseases (STDs)
SFGR (Spotted Fever Group Rickettsia)
Shigella Infection
Shigellosis
Shingles
Sickle Cell Disease
Sinus Infection
Sinusitus
Skin Cancer
SLE (Lupus)
Sleep and Sleep Disorders
Sleeping Sickness
Smallpox
Soil Transmitted Helminths
Sore Mouth Infection
Sore Throat
Southern Tick-Associated Rash Illness (STARI)
Spirillum minus Infection
Sporothrix schenckii infection
Sporotrichosis
Spotted Fever Group Rickettsia (SFGR)
Staph
Staphylococcus aureus Infection
STARI (Southern Tick-Associated Rash Illness)
STDs (Sexually Transmitted Diseases)
Strep Infection, Group A
Strep Infection, Group B
Strep Throat
Streptobacillus moniliformis Infection
Streptococcus pneumoniae Infection
Stress
Stroke
Strongyloidiasis
Surgical site infection (SSI)
Swimmer's Itch
Swine Influenza
Symptom Relief for Upper Respiratory Infections
Syphilis
Systemic lupus erythematosus
Taenia Infection
Tapeworm Infection
TB (Tuberculosis)
TBI (Traumatic Brain Injury)
Testicular Cancer
Tetanus (Lockjaw) Infection
Tetanus Disease
Thalassemia
Thoracic Aortic Aneurysm
Throat, Sore
Throat, Strep
Thrombophilia
Thrombosis
Thrush
Tickborne Diseases
Ticks
Tinea
Tourette Syndrome (TS)
Toxocara Infection
Toxocariasis
Toxoplasma Infection
Toxoplasmosis
Trachoma Infection
Transmissible spongiform encephalopathies
Trench fever
Treponema pallidum Infection
Trichinellosis (Trichinosis)
Trichomonas Infection
Trichomoniasis
Trichuriasis
Trisomy 21
Trypanosoma cruzi Infection
Trypanosomiasis, African
TS (Tourette Syndrome)
TSEs (Prion Diseases)
Tuberculosis (TB)
Tularemia
Typhoid Fever
Typhus Fevers
Ulcerative Colitis
Undulant Fever
Upper Respiratory Infection Symptom Relief
Uterine Cancer
Vaginal and Vulvar Cancers
Vaginal Candidiasis
Valley Fever
Vancomycin-Intermediate/Resistant Staphylococcus Aureus Infections
Vancomycin-resistant Enterococci Infection (VRE)
Variant Creutzfeldt-Jakob Disease (vCJD)
Varicella Disease
Varicella Zoster Virus (VZV)
Variola Major and Variola Minor
vCJD (Variant Creutzfeldt-Jakob Disease)
verruga peruana
VHF (Viral Hemorrhagic Fevers)
Vibrio cholerae Infection
Vibrio Illness
Vibriosis
Viral Hemorrhagic Fevers (VHF)
Viral Hepatitis
Viral Meningitis
VISA/VRSA Infections
VRE (Vancomycin-resistant Enterococci Infection)
Vulvovaginal Candidiasis
VVC (Genital Candidiasis)
VZV (Varicella Zoster Virus)
West Nile Virus Infection (WNV Infection)
Whipworm Infection
Whitmore's Disease
Whooping Cough
WNV Infection (West Nile Virus Infection)
Women's Bleeding Disorders
XDR TB (Extensively Drug-Resistant TB)
Xenotropic Murine Leukemia Virus-related Virus Infection
XMRV Infection
Yellow Fever
Yersinia enterocolitica Infection
Yersinia pestis Infection
Yersiniosis
Zoonotic enteric diseases
Zoonotic Hookworm
Zoster
Zygomycosis

Table 9 lists oral opioids in particular. The listed approved opioids are encompassed in the opioid-specific embodiments of the invention by reference and can benefit from an improved opioid safety profile. Again, the listing for each drug includes, by definition, each opioid's respective indication(s), strength, dosage form, route of administration, side-effect profile, drug interactions, mechanisms of action, manufacturer, etc.). In addition to Table 9, the opioid-specific embodiments incorporate by reference all marketed and in development oral opioids listed in: (i) Goodman & Gilman's, The Pharmacological Basis of Therapeutics (12th Ed) (Goodman et al. eds) (McGraw-Hill) (2011); (ii) 2015 Physician's Desk Reference; (iii) Cortellis™ Competitive Intelligence by Thomson Reuters; (iv) Adis R&D; and/or (v) Pharmaprojects by Citeline. The listing for each drug includes, by definition, each drug's respective indication(s), strength, dosage form, route of administration, side effect profile, drug interactions, mechanisms of action, manufacturer, etc.

TABLE 9
Approved Opioids

| | | Oxycodone | | | | Hydrocodone | | | Morphine Sulfate | | | | |
|---|---|---|---|---|---|---|---|---|---|---|---|---|---|
| | | | | w/ | Oxy- | | | w/ | | | | w/ | Hydrom. |
| Opioid Side | | | | APAP | codone | | w/ | Ibu- | Mor- | 12 | 24 | Naltraxone | hydro- |
| Effects | | OxyContin | HCL | ER | APAP | HCL | APAP | profen | phine | Hour | Hour | ER | morphone |
| 2 | mg | | | | | | | | | | | | X |
| 3 | mg | | | | | | | | | | | | ✓ |
| 4 | mg | | | | | | | | | | | | X |
| 5 | mg | | X | | | | | | | | | | |
| 7.5 | mg | | X | | | | | | | | | | |
| 8 | mg | | | | | | | | | | | | X |
| 10 | mg | X | X | | | X | | | X | | X | | |
| 12 | mg | | | | | | | | | | | | |
| 15 | mg | X | X | | | X | | | X | X | | | |
| 16 | mg | | | | | | | | | | | | |
| 20 | mg | X | X | | | X | | | X | | X | | |
| 24 | mg | | | | | | | | | | | | ✓ |
| 30 | mg | X | X | | | X | | | X | X | X | | |
| 32 | mg | | | | | | | | | | | | |
| 40 | mg | X | | | | X | | | X | | X | | |
| 45 | mg | | | | | X | | | X | | X | | |
| 50 | mg | | | | | X | | | X | | X | | |
| 60 | mg | X | | | | X | | | X | X | X | | |
| 70 | mg | | | | | | | | X | | X | | |
| 75 | mg | | | | | | | | X | | X | | |
| 80 | mg | X | | | | X | | | X | | X | | |
| 90 | mg | | | | | X | | | X | | X | | |
| 100 | mg | | | | | X | | | X | X | X | | |
| 120 | mg | | | | | X | | | X | | X | | |
| 130 | mg | | | | | | | | X | | X | | |
| 150 | mg | | | | | | | | X | | X | | |
| 160 | mg | ✓ | | | | | | | | | | | |
| 200 | mg | | | | | | | | X | X | X | | |
| 250 | mg | | | | | | | | | | | | ✓ |
| 2.5 MG; 200 MG | | | | | | | | X | | | | | |
| 5 MG; 200 MG | | | | | | | | X | | | | | |
| 7.5 MG; 200 MG | | | | | | | | X | | | | | |
| 10 MG; 200 MG | | | | | | | | X | | | | | |
| 20 MG; 0.8 MG | | | | | | | | | | | | X | |
| 30 MG; 1.2 MG | | | | | | | | | | | | X | |
| 50 MG; 2 MG | | | | | | | | | | | | X | |
| 60 MG; 2.4 MG | | | | | | | | | | | | X | |
| 80 MG; 3.2 MG | | | | | | | | | | | | X | |
| 100 MG; 4 MG | | | | | | | | | | | | X | |
| 250 mg-10 mg | | | | | | | ✓ | | | | | | |
| 300 mg-2.5 mg | | | | | X | | | | | | | | |
| 300 mg-5 mg | | | | | X | | X | | | | | | |
| 300 mg-7.5 mg | | | | | X | | X | | | | | | |
| 300 mg-10 mg | | | | | X | | X | | | | | | |
| 325 mg-2.5 mg | | | | | X | | X | | | | | | |
| 325 MG; 4.8355 MG | | | | | X | | | | | | | | |
| 325 mg-5 mg | | | | | X | | X | | | | | | |
| 325 mg-7.5 mg | | | | X | X | | X | | | | | | |
| 325 mg-10 mg | | | | | X | | X | | | | | | |
| 400 mg-2.5 mg | | | | | ✓ | | | | | | | | |
| 400 mg-5 mg | | | | | X | | ✓ | | | | | | |
| 400 mg-7.5 mg | | | | | ✓ | | ✓ | | | | | | |
| 400 mg-10 mg | | | | | ✓ | | ✓ | | | | | | |
| 500 mg-2.5 mg | | | | | | | ✓ | | | | | | |
| 500 mg-5 mg | | | | | ✓ | | ✓ | | | | | | |
| 500 mg-7.5 mg | | | | | ✓ | | ✓ | | | | | | |
| 500 mg-10 mg | | | | | ✓ | | ✓ | | | | | | |
| 650 mg-10 mg | | | | | ✓ | | ✓ | | | | | | |
| 650 mg-7.5 mg | | | | | ✓ | | ✓ | | | | | | |
| 660 mg-10 mg | | | | | | | ✓ | | | | | | |
| 750 mg-7.5 mg | | | | | | | ✓ | | | | | | |
| 750 mg-10 mg | | | | | | | ✓ | | | | | | |
| 100 | mcg | | | | | | | | | | | | |
| 200 | mcg | | | | | | | | | | | | |
| 300 | mcg | | | | | | | | | | | | |
| 400 | mcg | | | | | | | | | | | | |
| 600 | mcg | | | | | | | | | | | | |
| 800 | mcg | | | | | | | | | | | | |
| 1200 | mcg | | | | | | | | | | | | |
| 1600 | mcg | | | | | | | | | | | | |

1.4 MG; 0.36 MG
2 MG; 0.5 MG
2.9 MG; 0.71 MG
5.7 MG; 1.4 MG
8 MG; EQ 2 MG
8.6 MG; 2.1 MG
11.4 MG; 2.9 MG

| | | Hydrom. | Oxym. | | | | | | | |
|---|---|---|---|---|---|---|---|---|---|---|
| | | Hydro- | | Oxy- | Topent. | | Fentanyl | | Bupre. | |
| Opioid Side | | morpone | Oxy- | morphone | | Tapentadol | | Fentanyl | Bupre- | W/ |
| Effects | | ER | morphone | ER | Tapentadol | ER | Fentanyl | Lozanges | norphine | Noloxone |
| 2 | mg | | | | | | | | X | |
| 3 | mg | | | | | | | | | |
| 4 | mg | | | | | | | | | |
| 5 | mg | | X | X | | | | | | |
| 7.5 | mg | | | X | | | | | | |
| 8 | mg | X | | | | | | | X | |
| 10 | mg | | X | X | | | | | | |
| 12 | mg | X | | | | | | | | |
| 15 | mg | | | X | | | | | | |
| 16 | mg | X | | | | | | | | |
| 20 | mg | | | X | | | | | | |
| 24 | mg | | | | | | | | | |
| 30 | mg | | | X | | | | | | |
| 32 | mg | X | | | | | | | | |
| 40 | mg | | | X | | | | | | |
| 45 | mg | | | | | | | | | |
| 50 | mg | | | | X | X | | | | |
| 60 | mg | | | | | | | | | |
| 70 | mg | | | | | | | | | |
| 75 | mg | | | | X | | | | | |
| 80 | mg | | | | | | | | | |
| 90 | mg | | | | | | | | | |
| 100 | mg | | | | X | X | | | | |
| 120 | mg | | | | | | | | | |
| 130 | mg | | | | | | | | | |
| 150 | mg | | | | | X | | | | |
| 160 | mg | | | | | | | | | |
| 200 | mg | | | | | X | | | | |
| 250 | mg | | | | | X | | | | |
| 2.5 MG; 200 MG | | | | | | | | | | |
| 5 MG; 200 MG | | | | | | | | | | |
| 7.5 MG; 200 MG | | | | | | | | | | |
| 10 MG; 200 MG | | | | | | | | | | |
| 20 MG; 0.8 MG | | | | | | | | | | |
| 30 MG; 1.2 MG | | | | | | | | | | |
| 50 MG; 2 MG | | | | | | | | | | |
| 60 MG; 2.4 MG | | | | | | | | | | |
| 80 MG; 3.2 MG | | | | | | | | | | |
| 100 MG; 4 MG | | | | | | | | | | |
| 250 mg-10 mg | | | | | | | | | | |
| 300 mg-2.5 mg | | | | | | | | | | |
| 300 mg-5 mg | | | | | | | | | | |
| 300 mg-7.5 mg | | | | | | | | | | |
| 300 mg-10 mg | | | | | | | | | | |
| 325 mg-2.5 mg | | | | | | | | | | |
| 325 MG; 4.8355 MG | | | | | | | | | | |
| 325 mg-5 mg | | | | | | | | | | |
| 325 mg-7.5 mg | | | | | | | | | | |
| 325 mg-10 mg | | | | | | | | | | |
| 400 mg-2.5 mg | | | | | | | | | | |
| 400 mg-5 mg | | | | | | | | | | |
| 400 mg-7.5 mg | | | | | | | | | | |
| 400 mg-10 mg | | | | | | | | | | |
| 500 mg-2.5 mg | | | | | | | | | | |
| 500 mg-5 mg | | | | | | | | | | |
| 500 mg-7.5 mg | | | | | | | | | | |
| 500 mg-10 mg | | | | | | | | | | |
| 650 mg-10 mg | | | | | | | | | | |
| 650 mg-7.5 mg | | | | | | | | | | |
| 660 mg-10 mg | | | | | | | | | | |
| 750 mg-7.5 mg | | | | | | | | | | |
| 750 mg-10 mg | | | | | | | | | | |
| 100 | mcg | | | | | | X | | | |
| 200 | mcg | | | | | | X | X | | |
| 300 | mcg | | | | | | X | | | |
| 400 | mcg | | | | | | X | X | | |
| 600 | mcg | | | | | | X | X | | |
| 800 | mcg | | | | | | X | X | | |
| 1200 | mcg | | | | | | | X | | |
| 1600 | mcg | | | | | | | X | | |

| | |
|---|---|
| 1.4 MG; 0.36 MG | X |
| 2 MG; 0.5 MG | X |
| 2.9 MG; 0.71 MG | X |
| 5.7 MG; 1.4 MG | X |
| 8 MG; EQ 2 MG | X |
| 8.6 MG; 2.1 MG | X |
| 11.4 MG; 2.9 MG | X |

Legend:
X FDA Approved
✓ Approved Outside the United States

Each opioid's side effects are listed in the opioid's package insert. Each side effect can be tracked using the respective Patient Self-Assessment, Patient Self-Test, and/or Patient Self-Report screens or digitally captured diagnostic, monitoring, or patient maintained information. Table 10 is an exemplary listing of side effects that can be tracked by the prescriber using a selection on the prescription or other input media that enables a data capture screen to be incorporated into the Drug Specific App. The information may or may not be incorporated into the Patient Specific Dispensing Algorithm. The Drug Specific App can be customized to request the information each time or at specified intervals. This allows the Patient Drug Specific App to be tailored to each patient to assist in patient management. The drug specific side effects for each opioid are listed in each opioid's package insert and are hereby incorporated by reference.

TABLE 10
Sample Opioid Side Effects

| | | | | Hydro- | Oxy- | | | |
|---|---|---|---|---|---|---|---|---|
| | | Hydro- | Morphine | mor- | mor- | | Fen- | |
| Opioid Side Effects | OxyContin | codone | Sulfate | phone | phone | Topentadol | tanyl | Buprenorpphie |
| Abdominal or Stomach Pain | X | | | | X | | X | |
| Abdominal or Stomach Pain or Discomfort | | X | | | | | | |
| Abdominal Pain | X | | | | | | | |
| Abnormal Dreams | X | | X | | | X | | |
| Abnormal Gait | X | | | | | | | |
| Abnormal Vision | X | | | | | | | |
| Absent, Missed, Or Irregular Menstrual Periods | | | | | | | | |
| Accidental Injury | X | | | | | | | |
| Acid or Sour Stomach | | | | X | X | | | |
| Agitation | X | | X | X | | | | |
| Amnesia | X | | | | | | | |
| Anorexia | X | | | | | | | |
| Anxiety | X | | | | | X | | |
| Back Pain | | X | | X | | | X | X |
| Bad, Unusual or Unpleasant (After) Taste | | | | X | | | | |
| Bad, Unusual, Or Unpleasant (After) Taste | | | | | | | | |
| Being Forgetful | | | | X | | | | |
| Being Forgetful or Confused | | | | | | X | | |
| Belching | | | | X | X | | | |
| Black, Tarry Stools | | | | | | | X | |
| Bladder Pain | | X | | | | X | | |
| Bleeding After Defecation | | | | X | | | | |
| Blistering, Crusting, Irritation, Itching, Or Reddening | | | | | X | | | |
| of the Skin | | | | | | | | |
| Bloating or Swelling of the Face, Arms, Hands, Lower | X | X | | X | | | | |
| Legs, Or Feet | | | | | | | | |
| Bloody or Cloudy Urine | | X | | | | X | | |
| Bloody, Black, Or Tarry Stools | | | | X | | | | |
| Blue Lips and Fingernails | | X | | | | | | |
| Bluish Lips or Skin | | | | X | | | | |
| Blurred Vision | X | X | | X | X | | X | X |
| Body Aches or Pain | | X | | | | X | | |
| Bulging Soft Spot On the Head of an Infant | | | | | | | | |
| Burning, Crawling, Itching, Numbness, Prickling, “Pins | | | | X | | | | |
| and Needles”, Or Tingling Feelings | | | | | | | | |
| Change in Consciousness | X | X | | | | | | |
| Change in Taste | | | | X | | | | |
| Change in The Ability to See Colors, Especially Blue or | | | | X | | | | |
| Yellow | | | | | | | | |
| Change in Vision | | | | | | X | | |
| Change in Walking and Balance | | | | | | | X | |
| Change or Problem with Discharge of Semen | | | | | | | | |
| Change in Behavior | | | | X | | X | | |
| Changes in The Patterns and Rhythms of Speech | | | | X | | | | |
| Changes in Vision | | | | | | | X | |
| Chest Pain | X | | | | | | X | |
| Chest Pain or Discomfort | X | X | | X | X | | | |
| Chills | X | X | | X | X | X | | |
| Clumsiness | | | | X | | | | |
| Clumsiness or Unsteadiness | | | | | | X | X | |
| Cold and Clammy Skin | | X | | | X | | | |
| Cold Sweats | X | | | | X | | | |
| Cold, Clammy Skin | X | | | X | | | | |
| Confusion | X | X | | X | X | | X | X |
| Confusion as to Time, Place, Or Person | | | | | | | | |
| Constipation | | | | | | X | | |
| Constricted Pupil (Black Part of the Eye) | | X | | | | | | |
| Constricted, Pinpoint, Or Small Pupils (Black Part of | X | | | X | X | | | |
| the Eye) | | | | | | | | |
| Continuing Ringing or Buzzing or Other Unexplained | | | | X | | | | |
| Noise in The Ears | | | | | | | | |
| Convulsions | X | | | X | | X | X | |
| Cough | X | X | | X | X | X | X | |
| Cough Increased | X | | | | | | | |
| Cough or Hoarseness | | | | | | | X | X |
| Coughing That Sometimes Produces a Pink Frothy | | X | | | | | | |
| Sputum | | | | | | | | |
| Cracked, Dry, Scaly Skin | | | | | X | | | |
| Cramps | | | | | | | | |
| Crying | | | | X | | | | |
| Decrease in Consciousness | | | | | X | | | |
| Decrease in The Frequency of Urination | X | | | X | | | | |
| Decrease in Urine Volume | X | | | X | X | | | |
| Decreased Appetite | | | | | X | X | | |
| Decreased Awareness or Responsiveness | X | X | | X | X | | X | |
| Decreased Frequency of Urination | | | | | | | X | |
| Decreased Interest in Sexual Intercourse | | | | X | | | | |
| Decreased Urination | | | | X | X | | | |
| Decreased Urine | | | | | | | X | |
| Decreased Weight | | | | | X | | | |
| Deep or Fast Breathing with Dizziness | | | | X | | | | |
| Dehydration | X | | | | | | | |
| Delusions | | | | | | | | |
| Delusions of Persecution, Mistrust, Suspiciousness, Or | | | | X | | | | |
| Combativeness | | | | | | | | |
| Dementia | | | | | | | | |
| Depersonalization | X | | | | | | | |
| Depression | X | X | X | | | | | |
| Diarrhea | X | | | X | X | | X | X |
| Difficult or Labored Breathing | X | X | | | X | | X | |
| Difficult or Troubled Breathing | | | | | | | | X |
| Difficult, Burning, Or Painful Urination | | X | | | | X | | |
| Difficult, Fast, Noisy Breathing, Sometimes with | | X | | | | | | |
| Wheezing | | | | | | | | |
| Difficulty Having a Bowel Movement (Stool) | | X | | X | X | | X | X |
| Difficulty in Passing Urine (Dribbling) | X | | | X | X | | | |
| Difficulty with Breathing | | | | | | X | | |
| Difficulty with Moving | | | | X | | X | X | |
| Difficulty with Sleeping | | | | | X | | | |
| Difficulty with Swallowing | X | | | X | X | | | |
| Difficulty with Thinking or Concentrating | | | | | X | | | |
| Difficulty with Walking | | | | X | | | | |
| Discouragement | | | | X | X | | X | |
| Disorientation | | | | | X | | | |
| Disturbed Color Perception | | | | | X | | | |
| Dizziness | X | | | X | | X | X | |
| Dizziness, Faintness, Or Lightheadedness When Getting | X | X | | | X | | | X |
| Up Suddenly from A Lying or Sitting Position | | | | | | | | |
| Double Vision | | | | X | X | | | |
| Drowsiness | | | | X | | X | | X |
| Drowsiness to Profound Coma | | | | | X | | | |
| Dry Mouth | X | X | | X | X | | X | |
| Dry Skin | X | | | | | | | |
| Dyspepsia | X | | | | | | | |
| Dysphagia | X | | | | | | | |
| Dysphoria | X | | | | | | | |
| Dyspnea | X | | | | | | | |
| Dysuria | X | | | | | | | |
| Ear Congestion | | X | | | | | | |
| Edema | X | | | | | | | |
| Emotional Lability | X | | | | | | | |
| Eructation | X | | | | | | | |
| Euphoria | X | | | | | | | |
| Excess Air or Gas in The Stomach or Intestines | | | | X | X | | | |
| Excessive Muscle Tone | | | | | | | X | |
| Exfoliative Dermatitis | X | | | | | | | |
| Extra Heartbeats | | | | X | | | | |
| Extreme Drowsiness | X | | | | | | | |
| Extremely Shallow or Slow Breathing | | | | | | | X | |
| Face Is Warm or Hot to Touch | | | | | | | | |
| Facial Edema | X | | | | | | | |
| Fainting | X | | | | | | X | |
| False or Unusual Sense of Well-Being | | | | X | X | | | |
| Fast or Irregular Heartbeat | | | | | | X | | |
| Fast, Pounding, Or Irregular Heartbeat or Pulse | | | | X | | | | |
| Fast, Pounding, Racing, Or Irregular Heartbeat or Pulse | | | | | X | | | |
| Fast, Weak Pulse | X | | | X | | | | |
| Fear or Nervousness | | X | | X | | | | |
| Feeding Drunk | | | | | | X | | |
| Feeling Faint, Dizzy, Or Lightheaded | | | | | | | | X |
| Feeling Jittery | | | | | X | | | |
| Feeling of Constant Movement of Self or Surroundings | | | | X | X | | X | |
| Feeling of Warmth | | | | X | X | | | |
| Feeling of Warmth or Heat | X | | | | | | X | X |
| Feeling Sad or Empty | | | | X | X | | X | |
| Fever | X | X | X | | X | X | | |
| Fever or Chills | | | | | | | X | X |
| Flatulence | X | | | | | | | |
| Floating Feeling | | | | | | | | |
| Flushing or Redness of the Skin, Especially On the Face | X | | | | | | X | X |
| and Neck | | | | | | | | |
| Frequent Urge to Urinate | | X | | | | | | |
| Full Feeling | | | | X | | | | |
| Full or Bloated Feeling | | | | | X | | | |
| Gastritis | X | | | | | | | |
| Gastroesophageal Reflux Disease | | X | | | | | | |
| Gastrointestinal Disorder | X | | | | | | | |
| General Feeling of Discomfort or Illness | | | | | | | | |
| Hallucination | X | | | | X | | | |
| Halos Around Lights | | | | | X | | | |
| Headache | X | X | | X | X | X | X | X |
| Hearing Loss | | | | X | | | | |
| Heart Stops | | | | X | | | | |
| Heartburn | | X | | X | X | | | |
| Heartburn or Indigestion | | | | | | X | | |
| Hematuria | X | | | | | | | |
| Hiccups | X | | | | | | | |
| Hives or Welts | | | | X | | | | |
| Hives, Itching, Or Skin Rash | X | | | | X | | | |
| Hoarseness | | | | | X | | | |
| Holding False Beliefs That Cannot Be Changed by Fact | | | | | | | | |
| Hyperkinesia | X | | | | | | | |
| Hyperventilation | | | | | X | | | |
| Hypoesthesia | X | | | | | | | |
| Hypotonic | X | | | | | | | |
| Impotence | X | | | | | | | |
| Inability to Have or Keep an Erection | | | | X | | | | |
| Incidence Not Known | X | | | X | | X | | |
| Incidence Not Known | X | | | X | | X | | |
| Increased Appetite | X | | | X | | | | |
| Increased Blood Pressure | | | | | | | | |
| Increased Sensitivity of the Eyes to Light | | | | X | | | | |
| Increased Sweating | | X | | | X | X | | |
| Increased Thirst | X | | | | | | X | |
| Indigestion | | | | X | X | | | |
| Insomnia | X | | | | | | | |
| Irregular Heartbeat | | | | | | | X | |
| Irregular, Fast or Slow, Or Shallow Breathing | | X | | X | | | | |
| Irregular, Fast, Slow, Or Shallow Breathing | X | | | | | | | X |
| Irregular, Slow, Or Shallow Breathing | | | | | X | | | |
| Irritability | | | | X | X | | X | |
| Irritability or Restlessness | | | | | | X | | |
| Irritation | | | | | X | | | |
| Irritation, Pain, Or Sores at The Site of Application | | | | | | | X | |
| Itching Skin | | X | | | | | X | |
| Joint Pain, Stiffness, Or Swelling | | | | X | X | X | | |
| Lack of Appetite | | | | | X | | | |
| Lack or Loss of Strength | | | | | | | X | X |
| Large, Hive-Like Swelling On the Face, Eyelids, Lips, | | | | | | X | | |
| Tongue, Throat, Hands, Legs, Feet, Or Sex Organs | | | | | | | | |
| Less Common | | X | | X | | | | |
| Less Common or Rare | | | | X | | | | |
| Lethargy | | | | | X | | | |
| Lightheadedness | X | | | | | | X | |
| Lightheadedness, Dizziness, Or Fainting | | X | | X | | | | |
| Loss in Sexual Ability, Desire, Drive, Or Performance | | | | X | | | | |
| Loss of Appetite | X | | | X | | | X | |
| Loss of Balance | | | | X | | | | |
| Loss of Consciousness | X | X | | | | | | |
| Loss of Interest or Pleasure | | | | X | X | | X | |
| Loss of Taste | | | | X | | | | |
| Loss of Vision | | | | | X | | | |
| Loss of Voice | | X | | | | X | | |
| Low Blood Pressure or Pulse | | | | | | | | |
| Low Body Temperature | | | | X | | | | |
| Lower Back or Side Pain | | X | | | | X | X | X |
| Lymphadenopathy | X | | | | | | | |
| Malaise | X | | | | | | | |
| Mental Depression | | | | | X | | | |
| Migraine | X | | | | | | | |
| Mood Changes | | | | | | | X | |
| Mood or Mental Changes | | | | X | | | | |
| Muscle Aches | | | | X | | X | | |
| Muscle Cramps or Spasms | | | | | | | | |
| Muscle Pain or Cramps | X | | | | | | X | |
| Muscle Pain or Stiffness | | | | X | | X | | |
| Muscle Spasms | | X | | X | | | | |
| Muscle Stiffness | | | | | | | X | |
| Muscle Stiffness or Tightness | | | | X | | | | |
| Muscle Tension or Tightness | | | | | | | X | |
| Muscle Twitching or Jerking | | | | X | | | X | |
| Muscle Weakness | | | | | X | | | |
| Nasal Congestion | | X | | | | | | |
| Nausea | | X | | X | | X | | X |
| Nausea or Vomiting | X | | | | X | | X | |
| Nervousness | X | | X | | X | | X | |
| Night Blindness | | | | | X | | | |
| Nightmare or Unusually Vivid Dreams | | | | | X | | | |
| No Blood Pressure or Pulse | | | | | X | | | |
| No Muscle Tone or Movement | X | X | | X | | | | |
| No Pulse or Blood Pressure | | | | X | | | | |
| Noisy Breathing | | | | X | | | | |
| Not Breathing | | | | X | X | | | |
| Numbness of The Feet, Hands, And Around the Mouth | | | | X | | | | |
| Numbness or Tingling in The Hands, Feet, Or Lips | | | | | | | X | |
| Over Bright Appearance of Lights | | | | | X | | | |
| Overactive Reflexes | | | | X | | | | |
| Pain in The Arms or Legs | | | | X | | | | |
| Pain in The Joints | | | | X | | | X | |
| Painful or Difficult Urination | | | | | | | X | X |
| Painful Urination | | | | X | X | | | |
| Pale or Blue Lips, Fingernails, Or Skin | X | | | X | X | X | | X |
| Pale Skin | | X | | | | | X | |
| Paresthesia | X | | | | | | | |
| Passing Gas | | | | X | X | | | |
| Peripheral Edema | X | | | | | | | |
| Pinpoint Pupils | | | | | | | | X |
| Pinpoint Red Spots On the Skin | | | | | | | | |
| Polyuria | X | | | | | | | |
| Postural Hypotension | X | | | | | | | |
| Pounding in The Ears | | | | | X | | X | |
| Pounding in The Ears | | | | | X | | X | |
| Pressure in The Stomach | | | | | X | | | |
| Problems with Memory | | | | | | | | |
| Problems with Muscle Control | | | | | | | | |
| Puffiness or Swelling of the Eyelids or Around the | X | | | | X | X | | |
| Eyes, Face, Lips, Or Tongue | | | | | | | | |
| Rapid Breathing | X | | | X | X | | X | |
| Rapid Weight Gain | X | X | | | | | | |
| Rare | | | | | | X | | |
| Rash | X | | | | | | X | |
| Rash, Hives, Or Itching | | | | | | X | | |
| Redness of The Face, Neck, Arms And Occasionally, | | | | X | X | X | | |
| Upper Chest | | | | | | | | |
| Redness of The Skin | | | | X | X | | | |
| Relaxed and Calm | | | | X | X | | | |
| Relaxed and Calm Feeling | | | | | | | | X |
| Restlessness | | | | | X | | X | |
| Rhythmic Movement of Muscles | | | | X | | | | |
| Rhythmic Movement of the Muscles | | | | | | | X | |
| Runny Nose | | X | | X | | | | X |
| Seeing Double | | | | X | | | | |
| Seeing, Hearing, Or Feeling Things That Are Not There | | | | X | | X | X | |
| Seizures | | | | | | | X | |
| Sensation of Heaviness | | | | | | X | | |
| Sensation of Spinning | | | | X | X | | X | |
| Severe Constipation | X | | | | X | | X | |
| Severe Sleepiness | X | X | | | | | X | |
| Severe Sleepiness or Unusual Drowsiness | | | | | X | | | |
| Severe Stomach Pain, Cramping, Or Burning | | | | X | | | | |
| Severe Vomiting | X | | | X | X | | | |
| Shakiness and Unsteady Walk | | | | | | | | |
| Shakiness in The Legs, Arms, Hands, Or Feet | X | | | | | | X | |
| Shakiness or Trembling | | | | | | X | | |
| Shaking | | | | X | X | | | |
| Shivering | | | | X | | | | |
| Shortness of Breath | | | | | | | | X |
| Skin Itching | | | | X | | | | |
| Skin Rash | | | | | | | | |
| Sleepiness | | | | | X | | | X |
| Sleepiness or Unusual Drowsiness | | X | | X | | | X | |
| Sleeplessness | | | | | | | | X |
| Slow Heartbeat | | | | | | | | |
| Show or Fast Heartbeat | | | | | | | X | |
| Slow or Irregular Heartbeat | X | X | | X | | | | |
| Slurred Speech | | | | X | | X | | |
| Sneezing | | X | | X | | | X | X |
| Sore Throat | | X | | | | | X | |
| Speech Disorder | X | | | | | | X | |
| ST Depression | X | | | | | | | |
| Stiff Neck | | | | X | | | | |
| Stomach Cramps | | | | | | | X | |
| Stomach Discomfort or Upset | | | | | X | | | |
| Stomach Discomfort, Upset, Or Pain | | | | X | | X | | |
| Stomach Pain | | | | | | | | X |
| Stomatitis | X | | | | | | | |
| Stopping of Heart | | | | | X | | | |
| Stuffy Nose | | | | | | | | X |
| Stupor | X | | | | | | | |
| Stupor tremor, Vertigo, Taste Perversion | X | | | | | | | |
| Sudden Sweating | | | | | X | | | |
| Sunken Eyes | X | | | X | X | | X | |
| Sweating | X | | | X | X | | X | X |
| Sweating or Chills | | | | | | | | |
| Swelling of The Abdominal or Stomach Area | | | | | X | | | |
| Swelling of The Eyelids, Face, Lips, Hands, Or Feet | | | | | | X | | |
| Swelling of The Face, Fingers, Or Lower Legs | | | | | | | | |
| Swelling of The Feet, or Lower Legs | | | | X | | | | |
| Swelling of The Hands, Ankles, Feet, Or Lower Legs | | | | | | | X | |
| Swelling of The Hands, Ankles, Or Feet | | | | | X | | | |
| Symptoms of Overdose | | X | | | | | | |
| Syncope | X | | | | | | | |
| Taste Perversion | X | | | | | | | |
| Thinking Abnormalities | | | | | | | X | |
| Thirst | X | | | | X | | | |
| Thought Abnormalities | X | | | | | | | |
| Thoughts of Killing Oneself | | | | X | | X | | |
| Tightness in The Chest | X | X | | X | X | X | X | |
| Tingling of The Hands or Feet | X | X | | X | | | | |
| Tinnitus | X | | | | | | | |
| Tiredness | | | | | X | | X | |
| Tooth Pain | | | | | | | X | |
| Trembling or Shaking of the Hands or Feet | X | | | | | | X | |
| Tremor | X | | | | | | | |
| Trouble Breathing | | | | X | | | | |
| Trouble Concentrating | | | | X | X | | X | |
| Trouble in Holding or Releasing Urine | | | | | X | | | |
| Trouble Sleeping | | | | X | | | X | X |
| Trouble with Gums | | | | | | | X | |
| Trouble with Speaking | | | | X | | | | |
| Trouble with Speaking or Hoarseness | | | | | | X | | |
| Trouble with Teeth | | | | | | | X | |
| Troubled Breathing or Swallowing | | | | | | X | | |
| Troubled Breathing with Exertion | | | | | | | X | |
| Tunnel Vision | | | | | X | | | |
| Twitching | X | | | | | | | |
| Ulcers, Sores, Or White Spots in The Mouth | | | | | | | X | |
| Unable to Sleep | | | | | | | | X |
| Uncomfortable Swelling Around the Anus | | | | X | | | | |
| Unconscious | | | | X | | | | |
| Uncontrolled Eye Movements | | | | X | | | | |
| Unsteadiness, Trembling, Or Other Problems with | | | | | | | | |
| Muscle Control or Coordination | | | | | | | | |
| Unusual Bleeding or Bruising | | | | | | | X | |
| Unusual Drowsiness, Dullness, Tiredness, Weakness, | | | | | | X | | |
| Or Feeling of Sluggishness | | | | | | | | |
| Unusual Excitement, Nervousness, Or Restlessness | | | | | | | | |
| Unusual Tiredness | | | | X | | | | |
| Unusual Tiredness or Weakness | X | X | | | X | X | X | X |
| Unusual Weight Gain or Loss | X | X | | X | | | | |
| Upper Abdominal or Stomach Pain | | | | X | | | | |
| Urinary Retention | X | | | | | | | |
| Vertigo | X | | | | | | | |
| Very Slow Heartbeat | X | | | | | | | |
| Voice Alteration | X | | | | | | | |
| Vomiting | | X | | | | X | | X |
| Vomiting Material That Looks Like Coffee Grounds, | | | | X | | | | |
| Severe and Continuing | | | | | | | | |
| Weak or Feeble Pulse | | | | X | | | | |
| Weakness | | | | | | | X | |
| Weight Gain | | | | | | | | |
| Weight Loss | | | | | | | X | |
| Welts | | | | | X | | | |
| Wheezing | | | | | | | | |
| Withdrawal Syndrome (with and Without Seizures) | X | | | | | | | |
| Wrinkled Skin | X | | | X | X | | X | |

Opioids in the development pipeline can utilize the Invention to capture required clinical trial information and control opioid dispensing for regulatory drug approval as well as to control opioid dispensing after regulatory approval. All in development opioids are encompassed in the embodiment of the invention by reference. The FDA and like regulatory agency general and opioid specific approval self-assessment, self-testing, self-reporting, digitally captured diagnostic testing, digital diary information, dispensing information, etc. drug approval recommended data capture and reporting guidelines are incorporated by reference. The listing for each opioid includes, by definition, each opioid's respective indication(s), strength, dosage form, route of administration, side effect profile, drug interactions, and regulatory approval guidelines, etc.

Oral opioid mechanisms of action for marketed oral opioid drugs, oral opioid drugs in developed, and efficacious oral opioid drugs withdrawn from the market due to dose related side effects or whose development was stopped due to a side effect(s) that can be addressed by the embodiment and thereby made approvable are incorporated by reference. The listed drugs in the following databases are encompassed in the embodiment of the invention by reference: Cortellis™ Competitive Intelligence by Thomson Reuters; Adis R&D; and Pharmaprojects by Citeline. The listing for each includes by definition each respective drug's respective indication(s), strength, dosage form, route of administration, side effect profile, drug interactions, mechanisms of action, etc.

Oral opioids listed in the following databases that (i) were in development but were discontinued due to dose related side effects whose safety concerns can be addressed by the Invention or (ii) drugs that were withdrawn from the market after approval due to dose related side effects whose safety concerns can be addressed by the Invention and contained in the following databases: (i) Cortellis™ Competitive Intelligence by Thomson Reuters, and/or (ii) Adis R&D, and/or (iii) Pharmaprojects by Citeline are all encompassed in the embodiment of the invention by reference. The listing for each drug includes each drug's respective indication(s), strength, dosage form, route of administration, side effect profile, drug interactions, mechanisms of action, regulatory approval guidelines, etc.

### III. System and Method to Control the Delivery of Oral Drugs

Various embodiments will be described hereinafter with reference to the accompanying drawings. These embodiments are illustrated and described by example only and are not intended to be limiting. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

The word “exemplary” is used herein to mean “serving as an example, instance, or illustration”. Any embodiment described herein as “exemplary” or “example” is not necessarily to be construed as preferred or advantageous over other embodiments. Likewise, the term “embodiments of the invention” does not require that all embodiments of the invention include the discussed feature, advantage or mode of operation.

Further, many embodiments are described in terms of sequences of actions to be performed by, for example, elements of a computing device. It will be recognized that various actions described herein can be performed by specific circuits (e.g., application specific integrated circuits (ASICs)), by program instructions being executed by one or more processors, or by a combination of both. Additionally, these sequences of actions described herein can be considered to be embodied entirely within any form of computer readable storage medium having stored therein a corresponding set of computer instructions that upon execution would cause an associated processor to perform the functionality described herein. Thus, the various aspects of the invention may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

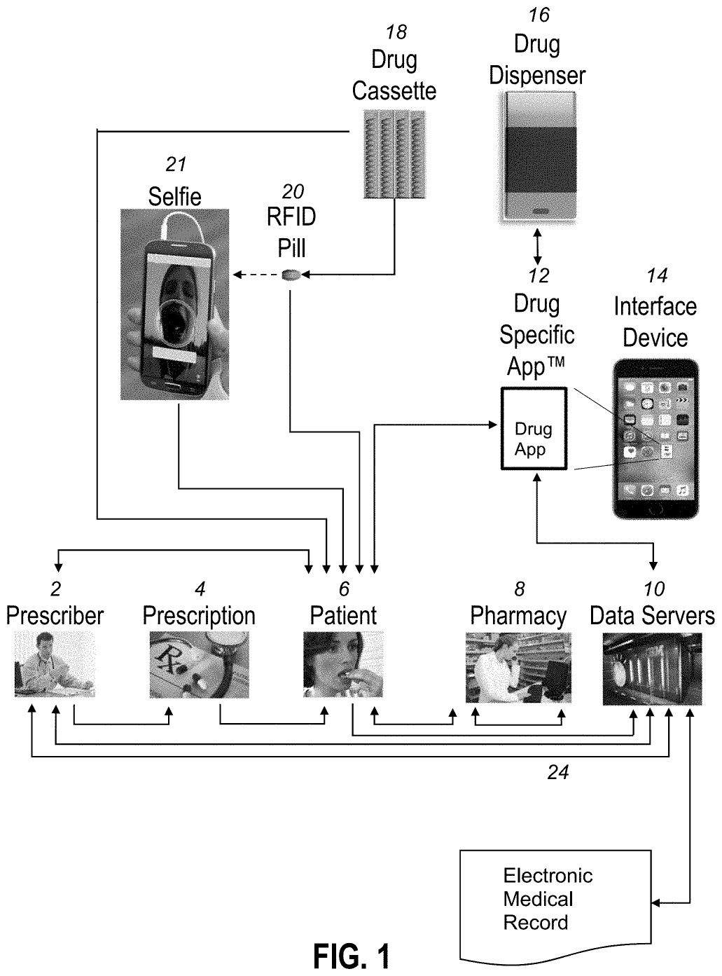

FIG. 1 illustrates an exemplary embodiment of the present invention of a closed loop system to control drug diversion, misuse, abuse, double dosing, addiction, dependence, overdose, and deaths.

The following are exemplary descriptions of the FIG. 1 embodiments of the invention and how different components relate one to the other and how together they contribute to a closed loop drug traceability and control system:

The Prescriber 2 is identified via his (i) National Provider Identification (NPI) number, (ii) DEA Registration Number, (iii) state medical license number, and/or (iv) other association and/or government identification number. This number is related to the Patient 6 on the Prescription 4 and the prescription fulfillment Pharmacy 8.

The Prescription 4 is specific to the Patient 6 and is related to the Prescriber 2, Pharmacy 8, the Patient's 6 prescription database file on the Data Servers 10, and the Drug Specific App 12. The Prescription 4 information is electronically or manually loaded and related to the Data Servers 10 and the Drug Specific App 12.

The Patient 6 is related to the Prescriber 2, Prescription 4, Pharmacy 8, the Data Servers 10, the Drug Specific App 12, the Interface Device 14, the Drug Dispenser 16, the individual Drug Cassette 18 (which in some embodiment of the invention may be an integral component of the Drug Dispenser 16), and any RFID 20 enabled device which is incorporated with the oral dosage form to indicate when the drug has been ingested, any program which allows the Patient 6 to take a Selfie 21 of themselves taking the drug that is automatically recognized by the program.

The Pharmacy 8 is related to the Patient 6 and the Prescriber 2 when the e-prescription is entered by the Prescriber 2 and/or the Pharmacy 8 receives the Prescription 4; and to the Data Servers 10 when the Prescription 4 is first filed by the Prescriber 2 and/or filled by the Pharmacy 8 and to the Drug Specific App 12 when it is subsequently created by the App Generation Program 200 and then when it is downloaded to the Interface Device 14.

The Data Servers 10 are related to the Patient 6 when the Prescriber 2 files an electronic Prescription 4 or when the Pharmacy 8 fills the Prescription 4 and enters the Prescriber's 2 Patient 6 specific Prescription 4 instructions. Upon receipt of the Patient's 6 and the Prescription 4 information, the App Generation Program 200 on the Data Servers 10 create a Drug Specific and Patient Tailored App 12 which contains a personalized Patient 6 tailored Drug Specific Dispensing Algorithm 13. In addition to the Patient 6, Prescriber 2, prescription number, pharmacy 8, drug, strength, and dosing schedule information, the Prescription 4 may contain specific Patient 6 self-assessment, self-test, self-report, or digitally captured Patient 6 information/data which may be incorporated into the Drug Specific Dispensing Algorithm 13 or which may be captured to assist the Prescriber 2 or other healthcare professionals in managing the Patient 6 or to capture information required for drug regulatory submissions during clinical trials. The data capture may include patient information requested by the Prescriber 2 which is not utilized by the Drug Specific Dispensing Algorithm 13 to make a dispense, or not, decision, i.e., keep the Drug Dispenser 16 locked or unlock the Drug Dispenser 16 and allow a drug dose to be dispensed decision.

The dispensing information for the RFID Pill 20 and/or the Selfie 21 may be captured using an API with the respective program to close the Closed Loop Drug Traceability and Control or the data can be aggregated by the Data Servers 10 from the Patient's 6 Electronic Medical Record 24. This would then provide complete pill by pill drug traceability from the time the drug is loaded into the Drug Cassette 18 until it is consumed by the Patient 6. If the data servers are authorized to access the Patient's 6 Electronic Medical Record in keeping with HIPPA and other governmental guidelines, an API can be utilized to access the consumption data in the Electronic Medical Record 24, update the Patient's 6 database on the Data Servers 10 and thereafter update the information on the related Drug Specific App 12. This will provide true drug traceability and accountability.

The Drug Specific and Patient Tailored App 12 is generated by the Data Servers 10 and specifically configured/personalized based upon the prescription dispensing instructions and the side effect(s) or values that the Prescriber 2 wants monitored. This information may or may not be factored into the Drug Specific Dispensing Algorithm 13 for the Patient 6. Once the Drug Specific App 12 is generated, it is given a specific Patient 6 identification number 160 that ties the Drug Specific App 12 to the Patient 6. When the Drug Specific App 12 is ready, the Data Servers 10 send an email and/or text message (and/or other similar communication) with the Drug Specific App 12 download link for the Patient 6 to download the App onto his/her Interface Device 14 or Devices 14 and/or Standalone Drug Dispenser 16. At the same time, the Data Servers 10 add the Patient 6 to the respective Data Server's 10 Patient Database 152 files to facilitate receipt of the Drug Specific App 12 aggregated and thereafter communicated Drug Dispenser 16 serial number, Drug Cassette 18 serial number, drug NDC number, batch number, Prescription 4 expiration/beyond-use date, drug storage requirements, and related Drug Dispenser Identification No. 158 and Drug Specific App 12 uploaded drug specific events, alerts, and information. Simultaneously, the Data Servers 10 define available Electronic Medical Record 24 links and create a relationship(s) between all Patient 6 related information/data files on the Data Servers 10 to facilitate data aggregation, analysis, querying, and reporting, etc.

The Interface Device 14 is where the Drug Specific App 12 resides for tethered Drug Dispensers 16. The Drug Specific App 12 is tied to the Interface Device 14 through its telephone number and/or IP address. The Drug Specific App 12 utilizes the Interface Device's 14 memory, processor, speaker, camera, biometric authentication, communications connections (Bluetooth, Wi-Fi, Internet, etc.), phone, and other features to enable the Drug Specific App 12 to control the Drug Dispenser 16 and communicate (e.g., through screen messages, vibration alerts, alarms, via sending emails, text messages, or making phone calls, etc.) with the Patient 6, the Data Servers 10, the Integrated Call Center 22, the Prescriber 2, other healthcare professionals and caregivers, the Pharmacy 6 (e.g., for refills, etc.), etc.

The Drug Dispenser 16 is related to the Drug Specific App 12 via the exchange of the Drug Dispenser's Identification Number 158 and/or unique serial number which is exchanged with the Drug Specific App 12 during the first wireless connection/handshake. Thereafter, the Drug Specific App 12 utilizes the Drug Dispenser's 16 serial number to ensure it is connected with the Patient's 6 Drug Dispenser 16 containing the desired drug. Once verified, the Drug Specific App 12 controls drug dispensing by the Drug Dispenser 16 and all other Drug Dispenser 16 functions and communications.

The Drug Cassette 18 has a unique cassette serial number that is related to the drug's NDC number and unique batch number along with drug specific information. The Drug Cassette 18 docks into the Drug Dispenser 16 or is an integral part of the Drug Dispenser 16, at which time the Drug Dispenser 16 and the Drug Cassette 18 use the Drug Dispenser's 16 same serial number, depending on the Drug Dispenser's 16 design (the drug cassette's 18 keep their own serial numbers in reusable, multi-drug drug dispensers 16). Upon docking, the Drug Cassette 18, which comes related to a specific drug and drug batch number, becomes related with the Drug Dispenser 16 which is in turn related to the Drug Specific App 12, the Data Servers 10, the Pharmacy 8, the Prescription 4, the Prescriber 2, and the Patient 6. The information related to the drug contained in the Drug Cassette 18 is utilized by the Drug Specific App's 12 Drug Specific Dispensing Algorithm 13. The Drug Cassette is in turn related to the Drug Dispenser 16 which is related to the Drug Specific App 12 which is related to the Patient 6, thereby providing a closed traceability and control loop. The ideal would be to have a Drug traceability and control loop that show that the specific dosage form in ingested by the Patient 6.

The RFID Pill 20 is an oral dosage form that contains a chip which transits a signal when it is ingested. The Drug Specific App 12 can be configured, using an API, to pick up the signal and to capture the ingestion information. The time interval between when the oral dosage form is dispensed and ingested is indicative of whether the dosage is being taken by the Patient 6 or potentially diverted. The RFID chip is related to the RFID Pill 20 which is in turn related to the Drug Cassette 18. The Drug Cassette is in turn related to the Drug Dispenser 16 which is related to the Drug Specific App 12 which is related to the Patient 6, thereby providing a closed traceability and control loop.

The Selfie 21 recognition software is designed to recognize both the Patient 6 and the solid dose medication, drug, the Patient 6 is taking. The Patient 6 is tied to the Drug Dispenser 16 and the related Drug Cassette 18 and the related drug. The time interval between when the drug is dispensed by the Drug Dispenser 16 and the time the drug is captured as being taken by the Patient 6 is indicative of Patient 6 prescription compliance or drug divergence. If the drug is an RFID Pill 20, then there is full confirmation that the Patient 6 did in fact take the dispensed drug—closing the traceability and control loop for the individual drug pill.

An Electronic Medical Record 24 is created by the Data Servers 10 at the same time that the first Drug Specific App 12 is created for the Patient 6. The Electronic Medical Record 24 is configured to be discoverable by authorized care givers using a National Record Locator Service (NRLS) like SureScripts. The Electronic Medical Record 24 is configured for dispensing information and to present any data captured by the Drug Specific App 12 and any medically pertinent communications between the Patient 6 and the Integrated Support Center 22 from the interactions logs. This data is automatically updated by the Data Servers 10. The Electronic Medical Record 24 can also be utilized to check to ascertain if any other prescriptions are current for medications which may be in conflict with any new or existing drug Prescription 6.

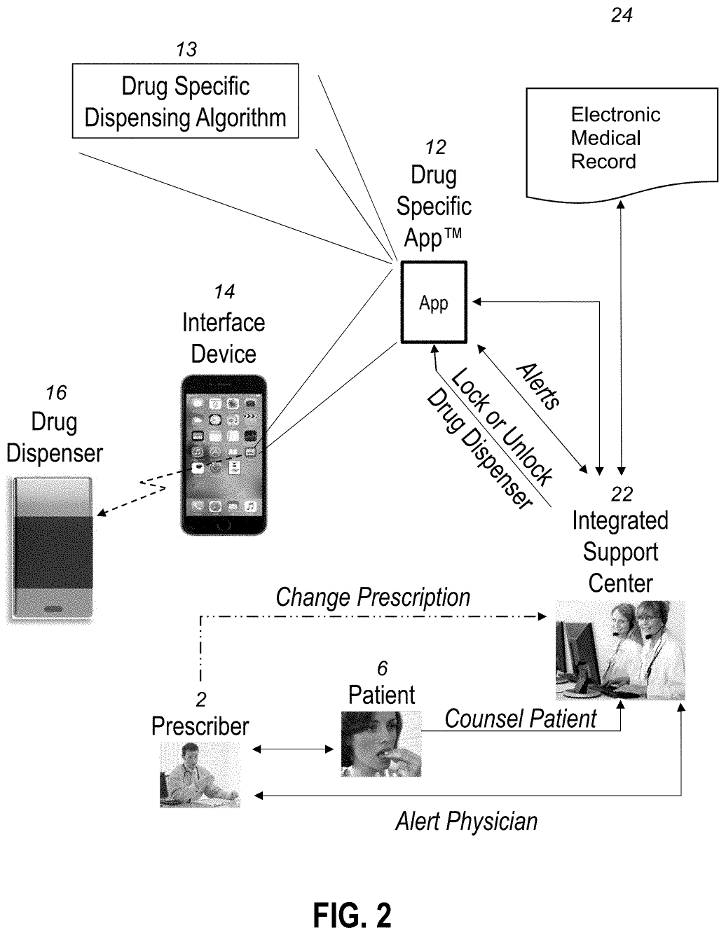

FIG. 2 illustrates an exemplary embodiment of the present invention, an integrated drug dispensing and patient management system composed of a Drug Specific App 12 which contains a Drug Specific Dispensing Algorithm 13 resident on an Interface Device (Smartphone, computer Tablet, portable or desktop computer, standalone drug dispenser, etc. with Bluetooth, Wi-Fi, and/or Internet communications capabilities) 14 used to control dispensing by a (single or multidrug) Drug Dispenser 16; an Integrated Support Center 22; a Patient 6; a Prescriber 2; and the Patient's Electronic Medical Record 24.

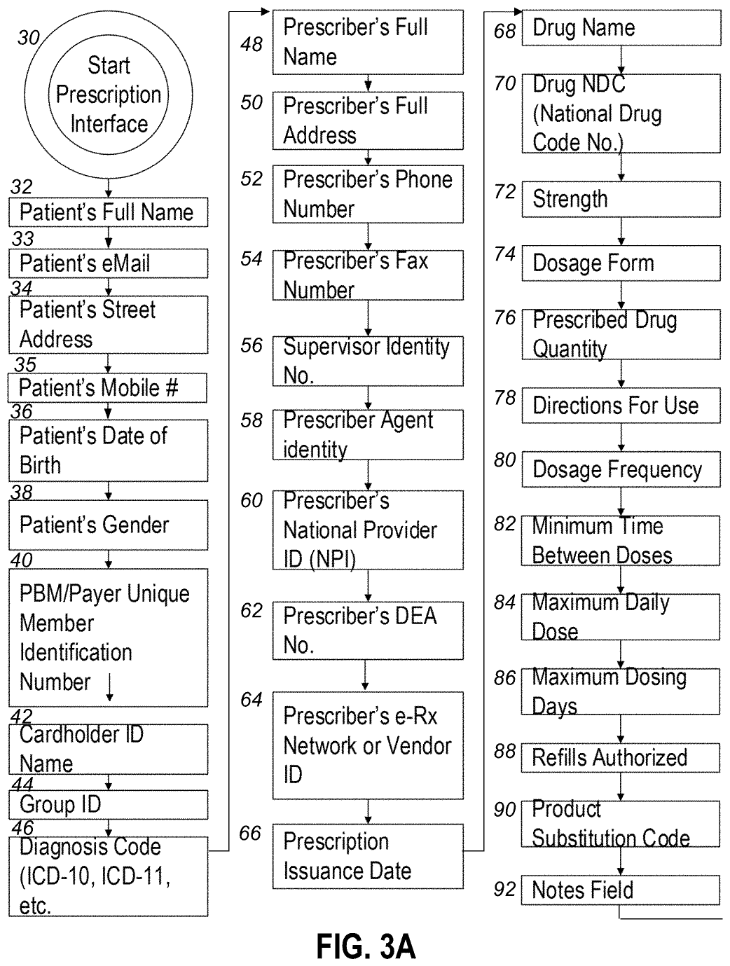

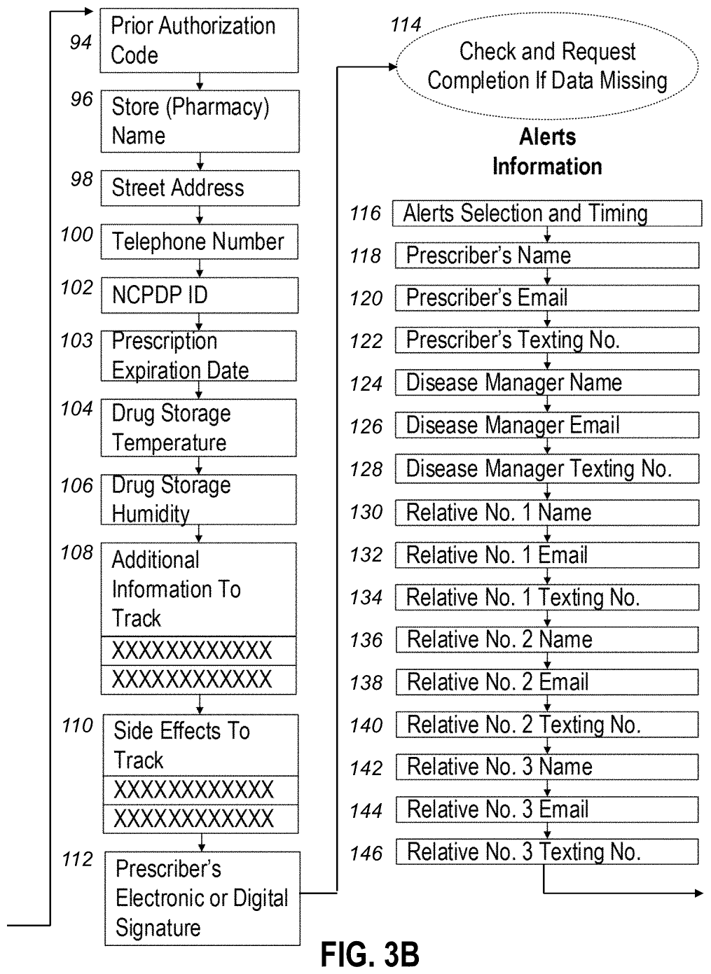

FIG. 3 is an exemplary embodiment depicting the Prescription Interface Module 30 which is designed to integrate data entry with the respective generally used e-prescribing programs (e.g., ADSC MedicsRx, Allscripts, DAW Systems, DrFirst Rcopia, MDT Toolbox-Rx, Medtab RxCure, OA Systems, Practice Fusion, RxNT eRx, Spectra eRx, etc.) and/or pharmacy prescription systems (e.g., OmniCare, QS/1, PharMerica, Frameworks, etc.) and/or national record locator services (NRLS), such as the one offered by SureSripts, via an application program interface (API). The requested information can be directly entered by the Prescriber 2 and simultaneously submitted to Pharmacy 8 and its Pharmacy Database 164 as well as the Integrated Support Center's 22 Patient Database 152. Any data that was not entered by the Prescriber 2 can be entered at the Pharmacy 8. These updates are automatically uploaded to the Patient Database 152. Any data that was not entered by either the Prescriber 2 or the Pharmacy 8 can be entered by the Patient 6. However, the information that can be entered by the Patient 6 using direct data entry onto the Drug Specific App's 12 prescription information screen is limited to Alerts Information 116 to 146. The Patient 6 entered data is automatically uploaded and synchronized by the Drug Specific App 12 with the Patient Database 152.

The Prescription Interface 30 captures the Patient's Full Name 32, Patient's Street Address 34, Patient's Date of Birth 36, Patient's Gender 38, PBM/Payer Unique Member Identification Number 40, Cardholder's ID Name 42, Group ID 44, Diagnosis Code (ICD-10, ICD-11, etc.) 46, Prescriber's Full Name 48, Prescriber's Full Address 50, Prescriber's Phone Number 52, Prescriber's Fax Number 54, Supervisor Identity No. 56, Prescriber's Agent Identity 58, Prescriber's National ID (NPI) 60, Prescriber's DEA No. 62, Prescriber's e-Prescription Network or Vendor ID 64, Prescription Issuance Date 66, Drug Name 68, Drug NDC (National Drug Code No.) 70, (Drug) Strength 72, Dosage Form 74, Prescribed Drug Quantity 76, Directions For Use 78, Dosage Frequency 80, Minimum Time Between Doses 82, Maximum Daily Dose 84, Maximum Dosing Days 86, Refills Authorized 88, Product Substitution Code 90, Notes Field 92, Prior Authorization Code 94, Store (Pharmacy) Name 96, (Store) Street Address 98, (Store) Telephone Number 100, NCPDP (National Council for Prescription Drug Programs) ID 102, Prescription Expiration Date (Beyond-use date which is the earlier of the drug expiration date or the Prescription Expiration Date) 103, Drug Storage Temperature (Range) 104, Drug Storage Humidity (Range) 106, Additional Information To Track (these are additional digitally captured patient values and/or self-assessment, self-test, and/or self-reported patient information the Prescriber 2 wants tracked for better Patient 6 management and/or to capture clinical trial required information for regulatory submissions) 108, Side Effects To Track (this represents specific drug mediated side-effect(s) digitally captured patient values and/or self-assessment, self-test, and/or self-reported Patient 6 information the Prescriber 2 wants tracked to better manage the Patient 6) 110, Prescriber's Electronic or Digital Signature 112,

Check and Request Completion if Data Missing 114 is the program routine to ensure all the requisite Prescription 4 data is captured. The Alerts Information is used by the Standalone Drug Dispenser 16 or the Drug Specific App 12 to advise caregivers and/or family members when a Patient 6 dispenses or ingests (in the case of an RFID Pill 20 or Selfie 21 ingestion confirmation) the drug and for special advisories if some kind of intervention or action may be required. The type of alert sent to each individual can be designated in the Prescription Interface or when the information is directly entered by the Patient 6 into the Drug Specific App 12 or the Standalone Drug Dispenser 16. The following are examples of the data capture fields for the Alerts Information: Alerts Selection and Timing 116, Prescriber's Name 118, Prescriber's Email 120, Prescriber's Texting Telephone No. 122, Disease Manager Name 124, Disease Manager Email 126, Disease Manager Texting Telephone No. 128, Relative No. 1 Name 130, Relative No. 1 Email 132, Relative No. 1 Texting Telephone No. 134, Relative No. 2 Name 136, Relative No. 2 Email 138, Relative No. 2 Texting Telephone No. 140, Relative No. 3 Name 142, Relative No. 3 Email 144, Relative No. 3 Texting Telephone No. 146.

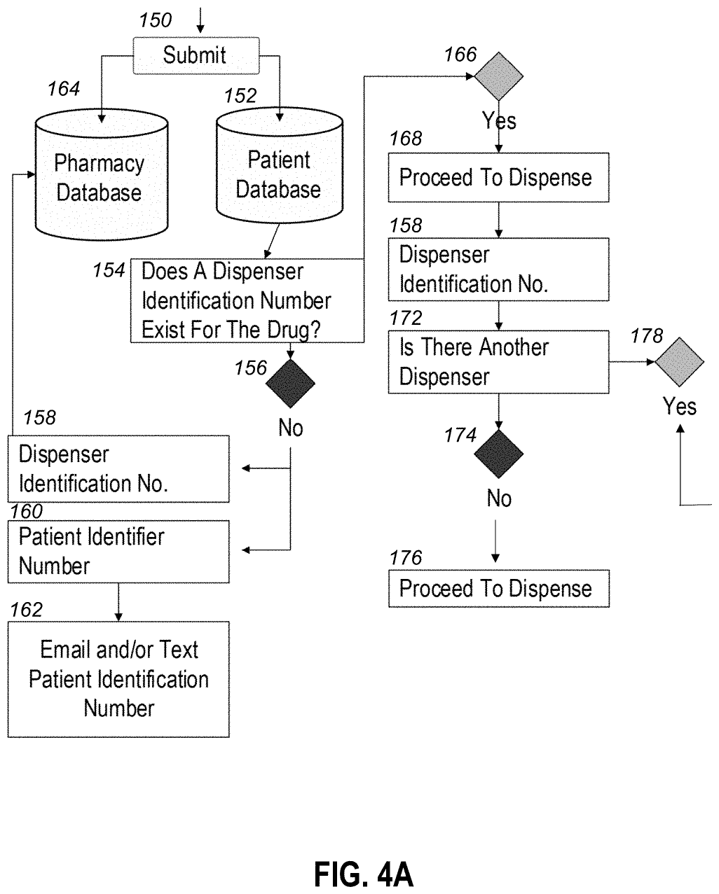

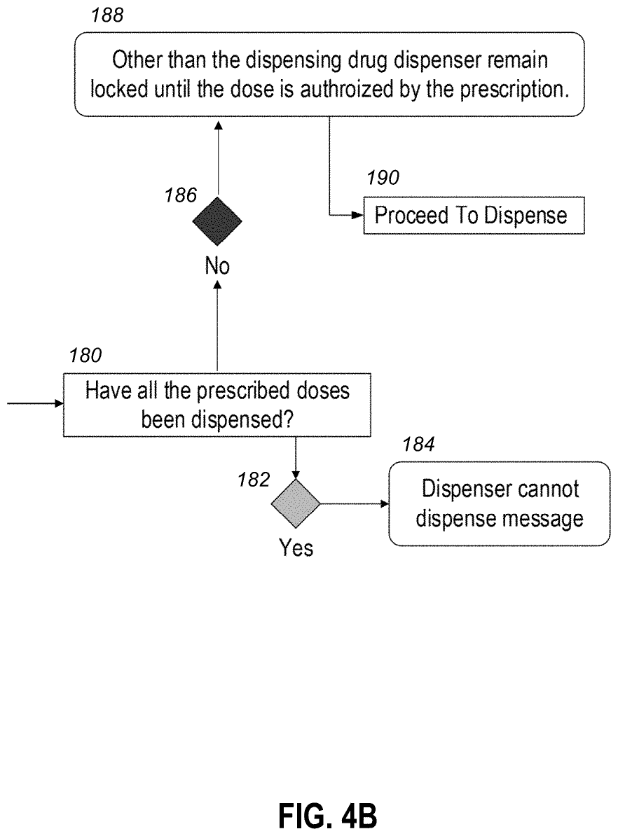

FIG. 4 is an exemplary embodiment of the logic to: (i) enter the Prescription 4 information into the Pharmacy Database 164 and (ii) into the Patient Database 152 and (iii) to generate a Dispenser Identification Number 158 and (iv) Patient Identification Number 160 which are required to create the Drug Specific App 12 and thereafter for dispensing the medication doses. The Dispenser Identification Number 158 is a unique identifier that is utilized to link the Prescription 4 to one or more prescribed Drug Dispenser 16 serial numbers. Once the Dispenser Identification Number 158 exists the logic proceeds to generate and the Patient 6 and Drug Specific App 12 and transmit it to the Patient 6 for downloading into their Interface Device 14 or Devices 14. After it is enabled by the Patient 6, the Drug Specific App 12 is ready to control Drug dose dispensing once the App 12 is tied to one or more Drug Dispensers 16.

The restricted Dispenser Identification Number 158 is issued in response to the type of medication prescribed. If the medication is one where, for example, divergence is a concern, as with Drug, then the restricted Dispenser Identification Number 158 is issued by the Data Servers 10 when the prescription is Submitted 150. The restricted Dispenser Identification Number 158 restricts opening of reusable Drug Dispensers 16 and docking of the Drug containing Drug Cassette 18 to authorized medical professionals.

When the Prescriber 2 or the Pharmacist (Pharmacy) 8 Submits 150 the ePrescription 4 it results in the Pharmacy Database 164 and Patient Database 152 being automatically updated/synchronized. Then it checks to see that the required Dispenser Identification Number 158 already exists for the prescribed Drug for the Patient 6 or if the Dispenser Identification Number 158 need to be generated for the prescribed Drug medication 154. If the answer is No 156, then the program issues the restricted Dispenser Identification Number 158 and automatically sends it to the Pharmacy Database 164 and Patient Database 152 for inclusion with the Prescription 4. It then checks for a Patient Identifier Number 160 which is used as the control number and identifier for all Patient 6 data stored on the Data Servers 10 and the Patient Database 152. If none exists, the program generates a Patient Identifier Number 160. The program automatically emails and/or texts the Patient Identifier Number 160 to the Patient 6 for use by the Patient 6 when initially setting up the Drug Specific App 12.

If a Dispenser Identification Number 158 exists, Yes 166, then the system is ready to Proceed To Dispense 168. Upon confirmation of the Dispenser Identification Number 158, then the program checks the Patient Database 152 to ascertain if there is another dispenser 172 for the prescribed medication. If there isn't another Drug Dispenser 16 for the specific medication, No 174, the Drug Specific App 12 on the Patient Database 152 is set to allow dispensing to proceed 176 per the Prescription Instructions 4.

If another Drug Dispenser 16 for the drug medication is identified, Yes 178, then the program checks to see if there is another dispenser 172, if No 174, then the program is ready to proceed with the Dispense 176 process. However, if there is another dispenser 172, Yes 178, then the program proceeds to check if all the doses in the first Drug Dispenser 16 have been dispensed 180. If the answer is Yes 182, then the Drug Specific App 12 is authorized to proceed with dispensing 184 from the new or additional Drug Dispenser 16 per the Prescription 4 instructions. If the answer is No 186 that not all the medication has been dispensed 180 from the first Drug Dispenser 16, then: (i) the second or subsequent Drug Dispenser 16 remains locked, restricted from dispensing, until the drug in the first Drug Dispenser 16 is fully dispensed, or (ii) the first Drug Dispenser 16 remains locked and the Second or Subsequent Dispenser 16 is allowed to begin dispensing 188 per the Prescription 4. The program guards against dispensing of more than one authorized dose from any one or multiple Drug Dispensers 16. Simultaneously, a message/alerts 190 are generated informing the Patient 6 that the drug in the first Drug Dispenser 16 needs to be fully dispensed before it expires.

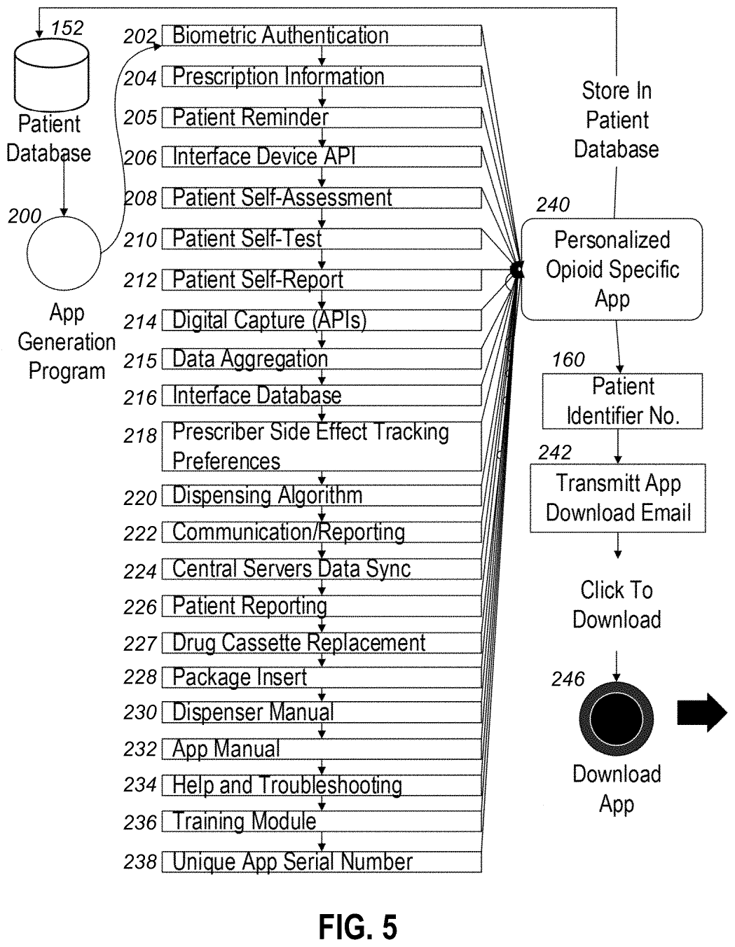

FIG. 5 is an exemplary embodiment depicting a Drug Specific App 12 which resides on an Interface Device 14 or on a Standalone Drug Dispenser 16 and controls drug dose dispensing from a Drug Dispenser 16. When the Patient 6 is prescribed a Drug, the Patient 6 is trained on the operation of the Drug Specific Dispensing App 12 and the related Drug Dispenser 16 using the App's Training Module 236.

In this embodiment of the invention, the Drug Specific App 12 is comprised of the following software modules: (i) Biometric Authentication 202, (ii) Prescription Information 204 module which can be programmed remotely by the Integrated Support Center 22, (iii) the Patient Reminder 205 module (iv) the Interface Device(s) API(s) 206 module, (v) Patient Self-Assessment 208 module which is unique for each drug, (vi) Patient Self-Test 210 module which is unique for each drug, (vii) Patient Self-Report 212 module which is unique for each drug, (viii) Digital Capture (APIs) 214 module, (ix) Data Aggregation 215 module (x) the Interface Database 216, (xi) the Prescriber Side Effect Tracking Preferences 218 module, (xii) the Dispensing Algorithm 220 module which is unique for each drug and which may be personalized for each patient, (xiii) Communication and Reporting 222 module, (xiv) the Central Servers Data Sync 224 module, (xv) the Patient Reporting 226 module, (xvi) Drug Replacement Cassette 227 module, (xvii) the Package Insert 228, (xviii) Dispenser Manual 230, (xix) the Drug Specific App Manual 232, (xx) the Help and Troubleshooting 234 module, (xxi) the Training Module 236, and (xxii) the Unique App Serial Number 238.

The following are exemplary descriptions of the FIG. 5 embodiments of the invention:

The App Generation Program 200 is automatically triggered when an e-Prescription is Submitted 150 to the Patient Database 152. It then proceeds to build each of the following modules, which are specific for each drug and each patient, into a Drug Specific App 12.

Biometric Authentication module 202 encompasses the utilization of a biometric authentication screen and/or digital interface which allows the patient, upon authentication, to automatically move to the first Drug Specific Dispensing Algorithm 13 screen. The Biometric Authentication 202 can constitute one or a combination of more than one biometric authentication method. Examples include Iris Scanning systems, Retinal Scanning systems, Fingerprint Scanning systems, Password Sign-on systems, Facial Scanning systems, Voice Scanning systems, Gesture Recognition systems, automatic server generated temporary password systems, etc.

Biometric Authentication 202 can also be utilized as a diagnostic input that can be used by the Drug Specific Dispensing Algorithm 13. As an example, Iris Scanning can be utilized for both Biometric Authentication 202 and as a Patient Self-Assessment 208 digital input. The Drug Specific App 12 could use the Patient's 6 base and/or trended iris scans to ascertain changes in pupil size to ascertain if the Patient 6 is evidencing the “pinpoint pupils” or “Mydriatic pupils” that are a sign of drug overdose (e.g., opioid overdose). The algorithm would make the appropriate adjustments for lighting conditions, distance, etc. and the Patient's 6 individual pupil dilation characteristics in making its determination.

As part of the Biometric Authentication 202 process, after the Biometric Authentication 202 recognizes the Patient 6, the program: (i) effectuates a handshake/connection with the Drug Dispenser 16, (ii) checks to ensure that the drug has not expired 103 (is not past its Expiration/Beyond-Use Date), (iii) that the drug has been stored within prescribed temperature 104 and humidity 106 parameters, (iv) checks the Prescription 4 information to ensure the Patient 6 is authorized to dispense the dose (the dose is within prescribed Prescription 4 parameters, (v) validates that the Drug Dispenser's 16 serial number 450 is registered to the Patient 6, and then effectuates handshakes/connections with designated digital devices to download specific digitally captured patient values or information 852, 854, 856, 857, 858, 859, 860.

If the Biometric Authentication 202 does not recognize the Patient 6, it asks the Patient 6 to try again. After a given number of tries, it alerts the patient 6 to contact the Integrated Support Center 22 and alerts the Integrated Support Center 22 of the failed attempts and lists the Patient 6 for a follow-up call by the Integrated Support Center 22 if the drug has not been properly dispensed within a specific timeframe.

If the Drug Specific App 12 does not recognize the Drug Dispenser 16, the Patient 6 gets an alert screen explaining why it does not recognize the Drug Dispenser 16, this may include but is not limited to: (i) unable to locate the Drug Dispenser 16, (ii) the Drug Dispenser 16 does not have the right serial number, (iii) the prescribed drug has not been fully dispensed from the other Drug Dispenser 16, etc. Simultaneously, if the Drug Specific App 12 ascertains that the Drug Dispenser 16 does not have the right serial number 450, it will send a message to the Integrated Support Center 22 indicating the serial number of the recognized Drug Dispenser 16 for follow-up action by the Integrated Support Center 22. One alternative for the Integrated Support Center 22 is to lock the Drug Specific App's 12 screen to only give the Patient 6 the choice of calling the Integrated Support Center 22 to resolve his dispensing issue, another is to lock the previous Drug Dispenser 16 which contains undispensed medication and authorize dispensing to resume using the new Drug Dispenser 16. The process can be automated or programmed to require intervention by an Integrated Support Center 22 representative.

The Prescription module 204, which is unique for each drug, encompasses the ability of the Prescriber 2, the Pharmacy 8, other authorized healthcare professionals, or the Integrated Support Center 22 to input some or all the Prescription 4 information. Clicking on Submit 150 in the respective API interfaced e-Prescription 4 program (e.g., ADSC MedicsRx, Allscripts, DAW Systems, DrFirst Rcopia, MDT Toolbox-Rx, Medtab RxCure, OA Systems, Practice Fusion, RxNT eRx, Spectra eRx, etc.) and/or pharmacy prescription programs/systems (e.g., OmniCare, QS/1, PharMerica, Frameworks, etc.) and/or Electronic Prescribing Networks (e.g., SureScripts—that seamlessly connects Prescribers 2 with Pharmacies 8) loads the data into the Patient Database 152. The Prescription 4 data is then automatically integrated into the Drug Specific App 12 and/or into any subsequent Drug Specific App 12 updates by the App Generation Program 200. Additional data input may be inputted by Prescribers 2 and/or the Pharmacy 8 through the application program interface (API) with the e-prescribing programs/systems. When the update is submitted, the App Generation Program 200 automatically syncs all inputs and updates the Patient's 6 Drug Specific App 12 in the Patient Database 152 as well as the Interface Database 278 if the Drug Specific App 12 has been previously downloaded onto the Interface Device 14 or Standalone Drug Dispenser 16.

When loading the Drug Cassette 18 into the Multiple Use Drug Dispenser 16, the application program interface (API) enables the Pharmacy 8 or another healthcare professional dispensing the drug to enter the required drug's Brand and/or generic name, strength/dosage, NDC number, Batch Number, drug temperature storage range, drug humidity storage range, and the drug's expiration/beyond-use date. This input can be done manually and/or via a barcode scan of the Individualized Drug Cassette 18.

The prescribing information defines the dosing strength and administration schedule (e.g., q.d., b.i.d., t.i.d., q.i.d., q.h.s., −X a day, −X per week, −X per month, q.4h, q.6h, q.o.d., a.c., p.c., prn, etc.). The prn dosing, and/or for example the patient self-analgesia dosing, can be designated to allow the Patient 6 to self-medicate using multiple smaller doses to a maximum cumulative dose over a specified time period. Once the maximum dose is dispensed, the Drug Dispenser 16 is locked by the Drug Specific App 12 until the next dosing period begins and the patient enters the requisite information to enable the Drug Specific App 12 to signal the Drug Dispenser 16 to dispense. This in effect allows for “Oral Patient Controlled Analgesia” and or patient symptomatic control for certain conditions, e.g., breakthrough pain.

Once the Prescription Information 204 is entered and the Drug Cassette 18 is loaded and locked into the Drug Dispenser 16, the App Generation Program 200 establishes the Patient 6 dose reminder schedule. Reminders include but are not limited to the ability of the Drug Specific App 12 to alert the Patient 6 using different methodologies. Examples include: (i) automatically bringing up the Biometric Logon screen 850 and buzzing the Interface Device 14, and/or (ii) initiating a phone call with a recorded reminder message, and/or (iii) sending an email message, and/or (iv) sending a text message, and/or (v) having the Integrated Support Center 22 call the Patient 6, and/or (vi) send an alert to a caregiver or family member if the medication has not been taken within a specific time after the dose was supposed to be taken/dispensed, etc. The sequence of reminders can be specified for each patient.

When it is time to dispense the medication and/or when a reminder to take the drug is initiated, the Drug Specific App 12 is shown on the Interface Device's screen. When the Interface Device 14, for example a smartphone, is turned on or unlocked, the screen automatically moves to the Biometric Authentication 850 screen. If the Drug Specific App 12 is clicked on the screen of an Interface Device 14 or on the screen of a Standalone Drug Dispenser's 16 screen opens to the Biometric Authentication 850 screen.

The Patient Reminder 205 module which utilizes the Dosing Frequency 80 to bring up the Drug Specific App 12 screen on the Interface Device 14 or Standalone Drug Dispenser 16 when it is time to dispense the medication. If the medication is not dispensed within a specified timeframe, the program will cause the Interface Device 14 to vibrate and/or send the Patient 6 an email or text message to remind him/her to take the medication. If after a designated time interval, if the Patient 6 has not dispensed the medication, then the program will send alert notifications to the designated and authorized Prescriber 118, Disease Manager 124, Relative 130, 136, 142, etc. If after an additional elapsed time the Patient 6 has not dispensed the medication, the program will send an alert to the Integrated Call Center 22 to call the Patient 6 to ascertain why he/she has not dispensed the medication.

The Interface Device API 206 module utilizes the Interface Device's 14 Bluetooth and/or Wi-Fi communications capability along with specifically written application program interfaces (APIs) to interface, handshake/communicate, with the Drug Dispenser 16, the Patient Database 152, the Integrated Call Center 22, third-party programs resident on the Interface Device 14 (e.g., Patient Diaries 852), and designated, prescribed, patient monitoring and/or diagnostic devices designed to capture digital patient values and/or observations 854, 856, 857, 858, 859, 860, etc. These APIs allow programs and devices to communicate with one another and enable the Drug Specific App 12 to aggregate prescribed information as well as the patient information required for the Drug Specific Dispensing Algorithm 13 to make a dispensing decision.

The App Generation Program 200 only adds independent diagnostic and/or monitoring device APIs, from the API library on the Data Servers 10, to the Drug Specific and Patient Tailored App 12 as required by the Prescription 4. Addition of a specific monitoring and/or diagnostic API also integrates the information into the App Manual 232 and the Training Module 236 and the initial Patient 6 Drug Specific App 12 setup instructions/requirements.

Patient Self-Assessment module 208 is compiled from the Data Servers 10 Self-Assessment Routines Database based upon: (i) specific drug requirements, and/or (ii) Prescribed 4 values/information the Prescriber 2 has designated he/she wants tracked 108, 110 (for better patient management or to manage specific side effects), and/or (iii) to capture Patient Reported Outcomes (PRO) information for clinical trials. Drug specific self-assessment is based upon, for example, the drug's side effects, potential drug interactions, implications of under and/or overdosing, efficacy measures, dosing schedule, drug strength, single or multidrug regimen, effects of weight gain, aging, development of comorbidities, etc. Certain Patient Self-Assessment 862 screens will, for example, incorporate known self-assessment scales, such as the Wong Baker Faces to indicate pain level, or will incorporate self-assessment screens specifically developed for the specific drug. As an example, for opioids, the object of the Patient Self-Assessment screens 862 is for drug titration and the management of opioid tolerance. The screens may also be those which are designed to capture Patient 6 specific information required by regulatory agencies for the subsequent approval of the drug and/or for post marketing studies.

Patient Self-Test 210 module is compiled from the Data Servers 10 Self-Test Routines Database based upon: (i) specific drug requirements, and/or (ii) Prescribed 4 values/information the Prescriber 2 has designated he/she wants tracked 108, 110 (for better patient management or to manage specific side effects), and/or (iii) to capture Patient Reported Outcomes (PRO) information for clinical trials. Patient Self-Testing 864 is specific for each drug based upon the drug's side effects. As an example, for opioids these include, but are not limited to tests to measure confusion, delirium, and/or pinpoint pupils. Certain Patient Self-Testing screens 864 will, for example, incorporate known self-tests or will incorporate self-testing screens specifically developed for the drug. Examples include a motor skills test, a cognitive impairment test, or having the Patient 6 take a selfie of their face with the Patient 6 ensuring his/her eyes are open, etc. For certain drugs with noted overdosing, misuse, abuse or dependency issues, including opioids, the objectives of the Patient Self-Testing screens 864 are to assist in patient management to preclude overdosing, under dosing, misuse, abuse, dependence, and/or addiction. The screens may also be those which are designed to capture Patient 6 specific information required by regulatory agencies for the subsequent approval of the drug and/or for post marketing studies.

Patient Self-Report module 212 is compiled from the Data Servers 10 Self-Report Routines Database based upon: (i) specific drug requirements, and/or (ii) Prescribed 4 values/information the Prescriber 2 has designated he/she wants tracked 108, 110 (for better patient management or to manage specific side effects), and/or (iii) to capture Patient Reported Outcomes (PRO) information for clinical trials. The Self-Reporting 866 screens are specific for each drug based upon the drug's side effects, potential drug interactions, implications of under and/or overdosing, efficacy measures, dosing schedule, drug strength, single or multidrug regimen, effects of weight loss or weight gain, aging, development of comorbidities, etc. The Self-Reporting screens 864 may (i) include well established reporting scales, such as the Bristol Stool Index to indicate if the patient is or is becoming constipated, (ii) a reporting on other drugs taken over a specific period of time and/or since taking the last drug dose which may precipitate drug-drug interactions/adverse events, (iii) or symptomology such as sedation, dizziness, nausea, vomiting, etc. As an example, for opioids, the objectives of the Patient Self-Reporting screens 866 are to help the Patient 6 and the Prescriber 2 to manage opioid side-effects, preclude opioid side-effects, and to preclude overdosing. The screens may also be those which are designed to capture Patient 6 specific information required by regulatory agencies for the subsequent approval of the drug and/or for post marketing studies.

The Digital Capture (APIs) module 214 is compiled from the Data Servers 10 Digital Capture API Routines Database based upon: (i) specific drug requirements, and/or (ii) Prescribed 4 values/information the Prescriber 2 has designated he/she wants tracked 108, 110 (for better patient management or to manage specific side effects), and/or (iii) to capture Patient Reported Outcomes (PRO) information for clinical trials. Digital capture encompasses, as an exemplary, digital information that is integrated via the Drug Specific App 12 via Digital Capture from, as examples, (i) a wearable monitoring device 859, (ii) a digital scale 858, (iii) a third-party monitoring App on a smartphone 852, 880 (iv) a hand held diagnostic device 882, (v) a lifestyle monitor 884, (vi) a digitalized home diagnostic or self-diagnostic 886, (vii) a swallowed tracking and/or diagnostic aid, (viii) a drug tracking chip, radio frequency identification device (RFID) 856 or Selfie 857, (ix) digital interfaces 860, and/or care giver or parent patient assessments and/or journal entries (which may be resident in other Apps on the Patient's 6 Interface Device 14, etc.

The Data Aggregation 215 module is designed to aggregate all one time and trended information from the Drug Dispenser 16, the Drug Cassette 18, other Drug Specific App 12 modules, the Patient Self-Assessment 208, Patient Self-Test 210, Patient Self-Report 212, and Digital Capture 214 modules and organize and aggregate the data so that it can be utilized by the Dispensing Algorithm 220.

The Interface Database module 216 is designed to populate the Interface Database 278 that resides on the Interface Device 14 or on the Standalone Drug Dispenser 16 and is designed to store information that is continuously synchronized with the Patient Database 152. As an example, it aggregates and synchronizes the following: Patient Information: (i) Patient's Full Name 440, (ii) Patient's Street Address 442, (iii) Patient's Date of Birth 444; (iv) Logon Data Repository 445; Drug Dispenser Information: (v) Dispenser Opening Codes 446 (accommodates more than one dispenser code), (vi) Tamper Attempt History 448, (vii) Dispenser Serial No. 450 (accommodates more than one dispenser serial number), (viii) Drug Cassette Serial No. 452 (accommodates more than one cassette serial number), (ix) Drug NDC No. 454, (x) Drug Batch No. 456 (accommodates more than one batch number), (xi) Drug Expiration Date 458 (each tied to a specific batch number), (xii) Drug Storage Temperature 460, (xiii) Drug Storage Humidity 462, (xiv) Last Dose Time and Date 464, (xv) Number of Pills Remaining 466; Prescription Information: (xvi) Drug Name 468, (xvii) Strength 470, (xviii) Dosage Form 472, (xix) Prescribed Drug Quantity 474, (xx) Refills Authorized 476, (xxi) Prescriber's Full Name 478, (xxii) Prescriber's National Provider ID (NPI) 480, (xxiii) Prescriber's DEA No. 482, (xxiv) Store (Pharmacy) Name 484, (xxv) Prescription Reference No. 486, (xxvi) Prescription Issuance Date 488; Values Tracking: (xxvii) Prescription (Dosing Schedule) 490, (xxviii) Digitally Tracked Values 492, (xxix) Self-Assessment Values 494, (xxx) Self-Test Values 496, (xxxi) Self-Reported Values 498, (xxxii) Concomitant Medications 499, (xxxiii) Side Effect Values 500, (xxxiv) Clinical Trial Information 501; Error Reporting: (xxxv) Unsuccessful Dispensing Attempts History 502, (xxxvi) Error Codes History 504; Alerts Information: (xxxvii) Alerts Selection and Timing 506, (xxxviii) Prescriber's Name 508, (xxxix) Prescriber's Email 510, (xl) Prescriber's Texting Telephone No. 512, (xli) Disease Manager's Name 514, (xlii) Disease Manager Email 516, (xliii) Disease Manager Texting Telephone No. 518, (xliv) Relative No. 1 Name 520, (xlv) Relative No. 1 Email 522, (xlvi) Relative No. 1 Texting Telephone No. 524, (xlvii) Relative No. 2 Name 526, (xlviii) Relative No. 2 Email 528, xlix) Relative No. 2 Texting Telephone No. 530, (l) Relative No. 3 Name 532, (li) Relative No. 3 Email 534, (lii) Relative No. 3 Texting Telephone No. 536.

The Prescriber Side Effect Tracking Preferences 218 module is compiled from the Data Servers 10 Drug Side Effect Routines Database based upon: (i) Prescribed 4 side effect information the Prescriber 2 has designated he/she wants tracked 108, 110 (for better patient management or to manage specific side effects), and/or (ii) to capture Patient Reported Outcomes (PRO) information for clinical trials. Each routine is side-effect specific and is based upon the drug's specific side effects identified during pre- and post-launch clinical trials as listed in references such as: Goodman & Gilman's, The Pharmacological Basis of Therapeutics (12th Ed) (Goodman et al. eds) (McGraw-Hill) (2011); and 2015 Physician's Desk Reference (PDR); Cortellis™ Competitive Intelligence by Thomson Reuters; Adis R&D; and/or Pharmaprojects by Citeline, etc.

The Dispensing Algorithm 220 module encompasses, as an example, the Product Expiration (beyond-use date) 103 date, Properly Stored information (for example, temperature, moisture, etc.) 104, 106, one or more Patient Self-Assessment 494, Patient Self-Testing 496, Patient Self-Reporting 498 and/or one or more Digitally Tracked 492 diagnosis or monitoring values, the Dispensing Algorithm 13, the Dispense Screen 868 and interface with the Drug Dispenser 16, and patient feedback and instruction screens 870, 872, 874, etc. The feedback and instruction screens which are presented are aggregated by the App Generation Program 200 from the Data Servers 10 Patient Feedback and Instructions Database. The instructions are drug specific.

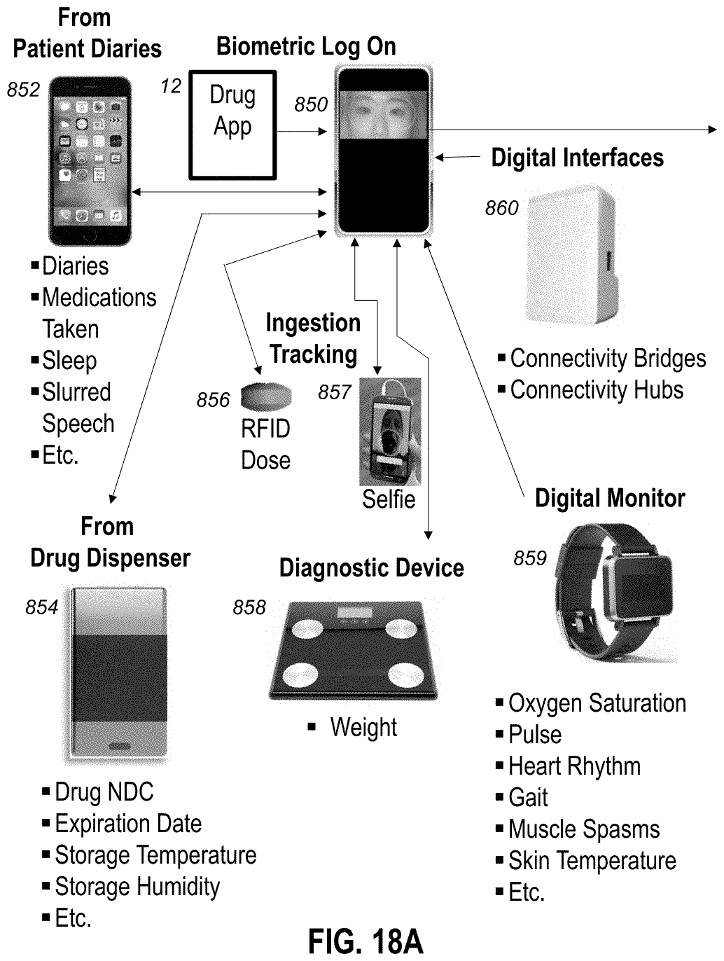

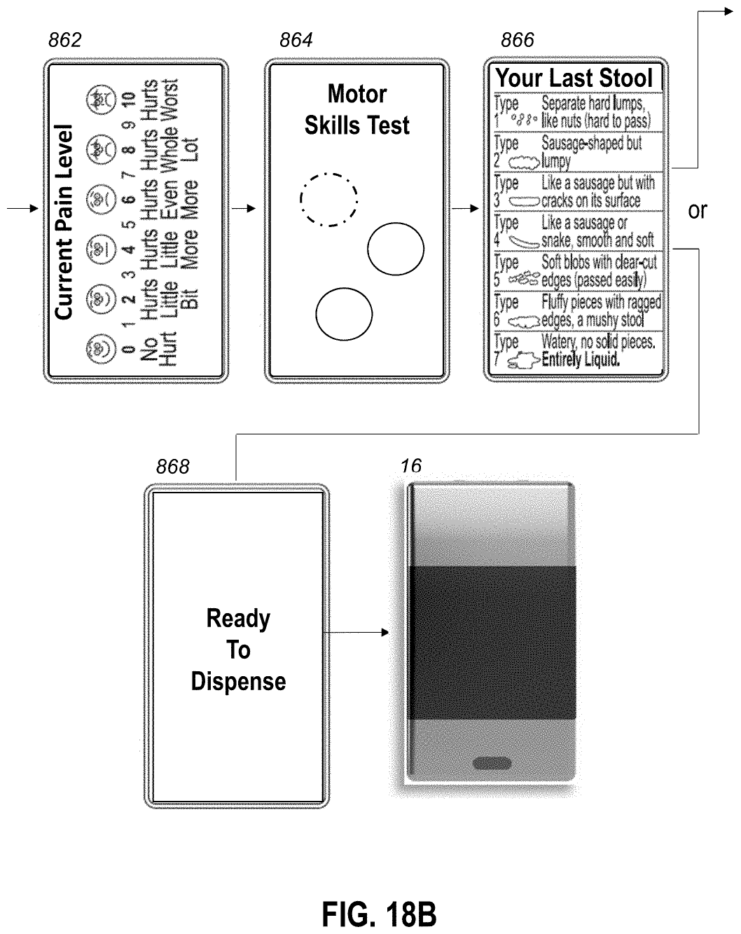

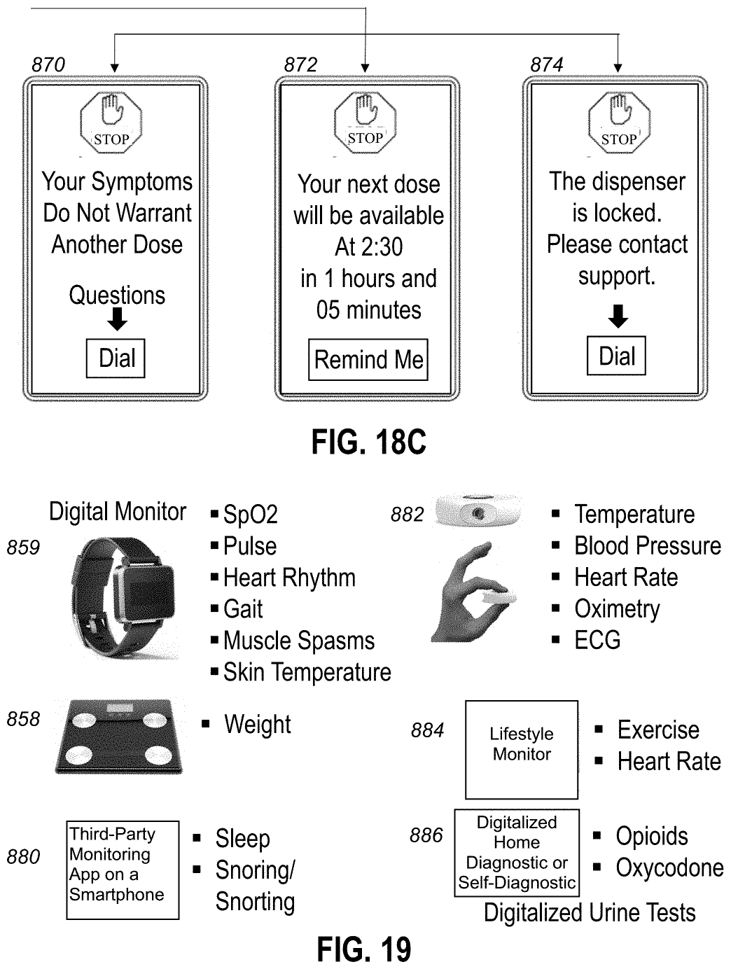

The Communications and Reporting 222 module encompasses, for example, the interface between: (i) the Drug Specific App 12 and the Drug Dispenser 16 via the Interface Device 14; (ii) the interfaces between the Drug Specific App 12 and any proprietary or third-party digital devices, data aggregation devices, computer databases, diagnostic devices, and medication tracking devices, etc., for example, those digital devices listed under FIG. 18 e.g., 852, 854, 856, 857, 858, 859, 860, and on FIG. 19, e.g., 858, 880, 882, 884, 886; (iii) the interface between the Drug Specific App 12 and the Data Servers 10 and the respective databases that store information captured by the Drug Specific App 12; (iv) the data and reports exchanges between the Data Servers 10, the Integrated Support Center 22 and accessed by the Drug Specific App 12; (v) the reminder and alerts notifications initiated by the Drug Specific App 12 and communicated to the Patient 6, Prescriber 2, 508, 510, 512, the Disease Manager 514, 516, 518, and Relatives 520, 522, 524, 526, 528, 530, 532, 534, 536, and/or other care givers, etc.; (vi) securely handshaking/connecting the Drug Specific App 12 to the Integrated Support Center 22 in keeping with Health Insurance Portability and Accountability Act (HIPAA) Patient 6 data security requirements; (vii) synchronization of the information captured by the Drug Specific App 12 with the Integrated Support Center's 22 Data Servers 10; (viii) sending to and receiving alerts from the Integrated Support Center 22; (ix) enabling the Integrated Support Center 22 to lock or unlock the Drug Dispenser 16; (x) alerting the Integrated Support Center 22 of unusual attempts to open the Drug Dispenser 16; (xi) the ability of the Integrated Support Center 22 to remotely update the Drug Specific App 12 software; (xii) in case of a drug recall, allows the Integrated Support Center 22 to immediately lock (preclude all dispensing) those Drug Dispensers 16 that contain Drug Cassettes 18 containing the drug and/or the drug batch which is being recalled and simultaneously notifying the Patient 6 what they need to do; (xiii) enables the Drug Specific App 12 to access patient 6 reports, charts, and graphs requested by the Patient 6; (ix) enables the patient 6 to request a refill prescription to be sent to his/her pharmacy 8 for refill; (x) enables the Integrated Support Center 22 to change the prescription on the Drug Specific App 12; (xi) enables the interface between the App Generation Program's 200 Drug Specific App 12 software download and update routines, etc.; (xii) enables the communications interface between the Drug Specific App 12, the Drug Dispenser 16, and the Integrated Support Center 22 which allows the Integrated Support Center 22 to troubleshoot problems with either the Drug Specific App 12 and/or the Drug Dispenser 16, etc.

Central Servers Data Sync 224 module is the software module that automatically synchronizes the Interface Database 278 and the Patient Database 152. An example of the types of data that is synchronized includes but is not limited to: Patient Information: (i) Patient's Full Name 440, (ii) Patient's Street Address 442, (iii) Patient's Date of Birth 444; (iv) Logon Data Repository 445; Dispenser Information: (v) Dispenser Opening Codes 446 (accommodates more than one dispenser code), (vi) Tamper Attempt History 448, (vii) Dispenser Serial No. 450 (accommodates more than one dispenser serial number), (viii) Drug Cassette Serial No. 452 (accommodates more than one cassette serial number), (ix) Drug NDC No. 454, (x) Drug Batch No. 456 (accommodates more than one batch number), (xi) Drug Expiration Date 458 for each batch, (xii) Drug Storage Temperature 460, (xiii) Drug Storage Humidity 462, (xiv) Last Dose Time and Date 464, (xv) Number of Pills Remaining 466; Prescription Information: (xvi) Drug Name 468, (xvii) Strength 470, (xviii) Dosage Form 472, (xix) Prescribed Drug Quantity 474, (xx) Refills Authorized 476, (xxi) Prescriber's Full Name 478, (xxii) Prescriber's National Provider ID (NPI) 480, (xxiii) Prescriber's DEA No. 482, (xxiv) Store (Pharmacy) Name 484, (xxv) Prescription Reference No. 486, (xxvi) Prescription Issuance Date 488; Values Tracking: (xxvii) Prescription (Dosing Schedule) 490, (xxviii) Digitally Tracked Values 492, (xxix) Self-Assessment Values 494, (xxx) Self-Test Values 496, (xxxi) Self-Reported Values 498, (xxxii) Concomitant Medications 499, (xxxiii) Side Effect Values 500, (xxxiv) Clinical Trial Information 501; Error Reporting: (xxxv) Unsuccessful Dispensing Attempts History 502, (xxxvi) Error Codes History 504; Alerts Information: (xxxvii) Alerts Selection and Timing 506, (xxxviii) Prescriber's Name 508, (xxxix) Prescriber's Email 510, (xl) Prescriber's Texting Telephone No. 512, (xli) Disease Manager's Name 514, (xlii) Disease Manager Email 516, (xliii) Disease Manager Telephone Texting No. 518, (xliv) Relative No. 1 Name 520, (xlv) Relative No. 1 Email 522, (xlvi) Relative No. 1 Telephone Texting No. 524, (xlvii) Relative No. 2 Name 526, (xlviii) Relative No. 2 Email 528, xlix) Relative No. 2 Telephone Texting No. 530, (l) Relative No. 3 Name 532, (li) Relative No. 3 Email 534, (lii) Relative No. 3 Telephone Texting No. 536.

The Patient Reporting 226 module encompasses, as an example: (i) the ability of the Patient 6 to request certain reports generated by the Drug Specific App 12, e.g., the last time the Patient 6 took the drug, prescription information details, drug details (e.g., brand and generics names, batch number, expiration/beyond-use date, doses remaining, reorder information, drug interactions, typical side effects, etc.); (ii) graphs and charts created by the Drug Specific App 12 based upon Interface Device 14 or Standalone Drug Dispenser 16 stored information; and (iii) graphs, charts and/or reports created by the Integrated Support Center's 22 Data Servers 10 for downloading by the Patient 6 via the Drug Specific App 12, etc.

The Drug Cassette Replacement 227 module is designed to use the Patient Identifier Number 160 and the drugs NDC 70 number to preclude the Patient 6 from dispensing duplicate doses for the same drug from one or more Prescriptions 4 prescribed by one or more Prescribers 2 and/or dispensing the drug 70 prescribed dose from one or more Drug Dispensers 16 or Drug Cassettes 18 containing the specified drug 70 (to eliminate duplicate dosing). The program accompanies this by prioritizing Disposable Drug Dispensers 16 with an integrated drug cassette and/or Drug Cassettes 18 containing the same drug 70 so the drug 70 in a given Disposable Drug Dispenser 16 and/or Drug Cassette 18: (i) must be fully dispensed per the Prescription 4 before a subsequent Drug Cassette 18 can begin dispensing the given drug 70, or (ii) the first Disposable Drug Dispenser 16 or Drug Cassette 18 is designated as locked (precluded from dispensing) and a subsequent Disposable Drug Dispenser 16 or Drug Cassette 18 is enabled to dispense the single drug dose in keeping with the Prescription 4. This enables dispensing from multiple Disposable Drug Dispensers 16 containing the same drug in the event a Disposable Drug Dispenser 16 is inoperable, lost, or left at another location. This also enables the utilization of multiple Drug Cassettes 18 (for Reusable Drug Dispensers 16) in the event a problem arises with a particular Drug Cassette 18 (e.g., it is damaged, improperly stored, the original reusable Drug Dispenser 16 is lost or damaged, etc.). This process affords the Prescriber 2 and Pharmacy 8 the ability to dispense sufficient drug doses to limit the number of Pharmacy 8 visits and/or mail order shipments between refills (within state/government prescribing guidelines).

The Package Insert 228 module pulls the respective drug's package insert in the appropriate language (as designated by the Patient's 6 language preference) from the Drug Package Insert Database on the Data Servers 10 and incorporates it into the Drug Specific App 12.

The Dispenser Manual 230 module pulls the respective Drug Dispenser 16 manual in the appropriate language from the Drug Dispenser Manual Database on the Data Servers 10 and incorporates it into the Drug Specific App 12.

The App Manual 232 module pulls the respective Drug Specific App 12 manual in the appropriate language from the Drug Specific App Manual Database on the Data Servers 10 and incorporates it into the Drug Specific App 12. The App manual program logic enables the system to download a personalized Patient 6 specific manual that incorporates the Drug Specific Dispensing Algorithm 13 and Prescriber 2 prescribed 4 and defined digital capture, self-assessment, self-testing, and self-reporting data capture and screen instructions and their respective troubleshooting instructions.

The Help and Troubleshooting 234 module is an artificial intelligence based query based module that allows the Patient 6 to enter key words or phrases to bring up a list of potential sections that may address the Patient's 6 issue. Sections applicable to a specific help and troubleshooting screen on the Interface Device 14 or Standalone Drug Dispenser 16 on the Drug Specific App 12 are hotlinked to allow instructions to be viewed without interrupting the Patient 6 input dispensing sequence. The base module answers most questions. However, a link to an expanded version of Help and Troubleshooting hosted on the Central Servers 10 is provided in each screen. The Central Servers 10 also host a User Group to allow Patient's 6 to ask questions of other users as well as to post recommended improvements and/or enhancements to the Drug Specific App 12 and/or the respective Drug Dispensers 16 and/or support services, etc. Links are also provided to video tutorials on video sharing websites (e.g., YouTube, Vimeo, etc.).

The Training Module 236 encompasses, as an example, (i) a hot link to the Dispenser Manual 230 and the App Manual 232 as well as a training and troubleshooting video library resident on the Training Videos Database resident on the Data Servers 10; (ii) videos posted, as an example, on YouTube, Vimeo, and/or other consumer video services covering all aspects of utilizing and troubleshooting the Drug Specific App 12 and the respective Drug Dispensers 16, (iii) a step by step tutorial resident on the Interface Device 14, (iv) a hot linked “help” button on each respective screen allowing the Patient 6 to bring up usage instructions for the respective screen without interrupting the sequence of entering the required information or selecting a particular command, etc.

A similar medical professional training module is available to assist the medical professional/Prescriber 2/Pharmacy 8 in all issues related to each respective Drug Dispenser 16, Drug Cassettes 18 and to troubleshoot documentation or software issues. These files and Videos, resident on the Central Servers 10, would be accessible, as an example, on popular video sharing websites (e.g., YouTube, Vimeo, etc.).

The Unique App Serial Number 238 is assigned by the App Generation Program when it is ready to finalize the aggregation of the Personalized Drug Specific App 240.

The Personalized Drug Specific App 240 is then linked to the Patient Identifier No. 160. Then the Patient Specific Drug Specific App 12 is: (i) compiled; stored in the Drug Specific App Database on the Data Servers 10, and (iii) is automatically transmitted to the Patient 6 in a download Email and/or text message. The Patient 6 clicks on the link on the Email or text message or pastes the link in the browser to be taken to the download screen where the Patient 6 clicks on the Download App 246 to download the Drug Specific App 12.

The Patient Identifier No. 160 links the Patient 6 with the Patient Specific Drug Specific App 12. The Patient Identifier No. 160 is utilized by the Patient 6 when accessing the Drug Specific App 12 for the first time to set up the Biometric Login 250.

The Drug Specific App 12 utilizes, for example, the Interface Device's 14 features, memory and computer power to; (i) facilitate the Drug Specific App's 12 interface with the Patient 6, (ii) uses the Interface Device's 14 Bluetooth and/or Wi-Fi communications capability to interface with the Drug Dispenser 16, (iii) its Internet communications capability to interface with the Data Center 10 and the Integrated Support Center 22, (iv) uses its Email and texting capabilities to send alerts to the Patient 6, the Prescriber 2, 508 other caregivers, the Patients 6 disease manager 514, and/or family members 520, 526, 532, (v) the phone to call the Integrated Support Center 22, (vi) the video player capabilities to play user instruction, troubleshooting, drug and disease information videos, (vii) its GPS capability if location is required for the capture of lifestyle information, (viii) the Interface Device's 14 memory to store the Drug Specific App 12, the Prescription 4, dispensing history 464, tamper alert history 448 (etc.), and most recent Patient 6 entered information and screen responses 494, 496, 498, 499, 500, 501 as well as digitally captured information 492 which was used to make a dispensing decision by the Drug Specific Dispensing Algorithm 13 or which was requested to be captured for future utilization by the Prescriber 2 or other medical professionals 108, 110, (ix) its voice recognition and communication capabilities, etc.

Utilization of an Interface Device 14 allows the Drug Dispenser 16 to: (i) be smaller than it would otherwise be; (ii) cost less to manufacture; (iii) decreases battery power requirements, etc. These same or a combination of the afore listed capabilities are incorporated into the Disposable Drug Dispensers 16 as well as the Standalone Drug Dispensers 16.

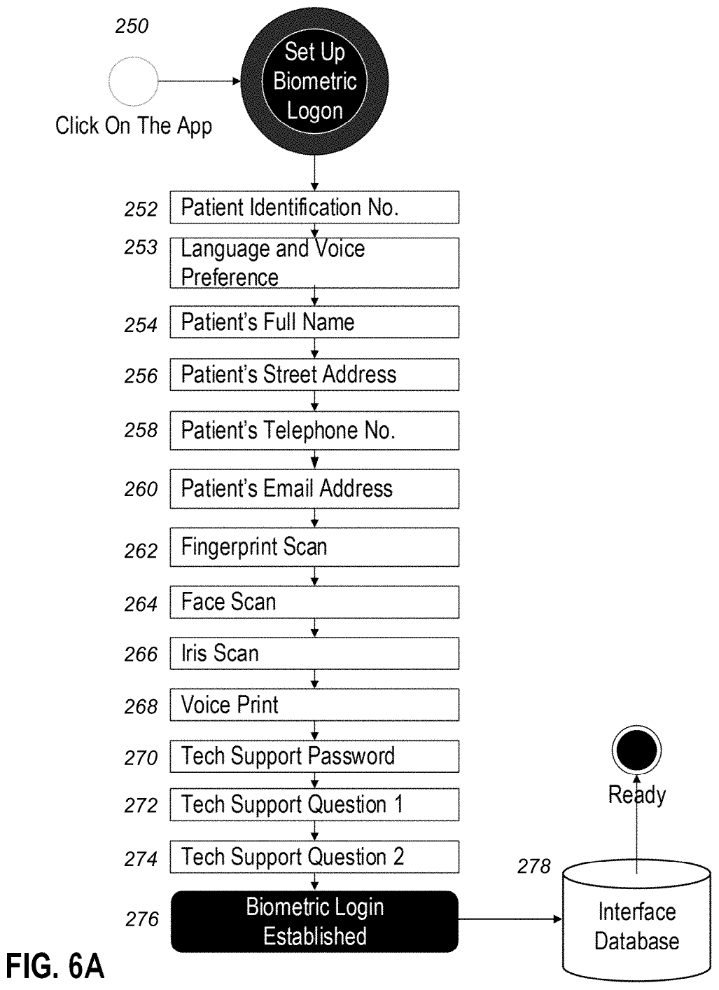

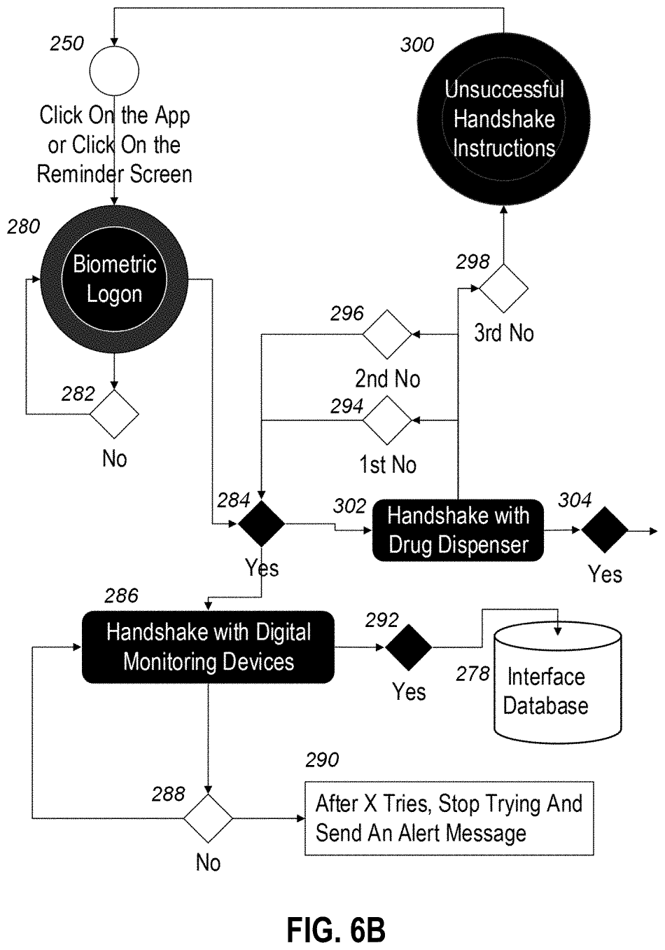

FIG. 6 is an exemplary embodiment of the Biometric Authentication interface which is designed to be compliant with the Health Insurance Portability and Accountability Act (HIPAA) or any governmental body that sets the standard for protecting sensitive patient data. This means that all the required physical, network, and process security measures are in place and followed and incorporated herein by reference.

When the Patient 6 clicks on the Drug Specific App 12 the first time, it brings up the Set Up Biometric Logon 250 screen. It asks the Patient 6 to enter the Patient Identifier Number 160 previously sent to the Patient 6, via Email or Text, into the Patient Identification No. 252 input box. It then establishes if the Patient's 6 language preference 253 and allows the Patient 6 to choose, if preferred, the Drug Specific App's 12 voice interface preference 253. It then requests the: (i) Patient's Full Name 254, (ii) Patient's Street Address 256, (iii) Patient's Telephone No. 258 (this is the require mobile phone/Interface Device 14 or Standalone Drug Dispenser 16 telephone number), and (iv) Patient's Email Address 260 (this will accommodate one or more Email addresses). Thereafter it goes into the routines to capture the biometric authentication reference information for one or more of the following: (v) Fingerprint Scan 262, (vi) Face Scan 264, (vii) Iris Scan 266, (viii) Voice Print 268, (ix) Tech Support Password 270, (x) Tech Support Challenge Question 1 272, (x) Tech Support Challenge Question 2 274. Once the Biometric login is established 276, the Drug Specific App 12 saves the Biometric Logon information in the Logon Data Repository 445 repository in the Interface Database 278. At that point, the Drug Specific App 12 is ready to begin authorizing the Drug Dispenser 16 to dispense medication.

Thereafter, when the Patient 6 clicks on the Drug Specific App 12 or clicks on the Reminder Screen 250, the Biometric Logon 280 screen appears. If the Biometric Logon 280 is not successful, No 282, then the Patient 6 is asked to try again. After a designated number of tries, the Patient 6 is given troubleshooting instructions. The troubleshooting instruction contains a single click call button to connect the Patient 6 with the Integrated Call Center 22. If the Biometric Logon 280 is successful, Yes 284, then the Drug Specific App 12 automatically tries to handshake with all designated digital monitoring devices 286. If successful, Yes 292, the Drug Specific App 12 downloads the designated information into the Interface Database 278. If the digital handshake 286 is not successful, No 288, then the Drug Specific App 12 retries to effectuate the handshake. If it is not able to effectuate the handshake after a specified number of tries, it stops trying and sends and alert message 290 and then proceeds to try to effectuate a handshake 302 with the Drug Dispenser 16. If the Biometric Logon 280 is successful, Yes 284, simultaneous to trying to handshake with digital devices 286, the routine tries to handshake with the Drug Dispenser 302. If it is successful, Yes 304, then the unit is ready to go through the Drug Specific Dispensing Algorithm 13 screens to allow the Drug Dispenser 16 to dispense a drug dose. If the handshake with the drug dispenser is not successful, No 294 or No 296, then the routine will attempt two more times to effectuate the handshake. If the third (3^{rd}) try is not successful, No 298, the routine shows an Unsuccessful Handshake Instructions 300 screen telling the Patient 6 how to resolve the problem.

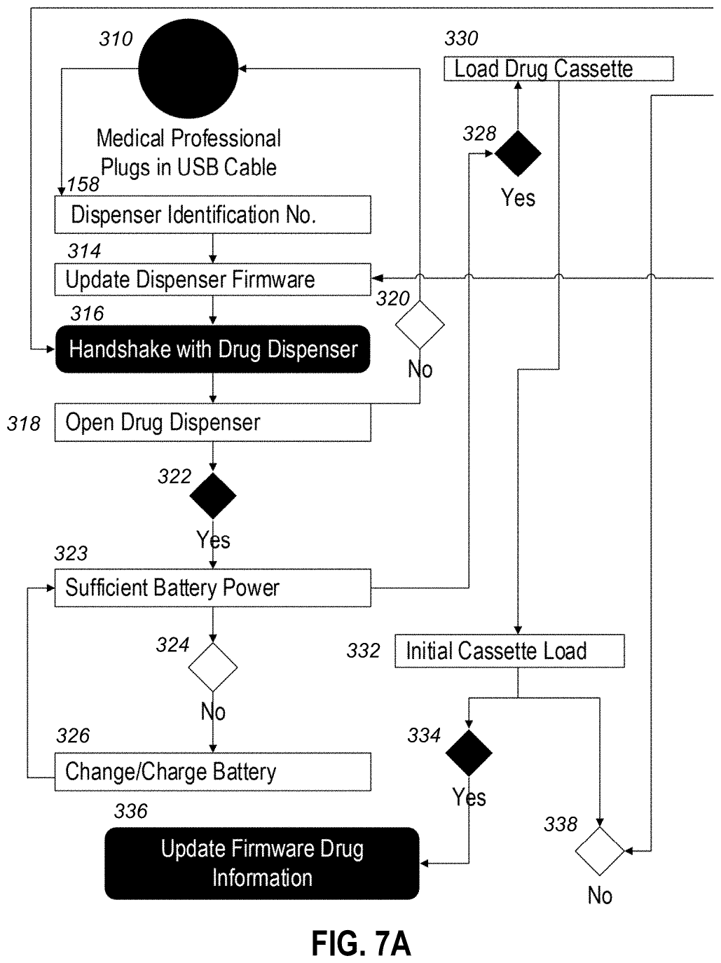

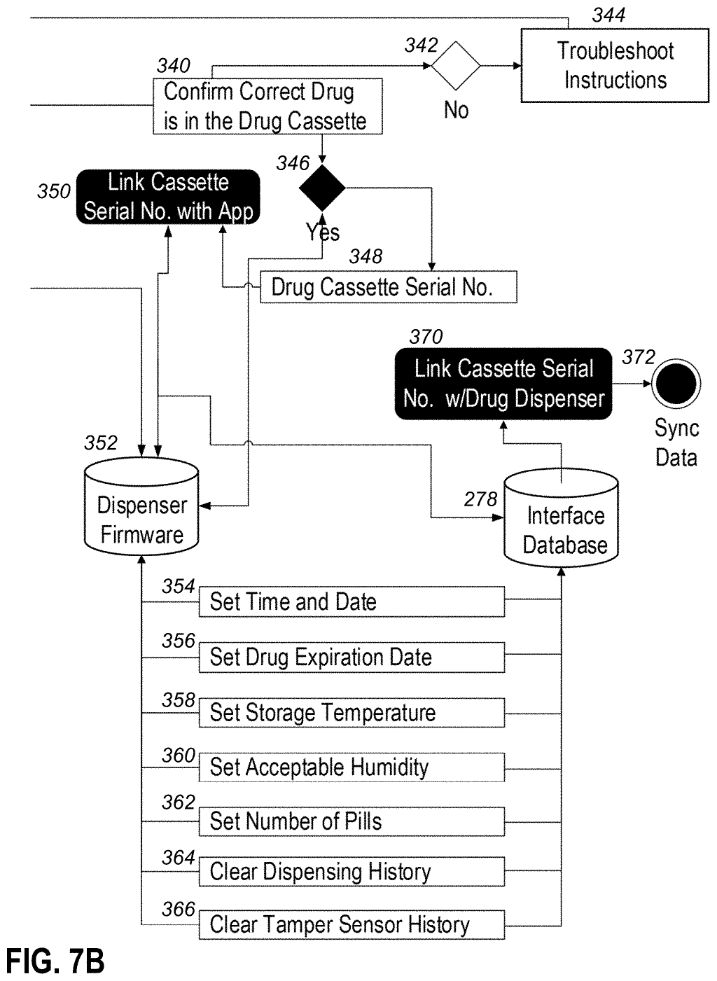

FIG. 7 is an exemplary embodiment of logic flow and process related to loading a Drug Cassette 18 into a Reusable Drug Dispenser 16. This process is not applicable for tamper proof Disposable Drug Dispensers 16 which contain an integrated Drug Cassette 18 and cannot be opened after the Drug Cassette 18 is loaded into the then sealed Disposable Drug Dispenser 16. The following description is an exemplary of how a Drug Cassette 18 for restricted drug would be loaded into the Reusable Drug Dispenser 16 by a medical professional. (A similar but less restrictive routine can be followed by the Patient 6 to load a Drug Cassette 18 into a Reusable Drug Dispenser 16 for medications where diversion, addiction, dependence, abuse, misuse, etc., are not of concern.)

To load the Drug Cassette 18, the Medical Professional opens his/her ePrescription 4 screen for the Prescribed 4 medication and thereafter plugs in a USB Cable from the Rx Computer into the Drug Dispenser 16. Alternatively, a USB Cable can be plugged in from a Computer with the requisite software to enable the entry of the requisite codes needed to open the Drug Dispenser 16.

The USB cable provides power to the Drug Dispenser's independent clamshell locking and unlocking mechanism 1028, 1030. Power for the clamshell locking and unlocking mechanism 1028, 1030 is not available from the Drug Dispenser's 16 battery.

After the program confirms that the Dispenser Identification No. 158 assigned to the medication is the same as, corresponds with, the Dispenser Identification No. 158 contained in the Drug Dispenser's Firmware 352, the Drug Dispenser 16 may be opened to allow loading of the Drug Cassette 18. If the Dispenser Identification No. 158 is blank on the Drug Dispenser's 16 firmware because this is the first time the Drug Dispenser 16 is being utilized for that medication, then the program inserts the Dispenser Identification No. 158 into the Drug Dispenser's Firmware 352.

The Dispenser Identification No. 158 cannot be copied into the Drug Specific App's 12 Interface Database 278. It is copied and retained in the Dispenser Opening Codes 446 data repository resident on the Patient Database 152 stored in the Data Servers 10.

The USB cable is the same cable that is used to charge the battery in rechargeable battery configuration Reusable Drug Dispensers 16.

If the Dispenser Identification No. 158 is the same as the code in the Dispenser Firmware 352, then the program proceeds to effectuate a handshake with the Drug Dispenser 316 and proceeds to try to Open the Drug Dispenser 318. If it cannot open the drug dispenser, No 320, then the program restarts the dispenser opening routine 310. Prior to unlocking the Reusable Drug Dispenser 16, the program checks to see if there is sufficient battery power 323. If the answer is No 324, then the program requires that the battery be changed or the unit be recharged 326 to a minimum level prior to allowing the Drug Cassette 18 to be loaded 330 into the Reusable Drug Dispenser 16.

The temperature within the Drug Cassette 18 cavity within the Drug Dispenser 16 is monitored during recharging to ensure it does not exceed the allowable medication storage temperature 104. If the temperature 104 is within, for example, one degree of the maximum storage temperature 104, the recharge is stopped until the temperature within the Drug Cassette 18 storage cavity is back to the acceptable recharge temperature range before charging.

If there is sufficient battery power or if the battery power has been restored to a sufficient battery power level, Yes 328, then the Drug Cassette 18 can be loaded 330 into the Reusable Drug Dispenser 16. If this is the initial drug cassette 332 for this medication, Yes 334, then the program updates the drug information on the drug dispenser's firmware 336. If this is a not the initial Drug Cassette 18 docking, No 338, for the medication, then the program confirms that the correct drug per the Prescription 4 is in the drug cassette 340 by comparing the prescribed drug in the Prescription 4 versus the drug indicated in the firmware 352. If it is not the correct drug, No 342, then the program provides the troubleshooting instructions 344. If it is the correct drug in the drug cassette, Yes 346, then the program copies the Drug Cassette Serial No. 348 and links it with the Drug Dispenser Serial No. 350 on the Drug Specific App 12 resident on the Interface Database 278. It then proceeds to synchronize: (i) the appropriate time and date 354, (ii) drug expiration date 356, (iii) proper storage temperature range 358, (iv) acceptable humidity range 360, (v) the number of pills available 362, and then (vi) clear the dispensing history on the firmware 364 for all values but the last dispensing date and time, and (vii) clear the firmware tamper sensor history 366, and (viii) ensure the same data is contained on both the Drug Dispenser 16 firmware 352 and the Drug Specific App 12 Interface Database 278. At this point, the Drug Cassette's 18 and the Drug Dispenser's 16 serial numbers are linked together 370 on the Drug Specific App 12. At that point, the Drug Specific App 12 data is synchronized 372 with the Patient Database 152 on the Data Servers 10. If immediate synchronization cannot be effectuated, the program stores the information for synchronization the next time a connection is available.

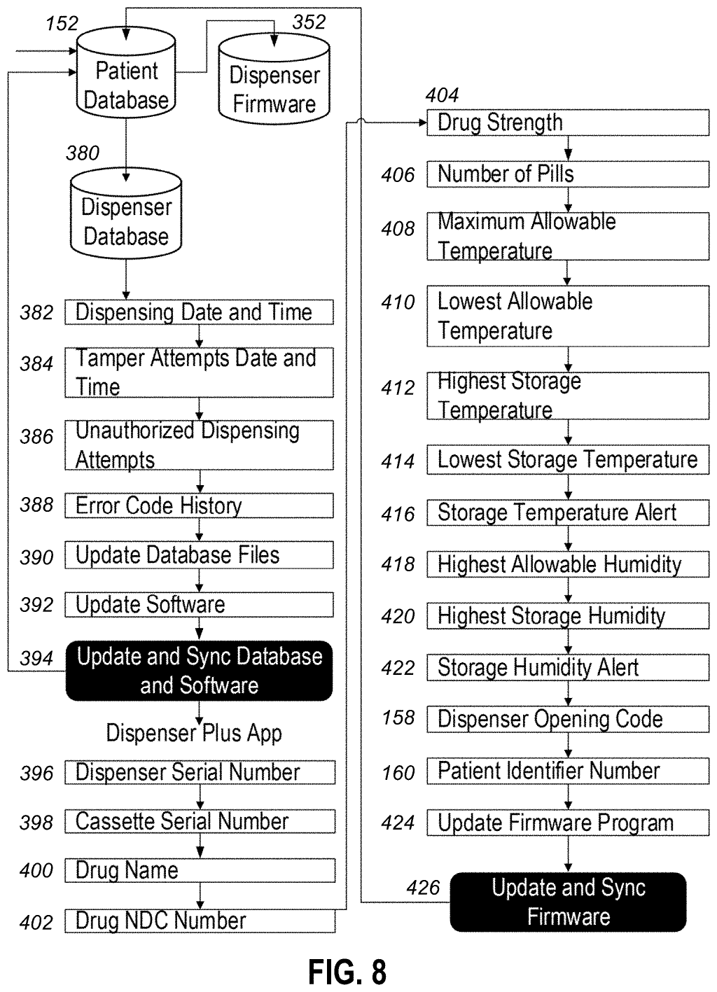

FIG. 8 is an exemplary embodiment depicting data synchronization and updating between the three Drug Specific App 12 related databases, (i) Patient Database 152 which is resident on the centralized Data Servers 10, (ii) the Dispenser Database 380 that is part of the Drug Specific App 12 that is resident on the Interface Device 14 or on the Standalone Drug Dispenser 16, and (iii) the Dispenser Firmware 352 in each Drug Dispenser 16.

When the Drug Cassette 18 is finished being loaded into the Reusable Drug Dispenser 16, a handshake is effectuated between the Drug Dispenser 16 and the Drug Specific App 12. Upon recognizing that there is a new Drug Cassette 18, the Drug Specific App 12 links with the Patient Database 152 and begins a data update process to ensure the respective data is synchronized and the most current. First the program synchronizes and updates the Dispenser Database 380 data 382, 384, 386, 388, 390, 392 and effectuates any updates 394 from the Dispenser Database 380 to the Patient Database 152. Then the update program 394 proceeds to utilize the Patient Database 152 to synchronize and update the Dispenser Firmware 352, 396, 398, 400, 402, 404, 406, 408, 410, 412, 414, 416, 418, 420, 422, 158, 160, 424, and 426. Any changes which are more current on the Dispenser Firmware 352 are updated on the Patient Database 152. This sequence is repeated every time that the patient dispenses a dose, or if the Internet connection is not available, as soon as an Internet connection is established between the Interface Device 14 or the Wi-fi enabled Standalone Drug Dispenser 16 and the Data Servers 10.

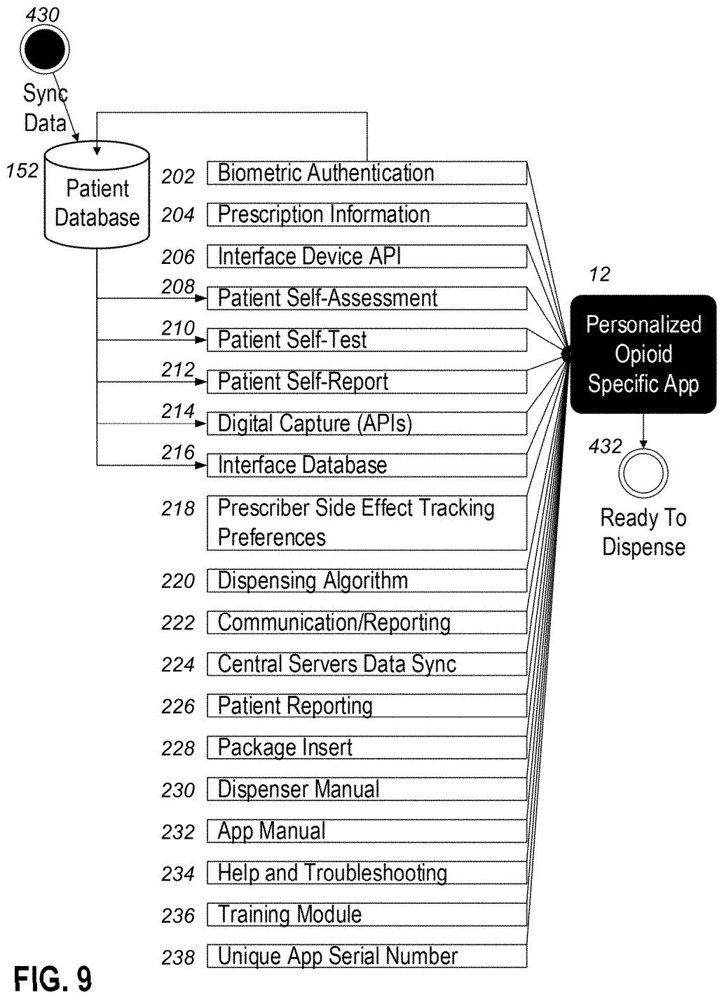

FIG. 9 is an exemplary embodiment of the database files which are synchronized when the Sync Data 430 routine for the Patient Database 152 is initiated: 202, 204, 206, 208, 210, 212, 214, 216, 218, 220, 222, 224, 226, 228, 230, 232, 234, 236, 238. Once they are synchronized, then the Personalized Drug Specific App 12 is fully updated and Ready To Dispense 432.

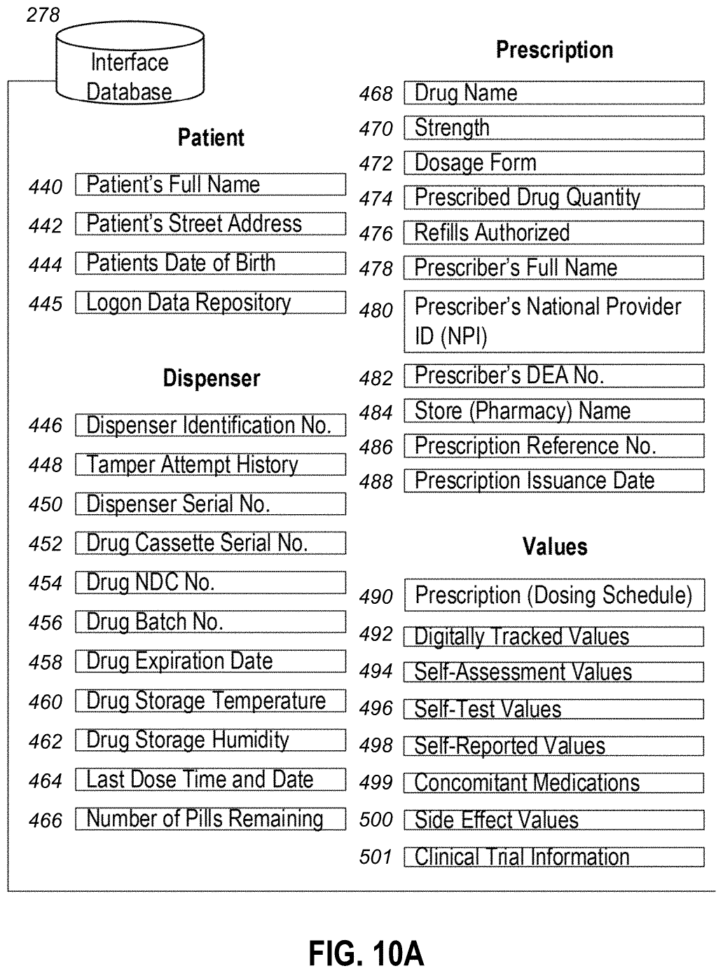

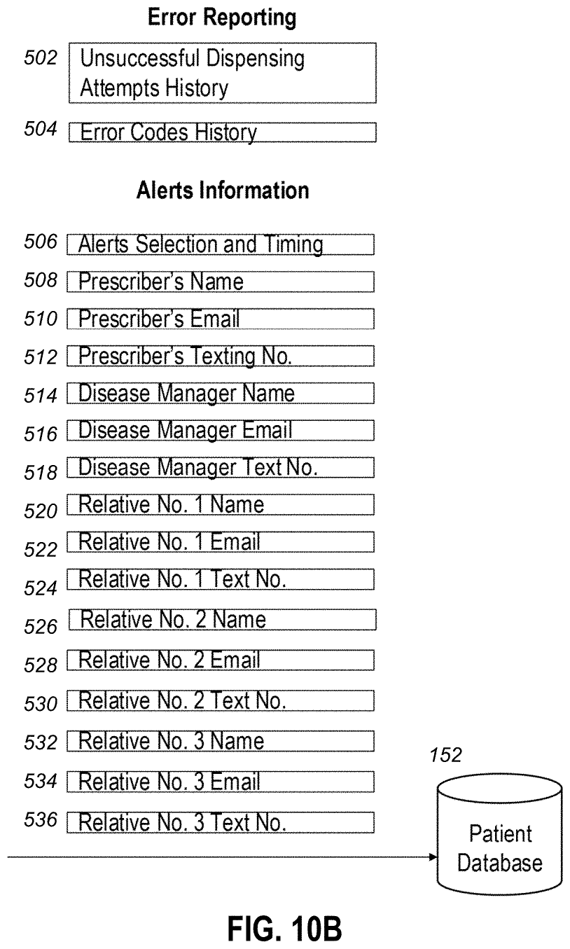

FIG. 10 is an exemplary embodiment of the Interface Database 278 types of data and data files/databases which are continually synchronized with the Patient Database 152. The Drug Specific App 12 data contained in the Interface Device Database 278 is synchronized real time with the centralized Patient Database 152. The Interface Database 278 retains information for a defined time period to enable the Drug Specific Dispensing Algorithm 13 and/or the App's Prescribed 4 Patient 6 values and side effects reporting routines to use and present trended data while minimizing the Drug Dispenser 16 and the Drug Specific App's 12 memory requirements. The Patient Database 152 resident on the Data Servers 10 retains an ongoing history without deleting prior patient data.

The following is a representation of the types of data files which are synchronized: Patient Information: (i) Patient's Full Name 440, (ii) Patient's Street Address 442, (iii) Patient's Date of Birth 444; (iv) Logon Data Repository 445; Dispenser Information: (v) Dispenser Opening Codes 446 (accommodates more than one dispenser code), (vi) Tamper Attempt History 448, (vii) Dispenser Serial No. 450 (accommodates more than one dispenser serial number), (viii) Drug Cassette Serial No. 452 (accommodates more than one cassette serial number), (ix) Drug NDC No. 454, (x) Drug Batch No. 456, (xi) Drug Expiration Date 458 (retains one expiration date per drug batch), (xii) Drug Storage Temperature 460, (xiii) Drug Storage Humidity 462, (xiv) Last Dose Time and Date 464, (xv) Number of Pills Remaining 466; Prescription Information: (xvi) Drug Name 468, (xvii) Strength 470, (xviii) Dosage Form 472, (xix) Prescribed Drug Quantity 474, (xx) Refills Authorized 476, (xxi) Prescriber's Full Name 478, (xxii) Prescriber's National Provider ID (NPI) 480, (xxiii) Prescriber's DEA No. 482, (xxiv) Store (Pharmacy) Name 484, (xxv) Prescription Reference No. 486, (xxvi) Prescription Issuance Date 488; Values Tracking: (xxvii) Prescription (Dosing Schedule) 490, (xxviii) Digitally Tracked Values¹³ 492, (xxix) Self-Assessment Values 494, (xxx) Self-Test Values 496, (xxxi) Self-Reported Values 498, (xxxii) Concomitant Medications 499, (xxxiii) Side Effect Values 500, (xxxiv) Clinical Trial Information 501; Error Reporting: (xxxv) Unsuccessful Dispensing Attempts History 502, (xxxvi) Error Codes History 504; Alerts Information: (xxxvii) Alerts Selection and Timing 506, (xxxviii) Prescriber's Name 508, (xxxix) Prescriber's Email 510, (xl) Prescriber's Texting Telephone No. 512, (xli) Disease Manager's Name 514, (xlii) Disease Manager Email 516, (xliii) Disease Manager Texting Telephone No. 518, (xliv) Relative No. 1 Name 520, (xlv) Relative No. 1 Email 522, (xlvi) Relative No. 1 Texting Telephone No. 524, (xlvii) Relative No. 2 Name 526, (xlviii) Relative No. 2 Email 528, xlix) Relative No. 2 Texting Telephone No. 530, (l) Relative No. 3 Name 532, (li) Relative No. 3 Email 534, (lii) Relative No. 3 Texting Telephone No. 536. ¹³ The patient values require that the database is able to accommodate capture data from the respective number of digital devices tracked as well as for the number data capture screens used.

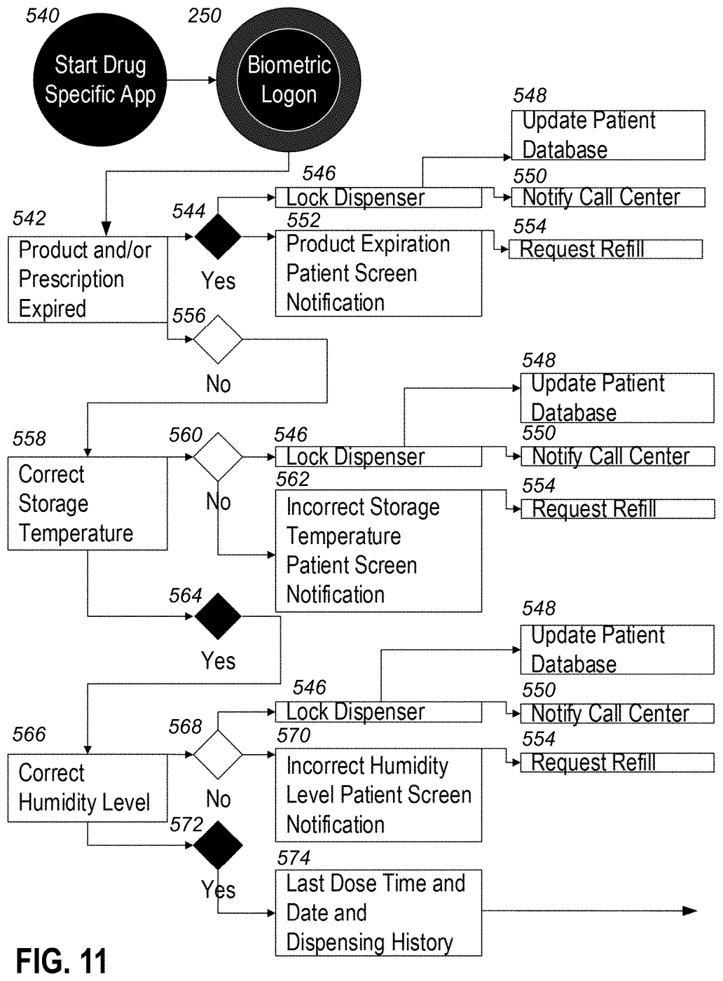

FIG. 11 is an exemplary embodiment of the Drug Specific App 12 logic. When the Patient 6 starts the Drug Specific App 540 by either clicking on the App or by responding to the dose due alert, the screen automatically moves to the Biometric Logon 250 screen. Upon successful biometric authentication, the program checks to see if the product has expired 542, if Yes 544, it presents a product expiration patient screen notification 552, and if a refill is authorized, automatically sends a refill request to the pharmacy 554. Simultaneously, the program locks the drug dispenser 546, updates the patient database 548, and notifies the Integrated Call Center 550. If the product has not expired, No 556, then it proceeds to check to see if the product has been stored in the correct storage temperature 558 since the product was last dispensed.

If the medication has not been stored at the correct storage temperature, No 560, it presents an incorrect storage temperature patient screen notification 562, and if a refill is authorized, automatically sends a refill request to the pharmacy 554. Simultaneously, the program locks the drug dispenser 546, updates the patient database 548, and notifies the Integrated Call Center 550. If the product has been stored at the correct storage temperature, Yes 564, then it proceeds to check to see if the product has been stored in the correct humidity range 566 since the product was last dispensed.

If the medication has not been stored at the correct humidity level, No 568, it presents an incorrect storage humidity patient screen notification 570, and if a refill is authorized, automatically sends a refill request to the pharmacy 554. Simultaneously, the program locks the drug dispenser 546, updates the patient database 548, and notifies the Integrated Call Center 550. If the product has been stored at the correct storage humidity, Yes 572, then it proceeds to check the last dose time and date and dispensing history 574 to establish the basis for checking the requisite information required to authorize dispensing.

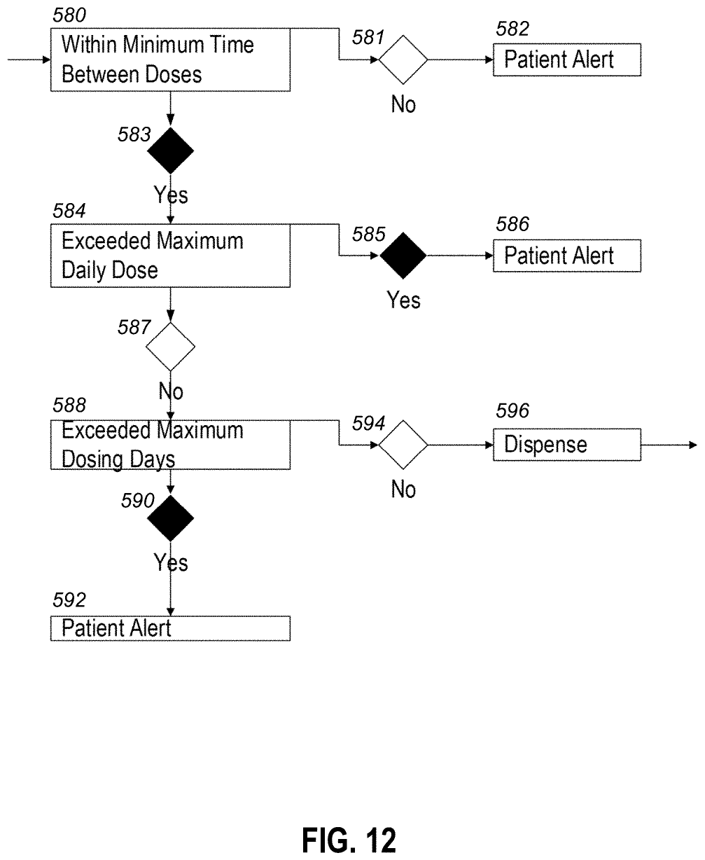

FIG. 12 is an exemplary embodiment of the prescription information the program checks prior to authorizing dispensing of a medication dose. The program sees if the prescribing request is within the Prescription's 4 time between doses 580 instructions, if No 581, the program issues a patient alert 582 indicating when the next dose will be available. If the time interval is within the prescribing guidelines, Yes 583, then the program checks to see if the maximum daily doses 584 have been exceeded. If the maximum number of daily doses have been exceeded, Yes 585, the program issues a patient alert 586 stating that the request exceeds the prescribed maximum number of daily doses and indicates when the next dose will be available. If the maximum number of daily doses has not been exceeded, No 587, the program checks to see if the maximum authorized dosing days 588, i.e., the number of days the prescription is in effect, have been exceeded. If Yes 590, a patient alert 592 is issued stating the prescription period has expired and instructing the Patient 6 to contact the Prescriber 2 if he/she has any questions. If the maximum number of authorized dosing days has not been exceeded, No 594, the program has ascertained that it is authorized to dispense 596 by the Prescription 4 and proceeds to aggregate digitally captured Patient 6 information.

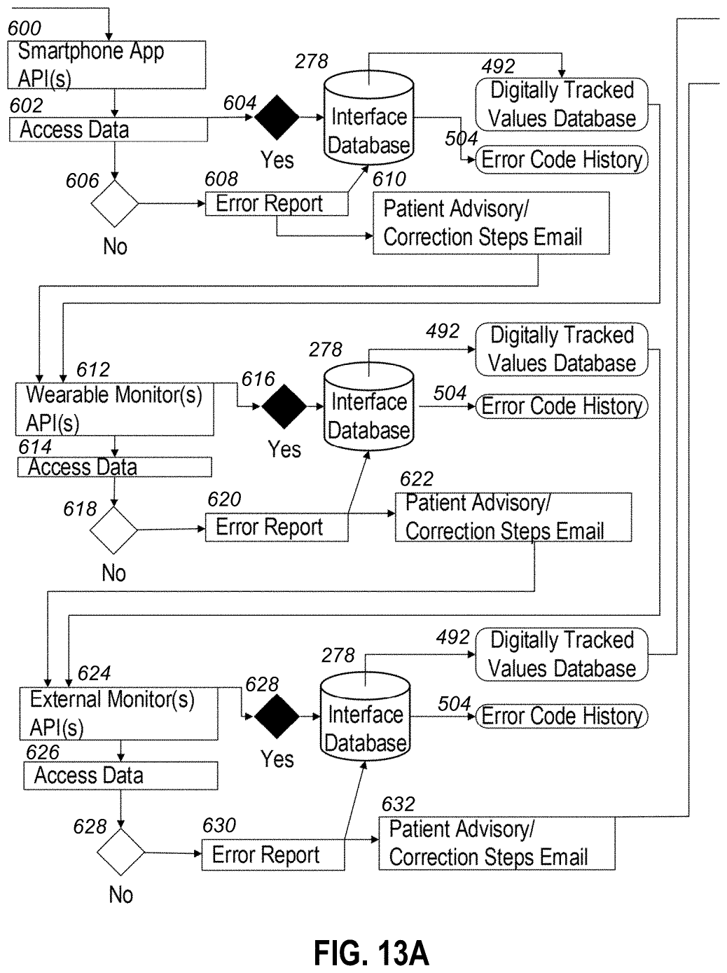

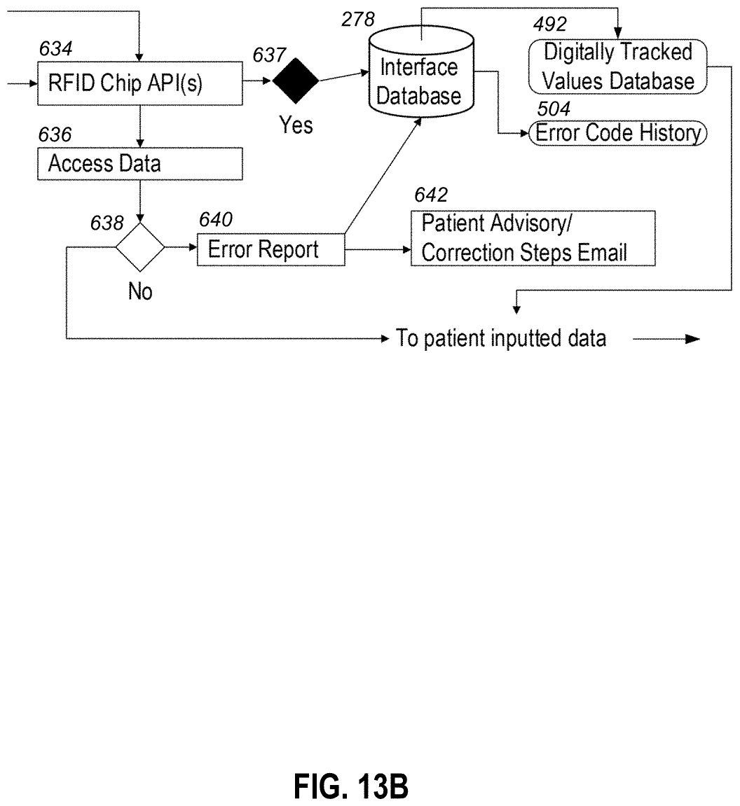

FIG. 13 is an exemplary embodiment of the program routines to aggregate digitally captured information from third party devices via application program interfaces, APIs. As an example, the information may be aggregated from the Patient's 6 smartphone (e.g., diet diaries, stress, oxygen saturation, heart rate, other medications taken, and Bluetooth connected drug dispenser information, etc.), wearable monitors (e.g., smart watch, exercise monitor, holter monitor, ECG, RFID chip tracker, wearable diagnostic monitor, etc.), external monitoring and diagnostic devices (e.g., scales, diagnostic devices, clothing, pill dispensers, etc.), connectivity bridges and connectivity hugs (e.g., Verily's Connectivity Bridge), and RFID chips whose API interface allows the signal to be picked up and the data stored on the Drug Specific App's 12 Interface Database 278, and subsequently on the Patient Database 152 on the Data Servers 10, etc.

Each tracked interface has its own API. The program is organized to aggregate the data on a sequential basis, e.g., it goes through all the Smartphone App APIs (more than one may be designated) before it goes to the Wearable Monitor APIs (more than one may be designated) and so forth.

The program begins gathering digitally captured Patient 6 information by accessing the respective Smartphone App API(s) 600 to access and aggregate the designated information, access the data 602. If it is successful in accessing the data, Yes 604, the program accesses the Interface Database 278 and enters the values in the Digitally Tracked Values Database 492. If it is unable to access the data, No 606, then it creates an error report 608, creates and sends a patient advisory correction steps 610 email to the Patient 6, accesses the Interface Database 278 and enters the Error Report 608 into the Error Code History 504 database. It then continues to the next Smartphone App API, or if no more are indicated, to the Wearable Monitor(s) API(s) 612.

It accesses the Wearable Monitor API(s) 612 to access and aggregate the designated information and then accesses the data 614. If it is successful in accessing the data, Yes 616, the program accesses the Interface Database 278 and enters the values in the Digitally Tracked Values Database 492. If it is unable to access the data, No 618, then it creates an error report 620, creates and sends a patient advisory correction steps 622 email to the Patient 6, accesses the Interface Database 278 and enters the Error Report 620 into the Error Code History 504 database. It then continues to the next Wearable Monitor API, or if no more are indicated, to the External Monitor(s) API(s) 624.

It accesses the External Monitor API(s) 624 to access and aggregate the designated information and then accesses the data 626. If it is successful in accessing the data, Yes 627, the program accesses the Interface Database 278 and enters the values in the Digitally Tracked Values Database 492. If it is unable to access the data, No 628, then it creates an error report 630, creates and sends a patient advisory correction steps 632 email to the Patient 6, accesses the Interface Database 278 and enters the Error Report 630 into the Error Code History 504 database. It then continues to the next External Monitor API, or if no more are indicated, to the RFID Chip API(s) 634.

It accesses the RFID Chip API(s) 634 to access and aggregate the designated information and then accesses the data 636. If it is successful in accessing the data, Yes 637, the program accesses the Interface Database 278 and enters the values in the Digitally Tracked Values Database 492. If it is unable to access the data, No 638, then it creates an error report 640, creates and sends a patient advisory correction steps 642 email to the Patient 6, accesses the Interface Database 278 and enters the Error Report 640 into the Error Code History 504 database. It then continues to the next External Monitor API, or if no more are indicated, to the Patient Self-Assessment Input Screen 624.

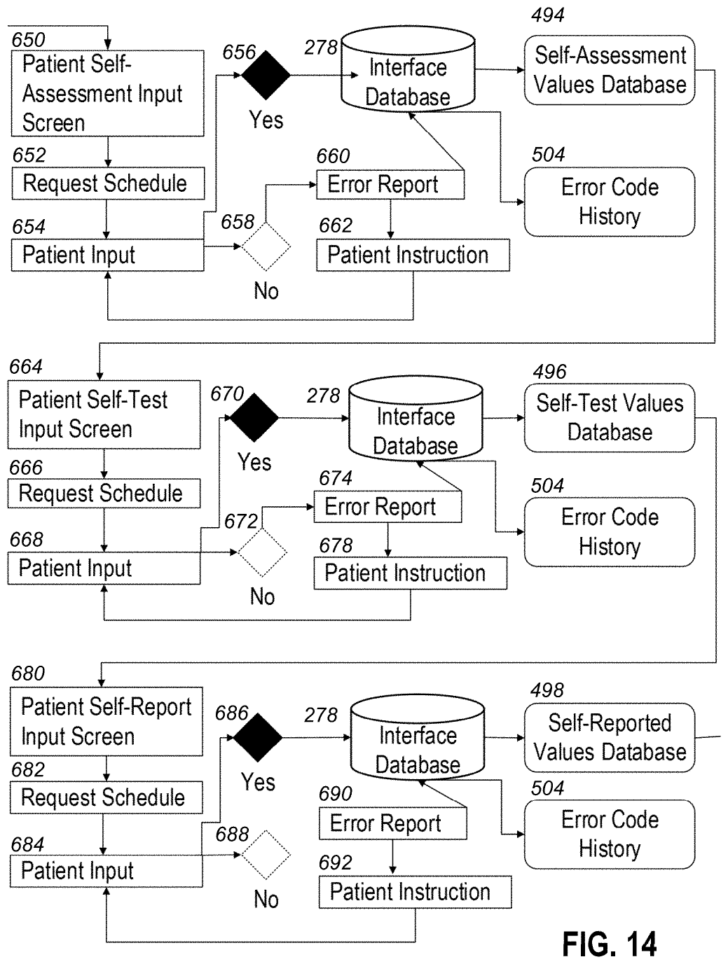

FIG. 14 is an exemplary embodiment of the program to capture patient self-assessment, patient self-test, and/or patient self-reported physiological, psychological, lifestyle, medications taken since the last drug dose and/or concomitant medications taken, and/or environmental information/values through designated Patient 6 input screens on the Drug Specific App 12. The data may be required by: (i) the Drug Specific Dispensing Algorithm 13, or (ii) as designated by the Prescriber 2 when he/she filled out the Prescription 4, or (iii) as required by regulatory agencies for the drug's regulatory approval if the product is in clinical trials. The number of screens presented versus the value of the collected data must be carefully weighed. A specific data screen may be presented each time the Patient 6 wants to dispense a dose or it may be presented on a predefined schedule or in response to a digitally captured value or a Patient 6 entered value/information.

The program is organized to aggregate the data on a sequential basis, e.g., it goes through all the patient self-assessment screens (more than one may be designated) before going to the patient self-test screens (more than one may be designated) and then to the patient self-report screens (more than one may be designated). The following represents examples of the data gathering logic for each.

After completing the RFID Chip API(s) routine, the program proceeds to the Patient Self-Assessment Input 650 screen. Before presenting the designated screen, it checks the request schedule 652. If the screen is designated, then it presents the screen and requests the patient input 654. If the Patient 6 is able to input his/her information, Yes 656, the program accesses the Interface Database 278 and enters the values in the Self-Assessment Values Database 494. If it is unable to capture the information, No 658, then it creates an error report 660, accesses the Interface Database 278, and enters the error code in the Error Code History 504 file. Simultaneously, it creates and presents patient instructions 662 on the Interface Device 14 or Standalone Drug Dispenser 16 interface screen and prompts the Patient 6 to begin the patient input process again. Once the values have been added to the Self-Assessment Values Database 494, the program proceeds to the next designated patient self-assessment routine, and if no more are called for, it proceeds to the Patient Self-Test Input Screen 664 routine.

Before presenting the designated Patient Self-Test Input Screen 664, the program routine checks the request schedule 666. If the screen is designated, then it presents the screen and requests the patient input 668. If the Patient 6 is able to input his/her information, Yes 670, the program accesses the Interface Database 278 and enters the values in the Self-Test Values Database 496. If it is unable to capture the information, No 672, then it creates an error report 674, accesses the Interface Database 278, and enters the error code in the Error Code History 504 file. Simultaneously, it creates and presents patient instructions 678 on the Interface Device 14 or Standalone Drug Dispenser 16 interface screen and prompts the Patient 6 to begin the patient input process again. Once the values have been added to the Self-Test Values Database 494, the program proceeds to the next designated patient self-test routine, and if no more are called for, it proceeds to the Patient Self-Report Input Screen 680 routine.

Before presenting the designated Patient Self-Report Input Screen 680, the program routine checks the request schedule 682. If the screen is designated, then it presents the screen and requests the patient input 684. If the Patient 6 is able to input his/her information, Yes 686, the program accesses the Interface Database 278 and enters the values in the Self-Reported Values Database 498. If it is unable to capture the information, No 688, then it creates an error report 690, accesses the Interface Database 278, and enters the error code in the Error Code History 504 file. Simultaneously, it creates and presents patient instructions 692 on the Interface Device 14 or Standalone Drug Dispenser 16 interface screen and prompts the Patient 6 to begin the patient input process again. Once the values have been added to the Self-Reported Values Database 498, the program proceeds to the next designated patient self-reported routine, and if no more are called for, it proceeds to the Patient Concomitant Medications Input Screen 700 routine.

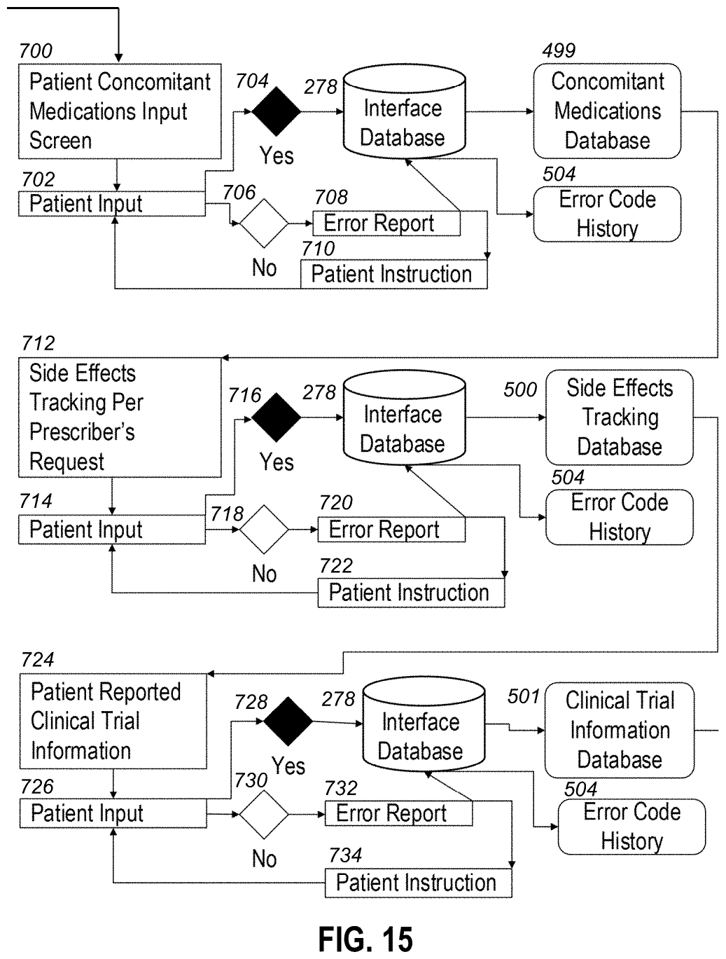

FIG. 15 is an exemplary embodiment of the program to capture patient concomitant medications, side effects, and/or patient reported clinical trial information on the designated Drug Specific App 12 data capture screens required by: (i) the Drug Specific Dispensing Algorithm 13, and/or (ii) as designated by the Prescriber 2 when he/she filled out the Prescription 4, and/or (iii) as required by regulatory agencies for the drug's regulatory approval if the product is in clinical trials. The number of screens presented versus the value of the collected data must be carefully weighed. A specific data screen may be presented each time the Patient 6 wants to dispense a dose or it may be presented on a predefined schedule or in response to a digitally captured value or a Patient 6 entered value/information.

The program is organized to aggregate the data on a sequential basis, e.g., it goes through, for example, the Patient Concomitant Medications Input Screen(s) 700, then the Side Effects Tracking 712 screen(s), and then the Patient Reported Clinical Trial Data 724 screen(s). The following represents examples of the data gathering logic for each.

Upon completion of the patient self-assessment, and/or the patient self-test, and/or the patient self-reported input screens, the program presents the Patient Concomitant Medications Input Screen 700 and requests the patient input 702. If the Patient 6 is able to input his/her information, Yes 704, the program accesses the Interface Database 278 and enters the values in the Concomitant Medications Database 499. If it is unable to capture the information, No 706, then it creates an error report 708, accesses the Interface Database 278, and enters the error code in the Error Code History 504 file. Simultaneously, it creates and presents patient instructions 710 on the Interface Device 14 or Standalone Drug Dispenser 16 interface screen and prompts the Patient 6 to begin the patient input process again. Once the values have been added to the Self-Reported Values Database 499, the program proceeds to the Side Effects Tracking Per Prescriber's Request 712 input routine.

The program presents the Side Effects Tracking Per Prescriber's Request 712 input screen and requests the patient input 714. If the Patient 6 is able to input his/her information, Yes 716, the program accesses the Interface Database 278 and enters the values in the Side Effects Tracking Database 500. If it is unable to capture the information, No 718, then it creates an error report 720, accesses the Interface Database 278, and enters the error code in the Error Code History 504 file. Simultaneously, it creates and presents patient instructions 722 on the Interface Device 14 or Standalone Drug Dispenser 16 interface screen and prompts the Patient 6 to begin the patient input process again. Once the values have been added to the Side Effects Tracking Database 500, the program proceeds to the Patient Reported Clinical Trial Information 724 input routine.

The program presents the Patient Reported Clinical Trial Information 724 input screen and requests the patient input 726. If the Patient 6 is able to input his/her information, Yes 728, the program accesses the Interface Database 278 and enters the values in the Clinical Trial Information Database 501. If it is unable to capture the information, No 730, then it creates an error report 732, accesses the Interface Database 278, and enters the error code in the Error Code History 504 file. Simultaneously, it creates and presents patient instructions 734 on the Interface Device 14 or Standalone Drug Dispenser 16 interface screen and prompts the Patient 6 to begin the patient input process again. Once the values have been added to the Clinical Trial Information Database 501, the program proceeds to the Dispensing Algorithm 740 routine.

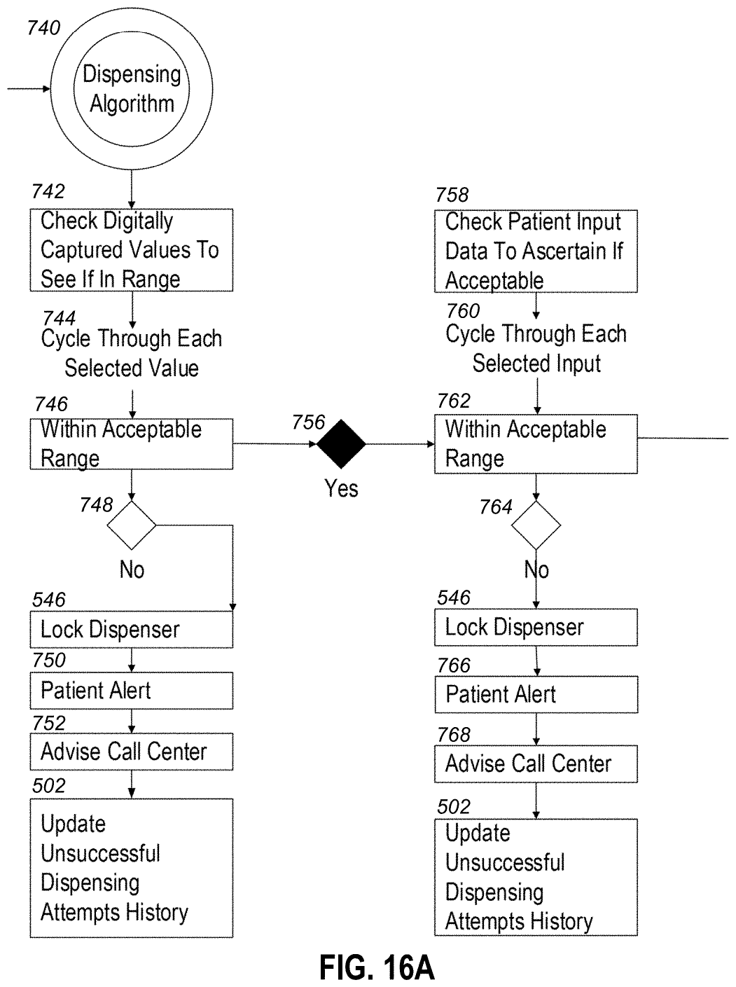

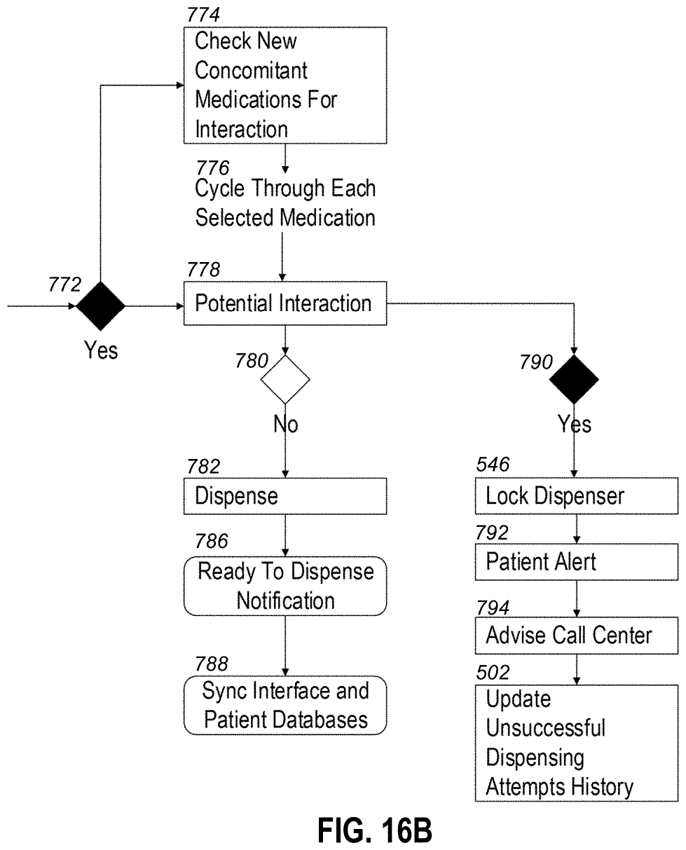

FIG. 16 is an exemplary embodiment of the logic followed by the Dispensing Algorithm 740. Any information requested by the prescriber or aggregated for use for clinical trial data capture that is not required by the Drug Specific Dispensing Algorithm 13 is not taken into account when making dispensing decisions. The algorithm sequentially cycles through the digitally captured values to the patient inputted data to the concomitant medications information to make a dispensing decision.

The Dispensing Algorithm's 740 program begins by checking each of the digitally captured values to see if they are in range 742. The routine cycles through each of the selected values 744 to ascertain if they are within the predefined acceptable range 746. If the answer is No 748 for any value, then the program locks the Drug Dispenser 546, issues a patient alert 750 to the Patient 6 and anyone else who is listed as an individual that should be alerted, advises the Integrated Call Center 752, and updates the unsuccessful dispensing attempts history 502. If all the digitally captured values are within the acceptable range(s), Yes 756, then the program proceeds to check the patient inputted data.

The program checks the respective Patient 6 self-assessment, self-test, and self-reported input data to ascertain if the data is acceptable 758, then cycles through each selected input 760 to ascertain if they are within the predefined acceptable range 762. If the answer is No 764 for any value, then the program locks the Drug Dispenser 546, issues a patient alert 766 to the Patient 6 and anyone else who is listed as an individual that should be alerted, advises the Integrated Call Center 768, and updates the unsuccessful dispensing attempts history 502. If all the self-assessment, self-test, and self-reported values are within the acceptable range(s), Yes 772, then the program proceeds to check the concomitant medications diary.

The logic then checks the new concomitant medications list 774 and cycles through each selected medication 776 to ascertain if there is a potential interaction 778 that could result in an adverse drug-drug mediated adverse event. If the answer is No 780 for each new medication, then the program is ready to dispense 782, brings up a ready to dispense notification 786 on the Interface Device's 14 or the Standalone Drug Dispenser's 16 screen, and then proceeds to synchronize the Interface and Patient Databases 788 to indicate the dispensing of the medication dose was authorized at a specific date and time (The program automatically makes adjustments for time travel differences for patients who travel). This information along with the date and time the Drug Dispenser 16 dispenses the dose can be used to ascertain if there may be possible medication diversion.). However, if a potential drug-drug interaction is flagged, Yes 790, then the program locks the Drug Dispenser 546, issues a patient alert 792 to the Patient 6 and anyone else who is listed as an individual that should be alerted, advises the Integrated Call Center 794, and updates the unsuccessful dispensing attempts history 502.

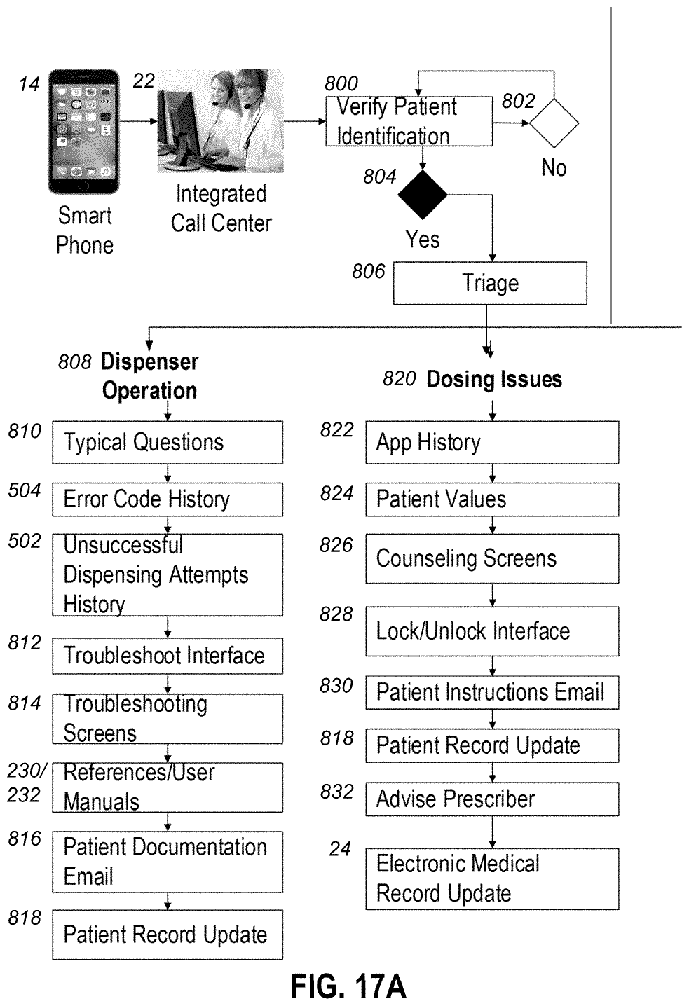

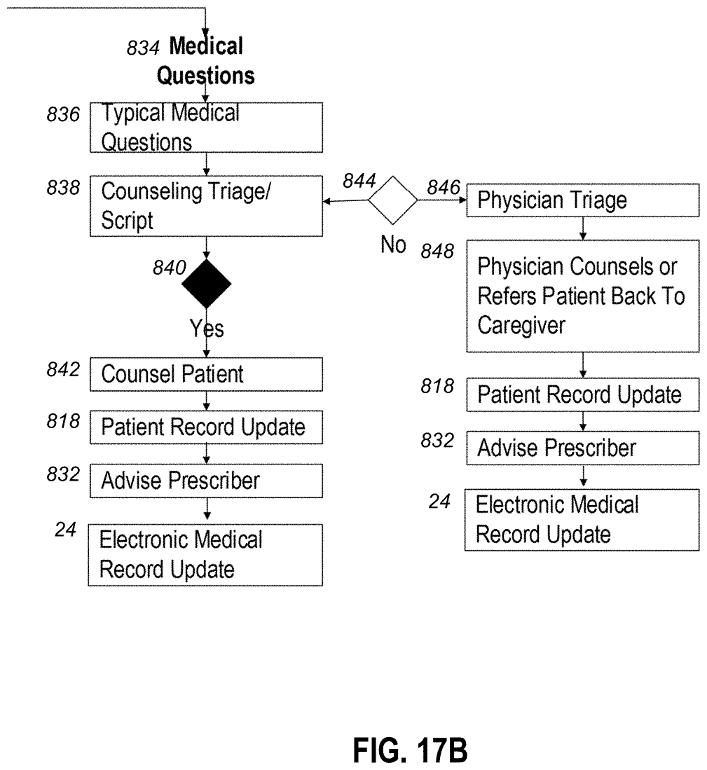

FIG. 17 is an exemplary embodiment of the Integrated Support Center 22 triage flow from Patient 6 contact through issue resolution. Triage is organized into three main types of Patient 6 support calls: dispenser operation, dosing issues, and medical questions.

Each of the patient alerts 290, 552, 562, 570, 582, 586, 592, 610, 622, 632, 642, 662, 678, 692, 710, 722, 734, 750, 766, 792, etc. presented on the Interface Device 14 or Standalone Drug Dispenser 16, contain an automatic dialing option to immediately contact the Integrated Call Center 22 to resolve the dispensing issue(s). The email and text message alerts contain clearly denoted Integrated Call Center 22 call numbers.

When the Patient 6 calls 14 the Integrated Call Center 22 the call center's security program brings up the patient identification verification routine 800. If the Integrated Call Center does not initially identify the Patient 6, No 802, it has alternatives available to verify the Patient's 6 identity within the HIPPA Privacy Rules for protection of the individuals' medical records and other personal health information.

Once the Patient's 6 identification is verified, Yes 804, the call center representative looks at the Patient's 6 profile generated by the call center software that draws the Patient 6 information from the Patient Database 152 resident on the Data Servers 10 to assist in triaging 806 the Patient's 6 support request into a dispenser operation 808 issue, dosing issue 820, or medical question 834/issue. If multiple issues present themselves, the representative follows a sequence that goes from the dispenser 808 to dosing 820 to medical issues 834. The following are exemplary issue resolution sequences.

To resolve a dispenser operation 808 issue, the representative defines the issue, selects the issue on her/his support screen and begins asking a number of typical questions 810 to narrow down the scope of the inquiry. Once sufficiently narrowed, he/she reviews the Error Code History 504, the Unsuccessful Dispensing Attempts History 502, and then proceeds to the Troubleshooting Interface 812. The Troubleshooting Interface 812 allows the representative to pick different troubleshooting screens 814 to work with the patient to troubleshoot the information. Each screen is linked to the Reference and User Manuals 230, 232. Once the issue is resolved, the representative prepares a documentation email 816 to be sent to the Patient 6 defining the issue and what was done to resolve it. The email may contain attachments from the references/user manuals 230, 232. The representative updates her/his notes in the Patient Record 818.

To resolve a dosing issue 820, the representative first reviews the Patient's 6 Drug Specific App's drug dispensing history 822 versus certain patient values 824. She/he then proceeds to select the appropriate counseling screens 826 which are designed to assist in and give guidance to the representative regarding how to proceed. They provide the decision tree relating to any decisions of whether to: (i) leave the Drug Dispenser 16 locked 828, (ii) lock the Drug Dispenser 16 until the Patient 6 consults with their Prescriber 2 and/or physician 828, or (iii) unlock the Drug Dispenser 16 and allow it to dispense the dose 828. Thereafter, the representative will document what was discussed and how the issue was resolved in an email 830 that is sent to the Patient 6. Then the Patient Record 818 is updated, if required, to advise the prescriber 832 on what transpired, and, again, if required, update the Patient's 6 Electronic Medical Record 24 resident on the Data Servers 10.

To resolve a medical question 834, the representative goes to his Typical Medical Questions 836 screen for the specific drug and/or symptom (e.g., type of pain). The representative selects the appropriate subject from the screen and reviews the appropriate counseling or triage script 838 to ascertain whether she/he should counsel the Patient 6 or port the Patient to a call center physician. If the representative can counsel the Patient 6, Yes 840, the representative discusses the issue with the patient and makes certain recommendations (counsels the patient) 842. Thereafter, the representative updates the patient record 818, if appropriate, advises the prescriber 832, and again, if appropriate, updates the Electronic Medical Record 24 resident on the Data Servers 10.

If the representative is instructed by the call center screen to triage the patient, No 844, then the representative ports the Patient 6 to the appropriate call center physician (or appropriate allied healthcare professional) 846. The physician counsels the Patient 6 or refers the Patient 6 back to their caregiver 848. Thereafter, the physician updates the patient record 818, advises the Prescriber 2 and/or any other designated caregivers 832, and updates the Patient's 6 Electronic Medical Record 24 resident on the Data Servers 10.

FIG. 18 is an exemplary embodiment of a flow chart of a Drug Specific App 12, standard prescription Biometric Authentication 850 log in, aggregation of digitally captured information 852, 854, 856, 857, 858, 859, 860, Patient Self-Assessment 862, Patient Self-Test 864, Patient Self-Report 866 input screens, and Drug Specific Dispensing Algorithm 13 controlled drug dispensing Patient 6 screens 868, 870, 872, and 874. The exemplary embodiment is an illustration of the Patient 6 interaction to dispense a drug (such as oxycodone, oxycodone/APAP, OxyContin®, hydrocodone, hydrocodone/APAP, hydromorphone, oxymorphone, morphine, buprenorphine, etc. in their respective formulations, e.g., abuse deterrent, immediate release, sustained release, extended release, combination product, prodrug, etc.) medication.

The process begins by the Drug Dispensing App 12 alerting the Patient 6 that it is time to take his/her medication. If the Drug Dispensing App 12 is resident on a smartphone, the Drug Dispenser App 12 graphic' automatically changes to the Biometric Authentication screen 850 when the Patient 6 unlocks the phone. Alternatively, the Patient 6 can tap on the Drug Specific App 12 at any time. A tap on the Drug Specific App 12 screen brings up the Biometric Authentication 850 screen. When the Patient 6 is authenticated, (i) the Drug Specific App 12 effectuates a handshake with the Drug Dispenser 16 and any digital devices for which an application programming interface (API) has been written, (ii) checks to ensure the drug has not expired 458 or exceeded its beyond-use date/prescription expiration date 103, (iii) confirms that it has been stored within temperature 104, 460 and humidity 106, 462 storage parameters, (iv) checks the prescription to ensure the drug can be dispensed within prescribing guidelines 70, 72, 74, 80, 82, 84, 86, and then (v) moves to the Patient Self-Assessment screen(s) 862. A click on a response value on the Patient 6 Self-Assessment Screen 862 automatically moves to the next self-assessment screen, if required, or to the Patient 6 Self-Test screen(s) 864. Completion of the Patient 6 Self-Test(s) automatically moves the process to the Patient 6 Self-Reporting 866 screen(s). Upon completion of the Self-Reporting information, the Drug Specific Dispensing Algorithm 13 processes the various inputs.

If nothing is found to block dispensing by the Drug Specific Dispensing Algorithm 13, then the Patient 6 sees the Dispense screen 868. By clicking, for example, on the Drug Dispenser 16 dispensing button, the patient is able to dispense a dose with a single click—after which the Drug Dispenser 16 goes back to the locked position unless the prescription allows the patient to dispense a number of doses on a PRN basis without waiting between doses. It the prescription is PRN, the Drug Dispenser 16 will be locked after the designed number of doses are dispensed within a specified period of time. If the Patient 6 wants to change a prior entry before dispensing, he/she can use the devices scroll back capabilities to return to the right screen and change the selection. If the Drug Specific Dispensing Algorithm 13 finds any reason not to allow dispensing, it selects from the appropriate drug specific screen to show why dispensing was rejected and to facilitate the Patient's 6 ability to avail himself/herself of the proper medication support 870, 872, 874.

The exemplary embodiment of the Patient Self-Assessment screen 862, the Patient Self-Testing screen 864, and the Patient Self-Reporting Screen 866 encompass, for example, a self-assessment pain measurement scale adapted from Wong Baker Faces 862; a self-testing motor skills test 864; and a self-reporting stool consistency observation utilizing the Bristol Stool Scale, a well-accepted stool measure 866. These are examples of screens that can be utilized in the embodiment as an input to the Drug Specific Dispensing Algorithm 13 to decide whether to dispense. The scales can be created, adapted, or integrated to capture the desired patient reported information. These can be used, for example, in addition for use by the Drug Specific Dispensing Algorithm 13, to titrate the drug, monitor increasing tolerance, gauge medication efficacy, for better patient management, to better manage drug-mediated side effects, for preventative health (e.g., avoid or proactively manage a medication mediated side-effect), for clinical trials, and/or for post marketing surveillance, etc.

The embodiment is applicable for, as an example, clinical trials, post-launch surveillance, for the FDA's Risk Evaluation and Mitigation Strategy (REMS) programs, and to control and ensure drugs are efficacious and safe as dispensed within the Drug Specific Dispensing Algorithm 13 as part of a prescribed drug regimen, etc. The Drug Specific App 12 can be configured to capture all the patient self-assessment, self-test, and/or self-report information which is required by the FDA, EMA or other like governmental agencies for approval or regulatory compliance of drugs. The only difference from the standard Drug Specific Algorithm 13 are the additional input screens required. The same Drug Specific Dispensing Algorithm 13, decision tree, would be used for normal and clinical trial prescribing.

FIG. 19 is an exemplary embodiment of the Digitally Captured information 859, 858, 880, 882, 884, 886 and is illustrative of the types of digital information, in addition to that captured on an Interface Device 14, such as a smartphone (e.g., diaries, medication lists, stress monitoring, heart rate, SpO₂, diet, exercise, blood pressure, EKG, holter values, environment temperature, altitude, etc.) which can be collected and integrated into the Decision Tree logic (illustrated in FIG. 12 through FIG. 16), in the respective Drug Specific Dispensing Algorithms 13. The availability of disease specific Apps and related disease or condition specific digitalized health information is rapidly emerging, making the examples in FIG. 19 wanting not only for the disease information but for physiologic, psychologic, lifestyle, diagnostic, medications being taken, digital medication ingestion, diagnostic and tracking devices, and environmental input, etc.

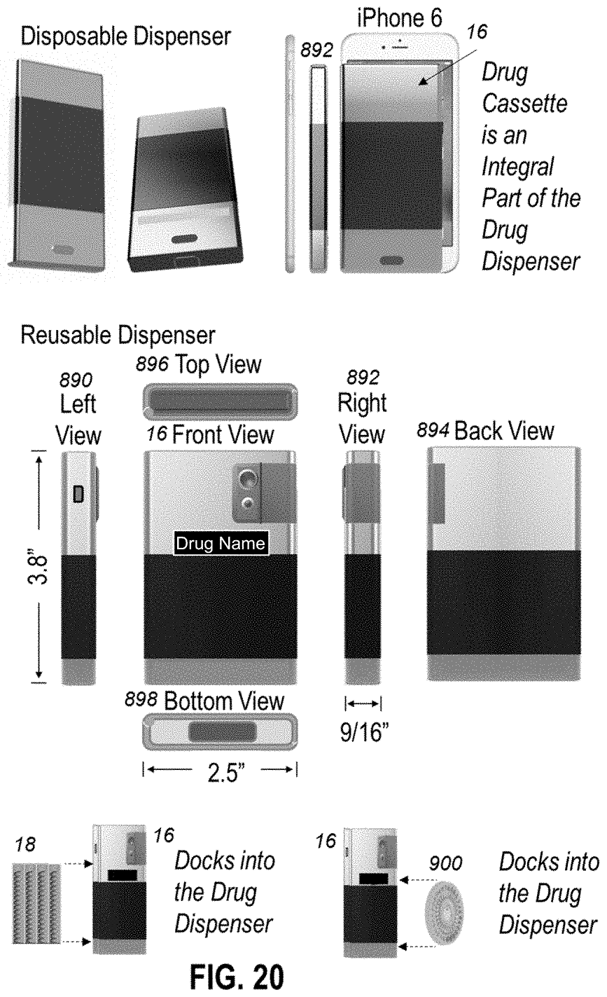

FIG. 20 is an exemplary embodiment illustration of the Drug Dispenser 16. There are many different permutations, from a fully disposable tamper resistant Disposable Drug Dispenser 16 controlled by a Drug Specific App 12, to a Reusable Drug Dispenser 16 controlled by a Drug Specific App 12, to a Standalone Drug Dispenser 16 with the Drug Specific App 12 incorporated in the Standalone Drug Dispenser 16, etc. Furthermore, each Drug Dispenser 16 can be configured from one to multiple drugs. The following and FIGS. 20, 21, 22, 23, 24, 25, 28, 29, and 30 are examples of the design alternatives available and the capabilities integrated into the novel system. The illustrations present a reusable Drug Dispenser 16 controlled by a Drug Specific App 12 as it represents the most complex Drug Dispenser 16 iteration versus the Disposable Drug Dispenser 16 which presents the least complex Drug Dispenser 16 design.

The Reusable Drug Dispenser 890, 892, 894, 896, 898, 16 is designed to be: (i) controlled by a Drug Specific App 12 resident on an Interface Device 14, (ii) water proof, (iii) tamper resistant, (iv) able to withstand being dropped and/or banged, to be rugged, (v) able to operate and withstand hot and cold temperatures within defined temperature ranges, (vi) reusable, (vii) rechargeable and/or to have its battery changed, and (viii) small enough to be carried in a pants pocket or purse. The Reusable Drug Dispenser 16 automatically recognizes the drug based upon the Drug Specific Drug Cassette 18, 900 docked into the Reusable Drug Dispenser (16). In the most secure configuration, which is designed to restrict drug access, other than authorized dispensed doses, to medical professionals, the Drug Cassette 18, 900 can only be docked or removed by a healthcare professional. The Reusable Drug Dispenser 16 remains locked from dispensing unless it receives an encrypted signal from the authorized Drug Specific App 12. The Drug Dispenser 16 dispenses the drug dose with one click.

The Reusable Drug Dispenser 16 when interfaced through a digital handshake with the Drug Specific App 12 transmits for example: (i) its (the Drug Dispenser's) serial number, (ii) the drug information on the Drug Cassette 16, 900, (iii) current and time tracked historic temperatures since the last dispense, (iv) time tracked humidity exposure since the last dispense, (iv) the date and time the drug was last dispensed, and any date stamped unauthorized attempts to open or tamper with the Drug Dispenser 16.

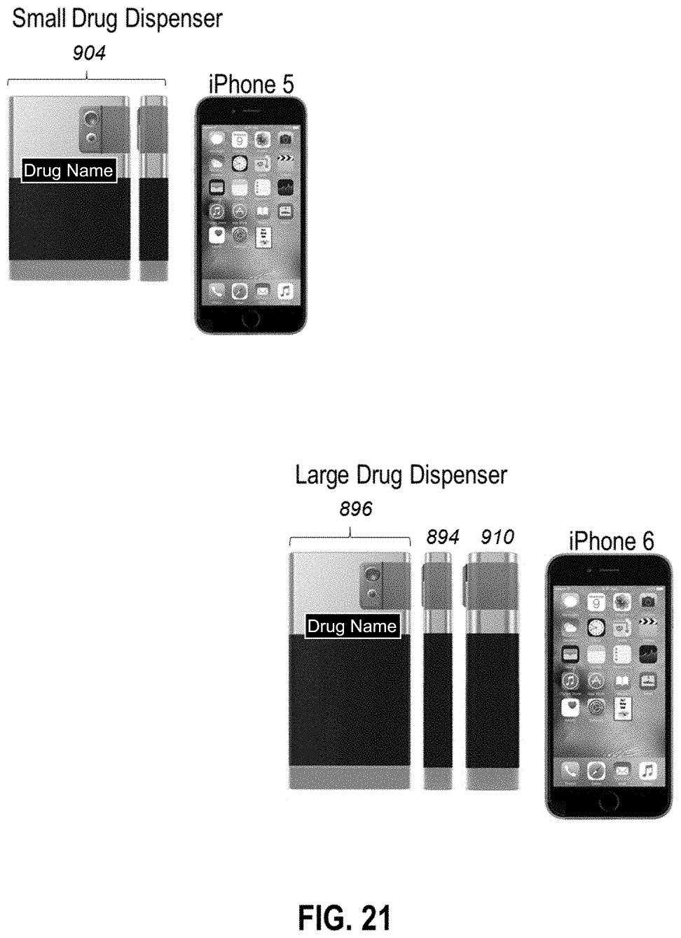

FIG. 21 is an exemplary embodiment illustration showing the size of an exemplary design for the smallest Drug Dispenser 904 and the ability to increase the height 896 or the width 894, 910 of the Drug Dispenser 16 to accommodate more pills and/or bigger pills and/or a second or more Drug Cassettes 18. The width can also be expanded to accommodate the dispensing of two or more different medications, each which is resident on its own Drug Cassette 18. Multidrug regimens can also be accommodated by individual cassettes as long as each drug is filled in a separate drug well to preclude cross contamination.

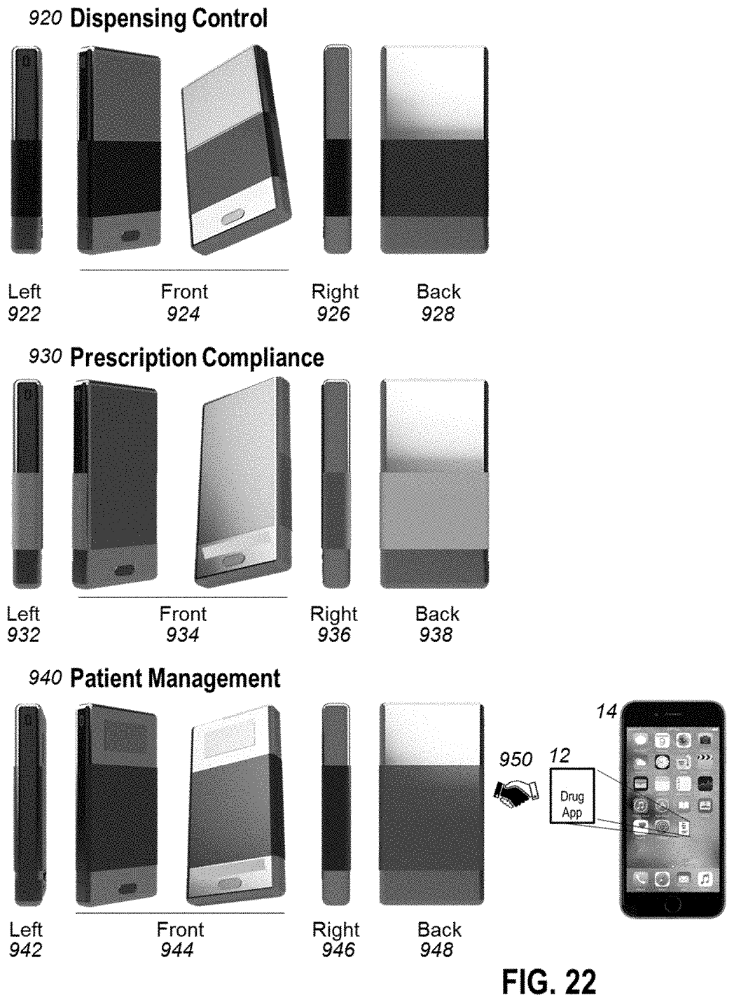

FIG. 22 presents design renderings for different drug dispenser configurations and designs with different features and benefits, each being designed to meet specific Patient 6 and drug specific requirements. Each is simple to operate with a shared biometric login and dispensing button and some with an interface screen.

For example, the Dispensing Control 920 configuration is a standalone drug dispenser lacking external communication capabilities simply designed to control: (i) access via a biometric logon, (ii) dispensing per the prescribed schedule, and (iii) Drug Cassette 18 loading, if desirable, only by a healthcare professional. It also may contain, by reference, some or all the capabilities presented in the various embodiments.

The Prescription Compliance 930 standalone drug dispenser has all the features of the Dispensing Control 920 drug dispenser plus Wi-fi, Internet, and/or Bluetooth communications capabilities to enable emailing of select alert communications to the Patient 6, defined caregivers and/or family members. The unit has a large interactive screen to enable the use of a Drug Specific App 12 to control data aggregation, input, email communications, drug dispensing, etc. A slightly thicker version of the standalone device could add certain phone capabilities which would provide this version with all the capabilities of the Patient Management 940 system but in a standalone dispenser configuration. It may contain some or all of the capabilities presented in the various embodiments.

The Patient Management 940 configuration is comprised of a Drug Dispenser 16 controlled by a Drug Specific App 12 which is resident on an Interface Device 14. It has all the capabilities of the standalone dispensers 920, 930 and by reference may incorporate some or all the capabilities presented in the various embodiments.

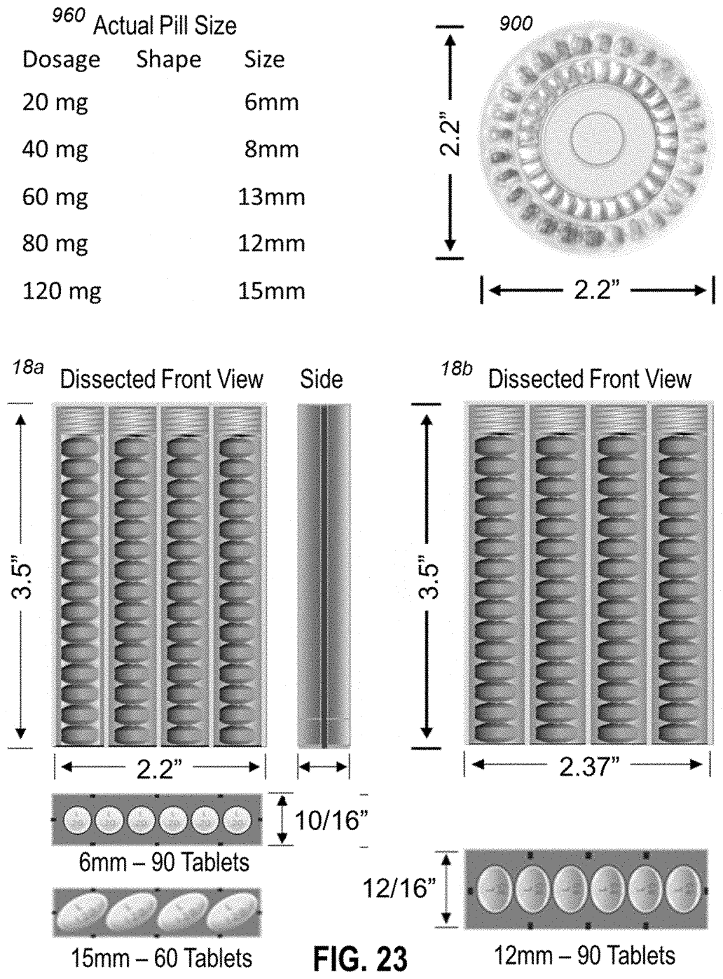

FIG. 23 is an exemplary embodiment illustration of different Drug Cassettes 960, 18a, 18b using, as an example, OxyContin® in its various dosage strengths and pill sizes 960. OxyContin® requires different Drug Cassette sizes and configurations, and depending on the number of pills required for the prescription, may require the larger of the two presented Drug Dispensers 904, 896. Each Drug Cassette 18, 18a, 18b, 900 is designed: (i) to use approved drug packaging materials, (ii) to dock into the Drug Dispenser 18, 920, 930, 940, and (iii) as a blank cartridge which can accommodate a number of different pills, caplets, capsules, etc. within a specified size range. The blank Drug Cassette 900, 18, 18a, 18b is designed to be proprietary to the Drug Dispensers 16, 920, 930, 940. Each is marked, as part of the automated cassette fill operation, to allow the Drug Dispenser 16, 920, 930, 940 to recognize the drug contained in the Drug Cassette 18, 18a, 18b, 900 (for an individual drug in a cassette or for each of the multiple drugs in a single Drug Cassette 18, 18a, 18b, 900; or multiple Drug Cassettes 18 in each respective multi-drug Drug Dispenser 1002, 1004, 1006, 1008). Each is linked to the: (i) name of the drug (brand and/or generic) 400, (ii) drug's NDC number 402, (iii) drug's batch number 456, (iv) drug's expiration 458/beyond-use date, etc. The Drug Cassette's 18 label is designed to meet all drug label regulatory requirements.

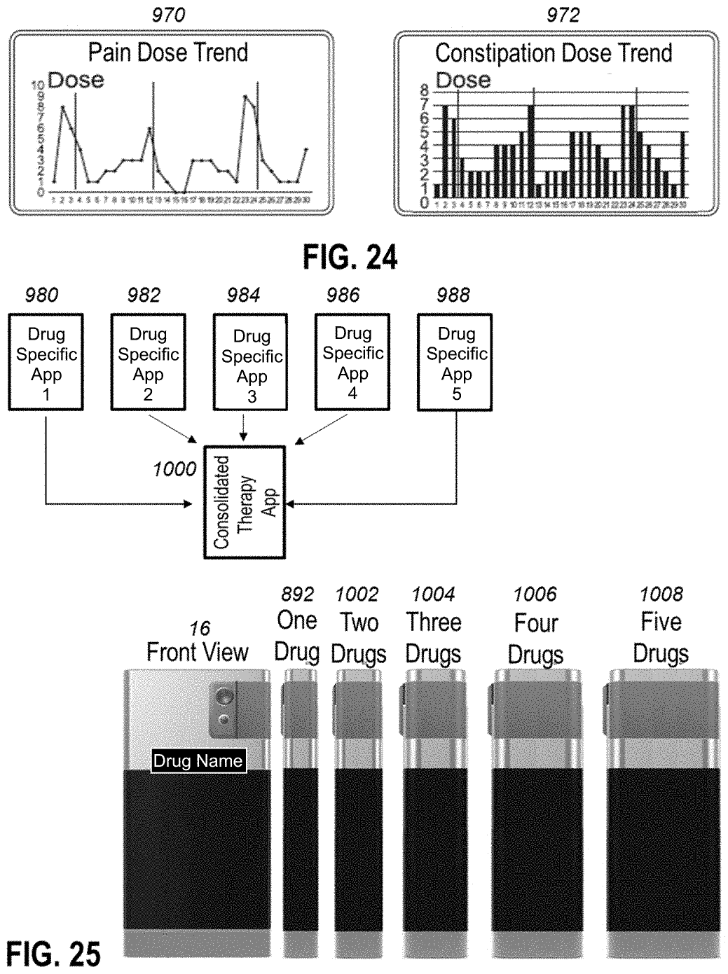

FIG. 24 presents exemplary embodiment of the Patient 6 specific charts 970, 972, which illustrate the relationship between when the Patient 6 took their medication versus his/her self-assessment, self-test, self-reported, or digitally captured symptoms and/or diagnostic values. This clearly shows the relationship between the medication and pain management 970 and the medication and symptoms/side effect management 972. The charts or tables, which can be requested and viewed by the Patient 6 on the Interface Device 14 or the Standalone Drug Dispenser 16 are designed to educate the patient 6 and promote Patient 6 prescription compliance and persistence.

Prescribers 2 can utilize the information to ensure the medication is efficacious for the individual Patient 6, to titrate dosing, personalize pain therapy, and to manage drug tolerance (for personalized medicine).

The respective charts, graphs, reports, etc. may be generated by the Drug Specific App 12 and/or by the Integrated Support Center's 22 centralized analytics platform 10 resident on the Data Servers 10.

FIG. 25 is an exemplary embodiment illustration of Drug Dispensers designed to serve the needs of most Patients 6. Approximately half of all Patients 6 take two medications and 20 percent take five or more. The Consolidated Therapy App 1000 automatically senses other Drug Specific Apps, for example 980, 982, 984, 986, 988, that are on the Interface Device 14. It consolidates from two to many Drug Specific Apps 12 into a single user interface for all drugs—eliminating duplicate logins, entries, and record keeping. It in turn digitally handshakes with the Multi-Drug Dispenser 1002, 1004, 1006, 1008 and uses the individual Drug Specific Dispensing Algorithms 13 to control dispensing of each individual medication. Furthermore, it coordinates the dispensing schedules to have as few dispensing times, within the respective prescriptions, as possible. The Multi-Drug Dispensers 1002, 1004, 1006, 1008 eliminate concerns about which drugs must be taken when.

Illustrations 1002, 1004, 1006, 1008 are exemplary of dispensing units containing from two drugs to five drugs. These units are standalone or can be docked into a Multi-Dispenser desktop unit dispenser.

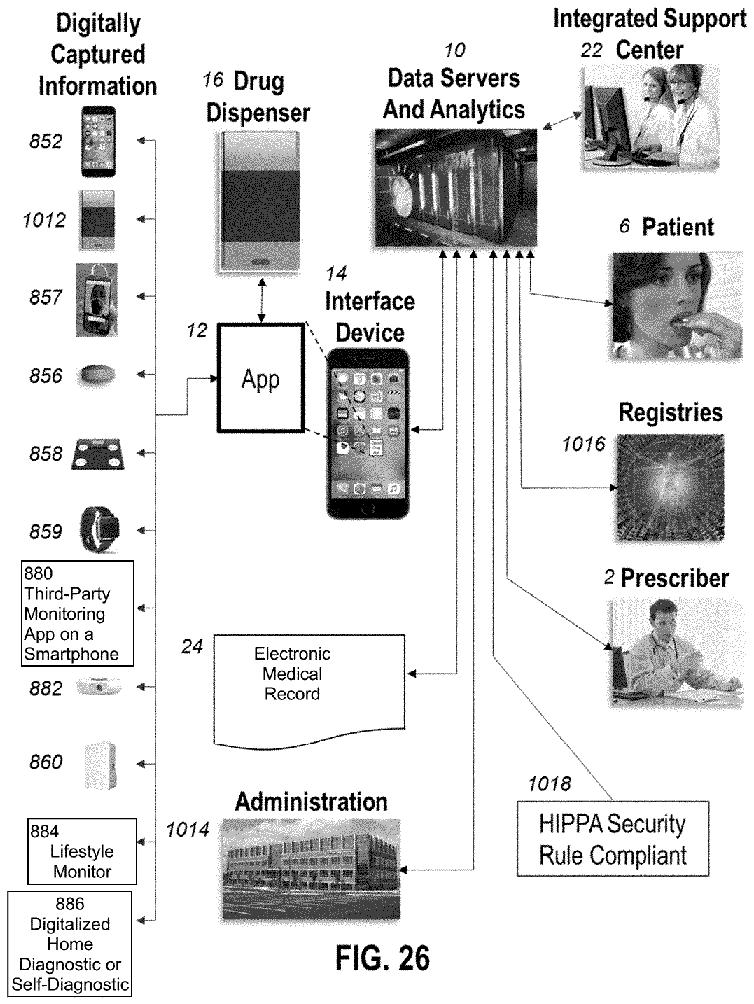

FIG. 26 is an exemplary embodiment of the integrated IT System 1010 that ties together the Drug Dispenser 16, the Drug Specific App 12, and the Interface Device 14 with the: (i) Data Servers 10, (ii) databases, and (iii) Analytics systems 10 to ensure the Patient 6 is receiving the best care, tailored to the Patient 6 (“personalized medicine”), for the prescribed Drug.

All the data collected by the Drug Specific App 12, from the Drug Dispenser 16, 920, 930, 940, Digitally Captured Information 852, 1012, 856, 857, 858, 859, 860, 880, 882, 884, 886, the Patient Self-Assessment screens 862, the Patient Self-Test screens 864, and the Self-Reporting screens 868 contained within the Drug Specific App 12, and the respective output of the Drug Specific Dispensing Algorithm 13 are transmitted by the Drug Specific App 12 through the Interface Device 14 (or the standalone drug dispenser's Internet connection) to the appropriate Patient 6 databases resident on the Data Servers 10. The data is utilized to update the respective patient screens used by the Integrated Support Center 22. The data is also made available to the respective Drug Registries 1016 and the related Electronic Medical Record 24. Any information that requires a communication with the Patient 6 and/or the Prescriber 2 is handled either automatically by the patient management software or by the Integrated Support Center 22.

The Patient's 6 information is continually analyzed by the analytical routines both individually for the Patient 6 as well as in comparison with treatment data from other like patients to ascertain if any changes in therapy may be warranted. This analytical capability is utilized by the Integrated Support Center 22 to assist Prescribers 2 when they are trying to develop a treatment plan for difficult patients. The Analytics 10 performed may include the Patient's 6 data, pooled patient information, as well as information from Electronic Medical Records 24 (resident on the Data Servers 10 or which reside in external databases), clinical studies, and publications, etc.

As further example of the embodiment, the Data Servers and Analytics 10 provide the following, as well as other, exemplary backbone support:

For the Drug Specific App 12: (i) assigns the App to a specific Patient 6, (ii) links the Drug Dispenser 16 to the Drug Specific App 12 which in turn limits the dispenser and App only to work with one another, (iii) stores the Drug Specific App 12 codes on server, (iv) issues, stores and links the Patient Identifier Number 160, and (v) enables and updates the Drug Specific App 12 software via communication with the Interface Device 14, etc.

For the Drug Dispenser 16: (i) issues, stores and links the Dispenser Identification No. 158, (ii) stores all reported data in the designated databases on the Data Servers 10, and syncs the patient data on all the respective Interface Devices 14 (smartphone, computer tablet, computer, standalone drug dispenser 16, 920, 930, 940 etc.); (iii) stores dispensing, dispensing attempts, lock, and malfunction data reported by the Drug Dispenser 16 via the Drug Specific App 12 and the Interface Device 14; (iv) transmits reports to the Patient 6 via the Drug Specific App 12 on request; (v) enables lock or unlock transmission from the Integrated Support Center 22; changes the Prescription 4 on the Drug Specific App 12 as inputted by the Integrated Support Center 22 representative per the Prescriber's 2 and/or authorized healthcare professional's instructions, and (vi) stores the authorized medical professional identification code required for the professional to open the Drug Dispenser 16 in order to change or load the Drug Cassette 18 into a Reusable Drug Dispenser 16, etc.

For the Integrated Support Center 22: (i) aggregates patient 6 data, (ii) presents and updates data on Patient 6 specific Integrated Support Center 22 screens, (iii) provides the ability to change a Patient's 6 prescription, (iv) enables the remote locking and unlocking of individual Drug Dispensers 16 via their Drug Specific App 12, (v) enables drug specific transmissions to all Patients 6, (vi) enables simultaneously locking of all Drug Dispensers 16 for a specific Drug in the event of a Drug recall, and (vii) enables medical professionals to open, load, and close the Reusable Drug Dispenser 16, (if so indicated, precludes the Patient 6, from opening the tamper resistant Reusable Drug Dispenser 16.) etc.

For the Patient 6: (i) prepares patient specific communications, (ii) creates personalized charts and reports, (iii) generates “Payer Outcomes Reports”, and required REMs reports, etc.

For Registries 1016: (i) maintains the Registry 1016, Electronic Medical Record 24 and Drug Specific App 12 databases and analytics. (ii) prepares Therapy efficacy reports, (iii) prepares best practices reports, and (iv) through the Integrated Support Center 22, provides Patient 6 specific diagnosis and therapy assistance to Prescribers 2 via the Integrated Support Center 22 representative or via HIPPA compliant access to certain Data Servers 10 queriable databases as requested.

For the Prescriber 2: (i) prepares and sends Patient 6 alerts, (ii) conducts meta-data analysis, prepares Patient 6 specific reports and shares the results with the Prescriber 2, (iii) provides the Prescriber 2, through the Integrated Support Center 22, assistance/guidance based upon Prescriber 2 requested database and analytics queries, and (iv) prepares best practices reports based upon patient 6 and Electronic Medical Records 24 meta-data analysis, etc.

For Electronic Medical Records 24: (i) interfaces with the Electronic Medical Record 24, (ii) updates Patient 6 dispensing, compliance, and persistence information, (iii) updates digitally captured and patient inputted physiological, psychological, lifestyle, concomitant medications, and environmental data collected by the Drug Specific App 12 that is required by the Drug Specific Dispensing Algorithm 13, requested be tracked by the Presciber 2, and/or that is required for clinical trial data submissions, (iii) updates any Integrated Support Center 22 counseling notes, and (iv) extracts patient 6 data, within HIPAA guidelines, for meta-data analysis, etc.

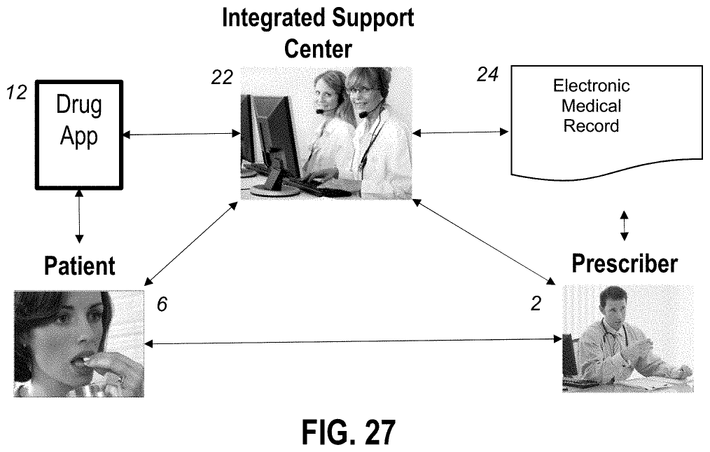

FIG. 27 is an exemplary embodiment illustration of how the Integrated Support Center 22 interfaces with the Drug Specific App 12, the Patient 6, the Prescriber 2, and the Electronic Medical Record 24.

The Integrated Support Center's 22 interactions with the Patient 6 can be instigated by a number of different scenarios and take on many different forms. Examples include but are not limited to: (i) receipt of a Patient 6 alert from the Patient's Drug Specific App 12; (ii) Patient 6 calls; (iii) answering Patient 6 questions about the Drug Dispenser 16, Drug Specific App 12, the drug, or their pain therapy; (iv) Patient 6 counseling within the support center's guidelines; (v) locking the individual patient's Drug Dispenser 16 based upon: (a) a Drug Specific App 12 alert, (b) an Integrated Support Center 22 Analytics alert, (c) a patient 6 conversation, etc.; (vi) unlocking the individual patient's 6 Drug Dispenser 16 based upon: (a) a conversation with the Patient 6, (b) a conversation with the Prescriber 2, etc.

In addition, as an example, the Integrated Support Center 22 provides: (i) “Compliance” and “Adherence” support; (ii) outbound patient 6 telephone calls; (iii) patient 6 monitoring; (iv) emails and/or calls the patient's 6 Prescriber 2 and/or physician to recommend a therapy change, etc.; (v) patient 6 disease management education; (vi) ensures patient 6 has access to their drug; (vii) as required, works with payers to obtain coverage for high cost medications; (viii) looks for prescription financial assistance programs; (ix) patient 6 education and reeducation; (x) patient 6 follow-up, and (xi) Medical Affairs support.

The Integrated Support Center's 22 interactions with the Prescriber 2 can be instigated by a number of different scenarios and take on many different forms. Examples include but are not limited to: (i) locking or unlocking a specific patient's Drug Dispenser 16; (ii) changing the prescription 4; (iii) patient 6 specific physician support using the Integrated Support Center's 22 Data Server's and Analytics 10 to ascertain patient specific treatment alternatives; (iv) assist with patient 6 specific data analysis; (v) provide disease/condition specific information; and (vi) Medical Affairs support, etc.

FIG. 28 is an exemplary embodiment illustration of the assembly and locking mechanism for the Disposable Drug Dispenser's 16 clamshell design. The interior of the top of the clamshell 1020 incorporates hinges that marry with the hinges on the inside of the bottom clamshell interior 1034. These are locked together with a hinge pin 1022 that is threaded through the holes in the respective hinges, much the same as the hinges are held together on most common entry door hinges.

The top 1020 and bottom 1034 clamshells are locked closed together by use of a micro actuator moved locking bar 1028. When the top of the clamshell is closed with the bottom clamshell, the locking bar is pulled down by the micro actuator and the hook's male members dock into the respective female orifices on the locking buttons 1030.

The design incorporates integrated supports to ensure the integrity and durability of the design. They are also instrumental in adding strength, as required, for adding anchors for the respective Disposable Drug Dispenser's 16 internal components.

The design eliminates the ability to open the Disposable Drug Dispenser 16 without an authorized signal to cause the micro actuator to unlock 1028. The Top Cap 1026 is fitted to close the top of the Bottom Clamshell 1034. The top of the Top Cap 1026 covers the top of the Hinge Pin 1022 and holds it in place. The Bottom Cap 1036 covers and provides a holding point for the bottom of the Hinge Pin 1022 and holds it in place.

The right interior to the Top Cap 1026 provides for a dock for the end of the Lock Bar 1028 and allows it to be supported when it moves up and down to lock or unlock, as required. The Bottom Cap 1036 provides the seat that supports the Microacturator 1028 that locks and unlocks the clamshell by moving the Lock Bar 1028 up and down.

The Top 1026 and Bottom 1036 Caps are secured to the Bottom Clamshell Interior 1034 by screws and/or glue that securely marry each of the pieces together from the interior (there are no exterior screws). The unit then forms a ridged bottom clamshell platform 1034 for the Top Clamshell Interior 1020 to dock with. When the Drug Dispenser 16 is closed, it forms a sturdy, tamper resistant housing for the Drug Cassette 18, 18a, 18b, 900.

In one design embodiment, in order to provide the requisite downward pressure to ensure the unit is both water and dust resistant and to contribute to its rugged design, the Drug Dispenser 16 has a Clasp Lock 1032 designed to exert the desired level of pressure on the respective closing clamshell 1020, 1034 joints to secure design integrity.

In this example, the Top Cap 1024 incorporates a one click dispensing button. The Bottom Cap 1038 houses the dispensing port. In the examples presented in FIG. 22, the top and bottom caps are solid enclosures. Dispensing is accomplished by a single click on the combination biometric logon, screen on, and dispensing button on the lower front center of each of the Drug Dispensers 16. Dispensing is accomplished by a drawer that opens out to the left at the bottom of the Drug Dispenser 922, 932, 942 when dispensing is authorized and the dispense button clicked. Dispensing in alternative designs can also be accomplished by dispensing from the bottom of the Drug Dispenser 16 when the one click dispensing button is depressed.

Another example includes the Disposable Drug Dispenser 16. The external case on the Disposable Drug Dispenser 16 is a sealed unit where there is no entry after the Drug Cassette 18 with the specified drug is docked into the Disposable Drug Dispenser 16 and the external case component parts are glued or laser bonded to form a unified case. Drug dispensing is controlled by the Drug Specific App 18. Dispensing takes place by clicking on the dispensing button. The drug is dispensed at the bottom of the Disposable Drug Dispenser 16 or via some kind of catchment area or dispensing drawer. The dispenser opening is covered from inside the Disposable Drug Dispenser 16 by an intrusion deterrent closure once the drug is dispensed. This creates a disposable housing that can only be penetrated via destruction of the dispenser housing which triggers a tampering alert to the Integrated Call Center 22.

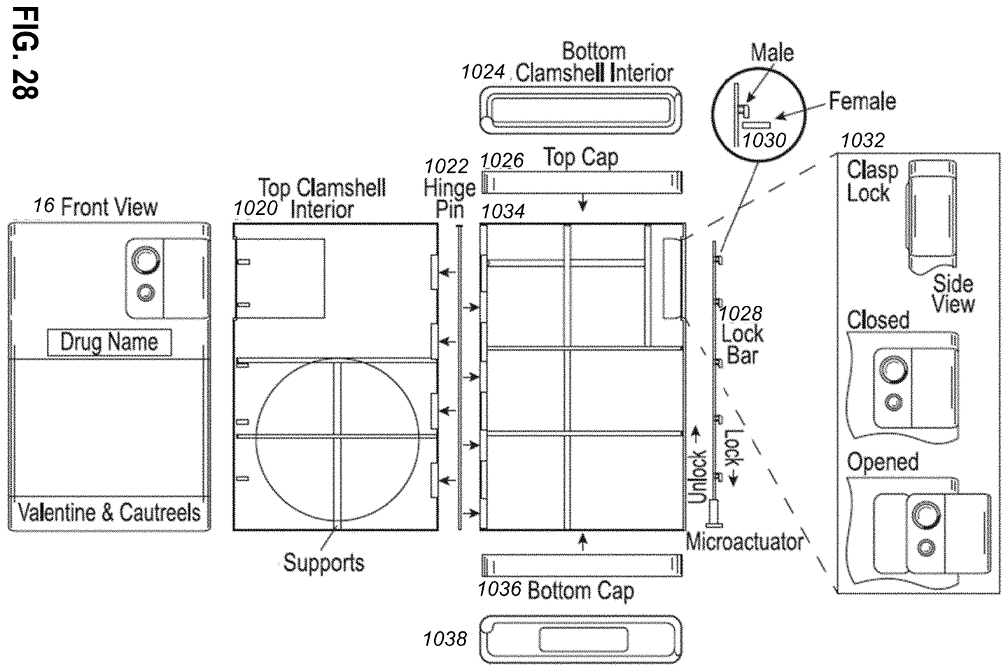

FIG. 29 is an exemplary embodiment of the Drug Dispenser's 16 electronics and features schematic. The Drug Dispenser's 16 system is comprised of an Applications Processor 1048 that contains the units Firmware, individual Drug Dispenser 16 serial number, and manages all functions. The main unit components are the: (i) communications connectivity 1042 module, (ii) its data transfer capability 1046, (iii) the units sensors and/or applications 1044 that allow the unit to authenticate the user, sense efforts to tamper/open the unit without authority, measure drug storage temperature and humidity, to time stamp an action or event (clock function), and locate the unit via GPS; (iv) the display module 1050; (v) the Power Management and recharge system 1056; (vi) Memory management 1054; (vii) Cassette Controller 1040 which enables dispensing as well as the ability to read specific drug cassette information; (viii) the Dosage Dispenser system 1052; and (ix) the various components designed to facilitate and protect the different system functions 1058, etc.

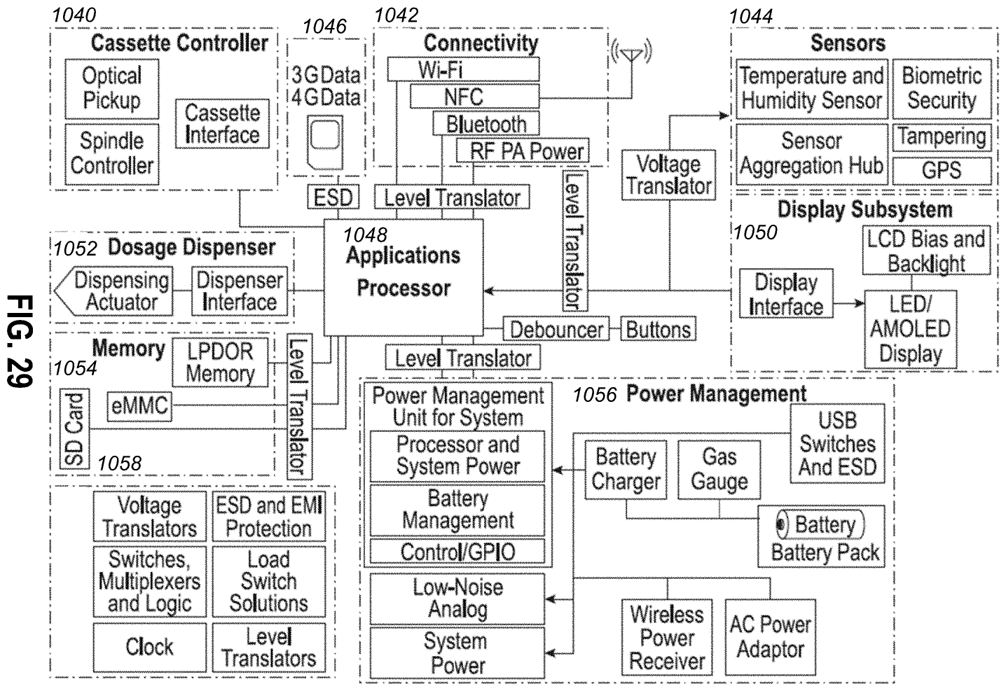

FIG. 30 is an exemplary embodiment of the placement of electronics and mechanical components on the outside and within the Disposable Drug Dispenser 16. The front of the Disposable Drug Dispenser 16 contains an On Off Button 1064 which the user can depress if the Drug Dispenser 16 does not automatically come on when the Drug Specific App 12 handshakes with the Drug Dispenser 16. When a handshake is effectuated or the On Off Button 1064 is pushed, a blue LED light comes on 1066. The LED light 1066 turns to green if the unit is ready to dispense, yellow 1066 if it is awaiting authority to dispense, and red 1066 if the unit is locked and will not dispense. The display 1068 resides on the center of the face, Front View, of the Disposable Drug Dispenser 16.

A number of components fit on the Top Clamshell Interior 1060; these include: (i) the On Off Button 1064 on the front of the Disposable Drug Dispenser 16 and the switch 1070 on the interior top clamshell 1060 (ii) the LED status light 1066 LED and electronics 1074; (iii) the battery, power management, Wi-Fi, Bluetooth, GPS and antenna systems 1072; (iv) the LED Screen 1068 and its electronics and management system 1076; and (v) the drug dispensing actuator arm and dispensing lock 1078. The Bottom Clamshell Interior 1062 houses the: (vi) single click Dispensing Button 1080; (vii) the Logic, Controls, Processor and Memory Board and its various components 1082; (viii) Temperature and Humidity sensors 1084; (iv) the Attempting Tampering Sensors 1090; (x) the Cassette Dispensing Motor and Controller 1088; (xi) the Drug Cassette Reader 1086; (xii) the Clamshell Lock micro actuator controller 1094, and (xiii) the Dispensing Door Controller 1092.

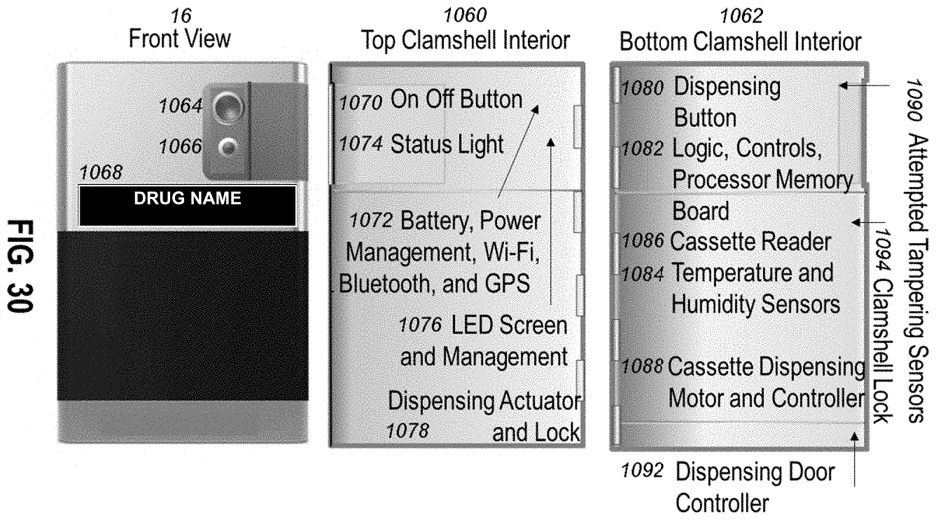

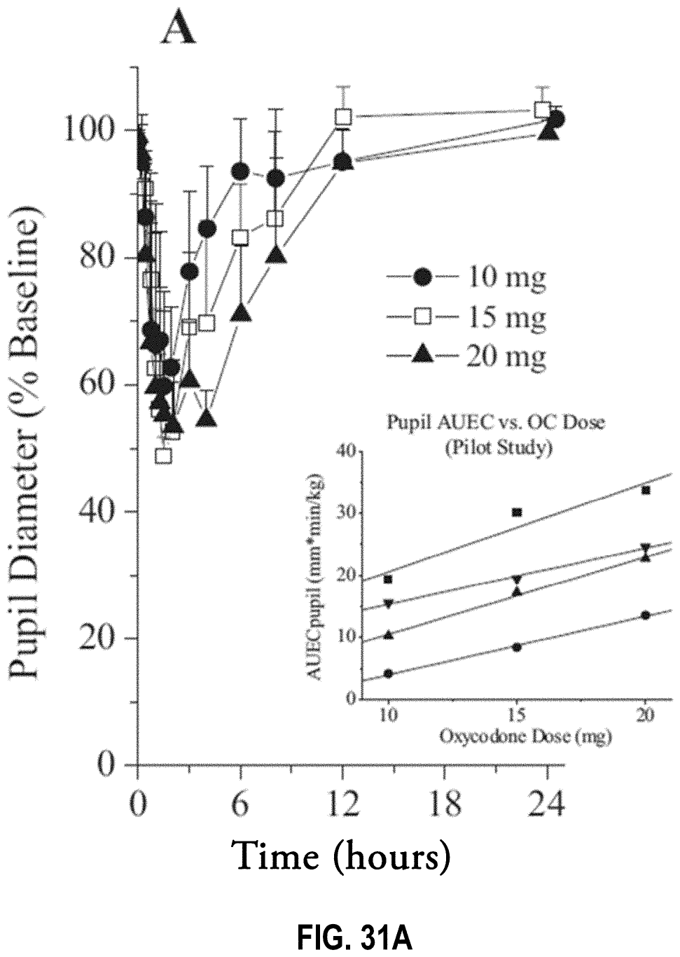

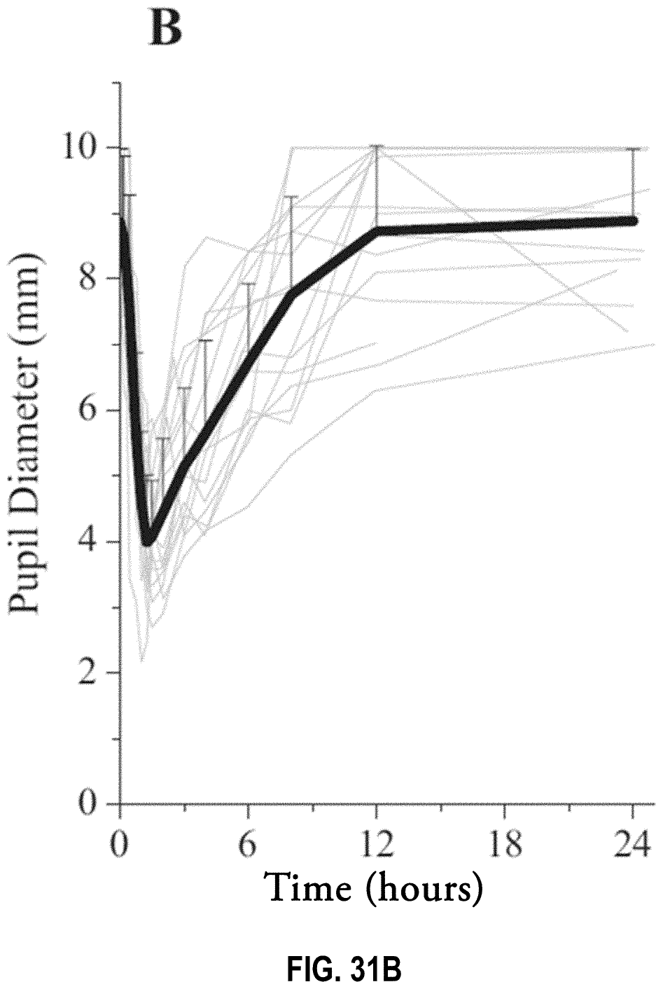

FIG. 31 is an exemplary embodiment of the correlation of opioid plasma levels and pupil size using oxycodone as the example. Each opioid has its own pupil size and plasma level relationship. Pupil size, when normalized for the individual patient, can be an indicator of opioid plasma level. Pinpoint pupils are a sign of opioid overdose but are not pathognomonic (e.g., pontine lesions of hemorrhagic or ischemic origins may produce similar findings). Marked mydriasis rather than miosis may be seen due to hypoxia in opioid overdose situations. So, pupil size can be utilized as a screening indicator for overdose.

As an example, the relationship between the plasma level of oxycodone and the analgesic response will depend on the patient's age, state of health, medical condition, extent of previous opioid treatment, and concomitant medications which may affect pupil dilation. The minimum effective plasma concentration of oxycodone to achieve analgesia will vary widely among patients, especially among patients who have been previously treated with potent agonist opioids. Thus, patients need to be treated with individualized titration of oxycodone dosage to the desired effect. The minimum effective analgesic concentration of the oxycodone for any individual patient may increase with repeated dosing due to an increase in pain and/or development of tolerance.

A 1100 depicts the time course of pupil diameter over 24-hour period in 4 subjects receiving 10-, 15-, and 20-mg oral dose of oxycodone (pilot study). The inset 1102 depicts the linearity of the area under the effect curve (AUEC) for pupil response across the 3 doses in individual subjects. The indication is that there is a maximum percent (%) change from baseline in pupil diameter within time ranges after an oxycodone dose (applies to all opioids).

B 1104 presents the individual time course of pupil diameter in 16 subjects after 15-mg dose of oxycodone (gray lines) in both pilot and main studies. This shows the range differences between individuals. It illustrates that the pupil does not move into “pinpoint pupils”, overdose, or mydriasis under normal dosing. The black line represents the mean pupil diameter over time. Thus, a personalized Patient 6 baseline can be used as a basis for future pupil size comparison to ascertain if the Patient 6 is: (i) overdosed, (ii) could become overdosed if they take another opioid dose, and (iii) within normal range and should be allowed to take the opioid dose.

FIG. 32 is an exemplary embodiment of a how pinpoint and dilated pupils can be used as diagnostic indicators. Oxycodone pharmacokinetic-pharmacodynamic model prediction of pupil diameter (lines) and observed data (squares) illustrated in 4 representative subjects 1106, 1008, 1110, 1112 receiving 15 mg oxycodone illustrate the diagnostic accuracy. The four patients illustrate the oxycodone goodness of fit with the parent drug equilibration model to the observed data. The high correlation was also tested on multidose data. The results were not significantly different from the estimates obtained with the 15-mg dose.

Using the images captured by Iris scan biometric logon or Drug Specific App 12 logon to measure pupil size enable setting a pupil diameter opioid plasma level curve over time baseline against which future pupil size measurements can be compared. Iris scan are being incorporated into smartphones by Samsung, Apple, Microsoft, LG, HP, Fujitsu, Vivo, ZTE, Alcatel, UMI, etc. Using the iris scan to measure the pupil ensures the patient's pupil is the one being measured and provides further security against opioid diversion.

The Patient's 6 pharmacokinetic-pharmacodynamic opioid baseline curve can be established by taking pupil size measurements before taking the first opioid dose and then at specified time intervals during the Drug Specific App 12 set up process. Thereafter, any statistically significant indicator that there is a shift from the baseline can require a confirmatory set of pupil measurements and/or alert the Integrated Support Center 22.

Once personalized (required by individual differences as well as concomitant medications), the algorithm can use pupil diameters below or above a certain pupil diameter to preclude dispensing the dose. The use of the predictive curve and the effects of subsequent doses to ascertain if the patient would remain within the safe range can be used to allow the dose to be dispensed. If the range falls between the safe to dispense range versus the do not dispense value, then a confirmatory test can be utilized to educate the dispense or do not dispense the dose decision, regardless if the prescription would otherwise allow the dose. (The integrated support center would have access to this information to help educate their decision.) Doses below or above a certain pupil diameter would not be dispensed.

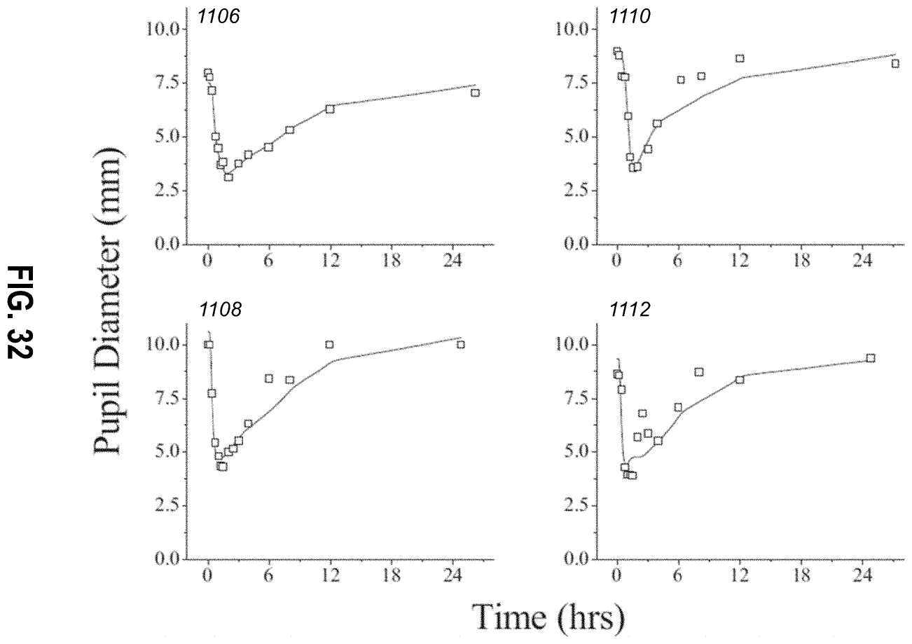

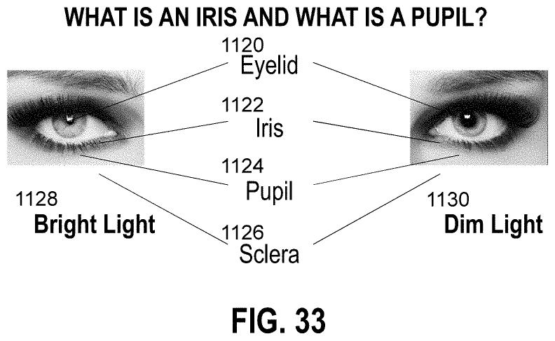

FIG. 33 illustrates an exemplary embodiment of the present invention that explains what an eyelid 1120, iris 1122, pupil 1124 and sclera 1126 of the eye are. It shows the difference in eye dilation under bright light 1128 and dim light 1130.

FIG. 34 is an exemplary embodiment of a method to detect, authenticate, and capture iris and pupil data to make a dispense or do not dispense screening decision.

The Eye Image Capture 1140 is accomplished, for example, by an IR (infrared) LED and a dedicated iris camera resident on the smart phone. The camera is designed with a special image filter which receives and recognizes the reflected picture of the irises with a red IR LED light.

The setup process basically involves lifting the phone to eye level at arm's length and waiting for the phone to capture your iris data. It's able to detect which part of the image is your iris, then deletes the rest of the information, like your eyelid, pupil and sclera (white area). Once the iris is registered, the phone stores the iris data as an encrypted piece of code in the Databases 1156. Thereafter, when the user tries to access the phone, or a drug specific app which incorporates the dispense or do not dispense pupil algorithm, the LED and camera work together to capture the iris, then extract the file and compare the pattern with the code to allow access 1158.

Once the Image of the eye is Captured 1142, then the program Detects the Iris and Eyelid 1144, and then extracts the Iris Area 1146. The program then removes any eyelid artifacts 1148 and then normalizes the Iris 1150. Then the pupil detection program normalizes the Pupil Areas 1150, 1152.

Normalization uses the size of the Iris versus the baseline of the Pupil stored in the Databases 1156. This allows adjustment of the pupil size for the difference in distance from the camera to the Iris 1140. The digital brightness values enable the brightness adjustment in order to correlate the pupil size to the brightness and normalize the pupil size against the baseline.

The normalized data is then translated into a format that may be utilized by the Authenticated 1158 patient specific program to compare the values to the established normalized patient specific miosis, and mydriasis data ranges to make a dispense or do not dispense decision 1160.

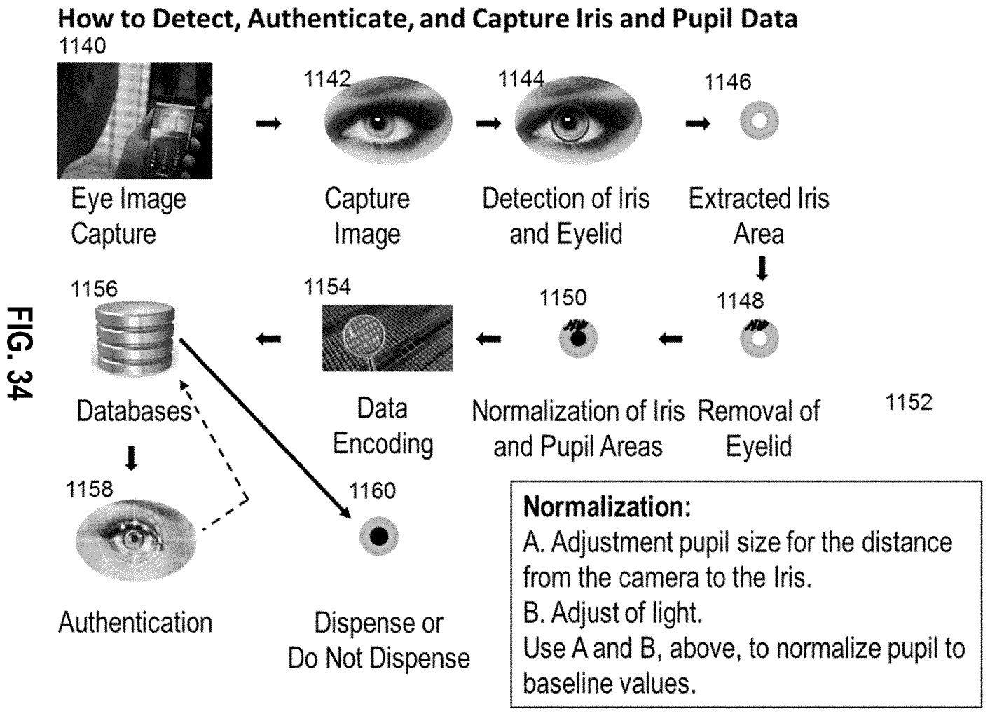

FIG. 35 is an exemplary embodiment of pupil size logic utilized to make a dispense or do not dispense decision. The process begins with a smart phone Iris Scan 1160 used to authenticate 1162 the right patient is accessing the system. If the patient is not authenticated, No 1164, he/she is required to try again 1160 until he/she is authenticated, Yes 1166. The program then accesses the normalized pupil measurement 1168 and ascertains if the pupil is larger than, for example, 8 mm. If Yes 1172, then the decision is Do Not Dispense 1174. If the pupil is smaller than 8 mm, No 1176, then the algorithm ascertains if the pupil is less than, for example 2.5 mm 1178. If it is not smaller than 2.5 mm, No 1180, then the decision is to Dispense 1182. However, if the pupils are less than 2.5 mm, Yes 1184, then the algorithm checks if the pupil size is less than 2.0 mm 1186, if Yes 1188, then the decision is Do Not Dispense 1174. If the pupil is not less than, for example, 2.0 mm, No 1190, then the algorithm checks the patient's pupil size versus the patient's traditional pupil sizes in order to ascertain if this is within historic norms or if there has been a rapid decrease in pupil size versus historic pupil size values 1192. If Yes 1194, then the decision is Do Not Dispense 1174. If the answer is No 1196 there has not been a rapid decrease in pupil size versus historic norms (some of the historic norms could be derived from iris scan data that is used by the patient to access his/her smart phone, etc., over time), the program confirms, for example, that the pupil size is between 2.0 mm and 2.5 mm 1198. If Yes 1200, then the program would proceed to a confirmatory test 1202 designed to decrease the chance of a false positive, i.e., a false decision not to dispense. In this exemplary, we will use a Motor Skills Self-Test. However, other values tied to a drug mediated physiologic or psychologic change can be utilized. Examples include things such as pupil reaction to light/pupil reactivity, pulse rate, blood pressure, heart rhythm, body temperature, amount of sleep, gait, balance, speech pattern, tone, eye movement, a specific diagnostic value, CO₂ saturation, respiratory rate, etc. Some may be digitally captured (passively monitored) by a device like a wearable monitor (e.g., FitBit), implanted monitor, wearable diagnostic, consumed diagnostic, smart watch, smart phone, etc. that would not require an elicited response from the patient.

Another basis for making a dispense versus do not dispense decision is the standard deviation from the Patient's 6 pharmacokinetic-pharmacodynamic opioid baseline curve.

The value used to ascertain whether an Oxycodone dose should or should not be dispensed may be based upon a change in normalized pupil size versus an established pupil size that is equal to or greater than a defined amount. It may also be a combination of a fixed number, e.g., for mydriasis and/or pinpoint pupils as well as a delta change from the baseline, whichever applies. The objective is to keep the Patient 6 from taking a drug dose that would result in an overdose.

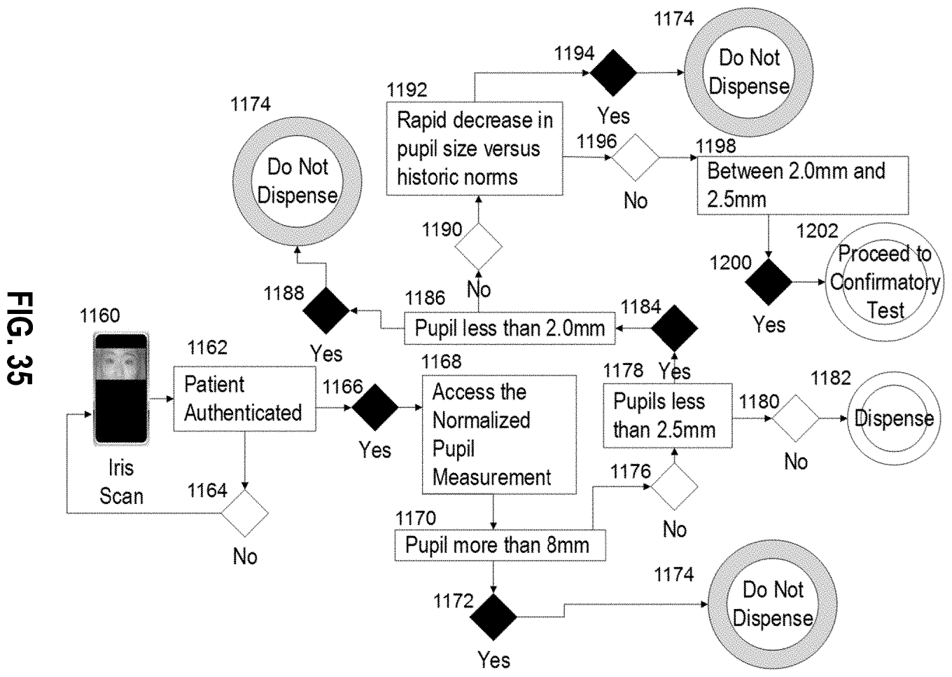

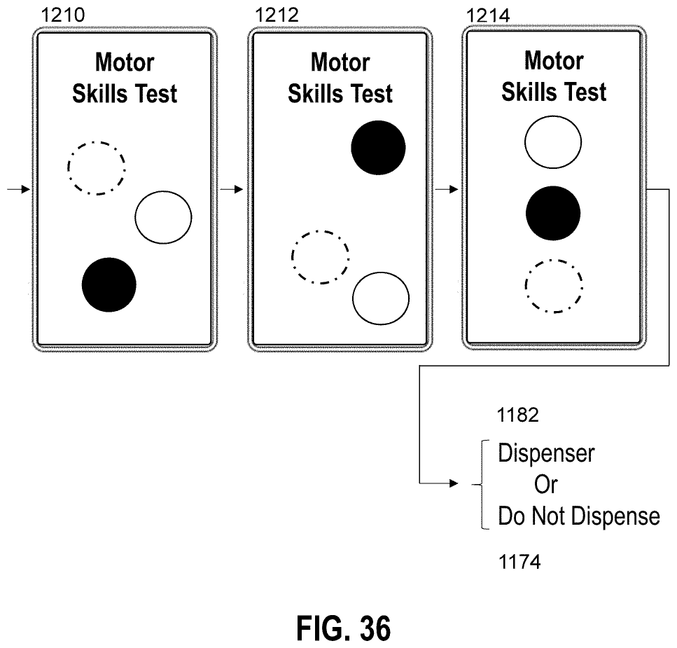

FIG. 36 is an exemplary embodiment of a three screen 1210, 1212, 1214 motor self-test. Each screen is comprised of three dots which are randomly placed on the screen. Each is a different randomized color. One randomized dot is solid and designated as the dot that must be clicked upon. The titles on each screen also change positions and colors randomly. Based on the program logic, a decision is made whether or not the patient's cognition, based upon the motor skills self-test, is sufficient to Dispense 1182 the drug dose or to preclude the drug from being dispensed, Do-Not-Dispense 1174. The logic behind the Motor Skills Self-Test is to measure response time and accuracy from baseline test values to ascertain if there is a sufficient deterioration in response time and accuracy to warrant a Do-Not Dispense 1174 decision.

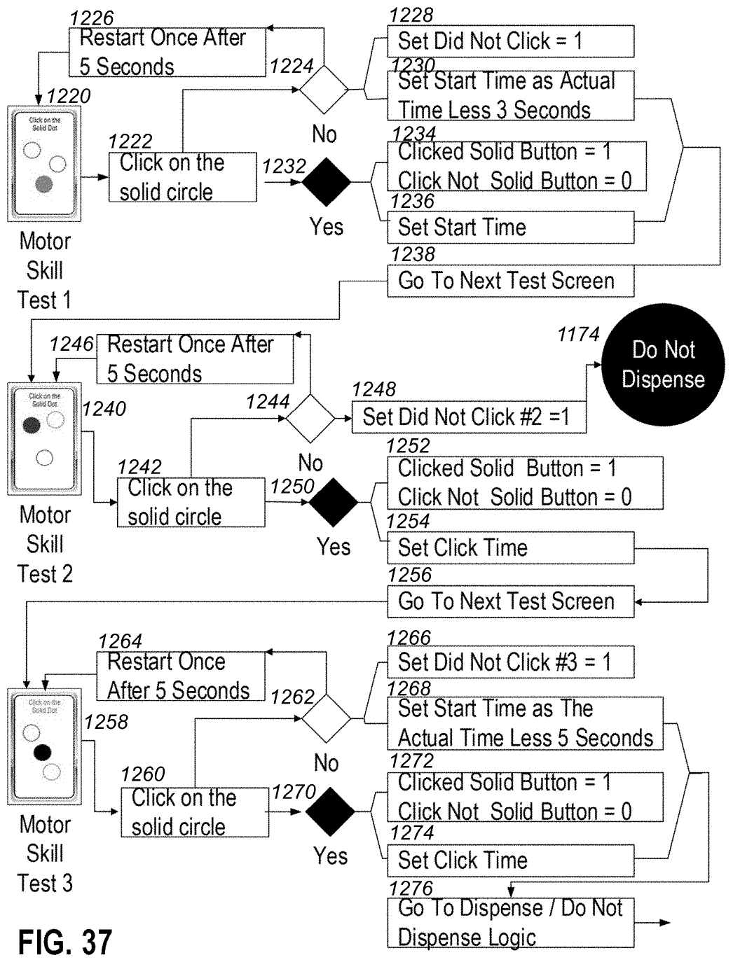

FIG. 37 is an exemplary embodiment of the Motor Skill Self-Test logic to make a dispense or do not dispense a drug dose decision. When the first Motor Skill Test 1 screen 1220 is presented, it asks the Patient 6 to click on the solid dot 1222. If no response is made, No 1224, after five seconds 1226, the screen for Motor Skill Test 1 1220 is refreshed with new button colors and locations and the test start time is reset to zero. If at the second attempt there is still no response after five seconds, No. 1224, then the program sets the did not click value to =1 1228 and sets the start time as the actual time less 3 seconds 1230 and proceeds to the next screen 1238. If the Patient 6 clicked on the screen or the solid circle 1222, Yes 1232, the program indicates the patient clicked on the solid button=1 1234 or did not click on the solid button=0 1234. Then the program sets the start time to the actual time 1236 and then proceeds to the next test screen 1238.

When the Motor Skill Test 2 screen 1240 is presented, it asks the Patient 6 to click on the solid dot 1242. If no response is made, No 1244, after five seconds 1246, the screen for Motor Skill Test 2 1240 is refreshed with new button colors and locations and the test start time for Skill Test 2 is reset once to zero. If at the second attempt there is still no response after five seconds, No. 1244, then the program sets the did not click value to =1 1248, and makes the Do Not Dispense the drug dose 1174 decision and stops the algorithm. If the Patient 6 clicked on the screen or the solid circle 1242, Yes 1250, the program indicates the patient clicked on the solid button=1 1252 or did not click on the solid button=0 1254. Then the program sets the start time to the actual time 1254 and then proceeds to the next test screen 1256.

When the Motor Skill Test 3 screen 1258 is presented, it asks the Patient 6 to click on the solid dot 1260. If no response is made, No 1262, after five seconds 1264, the screen for Motor Skill Test 3 1258 is refreshed with new button colors and locations and the test start time for Skill Test 3 is reset once back to zero. If at the second attempt there is still no response after five seconds, No. 1262, then the program sets the did not click value to =1 1266, and sets the Skill Test 3 start time as the actual time less 5 seconds 1268. If the patient clicked on the screen or the solid circle 1260, Yes 1270, the program indicates the patient clicked on the solid button=1 1272 or did not click on the solid button=0 1272. Then the program sets the click time to the actual time 1274 and then proceeds to the Dispense/Do Not Dispense logic 1276.

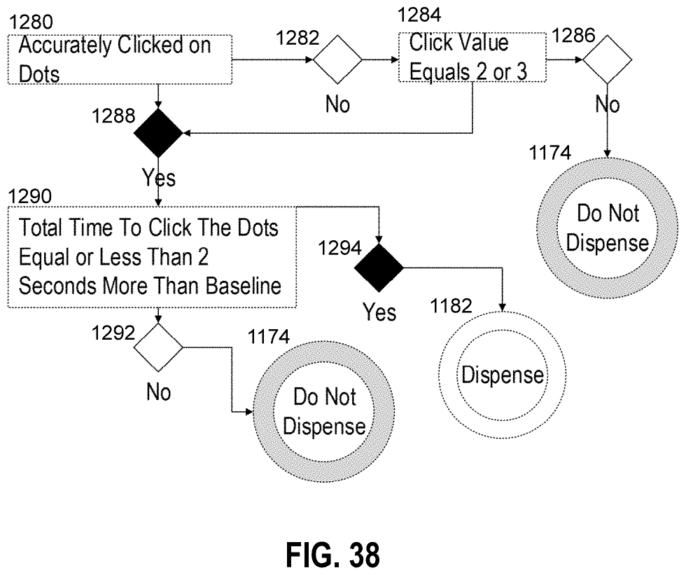

FIG. 38 is an exemplary embodiment of the dispense or do not dispense a drug dose motor skill test logic. The logic begins with the question if the patient accurately clicked on the solid dots, total=3 1280. If the answer is No 1282, then the formula checks to see if the total clicked value equals 2 or 3 1284. If No 1286, then the decision is Do Not Dispense the drug dose 1174.

If the Patient 6 accurately clicked on the solid dots 1280, Yes 1288, if the total time to click on the solid dots, for example, equals or is less than 2 seconds more than the baseline test and/or series of test responses 1290 (e.g., time less baseline must equal less than 2 seconds), Yes 1294, then the decision is to Dispense 1182 the drug dose. If the answer is No 1292, then the decision is Do Not Dispense 1174 the drug dose.

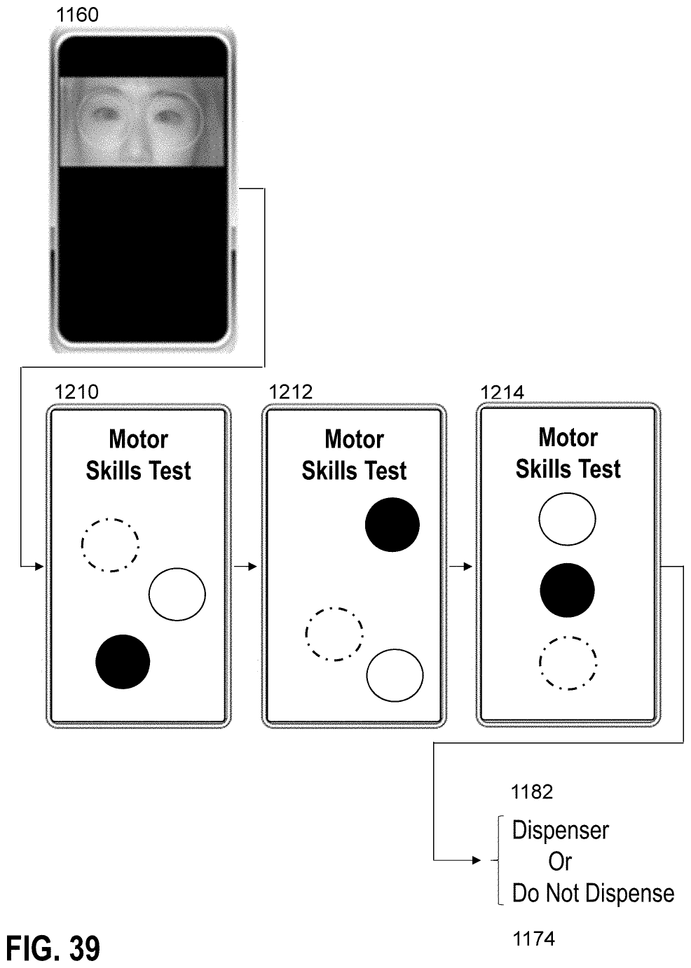

FIG. 39 is an exemplary embodiment of the opioid dispensing algorithm designed to reduce the number of false positives (e.g., decisions not to dispense). In this case, if the pupil scan decision is to dispense 1182, the decision is then to dispense the opioid dose 1182.

However, if the result of the pupil scan 1160 was to proceed to a confirmatory test 1202, in this case the Motor Skill Self-Test 1210, 1212, 1214, then the outcome of the confirmatory self-test will either be to Dispense 1182 or Do Not Dispense 1174 the opioid dose.

There can be more than one confirmatory test. As an example, opioid confirmatory tests could include one or more digitally captured diagnostics and/or biomarkers such as pupil reactivity, respiratory rate, oxygen saturation level, change in body temperature, activity level, amount of sleep/naps, heart rate, blood pressure, gait, dizziness, constipation, changes in weight, etc. They could also include certain physiological and/or psychological self-assessments, self-tests, or self-reported information/observations, etc.

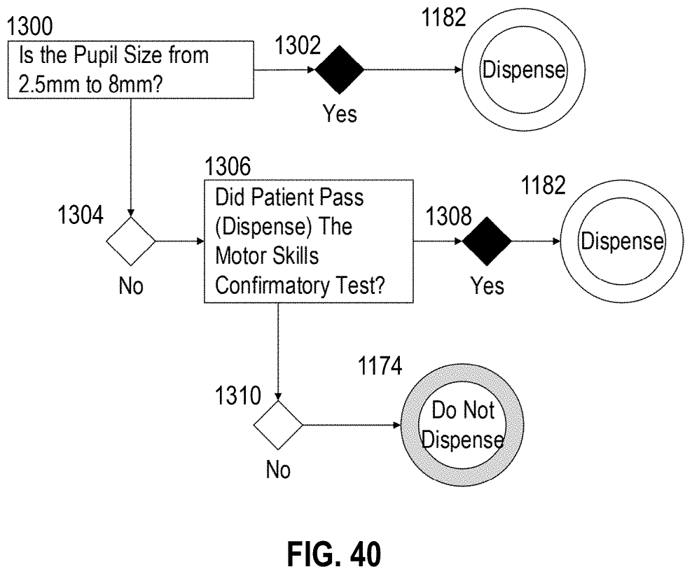

FIG. 40 is an exemplary embodiment of algorithm logic designed to reduce the number of false positives (e.g., do not dispense). It begins by ascertaining if the pupil diameter is within the normal range of, for example, from 2.5 mm to 8 mm 1300. If Yes 1302, then the program indicates that the medication should be dispensed 1182. If the answer is No 1304, then the program proceeds to ascertain if the patient passed the confirmatory test, in this case The Motor Skills Confirmatory Test 1306. If the patient passed the confirmatory test, Yes 1308, then the program indicates that the opioid dose should be Dispensed 1182. If the answer is No 1310, then the program indicates the opioid dose should not be dispensed 1174.

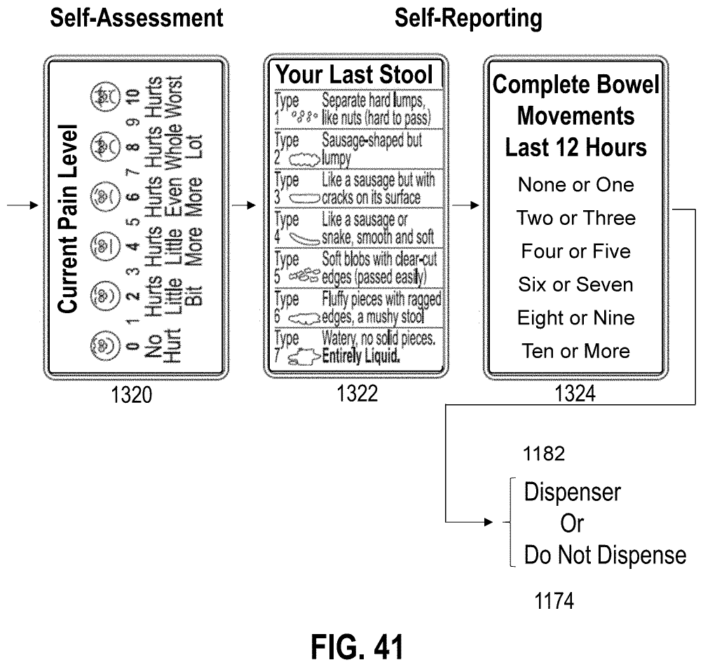

FIG. 41 is an exemplary embodiment of using patient self-assessment and self-reporting information to make a Dispense 1182 versus a Do Not Dispense 1174 the drug dose decision. As an example, if the Patient 6 is taking a drug, the patient may want to avoid taking more medication if the Patient 6 is or is becoming severely constipated. In this case, for example, a combination of a self-assessment of the patient's abdominal pain level 1320, and self-reported consistency of the patient's last stool 1322, and the number of bowel movements 1324, would allow an informed Dispense 1182 or Do Not Dispense 1174 the drug dose decision, even if the Prescription 4 would otherwise allow the medication to be dispensed before talking with a medical professional. Under this embodiment this would be before talking with the Integrated Support Center 22. This routine can also be utilized to alert the Patient 6 that he/she may be getting constipated and that they should consider taking a laxative or talking with their Prescriber 2, etc.

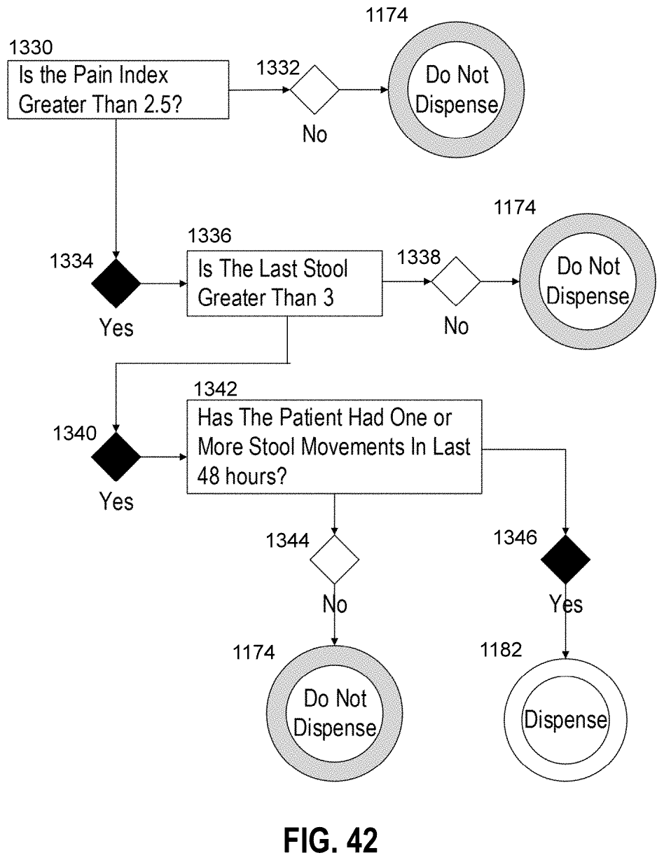

FIG. 42 is an exemplary embodiment of the program routines that can utilize patient self-assessment and self-reported information to make a dispense or do not dispense decision. To make a dispense 1182 or do not dispense 1174 the drug dose decision the program would ascertain if the pain level is greater than a predefined level, for example level 2 1330; if No 1332, then the decision is Do Not Dispense 1174. If the pain level is greater than 2 1330, Yes 1334, then the program proceeds to ascertain if the stool composition of the last bowel movement was greater than 3 1336; if No 1338, then the decision is Do Not Dispense 1174. If the last stool consistency was greater than 3 1336, Yes 1340, then the program ascertains if the patient had one or more stool movements in the last 48 hours 1342; if No 1334, then the decision is Do Not Dispense 1174. If the answer is Yes 1346, then the decision would be to Dispense 1182 the drug dose.

### IV. Examples

The embodiment of the invention can be utilized, for among other uses, 1) to improve the drug's safety profile by ensuring proper, personalized drug prescribing and prescription management (e.g., personalized dispensing), 2) to ensure the medication is efficacious, 3) as a diagnostic aid/tool, 4) to titrate the medication, 5) to preclude drug mediated adverse events, 6) to preclude over dosing, 7) to preclude under dosing, 8) to decrease the chance of misuse, 9) to decrease the chance of abuse, 10) to decrease the chance of overdosing, 11) to increase compliance with the medication prescription, 12) to prevent the patient from inadvertently taking a duplicate dose, 13) to decrease the chance of addiction, 14) to decrease the chance of dependence, 15) to better manage drug tolerance, 16) to manage medication withdrawal, 17) to enable PRN dosing, e.g., manage “Patient Controlled Oral Analgesia™” (PCOA™), 18) to preclude drug divergence, 19) to guard against accidental ingestion of the drug by a child, 20) to avoid drug-drug interactions, 21) to better manage medication mediated side-effects, 22) to promote prescription persistence, 23) to shift any potential liability from the drug manufacturer or the drug Prescriber 2 to the Patient 6, 24) to capture patient self-assessment, self-test, self-reported, and digitally captured information required to control drug dispensing and/or to meet clinical trial regulatory agency reporting requirements, 25) to capture drug related side-effects to assist in better patient management, 26) to streamline drug Risk Evaluation and Mitigation Strategy (REMS) reporting, 27) to streamline Prescription Drug Monitoring Programs (PDMPs) record keeping and reporting, etc. In short, to personalize drug therapy by improving the drug's drug/safety profile.

Ideal drug management would have the physician evaluate the patient each time prior to allowing the patient to take his prescribed drug dose/medication. The physician would ascertain how well the current dose manages the patient's pain/symptoms and would be looking for signs of drug side effects, addiction, dependence, misuse, abuse, possible bad drug-drug combinations, etc. prior to authorizing the patient to take the dose. That way, the physician could change/refine the dosage for the patient and/or preclude the patient from taking the drug in the event the physician ascertained that the dose was not warranted and/or that the dose could lead to a serious drug mediated event and/or undesirable side effect. Having a physician approve each dose before it is taken by the Patient 6 is neither realistic nor cost effective, the embodiment of the invention incorporates many of the consideration a physician would consider prior to allowing a patient to dispense a drug dose from the Drug Dispenser 16.

### A. Example: Opioids

Opioids serve as an excellent exemplary of how the embodiment of the invention can be utilized to improve drug and other medications management and patient outcomes. A person of ordinary skill in the art, however, will appreciate that the drug plus device combination products disclosed herein are not limited to opioids, and that the concepts and functionality described in the following example with respect to opioids may be readily applied to other drugs.

### B. Background

The following describes some, but not all, of the key opioid side effects as background for the subsequent opioid examples of the embodiment:

Cognitive Impairment—It is well known that larger doses of opioids are markedly impairing, leading to drowsiness, lethargy, and even death. At least one prospective study has demonstrated that those with chronic pain on opioid therapy have cognitive deficits including reduced spatial memory capacity and impaired performance in working memory assessment (Schiltenwolf et al., 2014).

Respiratory depression—Opioids adversely affect the respiratory system. Carbon dioxide (CO₂) levels in the blood stimulate our respiratory drive. As breathing slows down, CO₂ levels increase, which stimulates the brainstem to increase the respiratory rate.

Low oxygen levels do not stimulate breathing so sensitivity to CO₂ levels is an important function of nerve cells in the brainstem. Opioids block that feedback loop. When an individual overdoses on a drug, the high levels of opioid will decrease alertness and induce sleep. During sleep, it is the CO₂ feedback loop that keeps people breathing. However, when blocked by the high levels of a drug, breathing slows or stops and the person who has overdosed literally suffocates.

Heart Rate—Heart rate may become either rapid or very slow. Some opioid users may also develop postural hypotension or a severe fall in blood pressure on standing up from a sitting or lying position.

This is also problematic for individuals with lung disease or sleep apnea. People with chronic lung disease often need elevated carbon dioxide levels to stimulate them to breathe more deeply. Taking opioids will blunt this response, causing people with lung disease to breathe slower and therefore have low oxygen levels.

Sleep apnea is similar as people periodically stop breathing at night until their carbon dioxide levels get high enough to stimulate their brain to signal them to gasp for breath. When opioids interfere with this response the effect can be life threatening. Opioids have been shown to worsen the apnea episodes in those with sleep apnea (Jungquist, Flannery, Perlis, & Grace, 2012).

Miosis—Opioid use results in the formation of small, constricted pupils, similar to how pupils respond to bright light.

Constipation—Opioids cause sluggish peristaltic movements in the digestive tract. This causes stasis or loss of movement of the intestinal contents and leads to severe constipation, especially in the case of long-term use.

Drowsiness or Sedation—Opioids, and in particular morphine, are known to cause severe sedation and drowsiness.

Myoclonus—High doses of opioids can result in muscle rigidity and abnormal movement of the limbs and muscles.

Hyperalgesia—Opioid-induced hyperalgesia (OIH) is another side effect of the use of opioids. Opioid hyperalgesia is a phenomenon where the body develops an increased sensitivity to pain secondary to opioid use (hyper—over or excess, algesia—sensitivity to pain).

Pain is an important part of our body's defense system, warning us of current or impending damage or injury. As opioids decrease our brain's sensitivity to pain signals coming from the rest of the body, our brain begins to compensate by increasing our recognition of and sensitivity to pain. The pain neurons going to the brain actually change to make them more responsive to pain and increase our perception of pain. This change is called neuroplasticity of the nerve cell. Many mechanisms are believed to be involved in these changes (M. Lee, Silverman, Hansen, Patel, & Manchikanti, 2011). The result of this change is that after opioid levels decrease, our pain fibers are more sensitive than before consuming the opioid which results in an increase in pain.

Unfortunately, increasing pain can also mean disease progression or the development of tolerance to the current opioid dose. For these conditions, opioid doses are usually increased. In contrast, the treatment of opioid hyperalgesia is the decrease or discontinuation of opioids.

Tolerance and withdrawal—Tolerance occurs when the body has developed physiologic (both neuroplastic and chemical) changes that result in decreased effectiveness of the medicine necessitating a higher dose to get the same effect. Withdrawal is the unpleasant symptoms that develop upon decrease or discontinuation of that medicine. Brain changes and measurable withdrawal symptoms can occur after one dose of opioid (Rothwell, Thomas, & Gewirtz, 2012).

In clinical practice, withdrawal symptoms can occur after five to seven days of opioid medication (Anand et al., 2010). Withdrawal symptoms may consist of myalgia (muscle pain), chills, sweats, anxiety, increased pain, rapid heartbeat, dilated pupils, yawning, diarrhea and nausea.

Withdrawal is extremely unpleasant and can be relieved by taking another opioid dose. Tolerance and withdrawal are not considered addiction. Tolerance occurs because of the physiologic changes resulting from exposure to opioids. Withdrawal is the unpleasant physical and emotional symptoms that occur upon withdrawal of the opioid after tolerance has developed.

Addiction—Addiction is characterized by inability to consistently abstain, impairment in behavioral control, craving, diminished recognition of significant problems with one's behaviors and interpersonal relationships, and a dysfunctional emotional response.

Death—There is one factor that is often not considered when prescribing opioid pain medications—the overall increase in deaths. Opioids account for more deaths than any other medication. The medical ethical principle of non-maleficence (do not harm) now extends beyond the exam room: the interaction between a physician and his/her patient may adversely affect the life and health of someone else who is not a part of the medical decision. Impairment from opioids may lead to unintentional death by motor vehicle crashes or workplace safety incidents. Opioid use may also lead to falls and increase mortality rates in the elderly.

Benzodiazepines—It is also important to realize that the risk of death is markedly increased when opioids are taken with benzodiazepine medications. The combination of opioids and benzodiazepines are the leading cause of overdose deaths when multiple medications are involved (Calcaterra, Glanz, & Binswanger, 2013). Despite this danger, benzodiazepines are prescribed to about 30% of people on chronic opioid therapy (Nowak, Abou-Nader, & Stettin, 2014).

### C. Drug Prescribing

Prescribing

The Drug Specific App 12 is designed to be customizable for each Patient 6. When the Prescriber 2 enters the prescription 4, he/she may select from a list which includes all the potential drug side effects and/or any information that is required by a regulatory agency, such as the FDA or EMA, for a clinical trial and/or for drug approval. The Prescriber 2 can also choose how often the patient will be prompted for each piece of information, e.g., every time before dispensing the drug, at specified intervals, when another value occurs, when another value occurs over or under a specified value or within a specified range, etc. These values may include digitally captured or patient entered physiologic, psychologic, lifestyle, concomitant medications taken since the last drug dose, and/or environmental information. The captured information may or may not be utilized by the Drug Specific Dispensing Algorithm 13 to make a dispense or do not dispense the dose decision.

The values are stored by the Drug Specific App 12 on its related Interface Database 278 files resident on the Interface Device 14, or on the Standalone Drug Dispenser 16, and synchronized with the Patient Database 152 stored on the Data Servers 10 for future reference. The information is organized for utilization by the Patient 6, the Drug Specific Dispensing Algorithm 13, care givers, the Patient's 6 physician and/or Prescriber 2 to better manage drug dosing and/or the Patient's treatment/therapy, and for trending and analysis by the Drug Specific App 12 and/or the analytics programs on the Data Servers 10 to assist in the development of optimal therapies.

Creating the Personalized Drug Dispensing App

Once the prescription is electronically entered, submitted 150, the App Generation Program 200 on the Data Servers 10 creates a personalized Patient 6 Drug Specific App 12 and creates and links together the related Patient 6 records/databases resident on the Data Servers 10. Thereafter, it sends a download link to the Patient 6, with copies to appropriate personnel, e.g., the caregiver, Prescriber 2 and the Pharmacy 8, etc., for the Patient 6 to download the Drug Specific App 12 onto the Interface Device 14 or Devices 14 or onto a standalone Disposable Drug Dispenser 16. If the Patient 6 has any problem downloading the Drug Specific App 12, the caregiver, the Prescriber 2 or her/his staff, the Integrated Support Center 22 and/or the Pharmacy 8 staff can assist the Patient 6 in resolving the problem.

Personalizing the Patient Specific Drug Specific App

Upon downloading the personalized Drug Specific App 12, the Patient 6 opens the Drug Specific App 12 and inputs the requisite Patient 6 information which enables the biometric login 280, links the Patient 6 to the Prescription 4 and to the Drug Specific App 12.

Loading the Drug Cassette into the Reusable Drug Dispenser

In the Reusable Drug Dispensers 16, opening the Drug Dispenser 16 requires a unique Dispenser Identification No. 158 which is automatically issued by the Patient Database 152 App Generation Program 200 when an e-Prescription 4 is Submitted 150. An Authorization Code (also referred to a s a Dispenser Opening Code 158) is required to open the Reusable Drug Dispenser 16 to load restricted medications. In one embodiment, a USB cable is required to open the Reusable Drug Dispenser 16. When a USB cable is plugged in to power the locking and unlocking of the Reusable Drug Dispenser 16 Microactuator 1028 locking mechanism is activated (alternatively, the power can beprovided by the battery in the Reusable Drug Dispenser negating the need for the USB cable; this operation is incorporated by reference in the Loading the Drug Cassette Discussion.).

Where the dispenser is combined with a drug that is not subject to medical concerns such as divergence or misuse/abuse (such as opioids), then an Authorization Code is provided to the Patient 6 by a provider for the Patient to be able to load an initial, a refill or replacement Drug Cassette 18 into the Reusable Drug Dispenser 16. Since the Patient 6 can load the Drug Cassette 18 into the Reusable Drug Dispenser 16, dispensing only requires that the person dispensing the medication select the box containing the medication filled Drug Cassette 18 and thereafter follow its normal dispensing process.

However, if the medication is designated as a medication which is subject to medical concerns such as divergence or misuse/abuse, such as opioids, then the issued Authorization Code restricts opening of the Drug Dispenser 16 to a healthcare professional authorized to handle such drug prescriptions in order to load a replacement Drug Cassette 18, generally at the Pharmacy 8. The Drug Dispenser 16 will only open if the required matching Authorization Code entered by the pharmacy program or the healthcare professional matches the code in the firmware of the Reusable Drug Dispenser 16.

When a USB cable is plugged in to the Reusable Drug Dispenser 16 Microactuator 1028, the Authorization Code must be entered to open the Reusable Drug Dispenser 16.

Once opened, the individual loads the Drug Cassette 18 containing the drug into the Reusable Drug Dispenser 16 and then checks to ensure that the Drug Specific App 12 recognizes the Reusable Drug Dispenser 16 and that the Reusable Drug Dispenser 16 is ready to dispense the medication. If it is a new prescription, the Drug Specific App 12 is immediately ready to dispense the medication with the authorized Biometric Authentication 850 and Patient 6 response information 862, 864, 866. If it is a refill for an existing drug, the Drug Specific App 12 will authorize dispensing the medication per the prescription upon Biometric Authentication 850, input of the requisite information, and checking to ensure the prescribed drug has not been previously dispensed using a different Drug Dispenser 16. This eliminates duplicate dosing of an authorized drug dose. Additionally or alternatively, the Drug Specific App 12 may authorize dispensing only after ascertaining that all prior Drug Cassette 18 doses have been dispensed, or after locking another Drug Cassette 18 to preclude further dispensing while the current Drug Cassette 18 is chosen to dispense the medication.

If for any reason the Drug Specific App 12 does not recognize the Drug Dispenser 16 or the drug is not the specified drug, the Drug Specific App 12 will show an alert and present the issue so that it may be resolved. A click on the Alert Screen on the interface device 14 connects the individual with the Integrated Support Center 22. For Standalone Drug Dispensers 16 that do not have a phone capability, the advisory presents the number to call to obtain support.

When the Drug Specific App 12 recognizes the Reusable Drug Dispenser 16 and the drug in the Drug Cassette 18 as being a refill for a prescribed drug, the Drug Specific App 12 checks to see if the medication in the first drug cassette was fully dispensed. If it has not been fully dispensed, then the medical professional loading the Drug Cassette 18 is advised that: 1) the second Drug Cassette 18 for the same drug will not be allowed to begin dispensing of the medication until the doses in the first Drug Cassette 18 have all been dispensed, or 2) that the ability to dispense from the first or previous Drug Cassette 18 will be blocked and that the medical professional should discard the first Drug Cassette 18. The replacement of a Drug Cassette 18 before it is fully consumed per the authorized Prescription 4 is noted in the Central Servers 10 to assist in tracking potential drug divergence. The alert also provides requisite troubleshooting/reset instructions designed to ensure that a given patient cannot dispense more drug within a specified period of time than is allowed by the Prescription 4, regardless of the number of Drug Dispensers 16 containing the prescribed medication the Patient 6 may have.

When ready, the ready to dispense screen appears. Then, if the Drug Cassette 18 is being loaded by a medical professional, the individual will provide the Patient 6 the closed, loaded, and locked tamper resistant Drug Dispenser 16 containing the prescribed drug in the Drug Cassette 18 with the related package insert, prescription and patient instructions much the same as with any other prescription.

Dispensing the Disposable Drug Dispenser

In the case of a Disposable Drug Dispenser 16, dispensing only requires that the Pharmacy 8 dispenses the drug much the same as any other prepackaged medication. The Drug Specific App 12 will effectuate a handshake with the Disposable Drug Dispenser 16 and automatically preclude double dispensing the same prescribed drug dose from more than one Drug Dispensers 16 simultaneously. The Drug Specific App 12 ensures that all dispensing is restricted per the Prescription 4 instructions.

Once recognized by the Drug Specific App 12, the App 12 logs the Drug Dispenser's 16 unique serial number, Drug Cassette's 18 unique serial number, the medication's NDC number, drug batch number, drug's expiration 458 and Prescription Expiration Date 103 (beyond-use date), and unique storage parameters 460, 462 etc. Thereafter, the Drug Specific App 12 will not dispense the prescribed drug, e.g., oxycodone, from other than from authorized Drug Dispensers 16. No duplicate dosing will be allowed to occur. If the drug has been recalled, expired, or the drug has been improperly stored, etc., then early replacement requires an authorized medical professional or a representative from the Integrated Call Center 22 to input an override clearly showing the reason why the other Drug Cassette 18 was disposed of and a replacement Drug Cassette 18 loaded.

Closed Loop Tracking

The integrated system enables a closed loop tracking of the drug which includes the Prescriber 2, the Prescription 4, the Patient 6, the Pharmacy 8, Data Servers 10, Drug Specific App 12, Interface Device 14, Drug Dispenser 16, Drug Cassette 18, and if applicable, the RFID Pill 20, and or Selfie 21 capturing the Patient 6 ingesting the pill. The drug can be tracked from the time the Drug Cassette 18 is filled with the drug until it is dispensed or ingested.

Patient Values

Ensuring the proper utilization of a drug, such as opioids (e.g., OxyContin®), sedatives, hypnotics, stimulants, cardiovascular agents, etc., serve as examples of how the embodiment can be used to ensure medication efficacy, proper drug utilization and the avoidance of medication mediated adverse events, overdose, abuse, misuse, under dosing, over dosing, addiction, dependence, and adverse drug-drug interactions. They can be utilized for medication titration, withdrawal, prevention of side effects (e.g., opioid induced constipation), and better patient management by capturing certain drug related side effects. The information can also be utilized to capture patient reported outcomes (PRO) information required during clinical trials for regulatory agency submissions.

The information that is captured is medication and/or Drug Specific Dispensing Algorithm 13 dependent. As an example, the CDC recommends that physicians assess pain using validated instruments such as the 3-item (PEG) Assessment Scale, which can be incorporated by the embodiment of the invention, on a scheduled basis for reporting to the appropriate healthcare professionals, subject to Patient 6 approval:

1. What number best describes your pain on average in the past week? (from 0=no pain to 10=pain as bad as you can imagine)
2. What number best describes how, during the past week, pain has interfered with your enjoyment of life? (from 0=does not interfere to 10=completely interferes)
3. What number best describes how, during the past week, pain has interfered with your general activity? (from 0=does not interfere to 10=completely interferes)

Examples of values that may be tracked by the Drug Specific App 12 to assist the Drug Specific Dispensing Algorithm 13 in making drug dispensing decisions and/or to assist the Prescriber 2 in managing the Patient 6 or to capture clinical trial information include but are not limited to: (i) a decreased oxygen saturation level (SpO₂) as a result of drug induced slow and/or shallow breathing or changed CO₂ level, (ii) a rapid or very slow heart rate or heart range change, (iii) compromised cognition, (iv) slurred and/or changed speech pattern, (v) miosis, and/or (vi) weight loss, (vii) drowsiness or sedation, e.g., excessive sleeping, (viii) action myoclonus of the limbs, etc., (ix) medications taken since the last dose was dispensed or concomitant medications being taken, (x) drug mediated side effects, e.g., constipation, etc. An excellent example of an avoidable side effect is constipation.

One common approach to non-invasive pulse oximetry that can be incorporated into an Interface Device 14 uses a dual-wavelength sensor placed across a section of venous tissue such as the patient's digit (placed over the sensor) to measure the percentage of hemoglobin oxygenated in the arterial blood, and thereby measures the patient's oxygen saturation level. In addition, since the oxygenated hemoglobin at a specific tissue position is pulsatile in nature and synchronous with the overall circulatory system, the system indirectly measures the patient's pulse rate. A low SpO₂ is indicative of hypoxemia which may result from very slow and shallow breathing caused by too much drug, such as anopioid. Such patient values and information may be provided by a device communicatively coupled to the Drug Dispensing App, such as a wirelessly connected IoT device.

For detection of potential respiratory depression associated with the administration of certain drugs, like narcotic analgesics, such as OxyContin®, oxycodone, hydromorphone, hydrocodone, morphine, etc., a system which indicates a patient's respiratory and cardiac status without the need to invasively measure or sample the patient's blood is particularly desirable and useful. Today, these are available as Apps on smartphones and on real time fitness monitoring devices, e.g., FitBit, Jawbone, and Fuelband, etc. As an example, both (i) non-invasive pulse oximetry to monitor blood oxygen saturation, and (ii) pulse rate are now becoming readily available on smartphones (e.g., Samsung's Galaxy 6 S and 7 S with an S Health App that has a pulse, heart and oxygen saturation monitor, similar Apps for Apple's iPhone, iOximeter, etc.) and wearable diagnostic and/or monitoring devices. The embodiment has the ability import, for example, oxygen saturation and heart rate values from these devices through the development of APIs (application program interfaces).

SpO₂ can thus be measured as a Self-Test 84 on the Interface Device 14 and/or via digital capture from another App 852, 858, 859, 860 and or digitally captured values from an oximetry device 882.

Heart rate can be digitally captured on an ongoing basis by a Digital Monitor 859 and/or as a Self-Test 864.

Compromised cognition may be indicated by a manual dexterity reflex Self-Test 864. The result is then compared to base and trended values to ascertain the Patient's 6 changes in cognition.

A slurring and volume Self-Test 864 can be utilized to recognize changes in speech pattern. The result is then compared to base and trended values to ascertain the Patient's 6 changes in speech and volume. An IBM Watson tone test can be utilized by the Integrated Support Center 22 to listen to the Patient 6 when they call as an input to making a decision of whether or not to allow the Patient 6 to dispense a drug dose that has been disallowed by the Drug Specific Dispensing Algorithm. A two-way video conference can be utilized by the Integrated Support Center 22 to utilize IBM Watson's facial recognition software to use the Patient's 6 face in comparison to meta data facial comparison learning to ascertain if the Patient 6 is potentially overdose or borderline to educate any call center decisions, etc.

A miosis Self-Test 864 can be carried out by having the Patient 6 take a selfie of his/her eye or face. The result is then submitted to the appropriate light adjustment algorithm and the miosis measurement is then compared to base and trended values to ascertain the Patient's 6 changes in pupil constriction in order to ascertain if excessive constriction of the pupil is occurring which may be the result of opioid over dosing. The Iris Scan biometric can be utilized to automatically capture the pupil scan.

Weight loss can be tracked using a digital scale 858 either through digital capture via an API, from a Digital Interface 860, and/or through Self-Reporting 866, or through a communicatively coupled IoT device.

Measuring the amount of sleep or level of activity can be accomplished via a number of smartphone Apps 852, 880 and sleep monitoring devices. The level of activity or a Patient's 6 gait and/or action myoclonus of the limbs can be captured using wearable digital monitors 859, etc.

Utilization of Self-Reporting 866 regarding stool consistency, e.g., using the Bristol Stool Index, and frequency of bowel movements can indicate when the patient may be moving towards drug induced constipation and enable preventative use of the prescribed laxative.

Tracking of certain drug related side effects via self-assessment 862, self-test 864, and/or self-reporting 868 can assist the Prescriber 2 in managing certain Patients 6.

Digitally or self-reporting tracking of medications taken by the Patient 6 since they took their last drug dose can decrease the potential of drug-drug adverse events, overdoses.

A myriad of factors can interfere with how the medication is metabolized and alter the need or effectiveness of the drug over time. Continual monitoring as the Patient 6 takes the drug enables tracking of trends to ensure the drug continues to be efficacious for the patient and to monitor and manage any changes in patient medication tolerance, etc.

Dispensing

Information capture is of little value if it is not actionable. The Drug Specific Dispensing Algorithm 13 is designed to incorporate certain drug specific values to make a dispense or do not dispense decision, even if dispensing would otherwise be authorized by the prescription. This enables personalization of each medication prescription 4 to the individual Patient 6, significantly improving the medication's efficacy/safety profile.

Under the embodiment, the patient would be prescribed a drug in: (i) a Disposable Drug Dispenser 16 containing the specified drug, or (ii) the Drug Cassette 18 to be loaded into the Reusable Drug Dispenser 16 by a medical profession and dispensed to the Patient 6 using the Drug Specific App 12 controlled Reusable Drug Dispenser 16 with the Drug Cassette 18 docked in the tamper proof unit. When the Patient 6 clicks on the Drug Specific App 12 to take his/her next dose, the Drug Specific Dispensing Algorithm 13 would automatically check to ensure the drug has not expired 542, and if it has not, then to see if the drug has been stored within drug specific temperature 558 and humidity ranges 566, and if the Drug has been stored correctly, then it would check the Prescription 4 to ascertain when the Patient 6 may take his/her next dose 580, 584, 588, and, thereafter, it would handshake with designated devices to digitally capture, for example, the Patient's 6 heart rate (to see if it is slow and erratic), weight information (to see if the Patient 6 is losing weight), etc. Thereafter, it asks the Patient 6 at least one Patient Self-Assessment 862, Patient Self-Test 864, and/or Patient Self-Report 866 question by bringing up the respective screen(s). An example of a Patient Self-Assessment 862 screen is the Patient's 6 pain level using the Wong-Baker FACES scale. Examples of the Self-Test 864 screens may include: (i) a Motor Skills Test to ascertain cognition, (ii) a self-administered peripheral capillary oxygen saturation (SpO₂) test to estimate the amount of oxygen in the blood as an indicator of potential hypoxia which may be caused by drug induced respiratory depression, (iii) the Patient 6 taking a selfie of his/her eye or face (with her/his eyes open) to ascertain if his/her pupils are excessively constricted, possibly due to drug (e.g., opioid) over dosing, (iv) a speech print to ascertain if the Patient 6 has slurred speech, a change in how quickly the patient talks, and/or a change in normal volume, etc. Examples of Patient Self-Reporting 866 include but are not limited to: (i) stool composition, using a scale such as the Bristol Stool Scale, to ascertain if the patient may be getting constipated (possible opioid induced constipation), (ii) a bowel movement frequency screen to ascertain any changes in bowel movement that may be indicative of becoming constipated, (iii) a query regarding the new or altered use of medications which may interact with the drug, etc.

The Drug Specific Dispensing Algorithm 13 can use any combination of digitally captured 852, 854, 856, 857, 858, 859, 860, 880, 882, 884, 886, Self-Assessment 862, Self-Test 864, Self-Report 866 physiological, psychological, lifestyle, other medications taken, and environmental data to make the dispense 868 or do not dispense 870, 872, 874 decision. The data capture can be the same every time the Patient 6 wants to dispense a dose or (i) the data request may be triggered by a cascade of information where a value within a specific range, e.g., heart rate, can precipitate the request for a specific input, e.g., a SpO₂ Self-Test 864, or (ii) the data screens may be presented on the Interface Device 14 at pre-determined intervals, e.g., every so many dispenses, at predefined time intervals, if a value on another screen falls within a predefine range or if the Patient 6 answers a specific question in a certain way, etc.

As an example, after Biometric Authentication 850, the Drug Specific App's 12 Drug Specific Dispensing Algorithm 13 uses a Decision Tree, see FIG. 11 through FIG. 16. The algorithm can be as simple as using an Iris Scan for the biometric logon and then using light adjusted and trended Iris scan data to measure the pupil size and compare it to baseline values to ascertain if the Patient 6 has excessive constriction of the pupil of the eye which may be an indication of drug (e.g., opioid) over dosing or potential over dosing if another dose is taken. If the Iris scan is within an acceptable range, the algorithm could: (i) present the Wong Baker FACES® scale to track the Patient's 6 pain over time (or skip this step), and/or (ii) go to the Ready to Dispense 868 screen—allowing the Patient 6 to dispense the dose with a single click on the Drug Dispenser's 16 dispensing button.

Alternatively, it can use, for example, a fingerprint scan. After authentication, the algorithm can begin capturing Patient 6 input data by asking the Patient 6 to click on his/her pain level, using the Wong Baker FACES®, on the first Self-Assessment 862 screen. The pain level information is used to track the efficacy of the pain relieving drug (e.g, opioid) and to provide trending data to ascertain if the Patient 6 is developing drug tolerance. Upon tapping on the respective value, the algorithm automatically presents, as an example, the SpO₂ Self-Test 864 screen. Once the SpO₂ Self-Test 864 is complete, the Drug Specific Dispensing App 13 evaluates the trended SpO₂ data to ascertain if the Patient 6 is or may be moving toward hypoxia. If potential respiratory depression is suspected, a cognition Self-Test 864, which would not otherwise be presented, is automatically presented. If everything is within acceptable ranges, the Drug Specific Dispensing Algorithm 13 can present, for example, a constipation Self-Reporting 866 screen, can ask the CDC recommended 3 Item PEG Assessment Scale, ask for Prescriber 2 requested side effect information, and/or clinical trial PRO data, or go directly to the Ready to Dispense 868 screen. At this point, the Patient 6 only needs to click on the Drug Dispenser's 16 dispensing button to dispense the dose.

However, if, based upon the SpO₂ and cognition trended data, the algorithm suspects drug toxicity (overdose) it can ask for additional confirmatory information, e.g., a speech Self-Test 864, a miosis selfie, etc. Once the Drug Specific Dispensing Algorithm 13 is satisfied that a potential toxic event is or may occur, it locks the Drug Dispenser 16 and produces the respective screen 874 telling the Patient to contact the Integrated Support Center 22, or talk with their Prescriber 2 or a physician prior to being able to dispense the next dose, etc., even if the dose is within prescribing parameters.

After talking with the Patient 6, the Integrated Support Center 22 representative can decide, within their operating constraints, whether to remotely unlock the Drug Dispenser 16 and allow the Patient 6 to dispense the drug. If it does not appear appropriate to authorize the Patient 6 to dispense the drug, the representative can triage 806 the Patient 6 to an Integrated Support Center 22 physician or another medical professional. The physician can change the Prescription 4, if authorized, and can make the decision whether to unlock the Drug Dispenser 16 and allow the Patient 6 to dispense the medication. Whoever, has the last interface with the Patient 6, is responsible for: (i) terminating the call with the Patient 6, (ii) sending a summary email with instructions 830 to the Patient 6, (iii) updating the patient's record 818, (iv) advising the Prescriber 2 that dispensing has been stopped or the Prescription 4 changed 832, and (v) updating the Electronic Medical Record 24.

If there is a consultation between the Prescriber 2, the prescription can be changed by the Integrated Support Center 22 in the Patient's 6 Drug Specific App 12 resident on the Patient's 6 Interface Device 14 or Standalone Drug Dispenser 16 based upon the Prescriber's 2 instructions.

The embodiment personalizes drug treatment, improves the drug/medication's drug/safety profile, and enhances the Patient's 6 quality of life all while decreasing preventable side effects, accidents, and overdoses and thereby decreasing the number of physician interactions and ER visits—saving on the total cost of patient care and saving lives.

### D. Drug Recalls or Clinical Trial Medication Stoppage

In the event of a drug recall or in the event that a drug is in a clinical trial, the Integrated Support Center 22, within its procedures and controls, can remotely lock all Drug Dispensers 16 that contain a specified drug and simultaneously alert the Patient 6 that he/she cannot dispense the drug and what they should do.

### E. Titration

The embodiment of the Invention can also be utilized to assist in drug titration. Titration helps the body adapt to the medication and often reduces common side effects that can occur when one begins drug therapy. With respect to certain drugs prescriped for pain management, such as opioids, the doctor/Prescriber 6 will likely start with an initial low dose of the drug and carefully adjust the dose upwards to adequate levels. Titration helps find the optimal dose to improve daily functioning. For example, the drug dosage is increased slowly to the highest tolerable dose. Once there is no more improvement in symptoms as the dosage increases, the Prescriber 6 will lower the dose to the previous one. If a higher dose produces too many side effects, the dosage is lowered. Drugs management is based on an individual's own personal needs and responses. The optimal dose of drug is one in which daily function is significantly improved and side effects are minimized.

There are three simple goals for pain management;

(i) A good night's sleep,
(ii) Pain control during the day while at rest and
(iii) Pain control when the Patient 6 is active and ambulatory.
Where there is no previous history of drug intake, the starting dose is calculated by assessing the severity of the pain, patient's age, weight, sex and general physical condition.

The invention is designed to enable changes in how pain releaving drugs, such as opioids, are titrated using a combination of Oral Patient Controlled Analgesia and, for example, the Wong Baker FACES® 0 to 10 pain Self-Assessment index. The changed therapy would enable the Patient 6 to administer a low dose drug as needed within certain waiting times between doses. Once the Self-Assessed pain index level reaches a certain level of pain control, the interval between doses would be increased until the pain level once again begins to rise. At that point, the interval between doses could be decreased back to the previous interval. Review of the data would allow the Prescriber 2 to increase the prescribed dosage strength to decrease the number of doses the Patient 6 would have to take to control his/her pain, etc.

Pain Assessment

Pain assessment and re-assessment after administration of analgesics or pain management is regulated in healthcare facilities by accreditation bodies, like the Joint Commission. The Joint Commission began setting standards for pain assessment in 2001 stating that the route of analgesic administration dictates the times for pain reassessment, as different routes require different amounts of time for the medication to have a therapeutic effect. Oral immediate release (IR) medications require 45-69 minutes.

Most pain assessments are done in the form of a scale. The scale is explained to the Patient 6, who then chooses a pain score. A rating is taken before administering any medication and after the specified time frame to rate the efficacy of treatment. Patients rate pain on a scale from 0-10, 0 being no pain and 10 being the worst pain imaginable. A scale with corresponding faces, e.g., the Wong Baker FACES, depicting various levels of pain is shown to the Patient 6 and they select one. Patients who cannot verbalize/comprehend pain scales are assessed with different types of scales.

The embodiment of the invention would allow the pain assessment to be conducted over a period of time under actual living conditions. This would give a better assessment of the Patient's 6 pain level and enable better pain management.

Drug Titration

The Prescription 4 can be written to allow the Patient 6 to gradually increase their dose by prescribing a low dose of drug and allowing the Patient 6 to dispense the dose more frequently or to dispense a defined number of pills. As an example, the Patient 6 could dose every so many hours and/or fractions thereof or the Patient 6 could dose more frequently throughout the day, e.g., from b.i.d. go to t.i.d, from t.i.d. go to q.i.d., from q.i.d. to every two hours, and from every two hours to q.h. It can also be indicated to allow double dosing before bedtime . . . and to allow p.r.n. dosing at a point where the pain is under control based upon trended patient Self-Assessment 862 data and related decreases in Self-Reported 866 drug induced side effects.

Once steady state has been achieved, the Prescriber 2 can change the prescription to best manage the Patient's 6 pain.

Titration can be facilitated by the utilization of a special titration Drug Cassette 18, 18a, 18b, 900 configuration containing more than one strength of drug.

### F. Patient Controlled Oral Analgesia (PCOA™)

Attributes of the embodiment enable Patient Controlled Oral Analgesia (PCOA™) even for medications, such as Oxycontin® which are not approved for PRN dosing. Studies have shown that patients that can self-medicate as warranted, e.g., PRN with set prescription parameters, tend to use less medication, further mitigating potential side effects.

While it is generally recommended that analgesic medications for moderate to severe pain should be given on a regular schedule, the use of the embodiment allows for PCOA™ dosing. The Patient 6 can be allowed to dispense a certain number of pills, not to exceed a specified amount, during a defined time period. A minimum interval between doses can also be set. This can be further restricted to limit the total allowed dose, over a number of hours or for a given 24-hour period, be restricted to a dosage limitation.

PCOA™ allows prescribing to manage breakthrough pain while controlling the maximum allowable dose. PCOA™ would allow for the utilization of an immediate release drug in conjunction with an extended release drug to address issues with breakthrough pain. Conversely, it also allows for the elimination of extended release drugs, allowing the dosing schedule to coincide with the Patient's 6 pain.

### G. Diagnostic

The embodiment of the Invention can also be utilized to assist in diagnosis. As an example, there are many different types of pain and different types of headaches. Patients will generally begin by self-medicating with over the counter (OTC) analgesics such as aspirin. As the pain or discomfort increases, patients increase the number of tablets taken (i.e., the dosage), as well as the frequency of self-medication. At a certain point, they go to their doctor seeking adequate relief.

When the doctor talks with the Patient 6, he/she may describe many different types of pain, making it difficult to diagnose. Pain has multiple causes, and people respond to it in multiple and individual ways. The pain that one person pushes their way through might be incapacitating to someone else.

Headaches represents an example. It is important to figure out what type of headache is causing the pain. If the doctor knows the type of headache, he/she can treat it correctly. However, as was highlighted by a 2004 study, 80% of people who had a recent history of self-described or doctor-diagnosed sinus headache, but no signs of sinus infection, actually met the criteria for migraine. The following discusses the different types of headaches:

1) Tension headaches, the most common type of headache, can generally be adequately treated with over-the-counter treatments such as aspirin, ibuprofen, or acetaminophen (Tylenol). Experts believe these may be caused by the contraction of neck and scalp muscles (including in response to stress), and possibly changes in brain chemicals.

2) Cluster headaches, which affect more men than women, are recurring headaches that occur in groups or cycles. They appear suddenly and are characterized by severe, debilitating pain on one side of the head, and are often accompanied by a watery eye and nasal congestion or a runny nose on the same side of the face. During an attack, people often feel restless and unable to get comfortable; they are unlikely to lie down, as someone with a migraine might. The cause of cluster headaches is unknown, but there may be a genetic component. There is no cure, but medication can cut the frequency and duration.

3) Sinus headaches occur when a sinus becomes inflamed, often due to an infection. They can generally be diagnosed by symptoms or the presence of pus viewed through a fiber-optic scope. Headaches due to sinus infection can be treated with antibiotics, as well as antihistamines or decongestants.

4) Rebound headaches, ironically, can be caused by the overuse of painkillers for headaches. Culprits include over-the-counter medications like aspirin, acetaminophen (Tylenol), or ibuprofen (Motrin, Advil), as well as prescription drugs.

5) Migraine headaches can run in families and are diagnosed using certain criteria: (i) at least five previous episodes of headaches, (ii) last between 4-72 hours, (iii) at least two out of four headaches have one-sided pain, throbbing pain, moderate-to-severe pain, and pain that interferes with, is worsened by, or prohibits routine activity, and (iv) at least one of the following is associated with the pain: nausea and/or vomiting, or, if those are not present, then sensitivity to light and sound. A migraine may be foreshadowed by aura, such as visual distortions or hand numbness. (About 15 percent to 20 percent of people with migraines experience these.)

6) Mixed headache syndrome, also called transformed migraines, is a combination of migraine and tension headaches.

7) Acute headaches are headaches that occur suddenly and have symptoms that subside after a relatively short period of time.

8) Hormone headaches are often associated with women's changing hormone levels during menstruation, pregnancy, and menopause. Chemically induced hormone changes, such as with birth control pills, also trigger headaches in some women.

9) Chronic progressive headaches, also called traction or inflammatory headaches, are chronic progressive headaches that get worse and happen more often over time. These are the least common type of headache, accounting for less than five percent of all headaches in adults and less than two percent of all headaches in kids. Chronic progressive headaches may be the result of an illness or disorder of the brain or skull.

Diagnosis requires a headache evaluation that includes: (i) headache history, (ii) description of the headaches, (iii) headache symptoms, (iv) characteristics, (v) a list of things that cause the headache, (vi) aggravate the headache, and (vii) things the patient has done to relieve a headache. The patient is also requested to keep a headache diary.

The proper treatment will depend on several factors, including the type and frequency of the headache and its cause. There are many migraine and headache medications and other treatments are available. The appropriate treatment often depends on the type of headache.

Headache pain may need to be managed with medications. Headache drugs used to treat headache pain can be grouped into three different categories: symptomatic relief (drugs used to treat the headache pain or accompanying symptoms of migraines like nausea), abortive therapy (drugs used to stop a migraine headache), and preventive therapy (drugs used to prevent a migraine). Botox injections represents another migraine and headache treatment.

The way the body responds to migraine and headache medications may change over time, so medications may need to be adjusted.

The embodiment of the Invention enables the aggregation of Patient 6 specific dispensing information and Patient Self-Assessment 862 and/or Self-Reported 866 information specifically developed to assist in the diagnosis and management of headaches.

### H. Management of Complex Drug Therapy

The embodiment includes the use of a Multi-drug Drug Dispenser 1002, 1004, 1006, 1008 in conjunction with a Consolidation Therapy App 1000 that would allow for the dispensing and control of two or more medications to better manage complex drug therapies. As an example, to dispense a long acting drug, an immediate release drug, and a medication or medications to address drug induced side effects when certain Self-Assessment 862, Self-Testing 864, Self-Reporting 866 or digitally captured information indicates the symptomatic medication is warranted to treat the side effect, e.g., diarrhea and vomiting.

Cystic fibrosis (CF) serves as yet another example of how the system can be utilized to manage complex drug therapy. There is no cure for CF, but treatment can ease symptoms and reduce complications, physician office visits and hospitalizations. Close monitoring and early, aggressive intervention is recommended.

Managing CF is complex, so treatment is best if managed by a center that specializes in cystic fibrosis. The goals of treatment include: (i) preventing and controlling lung infections, (ii) loosening and removing mucus from the lungs, (iii) preventing and treating intestinal blockage, (iv) providing adequate nutrition, and (v) medications.

The patient must take multiple drugs, the schedule and combination which must be personalized for each patient. The medicines include those to help treat or prevent lung infections, reduce swelling and open up the airways, and thin mucus. If the patient has mutations in a gene called G551D, which occurs in about 5 percent of people who have CF, the doctor may prescribe the oral medicine ivacaftor (approved for people with CF who are 6 years of age and older). Adherence and persistence with each drug regimen is critical to avoid costly complications. The options include:
a. antibiotics to treat and prevent lung infections (Most people with CF have ongoing, low-grade lung infections. Sometimes, these infections become so serious that the patient may need to be hospitalized. Antibiotics are the primary treatment.)
b. mucus-thinning drugs to help the patient cough up the mucus, which improves lung function.
c. bronchodilators to help keep the airways open by relaxing the muscles around the bronchial tubes, and
d. oral pancreatic enzymes to help the digestive tract absorb nutrients.

The embodiment of the Invention enables the complex management of the CF Patient 6 via the utilization of the Multi-Drug Dispenser 1002, 1004, 1006, 1008. The Consolidated Therapy App 1000 consolidates from two to as many Drug Specific Apps 12 as are resident on the Interface Device 14 into a single user interface for all drugs—eliminating duplicate logins, entries, and record keeping. It in turn digitally handshakes with the Multi-Drug Dispenser 1002, 1004, 1006, 1008 and uses the individual Drug Specific Dispensing Algorithms 13 to control dispensing of each individual medication. Furthermore, it coordinates the dispensing schedules to have as few dispensing times, within the respective prescriptions, as possible. Multi-Drug Dispenser 1002, 1004, 1006, 1008 eliminates concerns about which drugs have to be taken when. It can also be programmed to provide alerts for the patient to take his/her related injectable and/or inhaled medications. In this way, the Dispensing System simplifies CF drug management, encourages prescription compliance and persistence, avoids complications, and thereby reduces the total cost of treating a CF patient by decreasing the number of physician interventions and hospitalizations.

### I. Countering Addiction and Withdrawal

Certain drugs are known to cause addiction and withdrawal. In this regard, opioids (examples include: codeine, fentanyl and analogs, hydrocodone, hydromorphone, methadone, oxycodone, Oxymorphone, etc.) are effective in controlling pain. However, physicians are reluctant to prescribe them due to their overdose, abuse, addiction and divergence potential and related REMS programs. Some patients are also reluctant to take them due to their addiction potential. The embodiment provides control and real time monitoring and thereby addresses these shortcomings.

Overdosing is addressed by: 1) a diagnostic screening diagnostic that precludes dispensing a drug dose if there is an indication the Patient 6 is overdosed or would likely overdose if the Patient 6 takes another drug dose, even if the dose would be authorized by the Prescription, and (ii) the inability of the patient to dispense a dose more frequently than allowed by the Prescription 4. This is handled by the Drug Specific Dispensing Algorithm 13 which controls dispensing by the Drug Dispenser 16.

Abuse is addressed by the design of the tamper resistant Drug Dispenser 16. For Reusable Drug Dispensers 16, The Drug Specific Drug Cassette 18 can only be docked with the Reusable Drug Dispenser 16 by an authorized medical professional. Any attempt by an unauthorized person to open the Drug Dispenser 16 triggers a signal to the Drug Specific App 12 which automatically locks the Drug Dispenser 16 and alerts the Integrated Support Center 22. Alerts are also generated if the Patient 6 is trying to use multiple Drug Dispensers 16 to double dose. When the Integrated Support Center 22 is alerted, it then calls the Patient 6 to ascertain why he/she is trying to open the Drug Dispenser 16 and/or trying to dispense duplicate doses. At this point, the Integrated Support Center 22 works with the Patient 6 to address any dispensing related issues and can unlock the Drug Dispenser 16 so a dose can be dispensed or, if attempted abuse is suspected, contacts the Prescriber 2 to alert them of the conversation with the Patient 6 and asks the Prescriber 2 whether the Drug Dispenser 16 should remain locked so no drug can be dispensed or if it should be unlocked to allow dispensing. If authorized, the Integrated Support Center 22 updates the Electronic Medical Record 24 related to the calls to the Patient 6 and the Prescriber 2.

Addiction potential is deceased by: (i) the patient's inability to dose more frequently than the prescribed medication schedule, regardless of the number of Drug Dispensers 16 that contain the drug that the Patient 6 has, (ii) by tracking attempted earlier than prescribed dosing events to enable early intervention, (iii) by capturing any attempts to open the Drug Dispenser 16, and (iv) through the use of patient Self-Assessment 862, Self-Testing 864, Self-Reporting 866 and/or digitally captured relevant information 852, 854, 856, 857, 858, 859, 860, 880, 882, 884, 886 trended over time, to ascertain the effectiveness of the drug in controlling the Patient's 6 pain. The centralized drug specific patient and population focused analytics programs on the Data Servers 10 are designed to use data analytics, on individual as well as metadata from all Patients 6, data captured by the Drug Specific App 12 to identify potential movement of the Patient 6 toward addiction. When potential addiction is identified, the analytics software alerts the Integrated Support Center 22 so they may alert the Prescriber 2 and update the patient's 6 Electronic Medical Record 24. The more data collected and analyzed the more accurate the predictive analytics will be.

Divergence is decreased by: (1) the tamper resistant Drug Dispenser 16 design which triggers an alert if unauthorized attempts to open the Drug Dispenser 16 are sensed; (2) restriction of Drug Cassette 18 docking into the Reusable Drug Dispenser 16 by authorized medical professionals; (3) restricting access to the drug except for single dispensed doses dispensed per the Patient's 6 prescription 4; (4) precluding dose dispensing to a single dose regardless of the number of Drug Dispenser's 16 the Patient may have; (5) the automated closed loop drug tracking system that: (i) correlates the serial number of the Drug Specific Drug Cassette 18 and the drug's batch number to the Drug Dispenser 16, (ii) the serial number of the Drug Dispenser 16 is linked to the Patient's 6 Drug Specific App 12, (iii) the use of the Drug Specific App 12 is restricted to a specific Patient 6, and (iv) the Drug Specific App 12 requires a biometric login 850 to access the Drug Specific App 12 in order to instruct the Drug Dispenser 16 to dispense the drug. Furthermore, additional control is supplied if the drug being taken can be tracked with RFID 856 tracking until ingested and/or a Selfie 857 is taken confirming the drug dose was in fact taken by the Patient 6. The time interval between the time the drug is dispensed and the time it is ingested, over time, provides an indication of compliance, dose stockpiling, or divergence. When coupled with metadata analytics conducted by the Data Servers 10, the probability of accurately identifying potential abusers and divergence is significantly increased.

Side Effects—The system may also be utilized to predict potential side effects. For example, drug related constipation can be predicted based upon the Self-Reporting 866 of the frequency of bowel movements since the previous dose or over a previous specified time period as well as the stool consistency of the last bowel movement, etc. If predicted, the Drug Specific App 12 can alert the Patient 6 to take a laxative at the appropriate time. If a multi-drug Drug Dispenser 1002, 1004, 1006, 1008 is utilized, the Consolidated Therapy App 1000 can dispense the laxative as well as the drug and/or other medications as prescribed.

REMS—The system is designed to comply with the respective REMS program and to virtually eliminate required data capture and automate patient specific tracking and dispensing report preparation. The Integrated Support Center 22 also supports the Prescriber 2 by preparing the required REMS reports encompassing all his/her patients.

Prescription Drug Monitoring Programs—The system also allows for the redefinition of Prescription Drug Monitoring Programs by closing the loop between the Pharmacy 8, the Prescriber 2 and the Patient 6 by controlling and tracking use on an individual Patient 6 and Prescriber 4 and dispensing Pharmacy 8 basis.

### J. Addiction and Withdrawal Treatment

Addiction is a global crisis. As an example, there are an estimated 2.4 million opioid-dependent people in United States, 1.3 million in Europe and twenty million in the rest of the world. Drug overdose is the second leading cause of accidental death in the United States. According to the CDC, overdoses from prescription opioids claimed over 18,000 lives in the United States alone in 2014.

Drug addiction can either be treated with buprenorphine and/or naloxone (examples of brand names include Butrans, Suboxone, Zubsolv). In cases of physical dependence, withdrawal must be managed through the gradual decrease of opioid doses of the dependent drug.

Appropriate precautions must be taken to minimize risk of misuse, abuse, or diversion, appropriate protection from theft, and unintended pediatric exposure. In addition, appropriate clinical monitoring as to the Patient's 6 level of stability is essential. The embodiment of the system provides dispensing control and real time monitoring and thereby address each of these shortcomings.

Decreased dosing for the treatment of addiction and withdrawal utilizes all the features of the embodiment and is controlled by the Prescription 4.

### K. Clinical Trials

The system is designed to capture, store, analyze, and act upon drug specific patient self-assessment 862, self-testing 864, self-reporting 866 and digitally captured 850, 852, 854, 856, 857, 858, 859, 860, 880, 882, 884, 886 physiological, psychological, lifestyle, other drugs currently being taken, and environmental information along with the drug's Prescription 4 and drug dispensing and Drug Dispenser 16 storage history in order for the Drug Specific App 12 to decide if the medication should or should not be dispensed. Dispensing can be precluded by the Drug Specific App 12 based upon defined clinical trial guidelines as well as the Prescription 4. In this way, patient monitoring information can be utilized to preclude dispensing a prescribed dose if a potential adverse event is identified.

Most of the time, clinical outcomes are held as the ultimate outcome in a clinical trial because they often provide more objective interpretation, increased reliability and greater simplicity of interpretation. However, certain disease conditions require consideration of subjective outcomes. Regulatory agencies, such as the FDA, are combining patient reported outcomes (PROs) and clinical outcomes in their approval decisions. Examples include the: (i) FDA's “Guidance for Industry, Irritable Bowel Syndrome—Clinical Evaluation of Drugs for Treatment”, dated May 2012 and (ii) the European Medicines Agency (EMA) “Guideline on the evaluation of medicinal products for the treatment of irritable bowel syndrome” dated April 2015. They utilize a combination of PROs and patient self-assessment reporting to measure primary and secondary endpoints required for regulatory approval of any 5HT3 drugs for irritable bowel syndrome (IBS).

Interest in developing and applying patient-reported outcomes (PROs) across the drug development and postmarket spectrum is growing—among sponsors, clinicians, payers, regulators and patients. A growing number of clinical trials now are going beyond conventional randomized control measurements to collect self-reported outcomes from patients—focusing on improving patients' involvement by including their perspectives throughout the drug development process. An analysis of sponsor-funded interventional studies listed on CenterWatch's Clinical Trials Listing Service found between 2005 and 2007, only 6.1% of total study procedures involved some type of subjective outcome assessment. That grew to 11.8% in the 2008 to 2010 timeframe and, most recently, between 2011 and 2013, increased to 16.3% of total study procedures. PROs can capture a range of information, from symptom changes and level of functioning, to health-related qualify of life and treatment satisfaction and adherence.

Although their value is widely recognized, PRO use often is inconsistent and underutilized in understanding how patients feel in relation to their diseases, such as cancer, cardiovascular disease, diabetes, etc. Generally, regulatory agencies do not require sponsors to consider PROs in clinical trials and, until recently, did not do much to encourage their use. However, signs point to that sentiment is changing. Janet Woodcock, M.D., director of the FDA's Center for Drug Evaluation & Research (CDER) stated: “We understand that people with chronic diseases are experts in that disease, as far as the symptoms and the impact on quality of life, and what might be acceptable tradeoffs on risk and uncertainty. The challenge for the FDA is incorporating that knowledge in a way that accurately informs regulatory decisions.” She asked, “how can we meaningfully collect that knowledge in a rigorous manner, given there's a spectrum of opinions and a spectrum of disease burden in any given disease?” PRO measurements often are used to evaluate products that treat chronic, disabling conditions, for which the goal of treatment is focused on alleviating the frequency, severity or duration of disease symptoms.

PROs generally are used as primary endpoints in clinical trials in indications such as migraines and irritable bowel syndrome, in which specific symptoms, such as pain, play a major role in treatment. PROs also are important in the final product labeling manufacturers are allowed to use to promote their products, and to clinicians seeking information to support their prescribing choices. Now, trials for psychiatric and age-related illnesses, among others, are including PROs as part of the protocol design.

Pain studies initially used PROs as a primary outcome in a clinical trial because attempts to obtain an objective measure of pain through a dolorimeter, a spring-loaded instrument with a gauge for measuring sensitivity to, or levels of, pain, or through a galvanic skin response lacked validity compared to simple pain scales. Other disease examples where PROs are preferable include neurology, depression, anxiety, and irritable bowel syndrome (IBS) which may utilize co-primary and/or key secondary PROs.

Keeping trial participants involved also is the hallmark of the publication and promotion of the FDA's PRO guidance at the end of 2009. In 2011, the FDA took the next step, seeking multiple ways to give the patient a clear voice in clinical research by ensuring all measurements and outcomes reflect what is happening with the patient through instruments or tools, along with PROs. Increasingly, we are seeing patients in clinical trials demanding to know what is going on and they want to be given a greater voice.

Generally, larger clinical sites can handle adding PROs more easily, while smaller sites, especially in more remote locations, can find it more challenging. Collecting data directly from the patient can provide stronger information. As an example, patients can be hesitant to report outcomes if they have been asked to take a medication a certain way and have not done so.

Furthermore, collecting data through specific data streams provides, in some cases, better quality. Patients will contact the independent group, such as the clinical trial CRO or in the embodiment, the Integrated Support Center 22, and not necessarily go back to their physicians for technical issues and concerns.

While using PROs is becoming critical in many clinical trials to prove safety and effectiveness to gain FDA approval, the next step for biopharmaceutical companies and payers will be to combine PROs with other observational studies to create real world evidence (RWE). RWE is becoming essential for sound medical coverage, payment and reimbursement decisions, according to the International Society for Pharmaeconomics Outcomes Research Real-World Data Task Force. RWE can be used with randomized clinical trials to design more efficient trials and understand a drug's benefit-risk profile, as well as to gain understanding of the market for launch planning, according to the task force. RWE shows how a drug is accepted from Patients 6 who have experience using it. It reveals how a drug is utilized in different geographies and can be used to help frame policy or regulatory decisions. It is a highly credible source of information.

The embodiment provides: (i) the requisite data capture, (ii) Patient 6 involvement, (iii) dispensing control, (iv) avoidance of certain drug related side effects, (v) real time reminders for the Patient 6 to take the medication, (vi) intervention alerts if the Patient 6 fails to take their medication within a predefined time interval, (vii) dispensing tracking (date and time), (viii) if an RFID chip is integrated, time of ingestion, (ix) if a Selfie is taken, confirmation of ingestion, (x) real time monitoring, and (xi) reporting. It addresses the shortcomings of current systems to capture and compile real time, Patient 6 and drug specific data to facilitate ongoing clinical trial data aggregation, analysis, and reporting while minimizing the number of calls to the clinical trial physician.

Under the current embodiment, the Patient 6 would be prescribed the opioid to be dispensed per a defined prescription using the Drug Specific App 12 controlled Drug Dispenser 16. When the Patient 6 clicks on the Drug Specific App 12 to take his/her next dose, the Drug Specific Dispensing Algorithm 13 automatically handshakes with the Drug Dispenser 16, handshakes with defined digital devices (e.g., blood pressure, heart rate, etc.) FIG. 18, FIG. 19 and downloads the latest data to the Interface Device's 14 Drug Specific App 12 data base, checks to ensure the drug has not expired 458, and if it has not, then to see if it has been stored correctly 460, 462. If the Drug has been stored correctly, then, for example, it automatically moves to the next screen and asks the Patient 6 to answer the specific questions. In this example, the Patient 6 would answer the PRO and data capture screens 862, 864, 866 required by the FDA and EMA to get approval for the opioid with expanded claims. The ability to capture the requisite PRO primary and secondary end-point data and the related compliance and persistence data are illustrated in FIG. 18. These screens can be configured to capture and aggregate drug specific information.

The Drug Specific Dispensing Algorithm 13 then utilizes its decision tree FIG. 11 through FIG. 16 to check the Prescription 4 instructions and when the drug was last dispensed 464 to ascertain if the drug can be dispensed. It then either generates a screen stating that the dose will not be authorized for a specific time period 870 or proceeds to ascertain if the designated digital and Patient 6 entered values allow the medication to be dispensed. If yes, then the screen shows a green dispense 868 notification and prepares the Drug Dispenser 16 to dispense the dose (turns the single click drug dispense button light green 1066). If the Drug Specific Dispensing Algorithm 13 indicates that the Patient 6 should not receive the opioid, even if it is within the Prescription 4 guidelines, then it will either generate, for example, a screen stating that the dose is not warranted 870 at the specific time and provide the Patient 6 the ability to click on “Dial” to call the Integrated Support Center 22 or if a problem is ascertained, it will either show a specifically designed screen or a screen that the Integrated Support Center 22 should be called 874. The type and sequence of screens is dictated by the drug's clinical trial data capture requirements. The algorithm can contain routines that only ask for specific information if certain predefined criteria are met.

Every non-fruitful event to dispense the medication is tracked. At a certain point the Drug Specific Dispensing Algorithm's 13 logic will send a message for the Integrated Support Center 22 to call the Patient 6.

The embodiment allows for better prescription compliance, an improved drug safety profile, increased prescription persistence, uniform data capture, facilitates data analysis, decreases required interventions by the clinical trial physician(s), decreases the cost of the trial, and provides real time data capture and analysis.

### L. Intermittent Chronic Conditions

There are a number of chronic conditions that come and go and do not always require treatment. Examples include IBS, pain, allergies, arthritis, certain heart conditions, anxiety, depression, intermittent claudication, etc. The Drug Specific App 12 is capable of being programmed to control PRN dosing in various configurations and schedules. This allows for real time data capture which is useful in in diagnosis, patient management, and dispensing control. As an example, if a pain medication is prescribed for PRN dosing, the Drug Specific Dispensing Algorithm 13 can ascertain if the time interval between the last dispensed dose and the current dose may be long enough that any tolerance to the drug that had built up is now decreased and either adjust the dispensing interval or sends an appropriate advisory to the Patient 6, the Integrated Support Center 22, the Prescriber 2, the Pharmacy 8, and/or designed care givers 514 and/or relatives 520, 526, 532, etc.

1. A Drug Specific App used to control a drug dispenser that allows the patient/user to designate his/her language preference and, if desired, voice prompts, commands and responses to interface with the Drug Specific App. The Drug Specific App is comprised of: (i) a biometric authentication module, (ii) a prescription module, (iii) a prescriber side effects tracking preferences module (iv) a patient reminder module, (v) an interface, API, between the Drug Specific App on the Interface Device and the Drug Dispenser module, (vi) application program interface(s), APIs, between the Interface Device (or Standalone Drug Dispenser) and digital data capture device(s), and/or data capture software and/or data aggregation devices, (vii) patent self-assessment screen(s) and data aggregation module, (viii) patient self-test screen(s) and data aggregation module, (ix) patient self-report screen(s) and data aggregation module, (x) an Interface Device Database Module (xi) a Drug Specific Dispensing Algorithm module, (xii) a Dispensing Communications and Reporting module, (xiii) an interface, API, between the Interface Device and the Integrated Support Center's servers module, (xiv) a patient reporting module, (xv) a security controlled drug cassette replacement module, (xvi) a GPS module, (xvii) a Package Insert module, (xviii) a Dispenser Manual module, (xix) an App Manual module, (xx) a Help and Troubleshooting module, (xxi) a language preference module, (xxii) a Voice Control module, (xxiii) an App Identifier module, and (xxiv) an App and Dispensing Unit Operation Training module. Said Drug Specific App is customized for the patient based upon the Drug Specific Dispensing Algorithm requirements and the side-effects, if any, the prescriber wants tracked plus a unique App Serial Number. The Personalized Drug Specific App is compiled (created) by the App Generation Program on that Data Servers when a medication prescription is electronically generated and transmitted to the data servers that contain the Patient Specific Drug Specific App generation program(s).

2. The Drug Specific App according to claim 1, wherein the patient/user can select from a list of languages that will be utilized by the Drug Specific App to interface with the patient/user.

3. The Drug Specific App according to claim 1, wherein the patient/user can select voice prompts, commands, and responses to interface with the Drug Specific App.

4. The Drug Specific App according to claim 1, wherein said App is specifically created for the patient by the Drug Specific App Creation Program when it receives an electronic copy of an e-prescription that is electronically submitted by the prescriber or when it is entered by the pharmacy or dispensing site from a written prescription.

5. The Drug Specific App according to claim 1, wherein said App copies the requisite Patient, Prescriber, Prescription, Pharmacy, Additional Information to Track, Side Effects to Track, and Alerts Selection, Timing, and Contact information from the copy of the ePrescription.

6. The Drug Specific App according to claim 1, wherein said App information is checked, and updated as required, every time a refill is dispensed for the prescribed drug.

7. The Drug Specific App according to claim 1, wherein said App requires biometric authentication, e.g., iris scan, finger print scan, voice print, name and password reconciliation, smell or chemical footprint, specific movements, biometric card, computer generated confirmation code, etc. or any combination thereof.

8. The Drug Specific App according to claim 1, wherein said App is configured to capture certain patient self-assessment, patient self-test, patient self-report, and/or digitally captured patient physiological, psychological, lifestyle, other drug(s) taken, and environmental values or information, and/or side effect information that are used by the Drug Specific Dispensing Algorithm to make a program based, decision tree, decision to dispense or not to dispense the drug dose, even if otherwise it would be authorized by the prescription.

9. The Drug Specific App according to claim 1, wherein said App can be configured, as instructed or indicated by the prescriber, to capture certain side-effect, concomitant medications, and patient self-assessment, patient self-test, patient self-report, and digitally captured patient information/values for better patient management and/or which may be required for clinical trials but which are not required to make dispensing decisions by the Drug Specific App dispensing algorithm.

10. The Drug Specific App according to claim 1, wherein the interval for capturing certain side effect and patient self-assessment, patient self-test, patient self-report, and digitally captured patient values/information can be set for each tracked value/information. The time intervals for capturing each piece of specified information is dictated by the Drug Specific Dispensing Algorithm and/or can be as dictated by the Prescriber, for example, every time before the medication is dispensed, every other time, once per day, every other day, weekly, when a certain value is reported, as a result of another value being reported within a defined range, etc.

11. A Drug Specific Dispensing Algorithm that is comprised of decision tree that utilizes prescription, drug dispenser, drug cassette, drug, and digitally captured, patient self-assessment, patient self-test, and/or patient self-reported physiological, psychological, lifestyle, concomitant medications, and/or environmental data in a novel Drug Specific Diagnostic Algorithm to: (i) preclude dispensing the drug to patients who are overdosed or may become overdosed if a drug dose is dispensed, even if the dose is otherwise authorized by the prescription, (ii) control dispensing of each drug dose by the drug dispenser, (iii) preclude drug double dosing, (iv) preclude dispensing of expired drugs, (v) preclude dispensing of an improperly stored drug, (vi) preclude dispensing after the drug prescription has expired, (vii) ensure drug prescription compliance, (viii) preclude the simultaneous dispensing of the same drug from multiple drug dispensers, (ix) control dispensing to preclude early dosing and to ensure maintenance of the minimum period between dispensed doses, (x) prevent tampering with the drug dispenser by locking dispensing in the event the drug dispenser senses unauthorized attempts to open or tamper with the drug dispenser, and (xi) track early attempts to dispense the drug as an indication of possible abuse and/or increasing drug tolerance.

12. The Drug Specific Dispensing Algorithm according to claim 13, wherein said Algorithm utilizes a Drug Overdose Diagnostic to preclude from dispensing a drug dose if the patient is overdosed or if the patient is at risk of becoming overdosed if he/she takes the drug dose, even if otherwise authorized by the prescription.

13. The Drug Specific Dispensing Algorithm according to claim 13, wherein said Algorithm utilizes the prescription information to ascertain if the drug in the drug dispenser is the prescribed drug.

14. The Drug Specific Dispensing Algorithm according to claim 13, wherein said Algorithm utilizes the drug dispenser's device tampering alerts to ascertain if the drug should be dispensed or if the drug dispenser should remain locked until the Drug Dispensing App is unlocked by the Integrated Support Center.

15. The Drug Specific Dispensing Algorithm according to claim 13, wherein said Algorithm evaluates the number of early dispensing attempts to ascertain if the drug should be dispensed or the drug dispenser locked until the patient talks with the Integrated Support Center who will make the decision of whether to keep the drug dispenser locked or to allow the patient to continue dispensing within prescribing limits.

16. The Drug Specific Dispensing Algorithm according to claim 13, wherein the drug dispensing algorithm checks for multiple drug dispensers authorized to dispense the prescribed drug to ensure that the prescribed dose is only dispensed once per the prescription instructions regardless of the number of drug dispensers the patient may have.

17. The Drug Specific Dispensing Algorithm according to claim 13, wherein said algorithm precludes double dosing unless authorized by the prescription or if overwritten by the Integrated Support Center.

18. The Drug Specific Dispensing Algorithm according to claim 13, wherein said Algorithm utilizes the prescription information to ascertain compliance with any special storage requirements, e.g., temperature range, humidity level, etc. before authorizing a dose to be dispensed

19. The Drug Specific Dispensing Algorithm according to claim 13, wherein said Algorithm utilizes the prescription information to ascertain the time interval between doses.

20-24. (canceled)

25. A Drug Specific App used to aggregate digitally captured and/or patient inputted self-assessment, self-test, and/or self-reported physiologic, psychological, lifestyle, concomitant medication, environmental, drug side effect, and/or PRO information/values/data not otherwise required by the Drug Specific Dispensing Algorithm to: 1) assist the prescriber and/or caregivers to better manage the patient and his/her drug therapy/pain management, and/or 2) to aggregate clinical trial information required for regulatory submission and/or reporting, and/or for REMS and/or Prescription Drug Monitoring Programs (PDMP) reporting.

26. (canceled)
